SEQUENCE LISTING (67 sequences)

SEQ ID NO: 1 - DNA, 15186 bp, Newcastle disease virus
  STRAIN MTH-68/H
accaaacagagaatccgtgagatacgataaaaggcgaaggagcaatcgaagtcgtacgggtagaaggtgtgaatctcgagtgcgagaccgaagcttaaactcgagagagccttctgccaacatgtcttccgtatttgacgagtatgaacagctcctcgcggctcagactcgccctaatggagctcacggaggaggagagaaggggagcaccttaaaagttgaagtcccggtattcactcttaacagtgacgacccagaagatagatggaactttgcagtattctgtcttaggattgctgttagcgaagatgccaacaaaccactcagacaaggtgctctcatatctctcttatgctcccattctcaagtgatgaggaaccatgtcgcccttgcagggaaacagaatgaggccacactggctgttcttgagatcgatggtttcaccaacagcgtgcctcaatttaacaacaggagtggagtgtctgaggagagagcacagagattcttgatgatagcagggtccctccctcgggcatgcagcaacggtaccccgttcatcacagctggggttgaagatgatgcaccagaagacatcactgatactctggaaagaatcatctctatccaggctcaagtatgggtcacggtagcaaaggccatgactgcatatgagacagcggatgagtcggaaacaagaagaatcaataagtatatgcagcaaggcagagtccaaaagaagtacatcctccaccccgtatgtaggagcgcaattcaactcacgattagacaatctctggcagtccgcattttcttggttagcgagcttaagagaggccgcaacacggcaggtggaagttccacttattatagcttggtaggggacatagactcatacatcaggaacaccgggcttactgcattcttcctgacactcaggtatggaattaacaccaagacatcagcccttgcactcagcagcctcgcaggcgatatccaaaaaatgaagcagctcatgcgtctatatcggatgaaaggagataatgcgccgtacatgacactgctcggtgacagtgaccagatgagctttgcaccggctgagtatgcacaactctactcttttgcaatgggtatggcatccgtcctagacaaaggaactggcaagtaccagttcgccagagactttatgagcacatcattctggagacttggagtagagtatgctcgggctcagggaagcagcatcaatgaggatatggctgccgagctaaagctgaccccagcggcaaggaggggcctggcagctgctgcccaacgagcgtccgaggagaccggcagcatggacattcccactcaacaagctggggtcctcacagggctcagcgacggaggccctcaagccccacaaggtggactgaacaggtcgcaagggcaaccggacgccggagatggggagacccaatttttagatttgatgagagcagtggcaaatagtatgagagaagcgccaaactctgtgcagaacaccactcagcaggagcctccatctactcctgggccatctcaagacaacgacaccgactggggatactgaccgacaacactcagcctgcctccatcgaattatctcgattcttctgcctgtaacctaacccctggtccacagtctcacacggccaaacccgcaaacgagcccccccctctctccccccccctccagccacacggctccaaccgcccaaaacaacacgggcacaactcgactcactcataatctacacagagccgaagatcttagaaaaaaatacgggtagaagagagacattcagagatcaggacaaatcatcagggtctccgctctcccctctacccagcagaccagagtgaagatggccaccttcacggatgcagagatcgacgatctatttgaaaccagtggaactgtcattgacagcataattacggcccaaggcaacccagtagagactgttggaaggagcgcaatcccacaaggcaaaactaaagcattgagtgcagcatgggagaaacatggaagcacccaaccgccggccggccaagacacccctgatcgaatggacagatcagacaaacaaccatccccacccgaacaggcaaccacacacaacagcccgccagccacatccaccgaccagccccccacccaggccgcaggcgaagccggtgacacacagctcaagactggagcaagcaactccctcctgtcgatgctcgacaagcttagcaataaatcgtccaatgctaaaaagggcccacggtcgagtccccaggaagggcaccaccaacctccgacccagcagcaagggagtcaacagagccgcggaaacagtcaggagagatcgcaaaaccaggtcaaggccgcctctggagaccagggcacagacgcgaacacagcatatcatggacaatggaaggagtcacaaccatcagctggtgcaatccctcatgctctccggtcagggcagagccaagccaatactcctgcatctgtggatcatgtccagctacctgtcgactttgtgcaggcgatgatgtctatgatggaggcgatatcacagaaggtaaataaagtcgactatcagctagaccttgtctcaaagcagacatcctccatccccatgatgcgatctgaaatccaacagctgaaaacatccgttgcggtcatggaagccaacctgggcatgatgaagattctggaccctggttgtgctaacgtttcatctttaagtgacctacgggcggtcgcccgctctcacccagttttagtttcaggccccggagacccatctccttatgtgacacaagggggtgaattgatactcaataaactctcgcaaccagtgcaacatccttctgagttgattaaacctgccacgataagcgggcctgatataggagtggagaaggacactgtccgtgcattgatcacctcacgcccaatgcatccgagctcttcagctaagctcctgagtaagctggatgcagccgggtcgatagaagaaattaggaaaatcaaacgccttgcgctgaatggctgatcactactacaacccgcaacgggttcccgtttatctattgtcacaaggaacccgccccgagccctcctctataaacccaagattcaaggctccaagcgataaccttctcccgcctcttccatcccactgaatgatcgcgcagctgcaattaatctagcaacattaaagattaagaaaaaatacgggtagaatcggagtgcaccgattgtgccaagatggactcatctaggacaattgggttatactttgattctgcccttccttctagcaacctgttagcattcccgattgtcttacgagatgtaggagatggaaagaagcaaattactccgcaatataggatccggcgtcttgactcgtggacagacagtaaagaagactcggtattcatcactacctacggattcatcttccaggttgggaatgaagaagtcactgtcggcatgatcaatgataatcccaagcgagagttactctccgctgcgatgctctgcctaggaagcgtcccgaatgttggagatcctgttgagctggcaagggcctgcctcactatggtggtaacatgcaagaagagtgcaactaatactgagagaatggtcttctcagtagtgcaggcaccccgggtgctgcaaagctgtagggtcgtggcagacaaatactcgtcagtgaatgcagttaagcacgtgaaagcaccagagaagatccctgggagtgaaaccctagagtacaaggtgaattttgtctctttgactgtggtgccgaagaaggatgtctacaagatcccaaccgcagtattgaaggtatccggctcgagcctgtataatcttgcgctcaatgtcactattgatgtggaggtagacccaaagagcccgttagtcaagtccctttctaggtccgacagtggatactatgctaatctcttcttacatatcggacttatgtccactgtagataagaggggaaagaaagtgacatttgaccagctggagaggaagataaggagacttgatctatctgtcgggctcagtgatgtgctcggaccttctgtgcttgtgaaggcgagaggtgcacggactaggctgctggcacctttcttctctaacagcgggacagcctgctaccctatagcaaatgcctctcctcaggtagctaagatactctggagtcagaccgcgtgcctgcggagtgtaaaaatcattattcaagcgggcacccaacgcgctgtcgcagtgactgctgaccatgaggttacctctactaagatagagaagaggcataccatcgctaaatacaatcccttcaagaaataggccgtatccctgagactgcaattcacccgccttcccaaaacaccatgacaccagataatgatctgtcttgattacttacagttagtttccctgtctatcaaattagaaaaaacacgggtagaagagttcggatcccggccggcgcaccaaaagcgcaagatgggccccagatcttctaccaggatcccagtacctctaatgctgaccatacggatcacgctggcactgagttatgtccgtctgacaagttctcttgatggcaggcctcttgcagccgcagggatcgtggtaacaggggataaagcagttaacatatacacctcatcccagacagggtcaatcatagtcaagttactcccaaatatgcccaaggacaaagaggcatgtgcaaaagccccattggaggcttacaacaggacactgactactttgcttaccccccttggtgattctatccgcaggatacaagagtctgtgactacatccggaggaaggagacagagacgctttataggtgccattattggcagtgtagctctaggggttgcaacagctgcacagataacggcagcctcggctctgatacaagccaaccagaatgctgctaacatcctccggcttaaggagagcattgctgcaaccaatgaagctgtacacgaggtcactggcggattgtcacagttagcagtggcagttggaaagatgcagcaatttgttaatgaccagtttaataacacagctcaagagttggactgtataaaaattacacagcaggttggtgtagaactcaacttatacttaactgaattgactacagtgttcgggccacaaatcacttcccctgccttaactcagttgactgttcaggctctttacaatctggctggtggtaatgtagattacttgttgactaagttaggtgtagggaacaaccagctcagctcattgattggtagcggcttgatcaccggtaaccctattttttacgactcacagactcaactcttgggcatacaggtgactttaccctcagtcgggaacctaaataatatgcgtgccacctacttggagaccttgtctgtaagcacaacaaagggatttgcctcagcacttgtcccaaaagtagtgacacaggtcggttctgtgatagaagagcttgacacctcatactgtatagaagctgatttggatttatattgtacaagaatagtgacattccctatgtcccctggtatttattcctgtttgagcggcaatacatcggcttgcatgtattcaaggactgaaggcgcacttactacaccatacatgactctcaaaggctcagttgttgccaattgccagatgacaacatgtagatgtgcagaccccccgggtatcatatcacaaaattatggagaggctgtgtctctaatagataagcactcatgcaatgtcgtatccttagacgggataactttgaggctcagtggggaatttgatgcaacttatcaaaagaatatctcaatattagattctcaagtactagtgacaggcaatctcgatatctcaactgagcttgggaatgtcaaccactcaataagtaatgctttggataagttagaggaaagcaacagcaaactagacaaagtcaatgtcaaactgaccagcacatctgctctcattacctatattgttttaactgtcatatctcttgttcttggtatgcttagcctggttctagcatgctatctgatgtacaagcaaaaagcgcaacgaaagaccttgttgtggcttggaaataataccctagatcagatgagagctactacaaaaatgtgaatgcagatgagaggcagaggtatccccaatagcaatctgtgtgtcaattctggcagcctgttaatcagaagaattaagaaaaaactaccggatgtaggtgaacaaaagggaatatacgggtagaacggcctgagaggccacccctcaatcgggagccaggccccactacgtccgctctaccgcatcaccaacagcagtcttcagtcatggacagcgcagttagccaagttgcgctagagaatgatagaagagaagcgaataatacatggcgcttggttttccggatcgcagccttacttttactggtaataaccttagccgtctctgcagtcgccctggcatatagtatggaggctagcacacctggcgaccttgtaagcataccgactgcgatctctagggcagaggaaaggattacatctgcactcggttccaatcaagatgtagtagataggatatacaagcaagtggcccttgagtctccattggcactgctaaataccgaatctataattatgaatgcaataacgtctctctcttatcaaatcaatggagccacaaataatagcgggtgtggggcacctgttcatgacccagattacatcggggggataggtcaagaacttattgtagatgatacgagtgacgtcacatcattttatccctctgcattccaagaacacctgaattttatcccggcgcccactacaggatcaggctgcactcggataccctcattcgacatgagtgctacccattactgttacactcacaatgtgatattgtctggctgcagagatcactcacactcacatcagtatttagcacttggtgtgcttcggacatctgcaacagggagggtattcttttctactctgcgttccatcaacttggatgacgcccaaaatcggaagtcttgcagtgtgagtgcaactcctttaggttgtgatatgctgtgctctaaaatcacagagactgaggaagaggattataaatcagttatccccacatcgatggtacatggaaggttagggtttgacggccaataccatgagaaggacctagacgtcacaacactatttagggactgggtggcaaattacccaggagtaggaggtgggtcttttattaacaatcgcgtatggttcccggtctacggagggctaaaacccagctcgcctagtgacactgcacaagaagggagatacgtaatatacaagcgatacaatgacacatgcccggatgagcaagattaccagattcggatggctaagtcatcgtataagcctaggcggtttggtggaaaacgcgtacagcaggccatcctatccatcaaggtgtcaacatccttgggtgaggacccggtgttgactgtaccgcctaatacggtcgcactcatgggggccgaaggcagagttctcacagtggggacatctcatttcttatatcagcgagggtcatcatacttctctcccgctttgttataccctatgacagtcaacaacaaaacagccactcttcataatccttatacattcaatgctttcactcggccaggtagtgtcccttgccaggcttcagcaagatgccccaactcatgtgttaccggagtctatactgatccatatcccttagtcttccataggaaccacaccttgcgaggggtattcggaacaatgcttgatgacgaacaagcaagactcaaccctgtatctgcagtatttgataacatatcccgcagtcgcataactcgggtaagttcaagcagtaccagggcagcatacacgacatcaacgtgttttaaagttgtcaagaccaataaaacctattgcctcagcattgcagaaatatccaataccctcttcggggaattcagaattgtccccttactagttgagattctcaaggatggtggggtttaagaagctaggaatggtcggttgagtcaaccgtgagaaggcggggaagatgatattgcatcatctatcttctgtaacaccaagaatcaaatcgaataccagtgcgagctcgaatcctacgctgccagtcagccataatcggctggtgctaatgtggttagcctgaatcttgtcgatagtcacttgattaagaaaaaatgtggacggtagtgaaatataaggcaaagcagctcacagaggacagcacgggtaggacatggcgggctccggacccgaaagggcagagcatcagattatcctaccagagtcacatctgtcctcaccattggtcaagcataaattgctttattactggaaattaactgggctaccgcttcctgacgaatgtgacttcgaccatcttattatcagccgacaatggaagaaggtacttgaatcggccacccctgacattgagagaatgataaaactagggcgggcagtacaccagactctcaaccacaattccaggataaccggagtactccatccccggtgtttagaggaactggctagtattgaggttcctgattcaaccaacaaatttcggaagatcgaaaaaaagatccagattcacaacacaaggtatggagaactgttcacaagattatgcacgcatgtagaaaagaaactattggggtcatcttggtctaacaatgtcccacggtcagaggaattcaacagcatccgtacagatccggcattttggtttcactcaaaatggtccacagccaaatttgcatggctccatataaaacaggtccagaggcatctgattgtagcagcaagaacaaggtccgcagtcaacaaattagtgacgctgacccataaggtaggccaaatctttgttactcctgagcttgtcattgtgacacatacagatgagaacaagttcacgtgtcttacccaggaacttgtgttgatgtatgcagatatgatggagggcagagatatggtcagcataatatcatccacggcggcacatcttaggagcttatcagagaaaattgatgatattctgcggttagtagatgctctggcaagagatttgggcaatcaagtctacgatgttgtagcactaatggagggattcgcatacggcgctgttcagctgcttgaaccgtcaggtacatttgcgggggatttcttcgcattcaacctgcaggagctcaaagatactctaaccggactcctccccaaggatatcgcagaatctgtgactcacgcaatcgcaaccatattctctggcttagaacaaaatcaagcagctgagatgttgtgcctgttgcgtctgtggggtcacccactactcgagtcccgtattgcagcaaaagcagttaggagtcaaatgtgcgcaccaaaaatggtagactttgatatgatcctccaggtattatctttctttaaaggaacaatcatcaatggatatagaaagaagaatgcaggcgtgtggccacgtgtcaagatagatacgatatacgggaaggtcatagggcagctacacgcagattctgcagagatttcacatgatgtcatgttgagggaatacaagagtttatctgcacttgaattcgagccatgtatagagtatgaccctgtcaccaatctgagcatgtttttaaaagacaaggcaatcgcacacccgaaagacaactggctcgcttcgtttaggcgaaaccttctctctgaggaccagaagaaacatgtaagagaggcaacctcaactaaccgcctcttgatagagttcttagagtcaaatgattttgatccatataaagagatggaatatctgacgacccttgagtacctaagagatgataatgtggcagtatcatactcactcaaagaaaaagaggtgaaagttaatgggcggattttcgcaaagctaacaaatagattaaggaattgtcaggtaatggcagaagggatcctagctgaccagattgcacctttcttccagggaaatggggtcattcaggatagcatatctttgactaaaagtatgctagcgatgagtcaactgtctttcaacagcaacaagaaacgtattactgactgcaaagaaagagtatcctcaaaccgcaatcatgatccgaagagcaagaatcgtcggagagttgccacttttgtaactaccgacctgcaaaagtattgtctcaattggagatatcagacagtcaagctgttcgctcatgccatcaatcagctgatgggcttacctcacttcttcgagtggattcatcttagactaatggatactacgatgtttgtaggggaccctttcaatcctccaagtgaccctacggactgtgatctatcaagagtcccaaatgatgacatatatattgtcagtgctaggggaggcattgagggattatgccagaagctatggacaatgatctcaattgctgcaatccagcttgctgcagcaagatcacattgtcgcgttgcctgtatggtacaaggcgacaatcaagtaatagctgtaacgagagaggtaagatcagatgactccccagagatggtgttaacacaattgcatcaagccagtgataatttcttcaaggaattgattcatgtcaatcatttgatcggccataatttgaaggatcgtgaaaccatcaggtcagacacgttcttcatatacagcaaacgaatattcaaagatggagcaatactcagtcaggtcctcaaaaactcatctaaattagtgctaatatcaggcgaccttagtgaaaacactgtaatgtcttgtgccaacattgcatctactgtagcacggttatgcgagaacgggcttcctaaggatttctgttattacttaaactacttaatgagttgcgtgcagacatacttcgattccgaattttccatcaccaacaactcgcaactcgattctaaccagtcgtggatagaggacatttcttttgtgcactcatatgtcctgacccctgctcaactggggggactgagtaaccttcaatactcaaggctctacacaaggaacatcggcgacccgggaaccactgctttcgcagagatcaagagattagaggcagtggggttactgagtcctagcattatgactaacatcttaactaggccgcctggaaatggagattgggccagtctgtgcaacgatccatactcctttaattttgagactgtcgcaagcccaaatattgtccttaagaaacatacacaaagagtcctatttgaaacttgctcgaatcccttattatctggagtgcacacagaggataatgaggcagaagagaaggcattggctgaattcttgctcaatcaagaaatgattcatccacgtgtcgcgcatgctatcatggaagcaagctctgtaggtaggagaaagcaaattcaggggcttgttgacacaacaaacaccgtgattaagattgcactgactaggaggccactcggcatcaagaggctgatgcggatagtcaattactcgagcatgcatgcaatgctatttagagatgatgttttctcgtctaatagatccaaccatcccttagtctcttctgatatgtgttctctgacactggcagactatgcacggaacagaagctggtcacctttgacagggggtagaaaaatactgggtgtatctaatcctgataccatagaacttgtagagggtgagatccttagtgtcagtggagggtgcactagatgtgacagtggggatgaacagtttacttggttccatcttccaagcaatatagagctaaacgatgacaccagcaagaatcctccaatgagagtgccatatctcgggtcaaagactcaagagaggagagccgcctcgcttgcgaaaatagctcatatgtcaccacacgtgaaggcggctctaagggcgtcatctgtgttaatctgggcttatggggacaacgaaataaactggactgctgcccttaagattgcaaggtctcggtgcaacataagctcagagtatcttcgactattgtcacccttgcctacagctgggaatctccaacatagattggacgacggcataactcagatgacattcacccctgcatctctatacagggtgtcaccttacattcatatatctaatgattctcaaaggctatttactgaagaaggagtcaaagaggggaatgtggtttatcagcaaatcatgctcttgggtttgtccttaattgagtcactcttcccaatgacaacaaccaagacatatgatgaaatcacattgcacctccacagtaaatttagctgctgtatcagggaagcacctgttgcagttcctttcgagctactcggggtggcaccggaactaagggcagtaacctcaaataagtttatgtatgatcctagccctgtatcagagggagactttgcgagacttgacttagctatctttaagagttatgaacttaatttagagtcatattccacaatagagctaatgaacgttctttcaatatctagtgggaagttgattggccagtctgtggtttcttatgatgaagatacctccataaagaatgacgctataatagtgtatgacaacacacggaattggatcagcgaagctcagaattcagatgtggtccgcctattcgagtatgcggcactcgaagtgctcctcgactgttcttatcaactctactatctgagagtaagaggcctaaacaatatcgtcctgtacatgagtgatttatacaagaatatgccaggaattctactctctaatattgcagccacaatatctcaccctgtcatccattcaaggttgaatgcagtaggtctggtcaaccatgacgggtcacaccaacttgcagacacagatttcattgaaatgtctgcaaagctgctagtctcttgcactcgacgcgtggtctcaggtttacatgcagggaataagtatgacctgctgtttccatctgtcttggatgataacctaagtgagaagatgcttcagttgatatcccaattatgctgtctgtatacggtgctctttgctacaacaagagaaatcccgaaaataagaggcttatctgcagaagagaaatgttcagtacttactgagtacctactgtcagatgctgtgaaaccgttacttgggtccgagcaagtgagctctatcatgtctcccaacatagttacgttcccagccaatctgtattacatgtctaggaagagccttaatttgatcagggagagagaggacagggatactatcttggcattgttgttccctcaagaaccgctgctcgagtttcctctggtacgagatattggtgctcgtgtaaaagatccatttacccgacaacctgcggcgtttttacaagagttagatttgagtgctccggcaaggtatgacgcattcacaatcagtcaggcgcattctgaacacatattgccaaacccagaggaagatcacttagtacgatacttgttcagaggaatagggactgcgtcctcctcttggtataaggcatcccatcttctttctgtacccgaggtcagatgtgcaaggcatgggaactccttatatttagcagaaggaagtggagccatcatgagtcttctcgaattgcatataccacacgaaactatctattacaatacacttttctcgaacgagatgaaccccccacagcgacatttcggaccaactccaacacagtttctgaattcggttgtttttaggaatttacaggcggaagtaccatgcaaggatggatttgtccaggagttccgtccgttatggagagagaatacagaagaaagcgatctgacctcagataaagcagtgggatatatcacatctgtagtgccgtacaggtctgtatcattgctgcattgtgacattgaaatccctccaggatctaatcaaagcttactagatcagctggctaccaatctgtctctgattgccatgcattctgtgaaggagggcggggtcgtgattatcaaagtactgtatgcaatgggatattacttccatctactcatgaacttgtttactccatgttccacgaaaggatatattctctctaatggctatgcctgtagaggggatatggagtgttacctgatatttgtcatgggctacctaggcgggcctacatttgtacatgaggtggtgaggatggcaaaaacgctagtacagcggcatggcacacttttgtctaagtcagatgagattacactgactaggttatttacttcacagcaacagcgtgtaacagacatcctatccagccctttaccgagactaatgaagtacttgagagagaatattgatactgcactgattgaagccgggggacagcctgtccgtccattctgtgcagagagtttagtgagcacactaacagacatgactcagacaacccagatcatcgccagccacattgatacagtcattcgatctgtgatctatatggaagcagagggtgatcttgctgacacagtattcttatttaccccttacaacctctctatggacgggaaaaagagaacatcacttaaacagtgcacaagacagatcttagaggtcacaatactgggtctcagagtcaaaaatctcaataaagtaggtgatgtaatcagcctagtactcagaggtatgatttctctggaggaccttctcccactgagaacctacttgaagtgtagtacctgccctaagtatttgaaggctgtcctaggtattaccaaactcaaagaaatgttcacagacgcctctttattatacttgactcgtgctcaacaaaaattctacatgaaaactataggcaatgcagtcaagggatactatagtaactgtgactcttaaaggcaaccacatatcaataggccctctttctagccgatcgtattcttgttgacttcattataccatattagaaaaaaattgaattccgaccctttaagactcgtattcggattcaaataattatctcagaaaaaaagtgcacgtagttgttcttgattatagtcccgtcattcaccaagtctttgtttggt

SEQ ID NO: 2 - DNA, 15186 bp, Newcastle disease virus
  Mut HN(F277L)/M(G165W)
accaaacagagaatccgtgagatacgataaaaggcgaaggagcaatcgaagtcgtacgggtagaaggtgtgaatctcgagtgcgagaccgaagcttaaactcgagagagccttctgccaacatgtcttccgtatttgacgagtatgaacagctcctcgcggctcagactcgccctaatggagctcacggaggaggagagaaggggagcaccttaaaagttgaagtcccggtattcactcttaacagtgacgacccagaagatagatggaactttgcagtattctgtcttaggattgctgttagcgaagatgccaacaaaccactcagacaaggtgctctcatatctctcttatgctcccattctcaagtgatgaggaaccatgtcgcccttgcagggaaacagaatgaggccacactggctgttcttgagatcgatggtttcaccaacagcgtgcctcaatttaacaacaggagtggagtgtctgaggagagagcacagagattcttgatgatagcagggtccctccctcgggcatgcagcaacggtaccccgttcatcacagctggggttgaagatgatgcaccagaagacatcactgatactctggaaagaatcatctctatccaggctcaagtatgggtcacggtagcaaaggccatgactgcatatgagacagcggatgagtcggaaacaagaagaatcaataagtatatgcagcaaggcagagtccaaaagaagtacatcctccaccccgtatgtaggagcgcaattcaactcacgattagacaatctctggcagtccgcattttcttggttagcgagcttaagagaggccgcaacacggcaggtggaagttccacttattatagcttggtaggggacatagactcatacatcaggaacaccgggcttactgcattcttcctgacactcaggtatggaattaacaccaagacatcagcccttgcactcagcagcctcgcaggcgatatccaaaaaatgaagcagctcatgcgtctatatcggatgaaaggagataatgcgccgtacatgacactgctcggtgacagtgaccagatgagctttgcaccggctgagtatgcacaactctactcttttgcaatgggtatggcatccgtcctagacaaaggaactggcaagtaccagttcgccagagactttatgagcacatcattctggagacttggagtagagtatgctcgggctcagggaagcagcatcaatgaggatatggctgccgagctaaagctgaccccagcggcaaggaggggcctggcagctgctgcccaacgagcgtccgaggagaccggcagcatggacattcccactcaacaagctggggtcctcacagggctcagcgacggaggccctcaagccccacaaggtggactgaacaggtcgcaagggcaaccggacgccggagatggggagacccaatttttagatttgatgagagcagtggcaaatagtatgagagaagcgccaaactctgtgcagaacaccactcagcaggagcctccatctactcctgggccatctcaagacaacgacaccgactggggatactgaccgacaacactcagcctgcctccatcgaattatctcgattcttctgcctgtaacctaacccctggtccacagtctcacacggccaaacccgcaaacgagcccccccctctctccccccccctccagccacacggctccaaccgcccaaaacaacacgggcacaactcgactcactcataatctacacagagccgaagatcttagaaaaaaatacgggtagaagagagacattcagagatcaggacaaatcatcagggtctccgctctcccctctacccagcagaccagagtgaagatggccaccttcacggatgcagagatcgacgatctatttgaaaccagtggaactgtcattgacagcataattacggcccaaggcaacccagtagagactgttggaaggagcgcaatcccacaaggcaaaactaaagcattgagtgcagcatgggagaaacatggaagcacccaaccgccggccggccaagacacccctgatcgaatggacagatcagacaaacaaccatccccacccgaacaggcaaccacacacaacagcccgccagccacatccaccgaccagccccccacccaggccgcaggcgaagccggtgacacacagctcaagactggagcaagcaactccctcctgtcgatgctcgacaagcttagcaataaatcgtccaatgctaaaaagggcccacggtcgagtccccaggaagggcaccaccaacctccgacccagcagcaagggagtcaacagagccgcggaaacagtcaggagagatcgcaaaaccaggtcaaggccgcctctggagaccagggcacagacgcgaacacagcatatcatggacaatggaaggagtcacaaccatcagctggtgcaatccctcatgctctccggtcagggcagagccaagccaatactcctgcatctgtggatcatgtccagctacctgtcgactttgtgcaggcgatgatgtctatgatggaggcgatatcacagaaggtaaataaagtcgactatcagctagaccttgtctcaaagcagacatcctccatccccatgatgcgatctgaaatccaacagctgaaaacatccgttgcggtcatggaagccaacctgggcatgatgaagattctggaccctggttgtgctaacgtttcatctttaagtgacctacgggcggtcgcccgctctcacccagttttagtttcaggccccggagacccatctccttatgtgacacaagggggtgaattgatactcaataaactctcgcaaccagtgcaacatccttctgagttgattaaacctgccacgataagcgggcctgatataggagtggagaaggacactgtccgtgcattgatcacctcacgcccaatgcatccgagctcttcagctaagctcctgagtaagctggatgcagccgggtcgatagaagaaattaggaaaatcaaacgccttgcgctgaatggctgatcactactacaacccgcaacgggttcccgtttatctattgtcacaaggaacccgccccgagccctcctctataaacccaagattcaaggctccaagcgataaccttctcccgcctcttccatcccactgaatgatcgcgcagctgcaattaatctagcaacattaaagattaagaaaaaatacgggtagaatcggagtgcaccgattgtgccaagatggactcatctaggacaattgggttatactttgattctgcccttccttctagcaacctgttagcattcccgattgtcttacgagatgtaggagatggaaagaagcaaattactccgcaatataggatccggcgtcttgactcgtggacagacagtaaagaagactcggtattcatcactacctacggattcatcttccaggttgggaatgaagaagtcactgtcggcatgatcaatgataatcccaagcgagagttactctccgctgcgatgctctgcctaggaagcgtcccgaatgttggagatcctgttgagctggcaagggcctgcctcactatggtggtaacatgcaagaagagtgcaactaatactgagagaatggtcttctcagtagtgcaggcaccccgggtgctgcaaagctgtagggtcgtggcagacaaatactcgtcagtgaatgcagttaagcacgtgaaagcaccagagaagatcccttggagtgaaaccctagagtacaaggtgaattttgtctctttgactgtggtgccgaagaaggatgtctacaagatcccaaccgcagtattgaaggtatccggctcgagcctgtataatcttgcgctcaatgtcactattgatgtggaggtagacccaaagagcccgttagtcaagtccctttctaggtccgacagtggatactatgctaatctcttcttacatatcggacttatgtccactgtagataagaggggaaagaaagtgacatttgaccagctggagaggaagataaggagacttgatctatctgtcgggctcagtgatgtgctcggaccttctgtgcttgtgaaggcgagaggtgcacggactaggctgctggcacctttcttctctaacagcgggacagcctgctaccctatagcaaatgcctctcctcaggtagctaagatactctggagtcagaccgcgtgcctgcggagtgtaaaaatcattattcaagcgggcacccaacgcgctgtcgcagtgactgctgaccatgaggttacctctactaagatagagaagaggcataccatcgctaaatacaatcccttcaagaaataggccgtatccctgagactgcaattcacccgccttcccaaaacaccatgacaccagataatgatctgtcttgattacttacagttagtttccctgtctatcaaattagaaaaaacacgggtagaagagttcggatcccggccggcgcaccaaaagcgcaagatgggccccagatcttctaccaggatcccagtacctctaatgctgaccatacggatcacgctggcactgagttatgtccgtctgacaagttctcttgatggcaggcctcttgcagccgcagggatcgtggtaacaggggataaagcagttaacatatacacctcatcccagacagggtcaatcatagtcaagttactcccaaatatgcccaaggacaaagaggcatgtgcaaaagccccattggaggcttacaacaggacactgactactttgcttaccccccttggtgattctatccgcaggatacaagagtctgtgactacatccggaggaaggagacagagacgctttataggtgccattattggcagtgtagctctaggggttgcaacagctgcacagataacggcagcctcggctctgatacaagccaaccagaatgctgctaacatcctccggcttaaggagagcattgctgcaaccaatgaagctgtacacgaggtcactggcggattgtcacagttagcagtggcagttggaaagatgcagcaatttgttaatgaccagtttaataacacagctcaagagttggactgtataaaaattacacagcaggttggtgtagaactcaacttatacttaactgaattgactacagtgttcgggccacaaatcacttcccctgccttaactcagttgactgttcaggctctttacaatctggctggtggtaatgtagattacttgttgactaagttaggtgtagggaacaaccagctcagctcattgattggtagcggcttgatcaccggtaaccctattttttacgactcacagactcaactcttgggcatacaggtgactttaccctcagtcgggaacctaaataatatgcgtgccacctacttggagaccttgtctgtaagcacaacaaagggatttgcctcagcacttgtcccaaaagtagtgacacaggtcggttctgtgatagaagagcttgacacctcatactgtatagaagctgatttggatttatattgtacaagaatagtgacattccctatgtcccctggtatttattcctgtttgagcggcaatacatcggcttgcatgtattcaaggactgaaggcgcacttactacaccatacatgactctcaaaggctcagttgttgccaattgccagatgacaacatgtagatgtgcagaccccccgggtatcatatcacaaaattatggagaggctgtgtctctaatagataagcactcatgcaatgtcgtatccttagacgggataactttgaggctcagtggggaatttgatgcaacttatcaaaagaatatctcaatattagattctcaagtactagtgacaggcaatctcgatatctcaactgagcttgggaatgtcaaccactcaataagtaatgctttggataagttagaggaaagcaacagcaaactagacaaagtcaatgtcaaactgaccagcacatctgctctcattacctatattgttttaactgtcatatctcttgttcttggtatgcttagcctggttctagcatgctatctgatgtacaagcaaaaagcgcaacgaaagaccttgttgtggcttggaaataataccctagatcagatgagagctactacaaaaatgtgaatgcagatgagaggcagaggtatccccaatagcaatctgtgtgtcaattctggcagcctgttaatcagaagaattaagaaaaaactaccggatgtaggtgaacaaaagggaatatacgggtagaacggcctgagaggccacccctcaatcgggagccaggccccactacgtccgctctaccgcatcaccaacagcagtcttcagtcatggacagcgcagttagccaagttgcgctagagaatgatagaagagaagcgaataatacatggcgcttggttttccggatcgcagccttacttttactggtaataaccttagccgtctctgcagtcgccctggcatatagtatggaggctagcacacctggcgaccttgtaagcataccgactgcgatctctagggcagaggaaaggattacatctgcactcggttccaatcaagatgtagtagataggatatacaagcaagtggcccttgagtctccattggcactgctaaataccgaatctataattatgaatgcaataacgtctctctcttatcaaatcaatggagccacaaataatagcgggtgtggggcacctgttcatgacccagattacatcggggggataggtcaagaacttattgtagatgatacgagtgacgtcacatcattttatccctctgcattccaagaacacctgaattttatcccggcgcccactacaggatcaggctgcactcggataccctcattcgacatgagtgctacccattactgttacactcacaatgtgatattgtctggctgcagagatcactcacactcacatcagtatttagcacttggtgtgcttcggacatctgcaacagggagggtattcttttctactctgcgttccatcaacttggatgacgcccaaaatcggaagtcttgcagtgtgagtgcaactcctttaggttgtgatatgctgtgctctaaaatcacagagactgaggaagaggattataaatcagttatccccacatcgatggtacatggaaggttagggcttgacggccaataccatgagaaggacctagacgtcacaacactatttagggactgggtggcaaattacccaggagtaggaggtgggtcttttattaacaatcgcgtatggttcccggtctacggagggctaaaacccagctcgcctagtgacactgcacaagaagggagatacgtaatatacaagcgatacaatgacacatgcccggatgagcaagattaccagattcggatggctaagtcatcgtataagcctaggcggtttggtggaaaacgcgtacagcaggccatcctatccatcaaggtgtcaacatccttgggtgaggacccggtgttgactgtaccgcctaatacggtcgcactcatgggggccgaaggcagagttctcacagtggggacatctcatttcttatatcagcgagggtcatcatacttctctcccgctttgttataccctatgacagtcaacaacaaaacagccactcttcataatccttatacattcaatgctttcactcggccaggtagtgtcccttgccaggcttcagcaagatgccccaactcatgtgttaccggagtctatactgatccatatcccttagtcttccataggaaccacaccttgcgaggggtattcggaacaatgcttgatgacgaacaagcaagactcaaccctgtatctgcagtatttgataacatatcccgcagtcgcataactcgggtaagttcaagcagtaccagggcagcatacacgacatcaacgtgttttaaagttgtcaagaccaataaaacctattgcctcagcattgcagaaatatccaataccctcttcggggaattcagaattgtccccttactagttgagattctcaaggatggtggggtttaagaagctaggaatggtcggttgagtcaaccgtgagaaggcggggaagatgatattgcatcatctatcttctgtaacaccaagaatcaaatcgaataccagtgcgagctcgaatcctacgctgccagtcagccataatcggctggtgctaatgtggttagcctgaatcttgtcgatagtcacttgattaagaaaaaatgtggacggtagtgaaatataaggcaaagcagctcacagaggacagcacgggtaggacatggcgggctccggacccgaaagggcagagcatcagattatcctaccagagtcacatctgtcctcaccattggtcaagcataaattgctttattactggaaattaactgggctaccgcttcctgacgaatgtgacttcgaccatcttattatcagccgacaatggaagaaggtacttgaatcggccacccctgacattgagagaatgataaaactagggcgggcagtacaccagactctcaaccacaattccaggataaccggagtactccatccccggtgtttagaggaactggctagtattgaggttcctgattcaaccaacaaatttcggaagatcgaaaaaaagatccagattcacaacacaaggtatggagaactgttcacaagattatgcacgcatgtagaaaagaaactattggggtcatcttggtctaacaatgtcccacggtcagaggaattcaacagcatccgtacagatccggcattttggtttcactcaaaatggtccacagccaaatttgcatggctccatataaaacaggtccagaggcatctgattgtagcagcaagaacaaggtccgcagtcaacaaattagtgacgctgacccataaggtaggccaaatctttgttactcctgagcttgtcattgtgacacatacagatgagaacaagttcacgtgtcttacccaggaacttgtgttgatgtatgcagatatgatggagggcagagatatggtcagcataatatcatccacggcggcacatcttaggagcttatcagagaaaattgatgatattctgcggttagtagatgctctggcaagagatttgggcaatcaagtctacgatgttgtagcactaatggagggattcgcatacggcgctgttcagctgcttgaaccgtcaggtacatttgcgggggatttcttcgcattcaacctgcaggagctcaaagatactctaaccggactcctccccaaggatatcgcagaatctgtgactcacgcaatcgcaaccatattctctggcttagaacaaaatcaagcagctgagatgttgtgcctgttgcgtctgtggggtcacccactactcgagtcccgtattgcagcaaaagcagttaggagtcaaatgtgcgcaccaaaaatggtagactttgatatgatcctccaggtattatctttctttaaaggaacaatcatcaatggatatagaaagaagaatgcaggcgtgtggccacgtgtcaagatagatacgatatacgggaaggtcatagggcagctacacgcagattctgcagagatttcacatgatgtcatgttgagggaatacaagagtttatctgcacttgaattcgagccatgtatagagtatgaccctgtcaccaatctgagcatgtttttaaaagacaaggcaatcgcacacccgaaagacaactggctcgcttcgtttaggcgaaaccttctctctgaggaccagaagaaacatgtaagagaggcaacctcaactaaccgcctcttgatagagttcttagagtcaaatgattttgatccatataaagagatggaatatctgacgacccttgagtacctaagagatgataatgtggcagtatcatactcactcaaagaaaaagaggtgaaagttaatgggcggattttcgcaaagctaacaaatagattaaggaattgtcaggtaatggcagaagggatcctagctgaccagattgcacctttcttccagggaaatggggtcattcaggatagcatatctttgactaaaagtatgctagcgatgagtcaactgtctttcaacagcaacaagaaacgtattactgactgcaaagaaagagtatcctcaaaccgcaatcatgatccgaagagcaagaatcgtcggagagttgccacttttgtaactaccgacctgcaaaagtattgtctcaattggagatatcagacagtcaagctgttcgctcatgccatcaatcagctgatgggcttacctcacttcttcgagtggattcatcttagactaatggatactacgatgtttgtaggggaccctttcaatcctccaagtgaccctacggactgtgatctatcaagagtcccaaatgatgacatatatattgtcagtgctaggggaggcattgagggattatgccagaagctatggacaatgatctcaattgctgcaatccagcttgctgcagcaagatcacattgtcgcgttgcctgtatggtacaaggcgacaatcaagtaatagctgtaacgagagaggtaagatcagatgactccccagagatggtgttaacacaattgcatcaagccagtgataatttcttcaaggaattgattcatgtcaatcatttgatcggccataatttgaaggatcgtgaaaccatcaggtcagacacgttcttcatatacagcaaacgaatattcaaagatggagcaatactcagtcaggtcctcaaaaactcatctaaattagtgctaatatcaggcgaccttagtgaaaacactgtaatgtcttgtgccaacattgcatctactgtagcacggttatgcgagaacgggcttcctaaggatttctgttattacttaaactacttaatgagttgcgtgcagacatacttcgattccgaattttccatcaccaacaactcgcaactcgattctaaccagtcgtggatagaggacatttcttttgtgcactcatatgtcctgacccctgctcaactggggggactgagtaaccttcaatactcaaggctctacacaaggaacatcggcgacccgggaaccactgctttcgcagagatcaagagattagaggcagtggggttactgagtcctagcattatgactaacatcttaactaggccgcctggaaatggagattgggccagtctgtgcaacgatccatactcctttaattttgagactgtcgcaagcccaaatattgtccttaagaaacatacacaaagagtcctatttgaaacttgctcgaatcccttattatctggagtgcacacagaggataatgaggcagaagagaaggcattggctgaattcttgctcaatcaagaaatgattcatccacgtgtcgcgcatgctatcatggaagcaagctctgtaggtaggagaaagcaaattcaggggcttgttgacacaacaaacaccgtgattaagattgcactgactaggaggccactcggcatcaagaggctgatgcggatagtcaattactcgagcatgcatgcaatgctatttagagatgatgttttctcgtctaatagatccaaccatcccttagtctcttctgatatgtgttctcagacactggcagactatgcacggaacagaagctggtcacctttgacagggggtagaaaaatactgggtgtatctaatcctgataccatagaacttgtagagggtgagatccttagtgtcagtggagggtgcactagatgtgacagtggggatgaacagtttacttggttccatcttccaagcaatatagagctaaacgatgacaccagcaagaatcctccaatgagagtgccatatctcgggtcaaagactcaagagaggagagccgcctcgcttgcgaaaatagctcatatgtcaccacacgtgaaggcggctctaagggcgtcatctgtgttaatctgggcttatggggacaacgaaataaactggactgctgcccttaagattgcaaggtctcggtgcaacataagctcagagtatcttcgactattgtcacccttgcctacagctgggaatctccaacatagattggacgacggcataactcagatgacattcacccctgcatctctatacagggtgtcaccttacattcatatatctaatgattctcaaaggctatttactgaagaaggagtcaaagaggggaatgtggtttatcagcaaatcatgctcttgggtttgtccttaattgagtcactcttcccaatgacaacaaccaagacatatgatgaaatcacattgcacctccacagtaaatttagctgctgtatcagggaagcacctgttgcagttcctttcgagctactcggggtggcaccggaactaagggcagtaacctcaaataagtttatgtatgatcctagccctgtatcagagggagactttgcgagacttgacttagctatctttaagagttatgaacttaatttagagtcatattccacaatagagctaatgaacgttctttcaatatctagtgggaagttgattggccagtctgtggtttcttatgatgaagatacctccataaagaatgacgctataatagtgtatgacaacacacggaattggatcagcgaagctcagaattcagatgtggtccgcctattcgagtatgcggcactcgaagtgctcctcgactgttcttatcaactctactatctgagagtaagaggcctaaacaatatcgtcctgtacatgagtgatttatacaagaatatgccaggaattctactctctaatattgcagccacaatatctcaccctgtcatccattcaaggttgaatgcagtaggtctggtcaaccatgacgggtcacaccaacttgcagacacagatttcattgaaatgtctgcaaagctgctagtcttttgcactcgacgcgtggtctcaggtttacatgcagggaataagtatgacctgctgtttccatctgtcttggatgataacctaagtgagaagatgcttcagttgatatcccaattatgctgtctgtatacggtgctctttgctacaacaagagaaatcccgaaaataagaggcttatctgcagaagagaaatgttcagtacttactgagtacctactgtcagatgctgtgaaaccgttacttgggtccgagcaagtgagctctatcatgtctcccaacatagttacgttcccagccaatctgtattacatgtctaggaagagccttaatttgatcagggagagagaggacagggatactatcttggcattgttgttccctcaagaaccgctgctcgagtttcctctggtacgagatattggtgctcgtgtaaaagatccatttacccgacaacctgcggcgtttttacaagagttagatttgagtgctccggcaaggtatgacgcattcacaatcagtcaggcgcattctgaacacatattgccaaacccagaggaagatcacttagtacgatacttgttcagaggaatagggactgcgtcctcctcttggtataaggcatcccatcttctttctgtacccgaggtcagatgtgcaaggcatgggaactccttatatttagcagaaggaagtggagccatcatgagtcttctcgaattgcatataccacacgaaactatctattacaatacacttttctcgaacgagatgaaccccccacagcgacatttcggaccaactccaacacagtttctgaattcggttgtttttaggaatttacaggcggaagtaccatgcaaggatggatttgtccaggagttccgtccgttatggagagagaatacagaagaaagcgatctgacctcagataaagcagtgggatatatcacatctgtagtgccgtacaggtctgtatcattgctgcattgtgacattgaaatccctccaggatctaatcaaagcttactagatcagctggctaccaatctgtctctgattgccatgcattctgtgaaggagggcggggtcgtgattatcaaagtactgtatgcaatgggatattacttccatctactcatgaacttgtttactccatgttccacgaaaggatatattctctctaatggctatgcctgtagaggggatatggagtgttacctgatatttgtcatgggctacctaggcgggcctacatttgtacatgaggtggtgaggatggcaaaaacgctagtacagcggcatggcacacttttgtctaagtcagatgagattacactgactaggttatttacttcacagcaacagcgtgtaacagacatcctatccagccctttaccgagactaatgaagtacttgagagagaatattgatactgcactgattgaagccgggggacagcctgtccgtccattctgtgcagagagtttagtgagcacactaacagacatgactcagacaacccagatcatcgccagccacattgatacagtcattcgatctgtgatctatatggaagcagagggtgatcttgctgacacagtattcttatttaccccttacaacctctctatggacgggaaaaagagaacatcacttaaacagtgcacaagacagatcttagaggtcacaatactgggtctcagagtcaaaaatctcaataaagtaggtgatgtaatcagcctagtactcagaggtatgatttctctggaggaccttctcccactgagaacctacttgaagtgtagtacctgccctaagtatttgaaggctgtcctaggtattaccaaactcaaagaaatgttcacagacgcctctttattatacttgactcgtgctcaacaaaaattctacatgaaaactataggcaatgcagtcaagggatactatagtaactgtgactcttaaaggcaaccacatatcaataggccctctttctagccgatcgtattcttgttgacttcattataccatattagaaaaaaattgaattccgaccctttaagactcgtattcggattcaaataattatctcagaaaaaaagtgcacgtagttgttcttgattatagtcccgtcattcaccaagtctttgtttggt

SEQ ID NO: 3 - DNA, 15198 bp, Newcastle disease virus
  Mut HN(F277L)/M(G165W)+ AfeI
accaaacagagaatccgtgagatacgataaaaggcgaaggagcaatcgaagtcgtacgggtagaaggtgtgaatctcgagtgcgagaccgaagcttaaactcgagagagccttctgccaacatgtcttccgtatttgacgagtatgaacagctcctcgcggctcagactcgccctaatggagctcacggaggaggagagaaggggagcaccttaaaagttgaagtcccggtattcactcttaacagtgacgacccagaagatagatggaactttgcagtattctgtcttaggattgctgttagcgaagatgccaacaaaccactcagacaaggtgctctcatatctctcttatgctcccattctcaagtgatgaggaaccatgtcgcccttgcagggaaacagaatgaggccacactggctgttcttgagatcgatggtttcaccaacagcgtgcctcaatttaacaacaggagtggagtgtctgaggagagagcacagagattcttgatgatagcagggtccctccctcgggcatgcagcaacggtaccccgttcatcacagctggggttgaagatgatgcaccagaagacatcactgatactctggaaagaatcatctctatccaggctcaagtatgggtcacggtagcaaaggccatgactgcatatgagacagcggatgagtcggaaacaagaagaatcaataagtatatgcagcaaggcagagtccaaaagaagtacatcctccaccccgtatgtaggagcgcaattcaactcacgattagacaatctctggcagtccgcattttcttggttagcgagcttaagagaggccgcaacacggcaggtggaagttccacttattatagcttggtaggggacatagactcatacatcaggaacaccgggcttactgcattcttcctgacactcaggtatggaattaacaccaagacatcagcccttgcactcagcagcctcgcaggcgatatccaaaaaatgaagcagctcatgcgtctatatcggatgaaaggagataatgcgccgtacatgacactgctcggtgacagtgaccagatgagctttgcaccggctgagtatgcacaactctactcttttgcaatgggtatggcatccgtcctagacaaaggaactggcaagtaccagttcgccagagactttatgagcacatcattctggagacttggagtagagtatgctcgggctcagggaagcagcatcaatgaggatatggctgccgagctaaagctgaccccagcggcaaggaggggcctggcagctgctgcccaacgagcgtccgaggagaccggcagcatggacattcccactcaacaagctggggtcctcacagggctcagcgacggaggccctcaagccccacaaggtggactgaacaggtcgcaagggcaaccggacgccggagatggggagacccaatttttagatttgatgagagcagtggcaaatagtatgagagaagcgccaaactctgtgcagaacaccactcagcaggagcctccatctactcctgggccatctcaagacaacgacaccgactggggatactgaccgacaacactcagcctgcctccatcgaattatctcgattcttctgcctgtaacctaacccctggtccacagtctcacacggccaaacccgcaaacgagcccccccctctctccccccccctccagccacacggctccaaccgcccaaaacaacacgggcacaactcgactcactcataatctacacagagccgaagatcttagaaaaaaatacgggtagaagagagacattcagagatcaggacaaatcatcagggtctccgctctcccctctacccagcagaccagagtgaagatggccaccttcacggatgcagagatcgacgatctatttgaaaccagtggaactgtcattgacagcataattacggcccaaggcaacccagtagagactgttggaaggagcgcaatcccacaaggcaaaactaaagcattgagtgcagcatgggagaaacatggaagcacccaaccgccggccggccaagacacccctgatcgaatggacagatcagacaaacaaccatccccacccgaacaggcaaccacacacaacagcccgccagccacatccaccgaccagccccccacccaggccgcaggcgaagccggtgacacacagctcaagactggagcaagcaactccctcctgtcgatgctcgacaagcttagcaataaatcgtccaatgctaaaaagggcccacggtcgagtccccaggaagggcaccaccaacctccgacccagcagcaagggagtcaacagagccgcggaaacagtcaggagagatcgcaaaaccaggtcaaggccgcctctggagaccagggcacagacgcgaacacagcatatcatggacaatggaaggagtcacaaccatcagctggtgcaatccctcatgctctccggtcagggcagagccaagccaatactcctgcatctgtggatcatgtccagctacctgtcgactttgtgcaggcgatgatgtctatgatggaggcgatatcacagaaggtaaataaagtcgactatcagctagaccttgtctcaaagcagacatcctccatccccatgatgcgatctgaaatccaacagctgaaaacatccgttgcggtcatggaagccaacctgggcatgatgaagattctggaccctggttgtgctaacgtttcatctttaagtgacctacgggcggtcgcccgctctcacccagttttagtttcaggccccggagacccatctccttatgtgacacaagggggtgaattgatactcaataaactctcgcaaccagtgcaacatccttctgagttgattaaacctgccacgataagcgggcctgatataggagtggagaaggacactgtccgtgcattgatcacctcacgcccaatgcatccgagctcttcagctaagctcctgagtaagctggatgcagccgggtcgatagaagaaattaggaaaatcaaacgccttgcgctgaatggctgatcactactacaacccgcaacgggttcccgtttatctattgtcacaaggaacccgccccgaagcgctgaccgagccctcctctataaacccaagattcaaggctccaagcgataaccttctcccgcctcttccatcccactgaatgatcgcgcagctgcaattaatctagcaacattaaagattaagaaaaaatacgggtagaatcggagtgcaccgattgtgccaagatggactcatctaggacaattgggttatactttgattctgcccttccttctagcaacctgttagcattcccgattgtcttacgagatgtaggagatggaaagaagcaaattactccgcaatataggatccggcgtcttgactcgtggacagacagtaaagaagactcggtattcatcactacctacggattcatcttccaggttgggaatgaagaagtcactgtcggcatgatcaatgataatcccaagcgagagttactctccgctgcgatgctctgcctaggaagcgtcccgaatgttggagatcctgttgagctggcaagggcctgcctcactatggtggtaacatgcaagaagagtgcaactaatactgagagaatggtcttctcagtagtgcaggcaccccgggtgctgcaaagctgtagggtcgtggcagacaaatactcgtcagtgaatgcagttaagcacgtgaaagcaccagagaagatcccttggagtgaaaccctagagtacaaggtgaattttgtctctttgactgtggtgccgaagaaggatgtctacaagatcccaaccgcagtattgaaggtatccggctcgagcctgtataatcttgcgctcaatgtcactattgatgtggaggtagacccaaagagcccgttagtcaagtccctttctaggtccgacagtggatactatgctaatctcttcttacatatcggacttatgtccactgtagataagaggggaaagaaagtgacatttgaccagctggagaggaagataaggagacttgatctatctgtcgggctcagtgatgtgctcggaccttctgtgcttgtgaaggcgagaggtgcacggactaggctgctggcacctttcttctctaacagcgggacagcctgctaccctatagcaaatgcctctcctcaggtagctaagatactctggagtcagaccgcgtgcctgcggagtgtaaaaatcattattcaagcgggcacccaacgcgctgtcgcagtgactgctgaccatgaggttacctctactaagatagagaagaggcataccatcgctaaatacaatcccttcaagaaataggccgtatccctgagactgcaattcacccgccttcccaaaacaccatgacaccagataatgatctgtcttgattacttacagttagtttccctgtctatcaaattagaaaaaacacgggtagaagagttcggatcccggccggcgcaccaaaagcgcaagatgggccccagatcttctaccaggatcccagtacctctaatgctgaccatacggatcacgctggcactgagttatgtccgtctgacaagttctcttgatggcaggcctcttgcagccgcagggatcgtggtaacaggggataaagcagttaacatatacacctcatcccagacagggtcaatcatagtcaagttactcccaaatatgcccaaggacaaagaggcatgtgcaaaagccccattggaggcttacaacaggacactgactactttgcttaccccccttggtgattctatccgcaggatacaagagtctgtgactacatccggaggaaggagacagagacgctttataggtgccattattggcagtgtagctctaggggttgcaacagctgcacagataacggcagcctcggctctgatacaagccaaccagaatgctgctaacatcctccggcttaaggagagcattgctgcaaccaatgaagctgtacacgaggtcactggcggattgtcacagttagcagtggcagttggaaagatgcagcaatttgttaatgaccagtttaataacacagctcaagagttggactgtataaaaattacacagcaggttggtgtagaactcaacttatacttaactgaattgactacagtgttcgggccacaaatcacttcccctgccttaactcagttgactgttcaggctctttacaatctggctggtggtaatgtagattacttgttgactaagttaggtgtagggaacaaccagctcagctcattgattggtagcggcttgatcaccggtaaccctattttttacgactcacagactcaactcttgggcatacaggtgactttaccctcagtcgggaacctaaataatatgcgtgccacctacttggagaccttgtctgtaagcacaacaaagggatttgcctcagcacttgtcccaaaagtagtgacacaggtcggttctgtgatagaagagcttgacacctcatactgtatagaagctgatttggatttatattgtacaagaatagtgacattccctatgtcccctggtatttattcctgtttgagcggcaatacatcggcttgcatgtattcaaggactgaaggcgcacttactacaccatacatgactctcaaaggctcagttgttgccaattgccagatgacaacatgtagatgtgcagaccccccgggtatcatatcacaaaattatggagaggctgtgtctctaatagataagcactcatgcaatgtcgtatccttagacgggataactttgaggctcagtggggaatttgatgcaacttatcaaaagaatatctcaatattagattctcaagtactagtgacaggcaatctcgatatctcaactgagcttgggaatgtcaaccactcaataagtaatgctttggataagttagaggaaagcaacagcaaactagacaaagtcaatgtcaaactgaccagcacatctgctctcattacctatattgttttaactgtcatatctcttgttcttggtatgcttagcctggttctagcatgctatctgatgtacaagcaaaaagcgcaacgaaagaccttgttgtggcttggaaataataccctagatcagatgagagctactacaaaaatgtgaatgcagatgagaggcagaggtatccccaatagcaatctgtgtgtcaattctggcagcctgttaatcagaagaattaagaaaaaactaccggatgtaggtgaacaaaagggaatatacgggtagaacggcctgagaggccacccctcaatcgggagccaggccccactacgtccgctctaccgcatcaccaacagcagtcttcagtcatggacagcgcagttagccaagttgcgctagagaatgatagaagagaagcgaataatacatggcgcttggttttccggatcgcagccttacttttactggtaataaccttagccgtctctgcagtcgccctggcatatagtatggaggctagcacacctggcgaccttgtaagcataccgactgcgatctctagggcagaggaaaggattacatctgcactcggttccaatcaagatgtagtagataggatatacaagcaagtggcccttgagtctccattggcactgctaaataccgaatctataattatgaatgcaataacgtctctctcttatcaaatcaatggagccacaaataatagcgggtgtggggcacctgttcatgacccagattacatcggggggataggtcaagaacttattgtagatgatacgagtgacgtcacatcattttatccctctgcattccaagaacacctgaattttatcccggcgcccactacaggatcaggctgcactcggataccctcattcgacatgagtgctacccattactgttacactcacaatgtgatattgtctggctgcagagatcactcacactcacatcagtatttagcacttggtgtgcttcggacatctgcaacagggagggtattcttttctactctgcgttccatcaacttggatgacgcccaaaatcggaagtcttgcagtgtgagtgcaactcctttaggttgtgatatgctgtgctctaaaatcacagagactgaggaagaggattataaatcagttatccccacatcgatggtacatggaaggttagggcttgacggccaataccatgagaaggacctagacgtcacaacactatttagggactgggtggcaaattacccaggagtaggaggtgggtcttttattaacaatcgcgtatggttcccggtctacggagggctaaaacccagctcgcctagtgacactgcacaagaagggagatacgtaatatacaagcgatacaatgacacatgcccggatgagcaagattaccagattcggatggctaagtcatcgtataagcctaggcggtttggtggaaaacgcgtacagcaggccatcctatccatcaaggtgtcaacatccttgggtgaggacccggtgttgactgtaccgcctaatacggtcgcactcatgggggccgaaggcagagttctcacagtggggacatctcatttcttatatcagcgagggtcatcatacttctctcccgctttgttataccctatgacagtcaacaacaaaacagccactcttcataatccttatacattcaatgctttcactcggccaggtagtgtcccttgccaggcttcagcaagatgccccaactcatgtgttaccggagtctatactgatccatatcccttagtcttccataggaaccacaccttgcgaggggtattcggaacaatgcttgatgacgaacaagcaagactcaaccctgtatctgcagtatttgataacatatcccgcagtcgcataactcgggtaagttcaagcagtaccagggcagcatacacgacatcaacgtgttttaaagttgtcaagaccaataaaacctattgcctcagcattgcagaaatatccaataccctcttcggggaattcagaattgtccccttactagttgagattctcaaggatggtggggtttaagaagctaggaatggtcggttgagtcaaccgtgagaaggcggggaagatgatattgcatcatctatcttctgtaacaccaagaatcaaatcgaataccagtgcgagctcgaatcctacgctgccagtcagccataatcggctggtgctaatgtggttagcctgaatcttgtcgatagtcacttgattaagaaaaaatgtggacggtagtgaaatataaggcaaagcagctcacagaggacagcacgggtaggacatggcgggctccggacccgaaagggcagagcatcagattatcctaccagagtcacatctgtcctcaccattggtcaagcataaattgctttattactggaaattaactgggctaccgcttcctgacgaatgtgacttcgaccatcttattatcagccgacaatggaagaaggtacttgaatcggccacccctgacattgagagaatgataaaactagggcgggcagtacaccagactctcaaccacaattccaggataaccggagtactccatccccggtgtttagaggaactggctagtattgaggttcctgattcaaccaacaaatttcggaagatcgaaaaaaagatccagattcacaacacaaggtatggagaactgttcacaagattatgcacgcatgtagaaaagaaactattggggtcatcttggtctaacaatgtcccacggtcagaggaattcaacagcatccgtacagatccggcattttggtttcactcaaaatggtccacagccaaatttgcatggctccatataaaacaggtccagaggcatctgattgtagcagcaagaacaaggtccgcagtcaacaaattagtgacgctgacccataaggtaggccaaatctttgttactcctgagcttgtcattgtgacacatacagatgagaacaagttcacgtgtcttacccaggaacttgtgttgatgtatgcagatatgatggagggcagagatatggtcagcataatatcatccacggcggcacatcttaggagcttatcagagaaaattgatgatattctgcggttagtagatgctctggcaagagatttgggcaatcaagtctacgatgttgtagcactaatggagggattcgcatacggcgctgttcagctgcttgaaccgtcaggtacatttgcgggggatttcttcgcattcaacctgcaggagctcaaagatactctaaccggactcctccccaaggatatcgcagaatctgtgactcacgcaatcgcaaccatattctctggcttagaacaaaatcaagcagctgagatgttgtgcctgttgcgtctgtggggtcacccactactcgagtcccgtattgcagcaaaagcagttaggagtcaaatgtgcgcaccaaaaatggtagactttgatatgatcctccaggtattatctttctttaaaggaacaatcatcaatggatatagaaagaagaatgcaggcgtgtggccacgtgtcaagatagatacgatatacgggaaggtcatagggcagctacacgcagattctgcagagatttcacatgatgtcatgttgagggaatacaagagtttatctgcacttgaattcgagccatgtatagagtatgaccctgtcaccaatctgagcatgtttttaaaagacaaggcaatcgcacacccgaaagacaactggctcgcttcgtttaggcgaaaccttctctctgaggaccagaagaaacatgtaagagaggcaacctcaactaaccgcctcttgatagagttcttagagtcaaatgattttgatccatataaagagatggaatatctgacgacccttgagtacctaagagatgataatgtggcagtatcatactcactcaaagaaaaagaggtgaaagttaatgggcggattttcgcaaagctaacaaatagattaaggaattgtcaggtaatggcagaagggatcctagctgaccagattgcacctttcttccagggaaatggggtcattcaggatagcatatctttgactaaaagtatgctagcgatgagtcaactgtctttcaacagcaacaagaaacgtattactgactgcaaagaaagagtatcctcaaaccgcaatcatgatccgaagagcaagaatcgtcggagagttgccacttttgtaactaccgacctgcaaaagtattgtctcaattggagatatcagacagtcaagctgttcgctcatgccatcaatcagctgatgggcttacctcacttcttcgagtggattcatcttagactaatggatactacgatgtttgtaggggaccctttcaatcctccaagtgaccctacggactgtgatctatcaagagtcccaaatgatgacatatatattgtcagtgctaggggaggcattgagggattatgccagaagctatggacaatgatctcaattgctgcaatccagcttgctgcagcaagatcacattgtcgcgttgcctgtatggtacaaggcgacaatcaagtaatagctgtaacgagagaggtaagatcagatgactccccagagatggtgttaacacaattgcatcaagccagtgataatttcttcaaggaattgattcatgtcaatcatttgatcggccataatttgaaggatcgtgaaaccatcaggtcagacacgttcttcatatacagcaaacgaatattcaaagatggagcaatactcagtcaggtcctcaaaaactcatctaaattagtgctaatatcaggcgaccttagtgaaaacactgtaatgtcttgtgccaacattgcatctactgtagcacggttatgcgagaacgggcttcctaaggatttctgttattacttaaactacttaatgagttgcgtgcagacatacttcgattccgaattttccatcaccaacaactcgcaactcgattctaaccagtcgtggatagaggacatttcttttgtgcactcatatgtcctgacccctgctcaactggggggactgagtaaccttcaatactcaaggctctacacaaggaacatcggcgacccgggaaccactgctttcgcagagatcaagagattagaggcagtggggttactgagtcctagcattatgactaacatcttaactaggccgcctggaaatggagattgggccagtctgtgcaacgatccatactcctttaattttgagactgtcgcaagcccaaatattgtccttaagaaacatacacaaagagtcctatttgaaacttgctcgaatcccttattatctggagtgcacacagaggataatgaggcagaagagaaggcattggctgaattcttgctcaatcaagaaatgattcatccacgtgtcgcgcatgctatcatggaagcaagctctgtaggtaggagaaagcaaattcaggggcttgttgacacaacaaacaccgtgattaagattgcactgactaggaggccactcggcatcaagaggctgatgcggatagtcaattactcgagcatgcatgcaatgctatttagagatgatgttttctcgtctaatagatccaaccatcccttagtctcttctgatatgtgttctcagacactggcagactatgcacggaacagaagctggtcacctttgacagggggtagaaaaatactgggtgtatctaatcctgataccatagaacttgtagagggtgagatccttagtgtcagtggagggtgcactagatgtgacagtggggatgaacagtttacttggttccatcttccaagcaatatagagctaaacgatgacaccagcaagaatcctccaatgagagtgccatatctcgggtcaaagactcaagagaggagagccgcctcgcttgcgaaaatagctcatatgtcaccacacgtgaaggcggctctaagggcgtcatctgtgttaatctgggcttatggggacaacgaaataaactggactgctgcccttaagattgcaaggtctcggtgcaacataagctcagagtatcttcgactattgtcacccttgcctacagctgggaatctccaacatagattggacgacggcataactcagatgacattcacccctgcatctctatacagggtgtcaccttacattcatatatctaatgattctcaaaggctatttactgaagaaggagtcaaagaggggaatgtggtttatcagcaaatcatgctcttgggtttgtccttaattgagtcactcttcccaatgacaacaaccaagacatatgatgaaatcacattgcacctccacagtaaatttagctgctgtatcagggaagcacctgttgcagttcctttcgagctactcggggtggcaccggaactaagggcagtaacctcaaataagtttatgtatgatcctagccctgtatcagagggagactttgcgagacttgacttagctatctttaagagttatgaacttaatttagagtcatattccacaatagagctaatgaacgttctttcaatatctagtgggaagttgattggccagtctgtggtttcttatgatgaagatacctccataaagaatgacgctataatagtgtatgacaacacacggaattggatcagcgaagctcagaattcagatgtggtccgcctattcgagtatgcggcactcgaagtgctcctcgactgttcttatcaactctactatctgagagtaagaggcctaaacaatatcgtcctgtacatgagtgatttatacaagaatatgccaggaattctactctctaatattgcagccacaatatctcaccctgtcatccattcaaggttgaatgcagtaggtctggtcaaccatgacgggtcacaccaacttgcagacacagatttcattgaaatgtctgcaaagctgctagtcttttgcactcgacgcgtggtctcaggtttacatgcagggaataagtatgacctgctgtttccatctgtcttggatgataacctaagtgagaagatgcttcagttgatatcccaattatgctgtctgtatacggtgctctttgctacaacaagagaaatcccgaaaataagaggcttatctgcagaagagaaatgttcagtacttactgagtacctactgtcagatgctgtgaaaccgttacttgggtccgagcaagtgagctctatcatgtctcccaacatagttacgttcccagccaatctgtattacatgtctaggaagagccttaatttgatcagggagagagaggacagggatactatcttggcattgttgttccctcaagaaccgctgctcgagtttcctctggtacgagatattggtgctcgtgtaaaagatccatttacccgacaacctgcggcgtttttacaagagttagatttgagtgctccggcaaggtatgacgcattcacaatcagtcaggcgcattctgaacacatattgccaaacccagaggaagatcacttagtacgatacttgttcagaggaatagggactgcgtcctcctcttggtataaggcatcccatcttctttctgtacccgaggtcagatgtgcaaggcatgggaactccttatatttagcagaaggaagtggagccatcatgagtcttctcgaattgcatataccacacgaaactatctattacaatacacttttctcgaacgagatgaaccccccacagcgacatttcggaccaactccaacacagtttctgaattcggttgtttttaggaatttacaggcggaagtaccatgcaaggatggatttgtccaggagttccgtccgttatggagagagaatacagaagaaagcgatctgacctcagataaagcagtgggatatatcacatctgtagtgccgtacaggtctgtatcattgctgcattgtgacattgaaatccctccaggatctaatcaaagcttactagatcagctggctaccaatctgtctctgattgccatgcattctgtgaaggagggcggggtcgtgattatcaaagtactgtatgcaatgggatattacttccatctactcatgaacttgtttactccatgttccacgaaaggatatattctctctaatggctatgcctgtagaggggatatggagtgttacctgatatttgtcatgggctacctaggcgggcctacatttgtacatgaggtggtgaggatggcaaaaacgctagtacagcggcatggcacacttttgtctaagtcagatgagattacactgactaggttatttacttcacagcaacagcgtgtaacagacatcctatccagccctttaccgagactaatgaagtacttgagagagaatattgatactgcactgattgaagccgggggacagcctgtccgtccattctgtgcagagagtttagtgagcacactaacagacatgactcagacaacccagatcatcgccagccacattgatacagtcattcgatctgtgatctatatggaagcagagggtgatcttgctgacacagtattcttatttaccccttacaacctctctatggacgggaaaaagagaacatcacttaaacagtgcacaagacagatcttagaggtcacaatactgggtctcagagtcaaaaatctcaataaagtaggtgatgtaatcagcctagtactcagaggtatgatttctctggaggaccttctcccactgagaacctacttgaagtgtagtacctgccctaagtatttgaaggctgtcctaggtattaccaaactcaaagaaatgttcacagacgcctctttattatacttgactcgtgctcaacaaaaattctacatgaaaactataggcaatgcagtcaagggatactatagtaactgtgactcttaaaggcaaccacatatcaataggccctctttctagccgatcgtattcttgttgacttcattataccatattagaaaaaaattgaattccgaccctttaagactcgtattcggattcaaataattatctcagaaaaaaagtgcacgtagttgttcttgattatagtcccgtcattcaccaagtctttgtttggt

SEQ ID NO: 4 - DNA, 15186 bp, Newcastle disease virus
  Mut HN (F277L)/M(G165W)/F(F117S)/F(F190L)/L(V757I)/L(F1551S)/L(R1700L)
accaaacagagaatccgtgagatacgataaaaggcgaaggagcaatcgaagtcgtacgggtagaaggtgtgaatctcgagtgcgagaccgaagcttaaactcgagagagccttctgccaacatgtcttccgtatttgacgagtatgaacagctcctcgcggctcagactcgccctaatggagctcacggaggaggagagaaggggagcaccttaaaagttgaagtcccggtattcactcttaacagtgacgacccagaagatagatggaactttgcagtattctgtcttaggattgctgttagcgaagatgccaacaaaccactcagacaaggtgctctcatatctctcttatgctcccattctcaagtgatgaggaaccatgtcgcccttgcagggaaacagaatgaggccacactggctgttcttgagatcgatggtttcaccaacagcgtgcctcaatttaacaacaggagtggagtgtctgaggagagagcacagagattcttgatgatagcagggtccctccctcgggcatgcagcaacggtaccccgttcatcacagctggggttgaagatgatgcaccagaagacatcactgatactctggaaagaatcatctctatccaggctcaagtatgggtcacggtagcaaaggccatgactgcatatgagacagcggatgagtcggaaacaagaagaatcaataagtatatgcagcaaggcagagtccaaaagaagtacatcctccaccccgtatgtaggagcgcaattcaactcacgattagacaatctctggcagtccgcattttcttggttagcgagcttaagagaggccgcaacacggcaggtggaagttccacttattatagcttggtaggggacatagactcatacatcaggaacaccgggcttactgcattcttcctgacactcaggtatggaattaacaccaagacatcagcccttgcactcagcagcctcgcaggcgatatccaaaaaatgaagcagctcatgcgtctatatcggatgaaaggagataatgcgccgtacatgacactgctcggtgacagtgaccagatgagctttgcaccggctgagtatgcacaactctactcttttgcaatgggtatggcatccgtcctagacaaaggaactggcaagtaccagttcgccagagactttatgagcacatcattctggagacttggagtagagtatgctcgggctcagggaagcagcatcaatgaggatatggctgccgagctaaagctgaccccagcggcaaggaggggcctggcagctgctgcccaacgagcgtccgaggagaccggcagcatggacattcccactcaacaagctggggtcctcacagggctcagcgacggaggccctcaagccccacaaggtggactgaacaggtcgcaagggcaaccggacgccggagatggggagacccaatttttagatttgatgagagcagtggcaaatagtatgagagaagcgccaaactctgtgcagaacaccactcagcaggagcctccatctactcctgggccatctcaagacaacgacaccgactggggatactgaccgacaacactcagcctgcctccatcgaattatctcgattcttctgcctgtaacctaacccctggtccacagtctcacacggccaaacccgcaaacgagcccccccctctctccccccccctccagccacacggctccaaccgcccaaaacaacacgggcacaactcgactcactcataatctacacagagccgaagatcttagaaaaaaatacgggtagaagagagacattcagagatcaggacaaatcatcagggtctccgctctcccctctacccagcagaccagagtgaagatggccaccttcacggatgcagagatcgacgatctatttgaaaccagtggaactgtcattgacagcataattacggcccaaggcaacccagtagagactgttggaaggagcgcaatcccacaaggcaaaactaaagcattgagtgcagcatgggagaaacatggaagcacccaaccgccggccggccaagacacccctgatcgaatggacagatcagacaaacaaccatccccacccgaacaggcaaccacacacaacagcccgccagccacatccaccgaccagccccccacccaggccgcaggcgaagccggtgacacacagctcaagactggagcaagcaactccctcctgtcgatgctcgacaagcttagcaataaatcgtccaatgctaaaaagggcccacggtcgagtccccaggaagggcaccaccaacctccgacccagcagcaagggagtcaacagagccgcggaaacagtcaggagagatcgcaaaaccaggtcaaggccgcctctggagaccagggcacagacgcgaacacagcatatcatggacaatggaaggagtcacaaccatcagctggtgcaatccctcatgctctccggtcagggcagagccaagccaatactcctgcatctgtggatcatgtccagctacctgtcgactttgtgcaggcgatgatgtctatgatggaggcgatatcacagaaggtaaataaagtcgactatcagctagaccttgtctcaaagcagacatcctccatccccatgatgcgatctgaaatccaacagctgaaaacatccgttgcggtcatggaagccaacctgggcatgatgaagattctggaccctggttgtgctaacgtttcatctttaagtgacctacgggcggtcgcccgctctcacccagttttagtttcaggccccggagacccatctccttatgtgacacaagggggtgaattgatactcaataaactctcgcaaccagtgcaacatccttctgagttgattaaacctgccacgataagcgggcctgatataggagtggagaaggacactgtccgtgcattgatcacctcacgcccaatgcatccgagctcttcagctaagctcctgagtaagctggatgcagccgggtcgatagaagaaattaggaaaatcaaacgccttgcgctgaatggctgatcactactacaacccgcaacgggttcccgtttatctattgtcacaaggaacccgccccgagccctcctctataaacccaagattcaaggctccaagcgataaccttctcccgcctcttccatcccactgaatgatcgcgcagctgcaattaatctagcaacattaaagattaagaaaaaatacgggtagaatcggagtgcaccgattgtgccaagatggactcatctaggacaattgggttatactttgattctgcccttccttctagcaacctgttagcattcccgattgtcttacgagatgtaggagatggaaagaagcaaattactccgcaatataggatccggcgtcttgactcgtggacagacagtaaagaagactcggtattcatcactacctacggattcatcttccaggttgggaatgaagaagtcactgtcggcatgatcaatgataatcccaagcgagagttactctccgctgcgatgctctgcctaggaagcgtcccgaatgttggagatcctgttgagctggcaagggcctgcctcactatggtggtaacatgcaagaagagtgcaactaatactgagagaatggtcttctcagtagtgcaggcaccccgggtgctgcaaagctgtagggtcgtggcagacaaatactcgtcagtgaatgcagttaagcacgtgaaagcaccagagaagatcccttggagtgaaaccctagagtacaaggtgaattttgtctctttgactgtggtgccgaagaaggatgtctacaagatcccaaccgcagtattgaaggtatccggctcgagcctgtataatcttgcgctcaatgtcactattgatgtggaggtagacccaaagagcccgttagtcaagtccctttctaggtccgacagtggatactatgctaatctcttcttacatatcggacttatgtccactgtagataagaggggaaagaaagtgacatttgaccagctggagaggaagataaggagacttgatctatctgtcgggctcagtgatgtgctcggaccttctgtgcttgtgaaggcgagaggtgcacggactaggctgctggcacctttcttctctaacagcgggacagcctgctaccctatagcaaatgcctctcctcaggtagctaagatactctggagtcagaccgcgtgcctgcggagtgtaaaaatcattattcaagcgggcacccaacgcgctgtcgcagtgactgctgaccatgaggttacctctactaagatagagaagaggcataccatcgctaaatacaatcccttcaagaaataggccgtatccctgagactgcaattcacccgccttcccaaaacaccatgacaccagataatgatctgtcttgattacttacagttagtttccctgtctatcaaattagaaaaaacacgggtagaagagttcggatcccggccggcgcaccaaaagcgcaagatgggccccagatcttctaccaggatcccagtacctctaatgctgaccatacggatcacgctggcactgagttatgtccgtctgacaagttctcttgatggcaggcctcttgcagccgcagggatcgtggtaacaggggataaagcagttaacatatacacctcatcccagacagggtcaatcatagtcaagttactcccaaatatgcccaaggacaaagaggcatgtgcaaaagccccattggaggcttacaacaggacactgactactttgcttaccccccttggtgattctatccgcaggatacaagagtctgtgactacatccggaggaaggagacagagacgctctataggtgccattattggcagtgtagctctaggggttgcaacagctgcacagataacggcagcctcggctctgatacaagccaaccagaatgctgctaacatcctccggcttaaggagagcattgctgcaaccaatgaagctgtacacgaggtcactggcggattgtcacagttagcagtggcagttggaaagatgcagcaatttgttaatgaccagcttaataacacagctcaagagttggactgtataaaaattacacagcaggttggtgtagaactcaacttatacttaactgaattgactacagtgttcgggccacaaatcacttcccctgccttaactcagttgactgttcaggctctttacaatctggctggtggtaatgtagattacttgttgactaagttaggtgtagggaacaaccagctcagctcattgattggtagcggcttgatcaccggtaaccctattttttacgactcacagactcaactcttgggcatacaggtgactttaccctcagtcgggaacctaaataatatgcgtgccacctacttggagaccttgtctgtaagcacaacaaagggatttgcctcagcacttgtcccaaaagtagtgacacaggtcggttctgtgatagaagagcttgacacctcatactgtatagaagctgatttggatttatattgtacaagaatagtgacattccctatgtcccctggtatttattcctgtttgagcggcaatacatcggcttgcatgtattcaaggactgaaggcgcacttactacaccatacatgactctcaaaggctcagttgttgccaattgccagatgacaacatgtagatgtgcagaccccccgggtatcatatcacaaaattatggagaggctgtgtctctaatagataagcactcatgcaatgtcgtatccttagacgggataactttgaggctcagtggggaatttgatgcaacttatcaaaagaatatctcaatattagattctcaagtactagtgacaggcaatctcgatatctcaactgagcttgggaatgtcaaccactcaataagtaatgctttggataagttagaggaaagcaacagcaaactagacaaagtcaatgtcaaactgaccagcacatctgctctcattacctatattgttttaactgtcatatctcttgttcttggtatgcttagcctggttctagcatgctatctgatgtacaagcaaaaagcgcaacgaaagaccttgttgtggcttggaaataataccctagatcagatgagagctactacaaaaatgtgaatgcagatgagaggcagaggtatccccaatagcaatctgtgtgtcaattctggcagcctgttaatcagaagaattaagaaaaaactaccggatgtaggtgaacaaaagggaatatacgggtagaacggcctgagaggccacccctcaatcgggagccaggccccactacgtccgctctaccgcatcaccaacagcagtcttcagtcatggacagcgcagttagccaagttgcgctagagaatgatagaagagaagcgaataatacatggcgcttggttttccggatcgcagccttacttttactggtaataaccttagccgtctctgcagtcgccctggcatatagtatggaggctagcacacctggcgaccttgtaagcataccgactgcgatctctagggcagaggaaaggattacatctgcactcggttccaatcaagatgtagtagataggatatacaagcaagtggcccttgagtctccattggcactgctaaataccgaatctataattatgaatgcaataacgtctctctcttatcaaatcaatggagccacaaataatagcgggtgtggggcacctgttcatgacccagattacatcggggggataggtcaagaacttattgtagatgatacgagtgacgtcacatcattttatccctctgcattccaagaacacctgaattttatcccggcgcccactacaggatcaggctgcactcggataccctcattcgacatgagtgctacccattactgttacactcacaatgtgatattgtctggctgcagagatcactcacactcacatcagtatttagcacttggtgtgcttcggacatctgcaacagggagggtattcttttctactctgcgttccatcaacttggatgacgcccaaaatcggaagtcttgcagtgtgagtgcaactcctttaggttgtgatatgctgtgctctaaaatcacagagactgaggaagaggattataaatcagttatccccacatcgatggtacatggaaggttagggcttgacggccaataccatgagaaggacctagacgtcacaacactatttagggactgggtggcaaattacccaggagtaggaggtgggtcttttattaacaatcgcgtatggttcccggtctacggagggctaaaacccagctcgcctagtgacactgcacaagaagggagatacgtaatatacaagcgatacaatgacacatgcccggatgagcaagattaccagattcggatggctaagtcatcgtataagcctaggcggtttggtggaaaacgcgtacagcaggccatcctatccatcaaggtgtcaacatccttgggtgaggacccggtgttgactgtaccgcctaatacggtcgcactcatgggggccgaaggcagagttctcacagtggggacatctcatttcttatatcagcgagggtcatcatacttctctcccgctttgttataccctatgacagtcaacaacaaaacagccactcttcataatccttatacattcaatgctttcactcggccaggtagtgtcccttgccaggcttcagcaagatgccccaactcatgtgttaccggagtctatactgatccatatcccttagtcttccataggaaccacaccttgcgaggggtattcggaacaatgcttgatgacgaacaagcaagactcaaccctgtatctgcagtatttgataacatatcccgcagtcgcataactcgggtaagttcaagcagtaccagggcagcatacacgacatcaacgtgttttaaagttgtcaagaccaataaaacctattgcctcagcattgcagaaatatccaataccctcttcggggaattcagaattgtccccttactagttgagattctcaaggatggtggggtttaagaagctaggaatggtcggttgagtcaaccgtgagaaggcggggaagatgatattgcatcatctatcttctgtaacaccaagaatcaaatcgaataccagtgcgagctcgaatcctacgctgccagtcagccataatcggctggtgctaatgtggttagcctgaatcttgtcgatagtcacttgattaagaaaaaatgtggacggtagtgaaatataaggcaaagcagctcacagaggacagcacgggtaggacatggcgggctccggacccgaaagggcagagcatcagattatcctaccagagtcacatctgtcctcaccattggtcaagcataaattgctttattactggaaattaactgggctaccgcttcctgacgaatgtgacttcgaccatcttattatcagccgacaatggaagaaggtacttgaatcggccacccctgacattgagagaatgataaaactagggcgggcagtacaccagactctcaaccacaattccaggataaccggagtactccatccccggtgtttagaggaactggctagtattgaggttcctgattcaaccaacaaatttcggaagatcgaaaaaaagatccagattcacaacacaaggtatggagaactgttcacaagattatgcacgcatgtagaaaagaaactattggggtcatcttggtctaacaatgtcccacggtcagaggaattcaacagcatccgtacagatccggcattttggtttcactcaaaatggtccacagccaaatttgcatggctccatataaaacaggtccagaggcatctgattgtagcagcaagaacaaggtccgcagtcaacaaattagtgacgctgacccataaggtaggccaaatctttgttactcctgagcttgtcattgtgacacatacagatgagaacaagttcacgtgtcttacccaggaacttgtgttgatgtatgcagatatgatggagggcagagatatggtcagcataatatcatccacggcggcacatcttaggagcttatcagagaaaattgatgatattctgcggttagtagatgctctggcaagagatttgggcaatcaagtctacgatgttgtagcactaatggagggattcgcatacggcgctgttcagctgcttgaaccgtcaggtacatttgcgggggatttcttcgcattcaacctgcaggagctcaaagatactctaaccggactcctccccaaggatatcgcagaatctgtgactcacgcaatcgcaaccatattctctggcttagaacaaaatcaagcagctgagatgttgtgcctgttgcgtctgtggggtcacccactactcgagtcccgtattgcagcaaaagcagttaggagtcaaatgtgcgcaccaaaaatggtagactttgatatgatcctccaggtattatctttctttaaaggaacaatcatcaatggatatagaaagaagaatgcaggcgtgtggccacgtgtcaagatagatacgatatacgggaaggtcatagggcagctacacgcagattctgcagagatttcacatgatgtcatgttgagggaatacaagagtttatctgcacttgaattcgagccatgtatagagtatgaccctgtcaccaatctgagcatgtttttaaaagacaaggcaatcgcacacccgaaagacaactggctcgcttcgtttaggcgaaaccttctctctgaggaccagaagaaacatgtaagagaggcaacctcaactaaccgcctcttgatagagttcttagagtcaaatgattttgatccatataaagagatggaatatctgacgacccttgagtacctaagagatgataatgtggcagtatcatactcactcaaagaaaaagaggtgaaagttaatgggcggattttcgcaaagctaacaaatagattaaggaattgtcaggtaatggcagaagggatcctagctgaccagattgcacctttcttccagggaaatggggtcattcaggatagcatatctttgactaaaagtatgctagcgatgagtcaactgtctttcaacagcaacaagaaacgtattactgactgcaaagaaagagtatcctcaaaccgcaatcatgatccgaagagcaagaatcgtcggagagttgccacttttgtaactaccgacctgcaaaagtattgtctcaattggagatatcagacagtcaagctgttcgctcatgccatcaatcagctgatgggcttacctcacttcttcgagtggattcatcttagactaatggatactacgatgtttgtaggggaccctttcaatcctccaagtgaccctacggactgtgatctatcaagagtcccaaatgatgacatatatattgtcagtgctaggggaggcattgagggattatgccagaagctatggacaatgatctcaattgctgcaatccagcttgctgcagcaagatcacattgtcgcgttgcctgtatggtacaaggcgacaatcaagtaatagctataacgagagaggtaagatcagatgactccccagagatggtgttaacacaattgcatcaagccagtgataatttcttcaaggaattgattcatgtcaatcatttgatcggccataatttgaaggatcgtgaaaccatcaggtcagacacgttcttcatatacagcaaacgaatattcaaagatggagcaatactcagtcaggtcctcaaaaactcatctaaattagtgctaatatcaggcgaccttagtgaaaacactgtaatgtcttgtgccaacattgcatctactgtagcacggttatgcgagaacgggcttcctaaggatttctgttattacttaaactacttaatgagttgcgtgcagacatacttcgattccgaattttccatcaccaacaactcgcaactcgattctaaccagtcgtggatagaggacatttcttttgtgcactcatatgtcctgacccctgctcaactggggggactgagtaaccttcaatactcaaggctctacacaaggaacatcggcgacccgggaaccactgctttcgcagagatcaagagattagaggcagtggggttactgagtcctagcattatgactaacatcttaactaggccgcctggaaatggagattgggccagtctgtgcaacgatccatactcctttaattttgagactgtcgcaagcccaaatattgtccttaagaaacatacacaaagagtcctatttgaaacttgctcgaatcccttattatctggagtgcacacagaggataatgaggcagaagagaaggcattggctgaattcttgctcaatcaagaaatgattcatccacgtgtcgcgcatgctatcatggaagcaagctctgtaggtaggagaaagcaaattcaggggcttgttgacacaacaaacaccgtgattaagattgcactgactaggaggccactcggcatcaagaggctgatgcggatagtcaattactcgagcatgcatgcaatgctatttagagatgatgttttctcgtctaatagatccaaccatcccttagtctcttctgatatgtgttctcagacactggcagactatgcacggaacagaagctggtcacctttgacagggggtagaaaaatactgggtgtatctaatcctgataccatagaacttgtagagggtgagatccttagtgtcagtggagggtgcactagatgtgacagtggggatgaacagtttacttggttccatcttccaagcaatatagagctaaacgatgacaccagcaagaatcctccaatgagagtgccatatctcgggtcaaagactcaagagaggagagccgcctcgcttgcgaaaatagctcatatgtcaccacacgtgaaggcggctctaagggcgtcatctgtgttaatctgggcttatggggacaacgaaataaactggactgctgcccttaagattgcaaggtctcggtgcaacataagctcagagtatcttcgactattgtcacccttgcctacagctgggaatctccaacatagattggacgacggcataactcagatgacattcacccctgcatctctatacagggtgtcaccttacattcatatatctaatgattctcaaaggctatttactgaagaaggagtcaaagaggggaatgtggtttatcagcaaatcatgctcttgggtttgtccttaattgagtcactcttcccaatgacaacaaccaagacatatgatgaaatcacattgcacctccacagtaaatttagctgctgtatcagggaagcacctgttgcagttcctttcgagctactcggggtggcaccggaactaagggcagtaacctcaaataagtttatgtatgatcctagccctgtatcagagggagactttgcgagacttgacttagctatctttaagagttatgaacttaatttagagtcatattccacaatagagctaatgaacgttctttcaatatctagtgggaagttgattggccagtctgtggtttcttatgatgaagatacctccataaagaatgacgctataatagtgtatgacaacacacggaattggatcagcgaagctcagaattcagatgtggtccgcctattcgagtatgcggcactcgaagtgctcctcgactgttcttatcaactctactatctgagagtaagaggcctaaacaatatcgtcctgtacatgagtgatttatacaagaatatgccaggaattctactctctaatattgcagccacaatatctcaccctgtcatccattcaaggttgaatgcagtaggtctggtcaaccatgacgggtcacaccaacttgcagacacagatttcattgaaatgtctgcaaagctgctagtctcttgcactcgacgcgtggtctcaggtttacatgcagggaataagtatgacctgctgtttccatctgtcttggatgataacctaagtgagaagatgcttcagttgatatcccaattatgctgtctgtatacggtgctctttgctacaacaagagaaatcccgaaaataagaggcttatctgcagaagagaaatgttcagtacttactgagtacctactgtcagatgctgtgaaaccgttacttgggtccgagcaagtgagctctatcatgtctcccaacatagttacgttcccagccaatctgtattacatgtctaggaagagccttaatttgatcagggagagagaggacagggatactatcttggcattgttgttccctcaagaaccgctgctcgagtttcctctggtacgagatattggtgctcgtgtaaaagatccatttaccctacaacctgcggcgtttttacaagagttagatttgagtgctccggcaaggtatgacgcattcacaatcagtcaggcgcattctgaacacatattgccaaacccagaggaagatcacttagtacgatacttgttcagaggaatagggactgcgtcctcctcttggtataaggcatcccatcttctttctgtacccgaggtcagatgtgcaaggcatgggaactccttatatttagcagaaggaagtggagccatcatgagtcttctcgaattgcatataccacacgaaactatctattacaatacacttttctcgaacgagatgaaccccccacagcgacatttcggaccaactccaacacagtttctgaattcggttgtttttaggaatttacaggcggaagtaccatgcaaggatggatttgtccaggagttccgtccgttatggagagagaatacagaagaaagcgatctgacctcagataaagcagtgggatatatcacatctgtagtgccgtacaggtctgtatcattgctgcattgtgacattgaaatccctccaggatctaatcaaagcttactagatcagctggctaccaatctgtctctgattgccatgcattctgtgaaggagggcggggtcgtgattatcaaagtactgtatgcaatgggatattacttccatctactcatgaacttgtttactccatgttccacgaaaggatatattctctctaatggctatgcctgtagaggggatatggagtgttacctgatatttgtcatgggctacctaggcgggcctacatttgtacatgaggtggtgaggatggcaaaaacgctagtacagcggcatggcacacttttgtctaagtcagatgagattacactgactaggttatttacttcacagcaacagcgtgtaacagacatcctatccagccctttaccgagactaatgaagtacttgagagagaatattgatactgcactgattgaagccgggggacagcctgtccgtccattctgtgcagagagtttagtgagcacactaacagacatgactcagacaacccagatcatcgccagccacattgatacagtcattcgatctgtgatctatatggaagcagagggtgatcttgctgacacagtattcttatttaccccttacaacctctctatggacgggaaaaagagaacatcacttaaacagtgcacaagacagatcttagaggtcacaatactgggtctcagagtcaaaaatctcaataaagtaggtgatgtaatcagcctagtactcagaggtatgatttctctggaggaccttctcccactgagaacctacttgaagtgtagtacctgccctaagtatttgaaggctgtcctaggtattaccaaactcaaagaaatgttcacagacgcctctttattatacttgactcgtgctcaacaaaaattctacatgaaaactataggcaatgcagtcaagggatactatagtaactgtgactcttaaaggcaaccacatatcaataggccctctttctagccgatcgtattcttgttgacttcattataccatattagaaaaaaattgaattccgaccctttaagactcgtattcggattcaaataattatctcagaaaaaaagtgcacgtagttgttcttgattatagtcccgtcattcaccaagtctttgtttggt

SEQ ID NO: 5 - DNA, 15198 bp, Newcastle disease virus
  Mut HN (F277L)/M(G165W)/F(F117S)/F(F190L)/L(V757I)/L(F1551S)/L(R1700L) +AfeI
accaaacagagaatccgtgagatacgataaaaggcgaaggagcaatcgaagtcgtacgggtagaaggtgtgaatctcgagtgcgagaccgaagcttaaactcgagagagccttctgccaacatgtcttccgtatttgacgagtatgaacagctcctcgcggctcagactcgccctaatggagctcacggaggaggagagaaggggagcaccttaaaagttgaagtcccggtattcactcttaacagtgacgacccagaagatagatggaactttgcagtattctgtcttaggattgctgttagcgaagatgccaacaaaccactcagacaaggtgctctcatatctctcttatgctcccattctcaagtgatgaggaaccatgtcgcccttgcagggaaacagaatgaggccacactggctgttcttgagatcgatggtttcaccaacagcgtgcctcaatttaacaacaggagtggagtgtctgaggagagagcacagagattcttgatgatagcagggtccctccctcgggcatgcagcaacggtaccccgttcatcacagctggggttgaagatgatgcaccagaagacatcactgatactctggaaagaatcatctctatccaggctcaagtatgggtcacggtagcaaaggccatgactgcatatgagacagcggatgagtcggaaacaagaagaatcaataagtatatgcagcaaggcagagtccaaaagaagtacatcctccaccccgtatgtaggagcgcaattcaactcacgattagacaatctctggcagtccgcattttcttggttagcgagcttaagagaggccgcaacacggcaggtggaagttccacttattatagcttggtaggggacatagactcatacatcaggaacaccgggcttactgcattcttcctgacactcaggtatggaattaacaccaagacatcagcccttgcactcagcagcctcgcaggcgatatccaaaaaatgaagcagctcatgcgtctatatcggatgaaaggagataatgcgccgtacatgacactgctcggtgacagtgaccagatgagctttgcaccggctgagtatgcacaactctactcttttgcaatgggtatggcatccgtcctagacaaaggaactggcaagtaccagttcgccagagactttatgagcacatcattctggagacttggagtagagtatgctcgggctcagggaagcagcatcaatgaggatatggctgccgagctaaagctgaccccagcggcaaggaggggcctggcagctgctgcccaacgagcgtccgaggagaccggcagcatggacattcccactcaacaagctggggtcctcacagggctcagcgacggaggccctcaagccccacaaggtggactgaacaggtcgcaagggcaaccggacgccggagatggggagacccaatttttagatttgatgagagcagtggcaaatagtatgagagaagcgccaaactctgtgcagaacaccactcagcaggagcctccatctactcctgggccatctcaagacaacgacaccgactggggatactgaccgacaacactcagcctgcctccatcgaattatctcgattcttctgcctgtaacctaacccctggtccacagtctcacacggccaaacccgcaaacgagcccccccctctctccccccccctccagccacacggctccaaccgcccaaaacaacacgggcacaactcgactcactcataatctacacagagccgaagatcttagaaaaaaatacgggtagaagagagacattcagagatcaggacaaatcatcagggtctccgctctcccctctacccagcagaccagagtgaagatggccaccttcacggatgcagagatcgacgatctatttgaaaccagtggaactgtcattgacagcataattacggcccaaggcaacccagtagagactgttggaaggagcgcaatcccacaaggcaaaactaaagcattgagtgcagcatgggagaaacatggaagcacccaaccgccggccggccaagacacccctgatcgaatggacagatcagacaaacaaccatccccacccgaacaggcaaccacacacaacagcccgccagccacatccaccgaccagccccccacccaggccgcaggcgaagccggtgacacacagctcaagactggagcaagcaactccctcctgtcgatgctcgacaagcttagcaataaatcgtccaatgctaaaaagggcccacggtcgagtccccaggaagggcaccaccaacctccgacccagcagcaagggagtcaacagagccgcggaaacagtcaggagagatcgcaaaaccaggtcaaggccgcctctggagaccagggcacagacgcgaacacagcatatcatggacaatggaaggagtcacaaccatcagctggtgcaatccctcatgctctccggtcagggcagagccaagccaatactcctgcatctgtggatcatgtccagctacctgtcgactttgtgcaggcgatgatgtctatgatggaggcgatatcacagaaggtaaataaagtcgactatcagctagaccttgtctcaaagcagacatcctccatccccatgatgcgatctgaaatccaacagctgaaaacatccgttgcggtcatggaagccaacctgggcatgatgaagattctggaccctggttgtgctaacgtttcatctttaagtgacctacgggcggtcgcccgctctcacccagttttagtttcaggccccggagacccatctccttatgtgacacaagggggtgaattgatactcaataaactctcgcaaccagtgcaacatccttctgagttgattaaacctgccacgataagcgggcctgatataggagtggagaaggacactgtccgtgcattgatcacctcacgcccaatgcatccgagctcttcagctaagctcctgagtaagctggatgcagccgggtcgatagaagaaattaggaaaatcaaacgccttgcgctgaatggctgatcactactacaacccgcaacgggttcccgtttatctattgtcacaaggaacccgccccgaagcgctgaccgagccctcctctataaacccaagattcaaggctccaagcgataaccttctcccgcctcttccatcccactgaatgatcgcgcagctgcaattaatctagcaacattaaagattaagaaaaaatacgggtagaatcggagtgcaccgattgtgccaagatggactcatctaggacaattgggttatactttgattctgcccttccttctagcaacctgttagcattcccgattgtcttacgagatgtaggagatggaaagaagcaaattactccgcaatataggatccggcgtcttgactcgtggacagacagtaaagaagactcggtattcatcactacctacggattcatcttccaggttgggaatgaagaagtcactgtcggcatgatcaatgataatcccaagcgagagttactctccgctgcgatgctctgcctaggaagcgtcccgaatgttggagatcctgttgagctggcaagggcctgcctcactatggtggtaacatgcaagaagagtgcaactaatactgagagaatggtcttctcagtagtgcaggcaccccgggtgctgcaaagctgtagggtcgtggcagacaaatactcgtcagtgaatgcagttaagcacgtgaaagcaccagagaagatcccttggagtgaaaccctagagtacaaggtgaattttgtctctttgactgtggtgccgaagaaggatgtctacaagatcccaaccgcagtattgaaggtatccggctcgagcctgtataatcttgcgctcaatgtcactattgatgtggaggtagacccaaagagcccgttagtcaagtccctttctaggtccgacagtggatactatgctaatctcttcttacatatcggacttatgtccactgtagataagaggggaaagaaagtgacatttgaccagctggagaggaagataaggagacttgatctatctgtcgggctcagtgatgtgctcggaccttctgtgcttgtgaaggcgagaggtgcacggactaggctgctggcacctttcttctctaacagcgggacagcctgctaccctatagcaaatgcctctcctcaggtagctaagatactctggagtcagaccgcgtgcctgcggagtgtaaaaatcattattcaagcgggcacccaacgcgctgtcgcagtgactgctgaccatgaggttacctctactaagatagagaagaggcataccatcgctaaatacaatcccttcaagaaataggccgtatccctgagactgcaattcacccgccttcccaaaacaccatgacaccagataatgatctgtcttgattacttacagttagtttccctgtctatcaaattagaaaaaacacgggtagaagagttcggatcccggccggcgcaccaaaagcgcaagatgggccccagatcttctaccaggatcccagtacctctaatgctgaccatacggatcacgctggcactgagttatgtccgtctgacaagttctcttgatggcaggcctcttgcagccgcagggatcgtggtaacaggggataaagcagttaacatatacacctcatcccagacagggtcaatcatagtcaagttactcccaaatatgcccaaggacaaagaggcatgtgcaaaagccccattggaggcttacaacaggacactgactactttgcttaccccccttggtgattctatccgcaggatacaagagtctgtgactacatccggaggaaggagacagagacgctctataggtgccattattggcagtgtagctctaggggttgcaacagctgcacagataacggcagcctcggctctgatacaagccaaccagaatgctgctaacatcctccggcttaaggagagcattgctgcaaccaatgaagctgtacacgaggtcactggcggattgtcacagttagcagtggcagttggaaagatgcagcaatttgttaatgaccagcttaataacacagctcaagagttggactgtataaaaattacacagcaggttggtgtagaactcaacttatacttaactgaattgactacagtgttcgggccacaaatcacttcccctgccttaactcagttgactgttcaggctctttacaatctggctggtggtaatgtagattacttgttgactaagttaggtgtagggaacaaccagctcagctcattgattggtagcggcttgatcaccggtaaccctattttttacgactcacagactcaactcttgggcatacaggtgactttaccctcagtcgggaacctaaataatatgcgtgccacctacttggagaccttgtctgtaagcacaacaaagggatttgcctcagcacttgtcccaaaagtagtgacacaggtcggttctgtgatagaagagcttgacacctcatactgtatagaagctgatttggatttatattgtacaagaatagtgacattccctatgtcccctggtatttattcctgtttgagcggcaatacatcggcttgcatgtattcaaggactgaaggcgcacttactacaccatacatgactctcaaaggctcagttgttgccaattgccagatgacaacatgtagatgtgcagaccccccgggtatcatatcacaaaattatggagaggctgtgtctctaatagataagcactcatgcaatgtcgtatccttagacgggataactttgaggctcagtggggaatttgatgcaacttatcaaaagaatatctcaatattagattctcaagtactagtgacaggcaatctcgatatctcaactgagcttgggaatgtcaaccactcaataagtaatgctttggataagttagaggaaagcaacagcaaactagacaaagtcaatgtcaaactgaccagcacatctgctctcattacctatattgttttaactgtcatatctcttgttcttggtatgcttagcctggttctagcatgctatctgatgtacaagcaaaaagcgcaacgaaagaccttgttgtggcttggaaataataccctagatcagatgagagctactacaaaaatgtgaatgcagatgagaggcagaggtatccccaatagcaatctgtgtgtcaattctggcagcctgttaatcagaagaattaagaaaaaactaccggatgtaggtgaacaaaagggaatatacgggtagaacggcctgagaggccacccctcaatcgggagccaggccccactacgtccgctctaccgcatcaccaacagcagtcttcagtcatggacagcgcagttagccaagttgcgctagagaatgatagaagagaagcgaataatacatggcgcttggttttccggatcgcagccttacttttactggtaataaccttagccgtctctgcagtcgccctggcatatagtatggaggctagcacacctggcgaccttgtaagcataccgactgcgatctctagggcagaggaaaggattacatctgcactcggttccaatcaagatgtagtagataggatatacaagcaagtggcccttgagtctccattggcactgctaaataccgaatctataattatgaatgcaataacgtctctctcttatcaaatcaatggagccacaaataatagcgggtgtggggcacctgttcatgacccagattacatcggggggataggtcaagaacttattgtagatgatacgagtgacgtcacatcattttatccctctgcattccaagaacacctgaattttatcccggcgcccactacaggatcaggctgcactcggataccctcattcgacatgagtgctacccattactgttacactcacaatgtgatattgtctggctgcagagatcactcacactcacatcagtatttagcacttggtgtgcttcggacatctgcaacagggagggtattcttttctactctgcgttccatcaacttggatgacgcccaaaatcggaagtcttgcagtgtgagtgcaactcctttaggttgtgatatgctgtgctctaaaatcacagagactgaggaagaggattataaatcagttatccccacatcgatggtacatggaaggttagggcttgacggccaataccatgagaaggacctagacgtcacaacactatttagggactgggtggcaaattacccaggagtaggaggtgggtcttttattaacaatcgcgtatggttcccggtctacggagggctaaaacccagctcgcctagtgacactgcacaagaagggagatacgtaatatacaagcgatacaatgacacatgcccggatgagcaagattaccagattcggatggctaagtcatcgtataagcctaggcggtttggtggaaaacgcgtacagcaggccatcctatccatcaaggtgtcaacatccttgggtgaggacccggtgttgactgtaccgcctaatacggtcgcactcatgggggccgaaggcagagttctcacagtggggacatctcatttcttatatcagcgagggtcatcatacttctctcccgctttgttataccctatgacagtcaacaacaaaacagccactcttcataatccttatacattcaatgctttcactcggccaggtagtgtcccttgccaggcttcagcaagatgccccaactcatgtgttaccggagtctatactgatccatatcccttagtcttccataggaaccacaccttgcgaggggtattcggaacaatgcttgatgacgaacaagcaagactcaaccctgtatctgcagtatttgataacatatcccgcagtcgcataactcgggtaagttcaagcagtaccagggcagcatacacgacatcaacgtgttttaaagttgtcaagaccaataaaacctattgcctcagcattgcagaaatatccaataccctcttcggggaattcagaattgtccccttactagttgagattctcaaggatggtggggtttaagaagctaggaatggtcggttgagtcaaccgtgagaaggcggggaagatgatattgcatcatctatcttctgtaacaccaagaatcaaatcgaataccagtgcgagctcgaatcctacgctgccagtcagccataatcggctggtgctaatgtggttagcctgaatcttgtcgatagtcacttgattaagaaaaaatgtggacggtagtgaaatataaggcaaagcagctcacagaggacagcacgggtaggacatggcgggctccggacccgaaagggcagagcatcagattatcctaccagagtcacatctgtcctcaccattggtcaagcataaattgctttattactggaaattaactgggctaccgcttcctgacgaatgtgacttcgaccatcttattatcagccgacaatggaagaaggtacttgaatcggccacccctgacattgagagaatgataaaactagggcgggcagtacaccagactctcaaccacaattccaggataaccggagtactccatccccggtgtttagaggaactggctagtattgaggttcctgattcaaccaacaaatttcggaagatcgaaaaaaagatccagattcacaacacaaggtatggagaactgttcacaagattatgcacgcatgtagaaaagaaactattggggtcatcttggtctaacaatgtcccacggtcagaggaattcaacagcatccgtacagatccggcattttggtttcactcaaaatggtccacagccaaatttgcatggctccatataaaacaggtccagaggcatctgattgtagcagcaagaacaaggtccgcagtcaacaaattagtgacgctgacccataaggtaggccaaatctttgttactcctgagcttgtcattgtgacacatacagatgagaacaagttcacgtgtcttacccaggaacttgtgttgatgtatgcagatatgatggagggcagagatatggtcagcataatatcatccacggcggcacatcttaggagcttatcagagaaaattgatgatattctgcggttagtagatgctctggcaagagatttgggcaatcaagtctacgatgttgtagcactaatggagggattcgcatacggcgctgttcagctgcttgaaccgtcaggtacatttgcgggggatttcttcgcattcaacctgcaggagctcaaagatactctaaccggactcctccccaaggatatcgcagaatctgtgactcacgcaatcgcaaccatattctctggcttagaacaaaatcaagcagctgagatgttgtgcctgttgcgtctgtggggtcacccactactcgagtcccgtattgcagcaaaagcagttaggagtcaaatgtgcgcaccaaaaatggtagactttgatatgatcctccaggtattatctttctttaaaggaacaatcatcaatggatatagaaagaagaatgcaggcgtgtggccacgtgtcaagatagatacgatatacgggaaggtcatagggcagctacacgcagattctgcagagatttcacatgatgtcatgttgagggaatacaagagtttatctgcacttgaattcgagccatgtatagagtatgaccctgtcaccaatctgagcatgtttttaaaagacaaggcaatcgcacacccgaaagacaactggctcgcttcgtttaggcgaaaccttctctctgaggaccagaagaaacatgtaagagaggcaacctcaactaaccgcctcttgatagagttcttagagtcaaatgattttgatccatataaagagatggaatatctgacgacccttgagtacctaagagatgataatgtggcagtatcatactcactcaaagaaaaagaggtgaaagttaatgggcggattttcgcaaagctaacaaatagattaaggaattgtcaggtaatggcagaagggatcctagctgaccagattgcacctttcttccagggaaatggggtcattcaggatagcatatctttgactaaaagtatgctagcgatgagtcaactgtctttcaacagcaacaagaaacgtattactgactgcaaagaaagagtatcctcaaaccgcaatcatgatccgaagagcaagaatcgtcggagagttgccacttttgtaactaccgacctgcaaaagtattgtctcaattggagatatcagacagtcaagctgttcgctcatgccatcaatcagctgatgggcttacctcacttcttcgagtggattcatcttagactaatggatactacgatgtttgtaggggaccctttcaatcctccaagtgaccctacggactgtgatctatcaagagtcccaaatgatgacatatatattgtcagtgctaggggaggcattgagggattatgccagaagctatggacaatgatctcaattgctgcaatccagcttgctgcagcaagatcacattgtcgcgttgcctgtatggtacaaggcgacaatcaagtaatagctataacgagagaggtaagatcagatgactccccagagatggtgttaacacaattgcatcaagccagtgataatttcttcaaggaattgattcatgtcaatcatttgatcggccataatttgaaggatcgtgaaaccatcaggtcagacacgttcttcatatacagcaaacgaatattcaaagatggagcaatactcagtcaggtcctcaaaaactcatctaaattagtgctaatatcaggcgaccttagtgaaaacactgtaatgtcttgtgccaacattgcatctactgtagcacggttatgcgagaacgggcttcctaaggatttctgttattacttaaactacttaatgagttgcgtgcagacatacttcgattccgaattttccatcaccaacaactcgcaactcgattctaaccagtcgtggatagaggacatttcttttgtgcactcatatgtcctgacccctgctcaactggggggactgagtaaccttcaatactcaaggctctacacaaggaacatcggcgacccgggaaccactgctttcgcagagatcaagagattagaggcagtggggttactgagtcctagcattatgactaacatcttaactaggccgcctggaaatggagattgggccagtctgtgcaacgatccatactcctttaattttgagactgtcgcaagcccaaatattgtccttaagaaacatacacaaagagtcctatttgaaacttgctcgaatcccttattatctggagtgcacacagaggataatgaggcagaagagaaggcattggctgaattcttgctcaatcaagaaatgattcatccacgtgtcgcgcatgctatcatggaagcaagctctgtaggtaggagaaagcaaattcaggggcttgttgacacaacaaacaccgtgattaagattgcactgactaggaggccactcggcatcaagaggctgatgcggatagtcaattactcgagcatgcatgcaatgctatttagagatgatgttttctcgtctaatagatccaaccatcccttagtctcttctgatatgtgttctcagacactggcagactatgcacggaacagaagctggtcacctttgacagggggtagaaaaatactgggtgtatctaatcctgataccatagaacttgtagagggtgagatccttagtgtcagtggagggtgcactagatgtgacagtggggatgaacagtttacttggttccatcttccaagcaatatagagctaaacgatgacaccagcaagaatcctccaatgagagtgccatatctcgggtcaaagactcaagagaggagagccgcctcgcttgcgaaaatagctcatatgtcaccacacgtgaaggcggctctaagggcgtcatctgtgttaatctgggcttatggggacaacgaaataaactggactgctgcccttaagattgcaaggtctcggtgcaacataagctcagagtatcttcgactattgtcacccttgcctacagctgggaatctccaacatagattggacgacggcataactcagatgacattcacccctgcatctctatacagggtgtcaccttacattcatatatctaatgattctcaaaggctatttactgaagaaggagtcaaagaggggaatgtggtttatcagcaaatcatgctcttgggtttgtccttaattgagtcactcttcccaatgacaacaaccaagacatatgatgaaatcacattgcacctccacagtaaatttagctgctgtatcagggaagcacctgttgcagttcctttcgagctactcggggtggcaccggaactaagggcagtaacctcaaataagtttatgtatgatcctagccctgtatcagagggagactttgcgagacttgacttagctatctttaagagttatgaacttaatttagagtcatattccacaatagagctaatgaacgttctttcaatatctagtgggaagttgattggccagtctgtggtttcttatgatgaagatacctccataaagaatgacgctataatagtgtatgacaacacacggaattggatcagcgaagctcagaattcagatgtggtccgcctattcgagtatgcggcactcgaagtgctcctcgactgttcttatcaactctactatctgagagtaagaggcctaaacaatatcgtcctgtacatgagtgatttatacaagaatatgccaggaattctactctctaatattgcagccacaatatctcaccctgtcatccattcaaggttgaatgcagtaggtctggtcaaccatgacgggtcacaccaacttgcagacacagatttcattgaaatgtctgcaaagctgctagtctcttgcactcgacgcgtggtctcaggtttacatgcagggaataagtatgacctgctgtttccatctgtcttggatgataacctaagtgagaagatgcttcagttgatatcccaattatgctgtctgtatacggtgctctttgctacaacaagagaaatcccgaaaataagaggcttatctgcagaagagaaatgttcagtacttactgagtacctactgtcagatgctgtgaaaccgttacttgggtccgagcaagtgagctctatcatgtctcccaacatagttacgttcccagccaatctgtattacatgtctaggaagagccttaatttgatcagggagagagaggacagggatactatcttggcattgttgttccctcaagaaccgctgctcgagtttcctctggtacgagatattggtgctcgtgtaaaagatccatttaccctacaacctgcggcgtttttacaagagttagatttgagtgctccggcaaggtatgacgcattcacaatcagtcaggcgcattctgaacacatattgccaaacccagaggaagatcacttagtacgatacttgttcagaggaatagggactgcgtcctcctcttggtataaggcatcccatcttctttctgtacccgaggtcagatgtgcaaggcatgggaactccttatatttagcagaaggaagtggagccatcatgagtcttctcgaattgcatataccacacgaaactatctattacaatacacttttctcgaacgagatgaaccccccacagcgacatttcggaccaactccaacacagtttctgaattcggttgtttttaggaatttacaggcggaagtaccatgcaaggatggatttgtccaggagttccgtccgttatggagagagaatacagaagaaagcgatctgacctcagataaagcagtgggatatatcacatctgtagtgccgtacaggtctgtatcattgctgcattgtgacattgaaatccctccaggatctaatcaaagcttactagatcagctggctaccaatctgtctctgattgccatgcattctgtgaaggagggcggggtcgtgattatcaaagtactgtatgcaatgggatattacttccatctactcatgaacttgtttactccatgttccacgaaaggatatattctctctaatggctatgcctgtagaggggatatggagtgttacctgatatttgtcatgggctacctaggcgggcctacatttgtacatgaggtggtgaggatggcaaaaacgctagtacagcggcatggcacacttttgtctaagtcagatgagattacactgactaggttatttacttcacagcaacagcgtgtaacagacatcctatccagccctttaccgagactaatgaagtacttgagagagaatattgatactgcactgattgaagccgggggacagcctgtccgtccattctgtgcagagagtttagtgagcacactaacagacatgactcagacaacccagatcatcgccagccacattgatacagtcattcgatctgtgatctatatggaagcagagggtgatcttgctgacacagtattcttatttaccccttacaacctctctatggacgggaaaaagagaacatcacttaaacagtgcacaagacagatcttagaggtcacaatactgggtctcagagtcaaaaatctcaataaagtaggtgatgtaatcagcctagtactcagaggtatgatttctctggaggaccttctcccactgagaacctacttgaagtgtagtacctgccctaagtatttgaaggctgtcctaggtattaccaaactcaaagaaatgttcacagacgcctctttattatacttgactcgtgctcaacaaaaattctacatgaaaactataggcaatgcagtcaagggatactatagtaactgtgactcttaaaggcaaccacatatcaataggccctctttctagccgatcgtattcttgttgacttcattataccatattagaaaaaaattgaattccgaccctttaagactcgtattcggattcaaataattatctcagaaaaaaagtgcacgtagttgttcttgattatagtcccgtcattcaccaagtctttgtttggt

SEQ ID NO: 6 - DNA, 15186 bp, Newcastle disease virus
  Mut HN(F277L)/M(G165W)/F(F117S)/F(F190L)/ F(L289A)/L(V757I)/L(F1551S)/L(R1700L)
accaaacagagaatccgtgagatacgataaaaggcgaaggagcaatcgaagtcgtacgggtagaaggtgtgaatctcgagtgcgagaccgaagcttaaactcgagagagccttctgccaacatgtcttccgtatttgacgagtatgaacagctcctcgcggctcagactcgccctaatggagctcacggaggaggagagaaggggagcaccttaaaagttgaagtcccggtattcactcttaacagtgacgacccagaagatagatggaactttgcagtattctgtcttaggattgctgttagcgaagatgccaacaaaccactcagacaaggtgctctcatatctctcttatgctcccattctcaagtgatgaggaaccatgtcgcccttgcagggaaacagaatgaggccacactggctgttcttgagatcgatggtttcaccaacagcgtgcctcaatttaacaacaggagtggagtgtctgaggagagagcacagagattcttgatgatagcagggtccctccctcgggcatgcagcaacggtaccccgttcatcacagctggggttgaagatgatgcaccagaagacatcactgatactctggaaagaatcatctctatccaggctcaagtatgggtcacggtagcaaaggccatgactgcatatgagacagcggatgagtcggaaacaagaagaatcaataagtatatgcagcaaggcagagtccaaaagaagtacatcctccaccccgtatgtaggagcgcaattcaactcacgattagacaatctctggcagtccgcattttcttggttagcgagcttaagagaggccgcaacacggcaggtggaagttccacttattatagcttggtaggggacatagactcatacatcaggaacaccgggcttactgcattcttcctgacactcaggtatggaattaacaccaagacatcagcccttgcactcagcagcctcgcaggcgatatccaaaaaatgaagcagctcatgcgtctatatcggatgaaaggagataatgcgccgtacatgacactgctcggtgacagtgaccagatgagctttgcaccggctgagtatgcacaactctactcttttgcaatgggtatggcatccgtcctagacaaaggaactggcaagtaccagttcgccagagactttatgagcacatcattctggagacttggagtagagtatgctcgggctcagggaagcagcatcaatgaggatatggctgccgagctaaagctgaccccagcggcaaggaggggcctggcagctgctgcccaacgagcgtccgaggagaccggcagcatggacattcccactcaacaagctggggtcctcacagggctcagcgacggaggccctcaagccccacaaggtggactgaacaggtcgcaagggcaaccggacgccggagatggggagacccaatttttagatttgatgagagcagtggcaaatagtatgagagaagcgccaaactctgtgcagaacaccactcagcaggagcctccatctactcctgggccatctcaagacaacgacaccgactggggatactgaccgacaacactcagcctgcctccatcgaattatctcgattcttctgcctgtaacctaacccctggtccacagtctcacacggccaaacccgcaaacgagcccccccctctctccccccccctccagccacacggctccaaccgcccaaaacaacacgggcacaactcgactcactcataatctacacagagccgaagatcttagaaaaaaatacgggtagaagagagacattcagagatcaggacaaatcatcagggtctccgctctcccctctacccagcagaccagagtgaagatggccaccttcacggatgcagagatcgacgatctatttgaaaccagtggaactgtcattgacagcataattacggcccaaggcaacccagtagagactgttggaaggagcgcaatcccacaaggcaaaactaaagcattgagtgcagcatgggagaaacatggaagcacccaaccgccggccggccaagacacccctgatcgaatggacagatcagacaaacaaccatccccacccgaacaggcaaccacacacaacagcccgccagccacatccaccgaccagccccccacccaggccgcaggcgaagccggtgacacacagctcaagactggagcaagcaactccctcctgtcgatgctcgacaagcttagcaataaatcgtccaatgctaaaaagggcccacggtcgagtccccaggaagggcaccaccaacctccgacccagcagcaagggagtcaacagagccgcggaaacagtcaggagagatcgcaaaaccaggtcaaggccgcctctggagaccagggcacagacgcgaacacagcatatcatggacaatggaaggagtcacaaccatcagctggtgcaatccctcatgctctccggtcagggcagagccaagccaatactcctgcatctgtggatcatgtccagctacctgtcgactttgtgcaggcgatgatgtctatgatggaggcgatatcacagaaggtaaataaagtcgactatcagctagaccttgtctcaaagcagacatcctccatccccatgatgcgatctgaaatccaacagctgaaaacatccgttgcggtcatggaagccaacctgggcatgatgaagattctggaccctggttgtgctaacgtttcatctttaagtgacctacgggcggtcgcccgctctcacccagttttagtttcaggccccggagacccatctccttatgtgacacaagggggtgaattgatactcaataaactctcgcaaccagtgcaacatccttctgagttgattaaacctgccacgataagcgggcctgatataggagtggagaaggacactgtccgtgcattgatcacctcacgcccaatgcatccgagctcttcagctaagctcctgagtaagctggatgcagccgggtcgatagaagaaattaggaaaatcaaacgccttgcgctgaatggctgatcactactacaacccgcaacgggttcccgtttatctattgtcacaaggaacccgccccgagccctcctctataaacccaagattcaaggctccaagcgataaccttctcccgcctcttccatcccactgaatgatcgcgcagctgcaattaatctagcaacattaaagattaagaaaaaatacgggtagaatcggagtgcaccgattgtgccaagatggactcatctaggacaattgggttatactttgattctgcccttccttctagcaacctgttagcattcccgattgtcttacgagatgtaggagatggaaagaagcaaattactccgcaatataggatccggcgtcttgactcgtggacagacagtaaagaagactcggtattcatcactacctacggattcatcttccaggttgggaatgaagaagtcactgtcggcatgatcaatgataatcccaagcgagagttactctccgctgcgatgctctgcctaggaagcgtcccgaatgttggagatcctgttgagctggcaagggcctgcctcactatggtggtaacatgcaagaagagtgcaactaatactgagagaatggtcttctcagtagtgcaggcaccccgggtgctgcaaagctgtagggtcgtggcagacaaatactcgtcagtgaatgcagttaagcacgtgaaagcaccagagaagatcccttggagtgaaaccctagagtacaaggtgaattttgtctctttgactgtggtgccgaagaaggatgtctacaagatcccaaccgcagtattgaaggtatccggctcgagcctgtataatcttgcgctcaatgtcactattgatgtggaggtagacccaaagagcccgttagtcaagtccctttctaggtccgacagtggatactatgctaatctcttcttacatatcggacttatgtccactgtagataagaggggaaagaaagtgacatttgaccagctggagaggaagataaggagacttgatctatctgtcgggctcagtgatgtgctcggaccttctgtgcttgtgaaggcgagaggtgcacggactaggctgctggcacctttcttctctaacagcgggacagcctgctaccctatagcaaatgcctctcctcaggtagctaagatactctggagtcagaccgcgtgcctgcggagtgtaaaaatcattattcaagcgggcacccaacgcgctgtcgcagtgactgctgaccatgaggttacctctactaagatagagaagaggcataccatcgctaaatacaatcccttcaagaaataggccgtatccctgagactgcaattcacccgccttcccaaaacaccatgacaccagataatgatctgtcttgattacttacagttagtttccctgtctatcaaattagaaaaaacacgggtagaagagttcggatcccggccggcgcaccaaaagcgcaagatgggccccagatcttctaccaggatcccagtacctctaatgctgaccatacggatcacgctggcactgagttatgtccgtctgacaagttctcttgatggcaggcctcttgcagccgcagggatcgtggtaacaggggataaagcagttaacatatacacctcatcccagacagggtcaatcatagtcaagttactcccaaatatgcccaaggacaaagaggcatgtgcaaaagccccattggaggcttacaacaggacactgactactttgcttaccccccttggtgattctatccgcaggatacaagagtctgtgactacatccggaggaaggagacagagacgctctataggtgccattattggcagtgtagctctaggggttgcaacagctgcacagataacggcagcctcggctctgatacaagccaaccagaatgctgctaacatcctccggcttaaggagagcattgctgcaaccaatgaagctgtacacgaggtcactggcggattgtcacagttagcagtggcagttggaaagatgcagcaatttgttaatgaccagcttaataacacagctcaagagttggactgtataaaaattacacagcaggttggtgtagaactcaacttatacttaactgaattgactacagtgttcgggccacaaatcacttcccctgccttaactcagttgactgttcaggctctttacaatctggctggtggtaatgtagattacttgttgactaagttaggtgtagggaacaaccagctcagctcattgattggtagcggcttgatcaccggtaaccctattttttacgactcacagactcaactcttgggcatacaggtgactgcaccctcagtcgggaacctaaataatatgcgtgccacctacttggagaccttgtctgtaagcacaacaaagggatttgcctcagcacttgtcccaaaagtagtgacacaggtcggttctgtgatagaagagcttgacacctcatactgtatagaagctgatttggatttatattgtacaagaatagtgacattccctatgtcccctggtatttattcctgtttgagcggcaatacatcggcttgcatgtattcaaggactgaaggcgcacttactacaccatacatgactctcaaaggctcagttgttgccaattgccagatgacaacatgtagatgtgcagaccccccgggtatcatatcacaaaattatggagaggctgtgtctctaatagataagcactcatgcaatgtcgtatccttagacgggataactttgaggctcagtggggaatttgatgcaacttatcaaaagaatatctcaatattagattctcaagtactagtgacaggcaatctcgatatctcaactgagcttgggaatgtcaaccactcaataagtaatgctttggataagttagaggaaagcaacagcaaactagacaaagtcaatgtcaaactgaccagcacatctgctctcattacctatattgttttaactgtcatatctcttgttcttggtatgcttagcctggttctagcatgctatctgatgtacaagcaaaaagcgcaacgaaagaccttgttgtggcttggaaataataccctagatcagatgagagctactacaaaaatgtgaatgcagatgagaggcagaggtatccccaatagcaatctgtgtgtcaattctggcagcctgttaatcagaagaattaagaaaaaactaccggatgtaggtgaacaaaagggaatatacgggtagaacggcctgagaggccacccctcaatcgggagccaggccccactacgtccgctctaccgcatcaccaacagcagtcttcagtcatggacagcgcagttagccaagttgcgctagagaatgatagaagagaagcgaataatacatggcgcttggttttccggatcgcagccttacttttactggtaataaccttagccgtctctgcagtcgccctggcatatagtatggaggctagcacacctggcgaccttgtaagcataccgactgcgatctctagggcagaggaaaggattacatctgcactcggttccaatcaagatgtagtagataggatatacaagcaagtggcccttgagtctccattggcactgctaaataccgaatctataattatgaatgcaataacgtctctctcttatcaaatcaatggagccacaaataatagcgggtgtggggcacctgttcatgacccagattacatcggggggataggtcaagaacttattgtagatgatacgagtgacgtcacatcattttatccctctgcattccaagaacacctgaattttatcccggcgcccactacaggatcaggctgcactcggataccctcattcgacatgagtgctacccattactgttacactcacaatgtgatattgtctggctgcagagatcactcacactcacatcagtatttagcacttggtgtgcttcggacatctgcaacagggagggtattcttttctactctgcgttccatcaacttggatgacgcccaaaatcggaagtcttgcagtgtgagtgcaactcctttaggttgtgatatgctgtgctctaaaatcacagagactgaggaagaggattataaatcagttatccccacatcgatggtacatggaaggttagggcttgacggccaataccatgagaaggacctagacgtcacaacactatttagggactgggtggcaaattacccaggagtaggaggtgggtcttttattaacaatcgcgtatggttcccggtctacggagggctaaaacccagctcgcctagtgacactgcacaagaagggagatacgtaatatacaagcgatacaatgacacatgcccggatgagcaagattaccagattcggatggctaagtcatcgtataagcctaggcggtttggtggaaaacgcgtacagcaggccatcctatccatcaaggtgtcaacatccttgggtgaggacccggtgttgactgtaccgcctaatacggtcgcactcatgggggccgaaggcagagttctcacagtggggacatctcatttcttatatcagcgagggtcatcatacttctctcccgctttgttataccctatgacagtcaacaacaaaacagccactcttcataatccttatacattcaatgctttcactcggccaggtagtgtcccttgccaggcttcagcaagatgccccaactcatgtgttaccggagtctatactgatccatatcccttagtcttccataggaaccacaccttgcgaggggtattcggaacaatgcttgatgacgaacaagcaagactcaaccctgtatctgcagtatttgataacatatcccgcagtcgcataactcgggtaagttcaagcagtaccagggcagcatacacgacatcaacgtgttttaaagttgtcaagaccaataaaacctattgcctcagcattgcagaaatatccaataccctcttcggggaattcagaattgtccccttactagttgagattctcaaggatggtggggtttaagaagctaggaatggtcggttgagtcaaccgtgagaaggcggggaagatgatattgcatcatctatcttctgtaacaccaagaatcaaatcgaataccagtgcgagctcgaatcctacgctgccagtcagccataatcggctggtgctaatgtggttagcctgaatcttgtcgatagtcacttgattaagaaaaaatgtggacggtagtgaaatataaggcaaagcagctcacagaggacagcacgggtaggacatggcgggctccggacccgaaagggcagagcatcagattatcctaccagagtcacatctgtcctcaccattggtcaagcataaattgctttattactggaaattaactgggctaccgcttcctgacgaatgtgacttcgaccatcttattatcagccgacaatggaagaaggtacttgaatcggccacccctgacattgagagaatgataaaactagggcgggcagtacaccagactctcaaccacaattccaggataaccggagtactccatccccggtgtttagaggaactggctagtattgaggttcctgattcaaccaacaaatttcggaagatcgaaaaaaagatccagattcacaacacaaggtatggagaactgttcacaagattatgcacgcatgtagaaaagaaactattggggtcatcttggtctaacaatgtcccacggtcagaggaattcaacagcatccgtacagatccggcattttggtttcactcaaaatggtccacagccaaatttgcatggctccatataaaacaggtccagaggcatctgattgtagcagcaagaacaaggtccgcagtcaacaaattagtgacgctgacccataaggtaggccaaatctttgttactcctgagcttgtcattgtgacacatacagatgagaacaagttcacgtgtcttacccaggaacttgtgttgatgtatgcagatatgatggagggcagagatatggtcagcataatatcatccacggcggcacatcttaggagcttatcagagaaaattgatgatattctgcggttagtagatgctctggcaagagatttgggcaatcaagtctacgatgttgtagcactaatggagggattcgcatacggcgctgttcagctgcttgaaccgtcaggtacatttgcgggggatttcttcgcattcaacctgcaggagctcaaagatactctaaccggactcctccccaaggatatcgcagaatctgtgactcacgcaatcgcaaccatattctctggcttagaacaaaatcaagcagctgagatgttgtgcctgttgcgtctgtggggtcacccactactcgagtcccgtattgcagcaaaagcagttaggagtcaaatgtgcgcaccaaaaatggtagactttgatatgatcctccaggtattatctttctttaaaggaacaatcatcaatggatatagaaagaagaatgcaggcgtgtggccacgtgtcaagatagatacgatatacgggaaggtcatagggcagctacacgcagattctgcagagatttcacatgatgtcatgttgagggaatacaagagtttatctgcacttgaattcgagccatgtatagagtatgaccctgtcaccaatctgagcatgtttttaaaagacaaggcaatcgcacacccgaaagacaactggctcgcttcgtttaggcgaaaccttctctctgaggaccagaagaaacatgtaagagaggcaacctcaactaaccgcctcttgatagagttcttagagtcaaatgattttgatccatataaagagatggaatatctgacgacccttgagtacctaagagatgataatgtggcagtatcatactcactcaaagaaaaagaggtgaaagttaatgggcggattttcgcaaagctaacaaatagattaaggaattgtcaggtaatggcagaagggatcctagctgaccagattgcacctttcttccagggaaatggggtcattcaggatagcatatctttgactaaaagtatgctagcgatgagtcaactgtctttcaacagcaacaagaaacgtattactgactgcaaagaaagagtatcctcaaaccgcaatcatgatccgaagagcaagaatcgtcggagagttgccacttttgtaactaccgacctgcaaaagtattgtctcaattggagatatcagacagtcaagctgttcgctcatgccatcaatcagctgatgggcttacctcacttcttcgagtggattcatcttagactaatggatactacgatgtttgtaggggaccctttcaatcctccaagtgaccctacggactgtgatctatcaagagtcccaaatgatgacatatatattgtcagtgctaggggaggcattgagggattatgccagaagctatggacaatgatctcaattgctgcaatccagcttgctgcagcaagatcacattgtcgcgttgcctgtatggtacaaggcgacaatcaagtaatagctataacgagagaggtaagatcagatgactccccagagatggtgttaacacaattgcatcaagccagtgataatttcttcaaggaattgattcatgtcaatcatttgatcggccataatttgaaggatcgtgaaaccatcaggtcagacacgttcttcatatacagcaaacgaatattcaaagatggagcaatactcagtcaggtcctcaaaaactcatctaaattagtgctaatatcaggcgaccttagtgaaaacactgtaatgtcttgtgccaacattgcatctactgtagcacggttatgcgagaacgggcttcctaaggatttctgttattacttaaactacttaatgagttgcgtgcagacatacttcgattccgaattttccatcaccaacaactcgcaactcgattctaaccagtcgtggatagaggacatttcttttgtgcactcatatgtcctgacccctgctcaactggggggactgagtaaccttcaatactcaaggctctacacaaggaacatcggcgacccgggaaccactgctttcgcagagatcaagagattagaggcagtggggttactgagtcctagcattatgactaacatcttaactaggccgcctggaaatggagattgggccagtctgtgcaacgatccatactcctttaattttgagactgtcgcaagcccaaatattgtccttaagaaacatacacaaagagtcctatttgaaacttgctcgaatcccttattatctggagtgcacacagaggataatgaggcagaagagaaggcattggctgaattcttgctcaatcaagaaatgattcatccacgtgtcgcgcatgctatcatggaagcaagctctgtaggtaggagaaagcaaattcaggggcttgttgacacaacaaacaccgtgattaagattgcactgactaggaggccactcggcatcaagaggctgatgcggatagtcaattactcgagcatgcatgcaatgctatttagagatgatgttttctcgtctaatagatccaaccatcccttagtctcttctgatatgtgttctcagacactggcagactatgcacggaacagaagctggtcacctttgacagggggtagaaaaatactgggtgtatctaatcctgataccatagaacttgtagagggtgagatccttagtgtcagtggagggtgcactagatgtgacagtggggatgaacagtttacttggttccatcttccaagcaatatagagctaaacgatgacaccagcaagaatcctccaatgagagtgccatatctcgggtcaaagactcaagagaggagagccgcctcgcttgcgaaaatagctcatatgtcaccacacgtgaaggcggctctaagggcgtcatctgtgttaatctgggcttatggggacaacgaaataaactggactgctgcccttaagattgcaaggtctcggtgcaacataagctcagagtatcttcgactattgtcacccttgcctacagctgggaatctccaacatagattggacgacggcataactcagatgacattcacccctgcatctctatacagggtgtcaccttacattcatatatctaatgattctcaaaggctatttactgaagaaggagtcaaagaggggaatgtggtttatcagcaaatcatgctcttgggtttgtccttaattgagtcactcttcccaatgacaacaaccaagacatatgatgaaatcacattgcacctccacagtaaatttagctgctgtatcagggaagcacctgttgcagttcctttcgagctactcggggtggcaccggaactaagggcagtaacctcaaataagtttatgtatgatcctagccctgtatcagagggagactttgcgagacttgacttagctatctttaagagttatgaacttaatttagagtcatattccacaatagagctaatgaacgttctttcaatatctagtgggaagttgattggccagtctgtggtttcttatgatgaagatacctccataaagaatgacgctataatagtgtatgacaacacacggaattggatcagcgaagctcagaattcagatgtggtccgcctattcgagtatgcggcactcgaagtgctcctcgactgttcttatcaactctactatctgagagtaagaggcctaaacaatatcgtcctgtacatgagtgatttatacaagaatatgccaggaattctactctctaatattgcagccacaatatctcaccctgtcatccattcaaggttgaatgcagtaggtctggtcaaccatgacgggtcacaccaacttgcagacacagatttcattgaaatgtctgcaaagctgctagtctcttgcactcgacgcgtggtctcaggtttacatgcagggaataagtatgacctgctgtttccatctgtcttggatgataacctaagtgagaagatgcttcagttgatatcccaattatgctgtctgtatacggtgctctttgctacaacaagagaaatcccgaaaataagaggcttatctgcagaagagaaatgttcagtacttactgagtacctactgtcagatgctgtgaaaccgttacttgggtccgagcaagtgagctctatcatgtctcccaacatagttacgttcccagccaatctgtattacatgtctaggaagagccttaatttgatcagggagagagaggacagggatactatcttggcattgttgttccctcaagaaccgctgctcgagtttcctctggtacgagatattggtgctcgtgtaaaagatccatttaccctacaacctgcggcgtttttacaagagttagatttgagtgctccggcaaggtatgacgcattcacaatcagtcaggcgcattctgaacacatattgccaaacccagaggaagatcacttagtacgatacttgttcagaggaatagggactgcgtcctcctcttggtataaggcatcccatcttctttctgtacccgaggtcagatgtgcaaggcatgggaactccttatatttagcagaaggaagtggagccatcatgagtcttctcgaattgcatataccacacgaaactatctattacaatacacttttctcgaacgagatgaaccccccacagcgacatttcggaccaactccaacacagtttctgaattcggttgtttttaggaatttacaggcggaagtaccatgcaaggatggatttgtccaggagttccgtccgttatggagagagaatacagaagaaagcgatctgacctcagataaagcagtgggatatatcacatctgtagtgccgtacaggtctgtatcattgctgcattgtgacattgaaatccctccaggatctaatcaaagcttactagatcagctggctaccaatctgtctctgattgccatgcattctgtgaaggagggcggggtcgtgattatcaaagtactgtatgcaatgggatattacttccatctactcatgaacttgtttactccatgttccacgaaaggatatattctctctaatggctatgcctgtagaggggatatggagtgttacctgatatttgtcatgggctacctaggcgggcctacatttgtacatgaggtggtgaggatggcaaaaacgctagtacagcggcatggcacacttttgtctaagtcagatgagattacactgactaggttatttacttcacagcaacagcgtgtaacagacatcctatccagccctttaccgagactaatgaagtacttgagagagaatattgatactgcactgattgaagccgggggacagcctgtccgtccattctgtgcagagagtttagtgagcacactaacagacatgactcagacaacccagatcatcgccagccacattgatacagtcattcgatctgtgatctatatggaagcagagggtgatcttgctgacacagtattcttatttaccccttacaacctctctatggacgggaaaaagagaacatcacttaaacagtgcacaagacagatcttagaggtcacaatactgggtctcagagtcaaaaatctcaataaagtaggtgatgtaatcagcctagtactcagaggtatgatttctctggaggaccttctcccactgagaacctacttgaagtgtagtacctgccctaagtatttgaaggctgtcctaggtattaccaaactcaaagaaatgttcacagacgcctctttattatacttgactcgtgctcaacaaaaattctacatgaaaactataggcaatgcagtcaagggatactatagtaactgtgactcttaaaggcaaccacatatcaataggccctctttctagccgatcgtattcttgttgacttcattataccatattagaaaaaaattgaattccgaccctttaagactcgtattcggattcaaataattatctcagaaaaaaagtgcacgtagttgttcttgattatagtcccgtcattcaccaagtctttgtttggt

SEQ ID NO: 7 - DNA, 15198 bp, Newcastle disease virus
  Mut HN(F277L)/M(G165W)/F(F117S)/F(F190L)/ F(L289A)/L(V757I)/L(F1551S)/L(R1700L)+AfeI
accaaacagagaatccgtgagatacgataaaaggcgaaggagcaatcgaagtcgtacgggtagaaggtgtgaatctcgagtgcgagaccgaagcttaaactcgagagagccttctgccaacatgtcttccgtatttgacgagtatgaacagctcctcgcggctcagactcgccctaatggagctcacggaggaggagagaaggggagcaccttaaaagttgaagtcccggtattcactcttaacagtgacgacccagaagatagatggaactttgcagtattctgtcttaggattgctgttagcgaagatgccaacaaaccactcagacaaggtgctctcatatctctcttatgctcccattctcaagtgatgaggaaccatgtcgcccttgcagggaaacagaatgaggccacactggctgttcttgagatcgatggtttcaccaacagcgtgcctcaatttaacaacaggagtggagtgtctgaggagagagcacagagattcttgatgatagcagggtccctccctcgggcatgcagcaacggtaccccgttcatcacagctggggttgaagatgatgcaccagaagacatcactgatactctggaaagaatcatctctatccaggctcaagtatgggtcacggtagcaaaggccatgactgcatatgagacagcggatgagtcggaaacaagaagaatcaataagtatatgcagcaaggcagagtccaaaagaagtacatcctccaccccgtatgtaggagcgcaattcaactcacgattagacaatctctggcagtccgcattttcttggttagcgagcttaagagaggccgcaacacggcaggtggaagttccacttattatagcttggtaggggacatagactcatacatcaggaacaccgggcttactgcattcttcctgacactcaggtatggaattaacaccaagacatcagcccttgcactcagcagcctcgcaggcgatatccaaaaaatgaagcagctcatgcgtctatatcggatgaaaggagataatgcgccgtacatgacactgctcggtgacagtgaccagatgagctttgcaccggctgagtatgcacaactctactcttttgcaatgggtatggcatccgtcctagacaaaggaactggcaagtaccagttcgccagagactttatgagcacatcattctggagacttggagtagagtatgctcgggctcagggaagcagcatcaatgaggatatggctgccgagctaaagctgaccccagcggcaaggaggggcctggcagctgctgcccaacgagcgtccgaggagaccggcagcatggacattcccactcaacaagctggggtcctcacagggctcagcgacggaggccctcaagccccacaaggtggactgaacaggtcgcaagggcaaccggacgccggagatggggagacccaatttttagatttgatgagagcagtggcaaatagtatgagagaagcgccaaactctgtgcagaacaccactcagcaggagcctccatctactcctgggccatctcaagacaacgacaccgactggggatactgaccgacaacactcagcctgcctccatcgaattatctcgattcttctgcctgtaacctaacccctggtccacagtctcacacggccaaacccgcaaacgagcccccccctctctccccccccctccagccacacggctccaaccgcccaaaacaacacgggcacaactcgactcactcataatctacacagagccgaagatcttagaaaaaaatacgggtagaagagagacattcagagatcaggacaaatcatcagggtctccgctctcccctctacccagcagaccagagtgaagatggccaccttcacggatgcagagatcgacgatctatttgaaaccagtggaactgtcattgacagcataattacggcccaaggcaacccagtagagactgttggaaggagcgcaatcccacaaggcaaaactaaagcattgagtgcagcatgggagaaacatggaagcacccaaccgccggccggccaagacacccctgatcgaatggacagatcagacaaacaaccatccccacccgaacaggcaaccacacacaacagcccgccagccacatccaccgaccagccccccacccaggccgcaggcgaagccggtgacacacagctcaagactggagcaagcaactccctcctgtcgatgctcgacaagcttagcaataaatcgtccaatgctaaaaagggcccacggtcgagtccccaggaagggcaccaccaacctccgacccagcagcaagggagtcaacagagccgcggaaacagtcaggagagatcgcaaaaccaggtcaaggccgcctctggagaccagggcacagacgcgaacacagcatatcatggacaatggaaggagtcacaaccatcagctggtgcaatccctcatgctctccggtcagggcagagccaagccaatactcctgcatctgtggatcatgtccagctacctgtcgactttgtgcaggcgatgatgtctatgatggaggcgatatcacagaaggtaaataaagtcgactatcagctagaccttgtctcaaagcagacatcctccatccccatgatgcgatctgaaatccaacagctgaaaacatccgttgcggtcatggaagccaacctgggcatgatgaagattctggaccctggttgtgctaacgtttcatctttaagtgacctacgggcggtcgcccgctctcacccagttttagtttcaggccccggagacccatctccttatgtgacacaagggggtgaattgatactcaataaactctcgcaaccagtgcaacatccttctgagttgattaaacctgccacgataagcgggcctgatataggagtggagaaggacactgtccgtgcattgatcacctcacgcccaatgcatccgagctcttcagctaagctcctgagtaagctggatgcagccgggtcgatagaagaaattaggaaaatcaaacgccttgcgctgaatggctgatcactactacaacccgcaacgggttcccgtttatctattgtcacaaggaacccgccccgaagcgctgaccgagccctcctctataaacccaagattcaaggctccaagcgataaccttctcccgcctcttccatcccactgaatgatcgcgcagctgcaattaatctagcaacattaaagattaagaaaaaatacgggtagaatcggagtgcaccgattgtgccaagatggactcatctaggacaattgggttatactttgattctgcccttccttctagcaacctgttagcattcccgattgtcttacgagatgtaggagatggaaagaagcaaattactccgcaatataggatccggcgtcttgactcgtggacagacagtaaagaagactcggtattcatcactacctacggattcatcttccaggttgggaatgaagaagtcactgtcggcatgatcaatgataatcccaagcgagagttactctccgctgcgatgctctgcctaggaagcgtcccgaatgttggagatcctgttgagctggcaagggcctgcctcactatggtggtaacatgcaagaagagtgcaactaatactgagagaatggtcttctcagtagtgcaggcaccccgggtgctgcaaagctgtagggtcgtggcagacaaatactcgtcagtgaatgcagttaagcacgtgaaagcaccagagaagatcccttggagtgaaaccctagagtacaaggtgaattttgtctctttgactgtggtgccgaagaaggatgtctacaagatcccaaccgcagtattgaaggtatccggctcgagcctgtataatcttgcgctcaatgtcactattgatgtggaggtagacccaaagagcccgttagtcaagtccctttctaggtccgacagtggatactatgctaatctcttcttacatatcggacttatgtccactgtagataagaggggaaagaaagtgacatttgaccagctggagaggaagataaggagacttgatctatctgtcgggctcagtgatgtgctcggaccttctgtgcttgtgaaggcgagaggtgcacggactaggctgctggcacctttcttctctaacagcgggacagcctgctaccctatagcaaatgcctctcctcaggtagctaagatactctggagtcagaccgcgtgcctgcggagtgtaaaaatcattattcaagcgggcacccaacgcgctgtcgcagtgactgctgaccatgaggttacctctactaagatagagaagaggcataccatcgctaaatacaatcccttcaagaaataggccgtatccctgagactgcaattcacccgccttcccaaaacaccatgacaccagataatgatctgtcttgattacttacagttagtttccctgtctatcaaattagaaaaaacacgggtagaagagttcggatcccggccggcgcaccaaaagcgcaagatgggccccagatcttctaccaggatcccagtacctctaatgctgaccatacggatcacgctggcactgagttatgtccgtctgacaagttctcttgatggcaggcctcttgcagccgcagggatcgtggtaacaggggataaagcagttaacatatacacctcatcccagacagggtcaatcatagtcaagttactcccaaatatgcccaaggacaaagaggcatgtgcaaaagccccattggaggcttacaacaggacactgactactttgcttaccccccttggtgattctatccgcaggatacaagagtctgtgactacatccggaggaaggagacagagacgctctataggtgccattattggcagtgtagctctaggggttgcaacagctgcacagataacggcagcctcggctctgatacaagccaaccagaatgctgctaacatcctccggcttaaggagagcattgctgcaaccaatgaagctgtacacgaggtcactggcggattgtcacagttagcagtggcagttggaaagatgcagcaatttgttaatgaccagcttaataacacagctcaagagttggactgtataaaaattacacagcaggttggtgtagaactcaacttatacttaactgaattgactacagtgttcgggccacaaatcacttcccctgccttaactcagttgactgttcaggctctttacaatctggctggtggtaatgtagattacttgttgactaagttaggtgtagggaacaaccagctcagctcattgattggtagcggcttgatcaccggtaaccctattttttacgactcacagactcaactcttgggcatacaggtgactgcaccctcagtcgggaacctaaataatatgcgtgccacctacttggagaccttgtctgtaagcacaacaaagggatttgcctcagcacttgtcccaaaagtagtgacacaggtcggttctgtgatagaagagcttgacacctcatactgtatagaagctgatttggatttatattgtacaagaatagtgacattccctatgtcccctggtatttattcctgtttgagcggcaatacatcggcttgcatgtattcaaggactgaaggcgcacttactacaccatacatgactctcaaaggctcagttgttgccaattgccagatgacaacatgtagatgtgcagaccccccgggtatcatatcacaaaattatggagaggctgtgtctctaatagataagcactcatgcaatgtcgtatccttagacgggataactttgaggctcagtggggaatttgatgcaacttatcaaaagaatatctcaatattagattctcaagtactagtgacaggcaatctcgatatctcaactgagcttgggaatgtcaaccactcaataagtaatgctttggataagttagaggaaagcaacagcaaactagacaaagtcaatgtcaaactgaccagcacatctgctctcattacctatattgttttaactgtcatatctcttgttcttggtatgcttagcctggttctagcatgctatctgatgtacaagcaaaaagcgcaacgaaagaccttgttgtggcttggaaataataccctagatcagatgagagctactacaaaaatgtgaatgcagatgagaggcagaggtatccccaatagcaatctgtgtgtcaattctggcagcctgttaatcagaagaattaagaaaaaactaccggatgtaggtgaacaaaagggaatatacgggtagaacggcctgagaggccacccctcaatcgggagccaggccccactacgtccgctctaccgcatcaccaacagcagtcttcagtcatggacagcgcagttagccaagttgcgctagagaatgatagaagagaagcgaataatacatggcgcttggttttccggatcgcagccttacttttactggtaataaccttagccgtctctgcagtcgccctggcatatagtatggaggctagcacacctggcgaccttgtaagcataccgactgcgatctctagggcagaggaaaggattacatctgcactcggttccaatcaagatgtagtagataggatatacaagcaagtggcccttgagtctccattggcactgctaaataccgaatctataattatgaatgcaataacgtctctctcttatcaaatcaatggagccacaaataatagcgggtgtggggcacctgttcatgacccagattacatcggggggataggtcaagaacttattgtagatgatacgagtgacgtcacatcattttatccctctgcattccaagaacacctgaattttatcccggcgcccactacaggatcaggctgcactcggataccctcattcgacatgagtgctacccattactgttacactcacaatgtgatattgtctggctgcagagatcactcacactcacatcagtatttagcacttggtgtgcttcggacatctgcaacagggagggtattcttttctactctgcgttccatcaacttggatgacgcccaaaatcggaagtcttgcagtgtgagtgcaactcctttaggttgtgatatgctgtgctctaaaatcacagagactgaggaagaggattataaatcagttatccccacatcgatggtacatggaaggttagggcttgacggccaataccatgagaaggacctagacgtcacaacactatttagggactgggtggcaaattacccaggagtaggaggtgggtcttttattaacaatcgcgtatggttcccggtctacggagggctaaaacccagctcgcctagtgacactgcacaagaagggagatacgtaatatacaagcgatacaatgacacatgcccggatgagcaagattaccagattcggatggctaagtcatcgtataagcctaggcggtttggtggaaaacgcgtacagcaggccatcctatccatcaaggtgtcaacatccttgggtgaggacccggtgttgactgtaccgcctaatacggtcgcactcatgggggccgaaggcagagttctcacagtggggacatctcatttcttatatcagcgagggtcatcatacttctctcccgctttgttataccctatgacagtcaacaacaaaacagccactcttcataatccttatacattcaatgctttcactcggccaggtagtgtcccttgccaggcttcagcaagatgccccaactcatgtgttaccggagtctatactgatccatatcccttagtcttccataggaaccacaccttgcgaggggtattcggaacaatgcttgatgacgaacaagcaagactcaaccctgtatctgcagtatttgataacatatcccgcagtcgcataactcgggtaagttcaagcagtaccagggcagcatacacgacatcaacgtgttttaaagttgtcaagaccaataaaacctattgcctcagcattgcagaaatatccaataccctcttcggggaattcagaattgtccccttactagttgagattctcaaggatggtggggtttaagaagctaggaatggtcggttgagtcaaccgtgagaaggcggggaagatgatattgcatcatctatcttctgtaacaccaagaatcaaatcgaataccagtgcgagctcgaatcctacgctgccagtcagccataatcggctggtgctaatgtggttagcctgaatcttgtcgatagtcacttgattaagaaaaaatgtggacggtagtgaaatataaggcaaagcagctcacagaggacagcacgggtaggacatggcgggctccggacccgaaagggcagagcatcagattatcctaccagagtcacatctgtcctcaccattggtcaagcataaattgctttattactggaaattaactgggctaccgcttcctgacgaatgtgacttcgaccatcttattatcagccgacaatggaagaaggtacttgaatcggccacccctgacattgagagaatgataaaactagggcgggcagtacaccagactctcaaccacaattccaggataaccggagtactccatccccggtgtttagaggaactggctagtattgaggttcctgattcaaccaacaaatttcggaagatcgaaaaaaagatccagattcacaacacaaggtatggagaactgttcacaagattatgcacgcatgtagaaaagaaactattggggtcatcttggtctaacaatgtcccacggtcagaggaattcaacagcatccgtacagatccggcattttggtttcactcaaaatggtccacagccaaatttgcatggctccatataaaacaggtccagaggcatctgattgtagcagcaagaacaaggtccgcagtcaacaaattagtgacgctgacccataaggtaggccaaatctttgttactcctgagcttgtcattgtgacacatacagatgagaacaagttcacgtgtcttacccaggaacttgtgttgatgtatgcagatatgatggagggcagagatatggtcagcataatatcatccacggcggcacatcttaggagcttatcagagaaaattgatgatattctgcggttagtagatgctctggcaagagatttgggcaatcaagtctacgatgttgtagcactaatggagggattcgcatacggcgctgttcagctgcttgaaccgtcaggtacatttgcgggggatttcttcgcattcaacctgcaggagctcaaagatactctaaccggactcctccccaaggatatcgcagaatctgtgactcacgcaatcgcaaccatattctctggcttagaacaaaatcaagcagctgagatgttgtgcctgttgcgtctgtggggtcacccactactcgagtcccgtattgcagcaaaagcagttaggagtcaaatgtgcgcaccaaaaatggtagactttgatatgatcctccaggtattatctttctttaaaggaacaatcatcaatggatatagaaagaagaatgcaggcgtgtggccacgtgtcaagatagatacgatatacgggaaggtcatagggcagctacacgcagattctgcagagatttcacatgatgtcatgttgagggaatacaagagtttatctgcacttgaattcgagccatgtatagagtatgaccctgtcaccaatctgagcatgtttttaaaagacaaggcaatcgcacacccgaaagacaactggctcgcttcgtttaggcgaaaccttctctctgaggaccagaagaaacatgtaagagaggcaacctcaactaaccgcctcttgatagagttcttagagtcaaatgattttgatccatataaagagatggaatatctgacgacccttgagtacctaagagatgataatgtggcagtatcatactcactcaaagaaaaagaggtgaaagttaatgggcggattttcgcaaagctaacaaatagattaaggaattgtcaggtaatggcagaagggatcctagctgaccagattgcacctttcttccagggaaatggggtcattcaggatagcatatctttgactaaaagtatgctagcgatgagtcaactgtctttcaacagcaacaagaaacgtattactgactgcaaagaaagagtatcctcaaaccgcaatcatgatccgaagagcaagaatcgtcggagagttgccacttttgtaactaccgacctgcaaaagtattgtctcaattggagatatcagacagtcaagctgttcgctcatgccatcaatcagctgatgggcttacctcacttcttcgagtggattcatcttagactaatggatactacgatgtttgtaggggaccctttcaatcctccaagtgaccctacggactgtgatctatcaagagtcccaaatgatgacatatatattgtcagtgctaggggaggcattgagggattatgccagaagctatggacaatgatctcaattgctgcaatccagcttgctgcagcaagatcacattgtcgcgttgcctgtatggtacaaggcgacaatcaagtaatagctataacgagagaggtaagatcagatgactccccagagatggtgttaacacaattgcatcaagccagtgataatttcttcaaggaattgattcatgtcaatcatttgatcggccataatttgaaggatcgtgaaaccatcaggtcagacacgttcttcatatacagcaaacgaatattcaaagatggagcaatactcagtcaggtcctcaaaaactcatctaaattagtgctaatatcaggcgaccttagtgaaaacactgtaatgtcttgtgccaacattgcatctactgtagcacggttatgcgagaacgggcttcctaaggatttctgttattacttaaactacttaatgagttgcgtgcagacatacttcgattccgaattttccatcaccaacaactcgcaactcgattctaaccagtcgtggatagaggacatttcttttgtgcactcatatgtcctgacccctgctcaactggggggactgagtaaccttcaatactcaaggctctacacaaggaacatcggcgacccgggaaccactgctttcgcagagatcaagagattagaggcagtggggttactgagtcctagcattatgactaacatcttaactaggccgcctggaaatggagattgggccagtctgtgcaacgatccatactcctttaattttgagactgtcgcaagcccaaatattgtccttaagaaacatacacaaagagtcctatttgaaacttgctcgaatcccttattatctggagtgcacacagaggataatgaggcagaagagaaggcattggctgaattcttgctcaatcaagaaatgattcatccacgtgtcgcgcatgctatcatggaagcaagctctgtaggtaggagaaagcaaattcaggggcttgttgacacaacaaacaccgtgattaagattgcactgactaggaggccactcggcatcaagaggctgatgcggatagtcaattactcgagcatgcatgcaatgctatttagagatgatgttttctcgtctaatagatccaaccatcccttagtctcttctgatatgtgttctcagacactggcagactatgcacggaacagaagctggtcacctttgacagggggtagaaaaatactgggtgtatctaatcctgataccatagaacttgtagagggtgagatccttagtgtcagtggagggtgcactagatgtgacagtggggatgaacagtttacttggttccatcttccaagcaatatagagctaaacgatgacaccagcaagaatcctccaatgagagtgccatatctcgggtcaaagactcaagagaggagagccgcctcgcttgcgaaaatagctcatatgtcaccacacgtgaaggcggctctaagggcgtcatctgtgttaatctgggcttatggggacaacgaaataaactggactgctgcccttaagattgcaaggtctcggtgcaacataagctcagagtatcttcgactattgtcacccttgcctacagctgggaatctccaacatagattggacgacggcataactcagatgacattcacccctgcatctctatacagggtgtcaccttacattcatatatctaatgattctcaaaggctatttactgaagaaggagtcaaagaggggaatgtggtttatcagcaaatcatgctcttgggtttgtccttaattgagtcactcttcccaatgacaacaaccaagacatatgatgaaatcacattgcacctccacagtaaatttagctgctgtatcagggaagcacctgttgcagttcctttcgagctactcggggtggcaccggaactaagggcagtaacctcaaataagtttatgtatgatcctagccctgtatcagagggagactttgcgagacttgacttagctatctttaagagttatgaacttaatttagagtcatattccacaatagagctaatgaacgttctttcaatatctagtgggaagttgattggccagtctgtggtttcttatgatgaagatacctccataaagaatgacgctataatagtgtatgacaacacacggaattggatcagcgaagctcagaattcagatgtggtccgcctattcgagtatgcggcactcgaagtgctcctcgactgttcttatcaactctactatctgagagtaagaggcctaaacaatatcgtcctgtacatgagtgatttatacaagaatatgccaggaattctactctctaatattgcagccacaatatctcaccctgtcatccattcaaggttgaatgcagtaggtctggtcaaccatgacgggtcacaccaacttgcagacacagatttcattgaaatgtctgcaaagctgctagtctcttgcactcgacgcgtggtctcaggtttacatgcagggaataagtatgacctgctgtttccatctgtcttggatgataacctaagtgagaagatgcttcagttgatatcccaattatgctgtctgtatacggtgctctttgctacaacaagagaaatcccgaaaataagaggcttatctgcagaagagaaatgttcagtacttactgagtacctactgtcagatgctgtgaaaccgttacttgggtccgagcaagtgagctctatcatgtctcccaacatagttacgttcccagccaatctgtattacatgtctaggaagagccttaatttgatcagggagagagaggacagggatactatcttggcattgttgttccctcaagaaccgctgctcgagtttcctctggtacgagatattggtgctcgtgtaaaagatccatttaccctacaacctgcggcgtttttacaagagttagatttgagtgctccggcaaggtatgacgcattcacaatcagtcaggcgcattctgaacacatattgccaaacccagaggaagatcacttagtacgatacttgttcagaggaatagggactgcgtcctcctcttggtataaggcatcccatcttctttctgtacccgaggtcagatgtgcaaggcatgggaactccttatatttagcagaaggaagtggagccatcatgagtcttctcgaattgcatataccacacgaaactatctattacaatacacttttctcgaacgagatgaaccccccacagcgacatttcggaccaactccaacacagtttctgaattcggttgtttttaggaatttacaggcggaagtaccatgcaaggatggatttgtccaggagttccgtccgttatggagagagaatacagaagaaagcgatctgacctcagataaagcagtgggatatatcacatctgtagtgccgtacaggtctgtatcattgctgcattgtgacattgaaatccctccaggatctaatcaaagcttactagatcagctggctaccaatctgtctctgattgccatgcattctgtgaaggagggcggggtcgtgattatcaaagtactgtatgcaatgggatattacttccatctactcatgaacttgtttactccatgttccacgaaaggatatattctctctaatggctatgcctgtagaggggatatggagtgttacctgatatttgtcatgggctacctaggcgggcctacatttgtacatgaggtggtgaggatggcaaaaacgctagtacagcggcatggcacacttttgtctaagtcagatgagattacactgactaggttatttacttcacagcaacagcgtgtaacagacatcctatccagccctttaccgagactaatgaagtacttgagagagaatattgatactgcactgattgaagccgggggacagcctgtccgtccattctgtgcagagagtttagtgagcacactaacagacatgactcagacaacccagatcatcgccagccacattgatacagtcattcgatctgtgatctatatggaagcagagggtgatcttgctgacacagtattcttatttaccccttacaacctctctatggacgggaaaaagagaacatcacttaaacagtgcacaagacagatcttagaggtcacaatactgggtctcagagtcaaaaatctcaataaagtaggtgatgtaatcagcctagtactcagaggtatgatttctctggaggaccttctcccactgagaacctacttgaagtgtagtacctgccctaagtatttgaaggctgtcctaggtattaccaaactcaaagaaatgttcacagacgcctctttattatacttgactcgtgctcaacaaaaattctacatgaaaactataggcaatgcagtcaagggatactatagtaactgtgactcttaaaggcaaccacatatcaataggccctctttctagccgatcgtattcttgttgacttcattataccatattagaaaaaaattgaattccgaccctttaagactcgtattcggattcaaataattatctcagaaaaaaagtgcacgtagttgttcttgattatagtcccgtcattcaccaagtctttgtttggt

SEQ ID NO: 8 - DNA, 12 bp, Artificial Sequence
  12bp insert containing a AfeI restriction site
agcgctgaccga

SEQ ID NO: 9 - DNA, 2238 bp, Artificial Sequence
  transgenic construct/anti-CTLA-4
ttaagaaaaaatacgggtagaagcagtttgagagctccgttcgccaccatggagtttgggctgtcatgggtctttctggtcgctctgtttaggggggtgcagtgtcaggtgcagctggtcgagtcaggggggggcgtggtgcagccaggcaggtccctgaggctgtcttgcgcagccagcggcttcacctttagctcctacacaatgcactgggtgcggcaggcacctggcaagggcctggagtgggtgaccttcatctcctatgacggcaacaataagtactatgccgattctgtgaagggccggtttaccatctccagagacaactctaagaatacactgtacctgcagatgaacagcctgagggccgaggacaccgccatctactattgcgcaaggacaggatggctgggacctttcgattattggggccagggcaccctggtgacagtgtctagcgcctctaccaagggcccaagcgtgtttcccctggccccttcctctaagtccacctctggaggcacagccgccctgggctgtctggtgaaggattacttccctgagccagtgaccgtgagctggaactccggcgccctgaccagcggagtgcacacatttccagccgtgctgcagagcagcggcctgtactccctgtctagcgtggtgaccgtgccctcctctagcctgggcacccagacatatatctgcaacgtgaatcacaagcctagcaatacaaaggtggacaagcgggtggagccaaagtcctgtgataagacccacacatgcccaccttgtccagcacctgagctgctgggaggaccatccgtgttcctgtttccacccaagcccaaggacaccctgatgatcagccggacccctgaggtgacatgcgtggtggtggacgtgagccacgaggaccccgaggtgaagttcaactggtacgtggatggcgtggaggtgcacaatgccaagaccaagccccgggaggagcagtacaactccacctatagagtggtgtctgtgctgacagtgctgcaccaggactggctgaacggcaaggagtataagtgcaaggtgtccaataaggccctgccagcccccatcgagaagaccatctctaaggcaaagggacagccacgggagccacaggtgtacacactgcctcccagccgggacgagctgaccaagaaccaggtgagcctgacatgtctggtgaagggcttctatcccagcgacatcgccgtggagtgggagtccaatggccagcctgagaacaattacaagaccacaccccctgtgctggactctgatggcagcttctttctgtattctaagctgaccgtggacaagagcagatggcagcagggcaacgtgtttagctgctccgtgatgcacgaggccctgcacaatcactacacacagaagtctctgagcctgtcccctggcaagcggagaaagaggggcagcggagagggaaggggctccctgctgacctgtggcgatgtggaggagaatcctggcccaatgaagtatctgctgccaacagctgccgccggcctgctgctgctggccgcacagcccgctatggccgagatcgtgctgacccagagcccaggcacactgtctctgagccctggagagagggccaccctgtcctgcagagcctcccagtctgtgggctcctcttacctggcctggtatcagcagaagcctggccaggccccaaggctgctgatctacggagccttcagcagggccaccggcatccccgaccgcttcagcggctccggctctggcacagacttcaccctgacaatctccaggctggagcccgaggacttcgccgtgtactattgccagcagtacggcagctccccttggacctttggccagggcacaaaggtggagatcaagaggaccgtggcagcaccatccgtgttcatctttccaccctctgacgagcagctgaagagcggcacagcctccgtggtgtgcctgctgaacaacttctacccccgcgaggccaaggtgcagtggaaggtggataacgccctgcagtccggcaattctcaggagagcgtgaccgagcaggactccaaggattctacatatagcctgtctagcaccctgacactgagcaaggccgattacgagaagcacaaggtgtatgcctgtgaggtcacccaccagggactgtcatcacccgtcaccaaaagtttcaatagaggagagtgctga

SEQ ID NO: 10 - DNA, 2214 bp, Artificial Sequence
  transgenic construct/Nivolumab
ttaagaaaaaatacgggtagaagcagtttgagagctccgttcgccaccatggagttcggcctgtcctgggtgttcctggtggccctgtttagaggcgtgcagtgccaggtgcagctggtggagagcggaggaggagtggtgcagccaggccggagcctgcgcctggactgtaaggccagcggcatcacctttagcaactccggaatgcactgggtgaggcaggcaccaggcaagggcctggagtgggtggccgtgatctggtacgacggctctaagcggtactatgccgatagcgtgaagggccggttcacaatctccagagacaactctaagaataccctgttcctccagatgaacagcctgagggccgaggatacagccgtgtactattgcgccaccaatgacgattactggggccagggcacactggtgaccgtgagcagcgccagcaccaagggaccaagcgtgttcccactggcaccttgctctcgcagcacatccgagtctaccgccgccctgggatgtctggtgaaggattatttccccgagcctgtgaccgtgagctggaacagcggcgccctgacatccggagtgcacacctttcccgccgtgctccagagcagcggcctgtacagcctgtcctctgtggtgacagtgcccagctcctctctgggcaccaagacatatacctgcaacgtggaccacaagcctagcaataccaaggtggataagcgggtggagtccaagtacggaccaccttgcccaccatgtccagcaccagagttcctgggaggaccaagcgtgttcctgtttcctccaaagcccaaggacaccctgatgatctccagaacaccagaggtgacctgcgtggtggtggacgtgtctcaggaggaccccgaggtgcagttcaactggtatgtggatggcgtggaggtgcacaatgccaagaccaagcccagggaggagcagtttaactccacataccgcgtggtgtctgtgctgaccgtgctgcaccaggactggctgaacggcaaggagtataagtgcaaggtgtccaataagggcctgcctagctccatcgagaagacaatcagcaaggcaaagggacagccaagggagccacaggtgtacaccctgcccccttctcaggaggagatgacaaagaaccaggtgagcctgacctgtctggtgaagggcttctatccttccgacatcgccgtggagtgggagtctaatggccagccagagaacaattacaagaccacaccacccgtgctggactctgatggcagcttctttctgtattccaggctgacagtggataagtctcgctggcaggagggcaacgtgtttagctgctccgtgatgcacgaggccctgcacaatcactacacccagaagtctctgagcctgtccctgggcaagcggagaaagaggggcagcggagagggaaggggctccctgctgacatgtggcgacgtggaggagaatcctggcccaatgaagtatctgctgcccaccgccgccgccggcctgctgctgctggcagcacagcctgctatggccgagatcgtgctgacacagtccccagccaccctgtctctgagccctggagagagggccaccctgagctgtagagcatcccagagcgtgagcagctacctggcctggtatcagcagaagcctggacaggcaccaaggctgctgatctacgatgcaagcaacagggcaacaggcatccccgcaagattcagcggctccggctctggcaccgactttacactgaccatctcctctctggagcctgaggatttcgccgtgtactattgccagcagagcagcaattggccacggacatttggccagggcaccaaggtggagatcaagagaacagtggccgcccctagcgtgttcatctttcctccatccgacgagcagctgaagagcggcaccgcatccgtggtgtgcctgctgaacaatttctacccacgggaggccaaggtgcagtggaaggtggataacgccctccagtccggcaattctcaggagagcgtgacagagcaggactccaaggattctacctatagcctgtctagcacactgaccctgtccaaggccgactacgagaagcacaaggtgtatgcctgcgaggtgacacaccagggcctgtcctctcccgtgaccaagagctttaacagaggcgagtgttgatga

SEQ ID NO: 11 - DNA, 2238 bp, Artificial Sequence
  transgenic construct/Atezolizumab
ttaagaaaaaatacgggtagaagcagtttgagagctccgttcgccaccatggagttcggcctgtcctgggtgttcctggtcgccctgttccggggagtgcagtgtgaggtgcagctggtcgagtctggggggggcctggtgcagccaggaggctccctgaggctgtcttgcgcagcaagcggcttcaccttttctgacagctggatccactgggtgagacaggcaccaggcaagggcctggagtgggtggcatggatcagcccttacggcggctccacctactatgccgacagcgtgaagggcaggtttacaatctccgccgatacctctaagaacacagcctatctgcagatgaattccctgcgcgccgaggataccgccgtgtactattgcgcacggagacactggcctggaggatttgattactggggccaggggaccctggtgacagtgagctccgcctctaccaagggaccaagcgtgttcccactggcaccttctagcaagtccacctctggaggaacagccgccctgggctgtctggtgaaggattactttcctgagccagtgaccgtgagctggaactccggcgccctgaccagcggagtgcacacattcccagccgtgctgcagtcctctggcctgtactccctgagctccgtggtgaccgtgccctctagctccctgggcacccagacatatatctgcaacgtgaatcacaagccatccaatacaaaggtggacaagaaggtggagcccaagtcttgtgataagacccacacatgcccaccttgtccagcacctgagctgctgggaggaccaagcgtgttcctgtttccacccaagcctaaggacaccctgatgatcagcaggacccctgaggtgacctgcgtggtggtggacgtgtcccacgaggatccagaggtgaagttcaactggtacgtggatggcgtggaggtgcacaatgccaagaccaagcctcgggaggagcagtacgcctccacctatagagtggtgtctgtgctgacagtgctgcaccaggactggctgaacggcaaggagtataagtgcaaggtgagcaataaggccctgccagcccccatcgagaagaccatctccaaggcaaagggacagccaagggagccacaggtgtacacactgcctccatctcgcgaggagatgaccaagaaccaggtgagcctgacatgtctggtgaagggcttttatccaagcgacatcgccgtggagtgggagtccaatggccagcccgagaacaattacaagaccacaccccctgtgctggactctgatggcagcttctttctgtattctaagctgaccgtggataagagcaggtggcagcagggcaacgtgttcagctgctccgtgatgcacgaggccctgcacaatcactacacacagaagtctctgagcctgtccccaggcaagaggaggaagaggggaagcggagagggaagaggctccctgctgacctgtggcgacgtggaggagaaccctggcccaatgaagtatctgctgcccacagcagccgccggcctgctgctgctggcagcacagcctgccatggccgatatccagatgacccagagcccatctagcctgtctgccagcgtgggcgacagggtgaccatcacatgcagggcaagccaggacgtgtctaccgccgtggcctggtaccagcagaagcctggcaaggccccaaagctgctgatctactccgcctctttcctgtattctggagtgccaagccgctttagcggatccggatctggaaccgacttcaccctgacaatctcctctctgcagcccgaggattttgccacatactattgccagcagtacctgtatcaccctgccaccttcggccaggggaccaaggtggagatcaagaggaccgtggcagcaccatccgtgttcatctttccaccctctgacgagcagctgaagtccggcacagcctctgtggtgtgcctgctgaacaatttctaccctagagaggccaaggtgcagtggaaggtggataacgccctgcagtccggcaattctcaggagagcgtgaccgagcaggactccaaggattctacatatagcctgagctccaccctgacactgtccaaggccgactacgagaagcacaaggtgtatgcctgtgaggtcacccatcaggggctgtcctcaccagtcacaaaatccttcaatcggggggaatgctaataa

SEQ ID NO: 12 - DNA, 2244 bp, Artificial Sequence
  transgenic construct/anti-KIR
ttaagaaaaaatacgggtagaagcagtttgagagctccgttcgccaccatggagttcggcctgtcctgggtgttcctggtggccctgttccgcggcgtgcagtgccaagtgcaactggtgcagagcggtgcagaggtgaagaagccaggatcctctgtgaaagtctcatgcaaagccagtggaggtacgttcagcttttacgcaatcagctgggtgagacaggctccgggccaaggtctggaatggatgggcggattcattccaattttcggcgcagcaaactacgcacagaagttccaggggcgggtcacgattactgctgatgagtcgacaagcacagcatacatggagctcagttctttgagaagcgatgatacagcagtttattactgtgctagaattccaagcggttcttattactatgactacgatatggatgtctggggacagggaacaacagtcaccgtatcttccgcgagcaccaagggaccttccgtgtttcccctggccccttgctccagatcaacctcagagtcgactgctgctctcggttgcttggtgaaagattattttccagaacccgttaccgtgtcttggaactccggtgcccttacttcaggggttcataccttccccgcagtcctacagtcatctggtctgtattctctcagctccgtggtcaccgtcccaagcagctcactgggcaccaagacctatacctgcaacgtcgaccataagcctagtaatacaaaggtcgacaagcgagtcgaatccaaatacggacctccttgtccaccttgccccgctcctgaattcctgggtggaccaagcgtctttctgtttccaccaaaacctaaagacacacttatgatttcccgcacacccgaagtcacctgcgtggtggtggatgtcagccaggaagaccctgaggtgcaattcaactggtacgttgatggcgtagaagtccacaacgccaagacaaaacccagagaggagcagtttaatagtacctatagggtggtgtccgtcctgaccgttctccaccaggattggttgaatggcaaggaatacaagtgtaaagtgagcaacaaaggcctaccttcgagcatagaaaagactatctccaaggcaaagggacagccaagggagccccaggtgtacacgctgccaccctctcaggaggagatgactaagaaccaggtgtccttgacctgcctcgtaaaggggttttacccctccgacatagccgtcgaatgggagagtaacggacaacccgagaacaattataaaactacacctcccgtgctcgactctgacggatccttcttcctgtactctaggctgacagtggataaatcccgttggcaggaaggaaatgtattttcctgtagtgtgatgcacgaggctctgcacaatcattatacacagaaatcactgtccttaagcctcggcaaacgccgcaagcgcggctccggcgagggccgcggctccctgctgacctgcggcgacgtggaggagaaccccggccccatgaagtacctgctgcccaccgccgccgccggcctgctgctgctggccgcccagcccgccatggccgaaatcgtcctcacacagagcccggttacgctttcattgtctccaggcgagcgcgctaccctcagctgtagggcgtctcagtccgtttccagctatttagcttggtaccagcaaaaacctggccaggcccctagactgcttatttacgacgcctctaaccgagcgactggtatacccgcaaggttcagcggctcgggctcaggcaccgactttactctcaccatttcatcactggagcctgaggacttcgccgtgtattactgtcagcagaggagtaactggatgtacaccttcggccaagggaccaaattggaaatcaaaaggacagtggccgcgccaagcgtgtttatatttcccccctccgacgaacagttaaagagtggaaccgcatctgtggtatgtctgcttaacaatttttaccctcgcgaggcaaaggttcaatggaaagtcgacaacgccttacagtctggtaacagtcaagagtctgttacggagcaagacagcaaggactccacctattcgctgtcttcaacactaaccctcagcaaggctgattacgagaagcacaaagtttacgcctgtgaggtcacccatcaggggctttctagccctgtcacaaagtcgttcaacaggggagagtgctaatga

SEQ ID NO: 13 - DNA, 2232 bp, Artificial Sequence
  transgenic construct/anti-LAG-3
ttaagaaaaaatacgggtagaagcagtttgagagctccgttcgccaccatggagttcggcctgtcctgggtgttcctggtggccctgttccgcggcgtgcagtgccaggtgcaactgcagcagtggggggccggactcctgaaaccatccgaaactctctcattgacttgcgccgtatacggcgggtcattttccgattactactggaattggatccgtcagcctccaggtaaaggacttgaatggataggtgaaataaatcatcgtggaagtactaacagcaatcctagtctgaaatcaagagtaacgctgagtttggacacatctaagaaccagttttcccttaaacttagaagtgttaccgctgctgacacagctgtatactactgtgctttcggttactccgattatgagtacaattggttcgacccttggggacagggcacactagtgacagtgtccagtgcttcaaccaaggggccaagcgtgtttcccttggccccctgttcacgctccactagcgaaagcacagccgcgttaggctgtctggtgaaagactattttccagagccagtcacggtgtcttggaactcgggtgccctgacatcaggtgttcatactttccccgctgtcctccagtccagtgggctgtactcattatcttcggtggtgactgtcccctcctcatcgctggggacgaagacctatacctgtaacgtggaccacaaaccaagcaacactaaggtggacaagcgcgtggaatccaaatacgggcccccatgcccaccgtgccccgcacctgagttcctgggcggtcctagcgtctttttgttcccaccgaaaccaaaggataccctcatgatttccaggacgccggaggtcacctgcgtggtcgtggacgtgagtcaggaggaccccgaagtgcaatttaactggtatgtggacggtgtggaagtccataatgcaaagaccaagcctagagaggaacaatttaatagcacgtaccgggtggttagtgtccttaccgtcttgcaccaggattggctgaacgggaaggagtataagtgcaaggtgtcaaacaagggactgccctcatcaatcgagaagacaataagtaaggcgaagggccagccacgggagcctcaggtctacacattgccgccaagtcaagaggagatgaccaaaaaccaggtgtctttgacttgcctcgtgaagggtttttacccaagtgacatcgccgtagagtgggagtctaacggccagccagagaataactataaaacgacacctccagtgcttgattctgatggatcttttttcttgtactcaaggttgaccgtggataagagcaggtggcaagaaggcaacgtttttagttgttccgttatgcacgaggccttgcataatcactatactcagaagtccctatccctgtctctcgggcgccgcaagcgcggctccggcgagggccgcggctccctgctgacctgcggcgacgtggaggagaaccccggccccatgaagtacctgctgcccaccgccgccgccggcctgctgctgctggccgcccagcccgccatggccgaaattgtgctgacacaatcgccagccactctgtctctgagccctggtgagcgggccactctgtcctgcagggccagtcagtctatttccagctacttggcttggtatcagcaaaaacccggccaagctcctagattgcttatttacgacgcgagcaatagggccaccggtattccagctcgctttagcggctccggaagtggtactgatttcacgctgacaatttcaagcttggaacccgaggattttgcagtgtattactgccagcagagaagcaattggccgctgaccttcggccaggggactaatcttgaaataaaacgcacggtggctgccccctccgtcttcatctttccaccttctgacgagcaattgaagagcggtaccgctagcgtagtgtgtcttctgaacaatttctacccacgcgaggccaaagtgcaatggaaagtcgacaatgctctgcagtcaggaaactcccaggagagcgtcacagagcaagattctaaagattccacttacagtctgtcatcgacattgacactgagcaaggccgattacgaaaagcataaagtgtacgcttgtgaggtcactcaccaaggactgagctcaccagttaccaaatcgtttaatagaggtgagtgttaatga

SEQ ID NO: 14 - DNA, 2250 bp, Artificial Sequence
  transgenic construct/anti-NKG2A
ttaagaaaaaatacgggtagaagcagtttgagagctccgttcgccaccatggagttcggcctgtcctgggtgttcctggtggccctgttccgcggcgtgcagtgccaggttcagttggtacagtctggcgcggaggtgaaaaaacctggagcatctgtcaaggtaagttgtaaagcttcgggctatacctttacctcatattggatgaactgggtgcggcaggcccccggtcaagggcttgaatggatgggcagaattgatccttatgattccgaaacacattacgctcagaagctgcagggcagggttaccatgacgacagacaccagcacttccactgcttacatggagctgcgatctctgcggtcagatgacacggctgtgtactattgcgccagaggcggatacgattttgacgtggggactctgtattggttctttgacgtctgggggcagggaaccactgtgacagtctcatcggcgtcaactaaagggccaagcgtgttccccttggcaccatgtagtcggtccaccagtgaaagtaccgctgcactcggttgtctggtaaaagattattttccagagcccgttactgtaagctggaacagtggggccttgacgtctggagtccatacctttccggcggtccttcagtcatctgggctgtacagcctttccagcgttgtcactgttccaagctcctccctgggcacgaagacctatacatgtaatgtcgaccataagccttcaaatacaaaagtagacaaacgtgtggaatcaaaatacggacccccatgcccgccctgtccagcgcctgaattcttgggaggcccgtctgtctttttattccccccgaaaccgaaggacaccctcatgatctcaaggacaccagaggtgacctgcgtggtggtggatgttagtcaagaggatccagaggtgcagtttaactggtatgtcgatggcgtcgaagtccacaacgctaaaaccaagccacgggaggagcagtttaactctacctatcgagtggtgtccgtactgactgtgttgcaccaggactggcttaatgggaaagaatacaagtgtaaagtttcaaacaaaggcctgccttcaagtatcgaaaagactattagcaaagctaaagggcagccaagagaaccacaggtgtacactttgcccccttcccaagaggagatgactaagaatcaagtttctctgacatgtctggtaaaaggcttctacccttctgatatagcagtggagtgggagtctaacggacagcccgaaaacaattacaagacgacaccccctgtgctagattcagacggctctttttttctttacagtcggctcacagtggataaatcacgctggcaggaagggaatgtctttagctgcagcgtcatgcatgaggctttgcacaaccattacactcagaaatctctgagcctctcacttggtaaacgccgcaagcgcggctccggcgagggccgcggctccctgctgacctgcggcgacgtggaggagaaccccggccccatgaagtacctgctgcccaccgccgccgccggcctgctgctgctggccgcccagcccgccatggccgatatccagatgacccagagcccctccagccttagcgcttcagtcggggatagagtgacaattacctgcagagcgtctgaaaatatctacagctacttggcctggtaccaacagaaaccaggcaaagctcctaagctcctgatttataacgcaaaaaccttggctgagggggtgcccagtcgcttctcgggctctggctcaggaaccgactttacactcaccatttcttcactgcagcctgaggacttcgctacgtactactgccagcatcactatggtaccccacgcacttttggaggggggactaaggtggaaatcaagaggaccgtggccgccccatccgtatttatcttccccccatccgatgagcagttaaaatcagggaccgcctcggttgtgtgtctgcttaacaacttttacccccgcgaagctaaggtgcagtggaaggttgataacgccttgcagtctggaaacagccaggagagcgtcacagaacaagattcaaaagacagtacttattccctgagtagtacgctgaccctgtccaaagccgattacgaaaagcacaaagtatacgcgtgtgaggtcactcaccagggactatcaagcccggtcactaaatcattcaacaggggggagtgctaatga

SEQ ID NO: 15 - DNA, 2238 bp, Artificial Sequence
  transgenic construct/anti-GITR
ttaagaaaaaatacgggtagaagcagtttgagagctccgttcgccaccatggagttcggcctgtcctgggtgttcctggtggccctgttccgcggcgtgcagtgccaagtgaccttacgggaatcaggtccagcgctggtaaagcccacccaaactctaacattgacctgtactttcagcggcttttccctttcgacaagtggcatgggggtgggatggatccggcaacctcctgggaaggccctcgaatggctggcccacatttggtgggacgatgacaagtactacaacccctcactgaagtcacggctgacgatttcaaaggacaccagcaaaaatcaagtggtactgacgatgaccaatatggatcccgtcgacactgctacatactactgcgctcgcacaaggcgatactttcccttcgcctattggggacagggaactctggttaccgtgagttccgcatccactaagggcccaagcgtcttcccgctggccccttccagtaagagcacctccggagggaccgctgcacttggatgcctggttaaagactattttcctgaacctgttacagtcagttggaactcgggggcactaacctctggagtgcataccttcccagctgtactccaatcatctggactatactcactttcgtctgtcgtgactgtgccctcttcttcacttgggacccagacatatatttgcaacgtcaatcataagccatcgaacaccaaggtggataagaaggtggagccgaaatcttgtgataagacccacacatgtcctccatgcccagcccctgagctgttgggaggtccctctgtgttcctgttccccccaaaaccaaaagacaccctcatgatctccagaacgcctgaggtcacctgtgtcgttgtggacgtttctcacgaagacccagaggtcaaattcaattggtatgtcgacggggtagaggtccacaatgctaagaccaagcctcgagaagagcaatatgcttccacttacagagtggtgtctgttctgaccgtcctgcaccaagactggctaaatggaaaagagtataaatgcaaggtcagcaacaaagctctgcccgccccaattgaaaagactatcagcaaagcgaaaggccagccacgggaacctcaggtctatacgctgcccccaagccgtgatgagctcacaaagaaccaggtgagtctgacttgtctagtgaaaggcttttaccccagtgacattgcagtggagtgggagtccaacggacaacccgaaaacaactataaaactaccccccccgttctcgattctgatggcagcttcttcttatacagcaaacttacggtggataaatcgcggtggcagcagggaaatgtgttttcctgctctgtcatgcatgaggccctacataatcactatacacaaaagtctctctctttgtcccccggaaaacgccgcaagcgcggctccggcgagggccgcggctccctgctgacctgcggcgacgtggaggagaaccccggccccatgaagtacctgctgcccaccgccgccgccggcctgctgctgctggccgcccagcccgccatggccgagatagtgatgactcagtcccccgccactctctcagtaagcccaggagagagagctacgctgagctgcaaggcctcccagaacgtggggaccaacgtagcctggtaccaacaaaagcccggtcaggccccccggctgctcatttattccgcaagttatagatattcaggcattccggccagatttagtggatccggctcaggcacggagtttaccttgaccattagctctctccaatctgaggatttcgcggtgtattattgtcagcagtataacacagaccccttgaccttcggcggtggcacaaaagtcgaaattaagaggacagtcgccgccccttccgtgttcattttccccccctcagacgaacagctgaaatctggcacagcttcgtggtgtttactgaacaacttctatcccagagaagcaaaggtccagtggaaggtcgataatgccctgcagtctggtaatagtcaggagtccgtaactgagcaggacagcaaggactccacctacagcctgtcctctaccctgacactgagcaaagcagactacgaaaaacacaaagtgtacgcctgtgaggtcactcaccaggggctgagctctccagtgactaagtcttttaatcgcggcgagtgctaatga

SEQ ID NO: 16 - DNA, 2232 bp, Artificial Sequence
  transgenic construct/anti-OX40
ttaagaaaaaatacgggtagaagcagtttgagagctccgttcgccaccatggagttcggcctgtcctgggtgttcctggtggccctgttccgcggcgtgcagtgcgaagtgcagctagtgcagagtgggggcgggctagtgcagccaggggggtctcttcgcttgtcttgcgccggtagcggttttaccttttcatcgtatgcaatgtactgggtgaggcaagcccctggcaagggattggagtgggtgagcgcgatagacactgacgccggtaccttctatgcggatagtgtaagggggcgcttcaccattagtcgcgacaacgccaagaactctctttatctccagatgaattcactccgagctgaggacaccgcagtctacttttgtgctaggctgggggaaggctacttctttgattactggggacagggaaccttggtgactgtgagctctgcctccacgaaaggtccttctgttttcccccttgccccctcctcaaaatctacctctggaggtaccgccgctctaggctgcctagtaaaggattacttccctgaacccgtgacggtgtcttggaacagtggtgctcttacaagtggcgtgcacacctttcctgccgtgctccaaagttctgggctttattctctgtcctccgtggtgaccgtgcccagcagctctcttggtacccagacttacatctgtaatgttaaccacaaaccctcaaatacaaaggtggacaagagagtggagccaaagagctgcgataaaacccacacctgtcccccctgtcccgcacctgagcttcttgggggccctagtgtgtttctgttcccacctaagcccaaagacaccctgatgatctctaggaccccggaggttacctgcgtagttgtggacgtctcccacgaggatcccgaggtgaaattcaattggtacgtcgacggcgttgaagtccataacgccaagaccaagcctcgcgaagaacagtataactctacctatcgcgtggtgtcggtcctgactgtgcttcaccaggactggcttaacgggaaggaatataaatgcaaggtcagcaataaggccctccccgctccaattgagaagaccatttccaaagctaaaggccagccacgggagccccaggtctacactctaccaccctcccgcgaggagatgaccaaaaaccaggtgtccctgacctgtttggtgaagggattttacccgtccgatatcgctgtggagtgggaaagtaatggccaacctgaaaacaactataagacaaccccgccagtcctagatagcgatggatccttcttcctgtatagtaaacttacagtggataaaagtaggtggcagcagggtaacgttttttcgtgtagtgtgatgcatgaagcactccacaatcactatacccaaaaaagcctttcgctgtcccctggccgccgcaagcgcggctccggcgagggccgcggctccctgctgacctgcggcgacgtggaggagaaccccggccccatgaagtacctgctgcccaccgccgccgccggcctgctgctgctggccgcccagcccgccatggccgagattgtgctgacgcagagcccagcaactttgagtctctcgcccggcgagcgcgccacactttcctgtcgagcctcccaatccgtgagcagttacctggcatggtaccaacagaagcctgggcaagcccccagattgctgatttatgatgcctccaatcgagcaacaggaataccagctcggttcagtgggagcggaagcggtacagatttcactctgacaatcagctcgctggagccagaggacttcgctgtttattactgccagcagcgttcgaactggcccccaacgtttggaggaggcaccaaagtggaaataaaacgaacagtagcagccccatctgtcttcatcttcccccccagcgatgaacagctgaagtcaggcactgcttccgtcgtctgcctactgaacaatttttatcctagagaggccaaggtccaatggaaggtagacaacgcactccaatcagggaactcacaagagtctgtcaccgagcaggactctaaggactcgacatactcattatctagtaccctgacactgagcaaggccgactacgaaaaacataaagtctacgcctgtgaagtgacgcaccagggactcagctctcccgtcactaagagcttcaatcggggggagtgttaatga

SEQ ID NO: 17 - DNA, 2250 bp, Artificial Sequence
  transgenic construct/anti-VEGF-A
ttaagaaaaaatacgggtagaagcagtttgagagctccgttcgccaccatggagttcggcctgtcctgggtgttcctggtggccctgttccgcggcgtgcagtgcgaggtgcagctggtggagagcggcggcggcctggtgcagcccggcggcagcctgaggctgagctgcgccgccagcggctacaccttcaccaactacggcatgaactgggtgaggcaggcccccggcaagggcctggagtgggtgggctggatcaacacctacaccggcgagcccacctacgccgccgacttcaagaggaggttcaccttcagcctggacaccagcaagagcaccgcctacctgcagatgaacagcctgagggccgaggacaccgccgtgtactactgcgccaagtacccccactactacggcagcagccactggtacttcgacgtgtggggccagggcaccctggtgaccgtgagcagcgccagcaccaagggccccagcgtgttccccctggcccccagcagcaagagcaccagcggcggcaccgccgccctgggctgcctggtgaaggactacttccccgagcccgtgaccgtgagctggaacagcggcgccctgaccagcggcgtgcacaccttccccgccgtgctgcagagcagcggcctgtacagcctgagcagcgtggtgaccgtgcccagcagcagcctgggcacccagacctacatctgcaacgtgaaccacaagcccagcaacaccaaggtggacaagaaggtggagcccaagagctgcgacaagacccacacctgccccccctgccccgcccccgagctgctgggcggccccagcgtgttcctgttcccccccaagcccaaggacaccctgatgatcagcaggacccccgaggtgacctgcgtggtggtggacgtgagccacgaggaccccgaggtgaagttcaactggtacgtggacggcgtggaggtgcacaacgccaagaccaagcccagggaggagcagtacaacagcacctacagggtggtgagcgtgctgaccgtgctgcaccaggactggctgaacggcaaggagtacaagtgcaaggtgagcaacaaggccctgcccgcccccatcgagaagaccatcagcaaggccaagggccagcccagggagccccaggtgtacaccctgccccccagcagggaggagatgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcaagctgaccgtggacaagagcaggtggcagcagggcaacgtgttcagctgcagcgtgatgcacgaggccctgcacaaccactacacccagaagagcctgagcctgagccccggcaagcgccgcaagcgcggctccggcgagggccgcggctccctgctgacctgcggcgacgtggaggagaaccccggccccatgaagtacctgctgcccaccgccgccgccggcctgctgctgctggccgcccagcccgccatggccgacatccagatgacccagagccccagcagcctgagcgccagcgtgggcgacagggtgaccatcacctgcagcgccagccaggacatcagcaactacctgaactggtaccagcagaagcccggcaaggcccccaaggtgctgatctacttcaccagcagcctgcacagcggcgtgcccagcaggttcagcggcagcggcagcggcaccgacttcaccctgaccatcagcagcctgcagcccgaggacttcgccacctactactgccagcagtacagcaccgtgccctggaccttcggccagggcaccaaggtggagatcaagaggaccgtggccgcccccagcgtgttcatcttcccccccagcgacgagcagctgaagagcggcaccgccagcgtggtgtgcctgctgaacaacttctaccccagggaggccaaggtgcagtggaaggtggacaacgccctgcagagcggcaacagccaggagagcgtgaccgagcaggacagcaaggacagcacctacagcctgagcagcaccctgaccctgagcaaggccgactacgagaagcacaaggtgtacgcctgcgaggtgacccaccagggcctgagcagccccgtgaccaagagcttcaacaggggcgagtgctga

SEQ ID NO: 18 - DNA, 2232 bp, Artificial Sequence
  transgenic construct/anti-VEGFR
ttaagaaaaaatacgggtagaagcagtttgagagctccgttcgccaccatggagttcggcctgtcttgggtgttcctggtcgccctgtttaggggagtgcagtgtgaagtgcagctggtgcagtctggggggggcctggtgaagccaggaggcagcctgaggctgtcctgcgcagcatctggcttcacctttagctcctacagcatgaactgggtgcgccaggcaccaggcaagggcctggagtgggtgtctagcatctcctctagctcctcttacatctactatgccgactccgtgaagggcaggttcaccatctctcgcgataacgccaagaatagcctgtatctgcagatgaattccctgagggccgaggacaccgccgtgtactattgcgcccgcgtgacagacgccttcgacatctggggccagggcaccatggtgacagtgagctccgcctctaccaagggaccaagcgtgctgccactggcaccatctagcaagtctaccagcggaggaacagccgccctgggatgtctggtgaaggattacttccccgagcctgtgacagtgtcttggaacagcggcgccctgaccagcggagtgcacacatttcccgccgtgctgcagtcctctggcctgtactccctgagctccgtggtgaccgtgccttctagctccctgggcacccagacatatatctgcaacgtgaatcacaagcctagcaatacaaaggtggacaagagggtggagccaaagtcctgtgataagacccacacatgcccaccttgtccagcaccagagctgctgggaggaccaagcgtgttcctgtttccacccaagcccaaggacaccctgatgatctctcgcacccctgaggtgacatgcgtggtggtggacgtgtcccacgaggatccagaggtgaagttcaactggtacgtggatggcgtggaggtgcacaatgccaagaccaagcctcgggaggagcagtacaactccacctatagagtggtgtctgtgctgacagtgctgcaccaggactggctgaacggcaaggagtataagtgcaaggtgagcaataaggccctgcctgccccaatcgagaagaccatctccaaggcaaagggacagcctagggagccacaggtgtacacactgcctccatccagagaggagatgaccaagaaccaggtgtctctgacatgtctggtgaagggcttctatccaagcgacatcgccgtggagtgggagtccaatggccagcccgagaacaattacaagaccacaccccctgtgctggactccgatggctctttctttctgtattctaagctgaccgtggataagagccggtggcagcagggcaacgtgttttcctgctctgtgatgcacgaggccctgcacaatcactacacacagaagagcctgtccctgtctcccggcaagcggagaaagaggggaagcggagagggaagaggctccctgctgacctgtggcgacgtggaggagaacccaggacctatgaagtatctgctgcctacagcagccgccggcctgctgctgctggcagcacagcccgccatggccgacatccagatgacccagagcccttctagcgtgagcgcctccatcggcgacagggtgaccatcacatgccgcgcctcccagggcatcgataactggctgggctggtaccagcagaagcccggcaaggcccctaagctgctgatctatgacgcctctaatctggataccggagtgccaagccggttctctggaagcggctccggcacatactttaccctgacaatctcctctctgcaggccgaggacttcgccgtgtatttttgccagcaggccaaggcattcccacccacctttggcggaggaacaaaggtggacatcaagaggaccgtggcagcaccttccgtgttcatctttcctccatctgatgagcagctgaagagcggcacagcctccgtggtgtgcctgctgaacaatttctacccaagagaggccaaggtgcagtggaaggtggacaacgccctgcagagcggcaattcccaggagtctgtgaccgagcaggacagcaaggattccacatattctctgagctccaccctgacactgagcaaggccgattacgagaagcacaaggtgtatgcctgtgaggtcactcaccaggggctgtcaagtccagtcaccaagtccttcaatcggggggaatgttgataa

SEQ ID NO: 19 - DNA, 708 bp, Artificial Sequence
  transgenic construct/NS1
ttaagaaaaaatacgggtagaagcagtttgagagctccgttcgccaccatggatagcaataccatgtcctcctttcaggttgactgtttcctgtggcatattcgcaagcgtttcgccgataatggtctgggtgacgccccgttcctggaccgtctgcgtcgcgatcagaagagcctgaaaggccgcggtaacaccctgggcctggacatcgaaaccgcgaccctggtgggcaagcagatcgtggagtggattctgaaagaggaaagcagcgaaaccctgcgtatgaccattgccagcgtgccgaccagccgttacctgagcgacatgaccctggaggaaatgagccgtgattggttcatgctgatgccgcgccagaagatcattggtccactgtgcgtgcgtctggaccaggctatcatggagaagaacattgtgctgaaagccaacttcagcgtgatctttaaccgtctggaaaccctgattctgctgcgcgcctttaccgaggaaggcgcgatcgtgggcgagattagcccgctgccgagcctgccaggtcacacctatgaagatgtgaaaaatgccgtgggcgtgctgattggcggtctggagtggaacggtaacaccgtgcgtgtgagcgaaaacattcagcgcttcgcatggcgtaactgtgatgagaatggtcgtccaagcctgccaccggaacaaaaataa

SEQ ID NO: 20 - DNA, 1104 bp, Artificial Sequence
  transgenic construct/B18R
ttaagaaaaaatacgggtagaatacaattaacgatctttatagccaccatgacgatgaaaatgatggtacatatatatttcgtatcattattgttattgctattccacagttacgccatagacatcgaaaatgaaatcacagaattcttcaataaaatgagagatactctaccagctaaagactctaaatggttgaatccagcatgtatgttcggaggcacaatgaatgatatagccgctctaggagagccattcagcgcaaagtgtcctcctattgaagacagtcttttatcgcacagatataaagactatgtggttaaatgggaaaggctagaaaaaaatagacggcgacaggtttctaataaacgtgttaaacatggtgatttatggatagccaactatacatctaaattcagtaaccgtaggtatttgtgcaccgtaactacaaagaatggtgactgtgttcagggtatagttagatctcatattagaaaacctccttcatgcattccaaaaacatatgaactaggtactcatgataagtatggcatagacttatactgtggaattctttacgcaaaacattataataatataacttggtataaagataataaggaaattaatatcgacgacattaagtattcacaaacgggaaaggaattaattattcataatccagagttagaagatagcggaagatacgactgttacgttcattacgacgacgttagaatcaagaatgatatcgtagtatcaagatgtaaaatacttacggttataccgtcacaagaccacaggtttaaactaatactagatccaaaaatcaacgtaacgataggagaacctgccaatataacatgcactgctgtgtcaacgtcattattgattgacgatgtactgattgaatgggaaaatccatccggatggcttataggattcgattttgatgtatactctgttttaactagtagaggcggtattaccgaggcgaccttgtactttgaaaatgttactgaagaatatataggtaatacatataaatgtcgtggacacaactattattttgaaaaaacccttacaactacagtagtattggagtaa

SEQ ID NO: 21 - DNA, 1662 bp, Artificial Sequence
  transgenic construct/IL-12
ttaagaaaaaatacgggtagaagcagtttgagagctccgttcgccaccatgggtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagtgttcctggagtaggggtacctggggtgggcgccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagctgctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcctaatga

SEQ ID NO: 22 - DNA, 1626 bp, Artificial Sequence
  transgenic construct/(ns)hIL-12
ttaagaaaaaatacgggtagaagcagtttgagagctccgttcgccaccatgagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagctgctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttccggctccggcgagggccgcggctccctgctgacctgcggcgacgtggaggagaaccccggccccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagttaa

SEQ ID NO: 23 - DNA, 924 bp, Artificial Sequence
  transgenic construct/CD80
ttaagaaaaaatacgggtagaagtggctttcacttttgaccctaagcatctgccaccatgggccacacacggaggcagggaacatcaccatccaagtgtccatacctcaatttctttcagctcttggtgctggctggtctttctcacttctgttcaggtgttatccacgtgaccaaggaagtgaaagaagtggcaacgctgtcctgtggtcacaatgtttctgttgaagagctggcacaaactcgcatctactggcaaaaggagaagaaaatggtgctgactatgatgtctggggacatgaatatatggcccgagtacaagaaccggaccatctttgatatcactaataacctctccattgtgatcctggctctgcgcccatctgacgagggcacatacgagtgtgttgttctgaagtatgaaaaagacgctttcaagcgggaacacctggctgaagtgacgttatcagtcaaagctgacttccctacacctagtatatctgactttgaaattccaacttctaatattagaaggataatttgctcaacctctggaggttttccagagcctcacctctcctggttggaaaatggagaagaattaaatgccatcaacacaacagtttcccaagatcctgaaactgagctctatgctgttagcagcaaactggatttcaatatgacaaccaaccacagcttcatgtgtctcatcaagtatggacatttaagagtgaatcagaccttcaactggaatacaaccaagcaagagcattttcctgataacctgctcccatcctgggccattaccttaatctcagtaaatggaatttttgtgatatgctgcctgacctactgctttgccccaagatgcagagagagaaggaggaatgagagattgagaagggaaagtgtacgccctgtataa

SEQ ID NO: 24 - DNA, 414 bp, Artificial Sequence
  transgenic construct/Apoptin
ttaagaaaaaatacgggtagaagcagtttgagagctccgttcgccaccatgaacgctctccaagaagatactccacccggaccatcaacggtgttcaggccaccaacaagttcacggccgttggaaacccctcactgcagagagatccggattggtatcgctggaattacaatcactctatcgctgtgtggctgcgcgaatgctcgcgctcccacgctaagatctgcaactgcggacaattcagaaagcactggtttcaagaatgtgccggacttgaggaccgatcaacccaagcctccctcgaagaagcgatcctgcgacccctccgagtacagggtaagcgagctaaaagaaagcttgattaccactactcccagccgaccccgaaccgcaaaaaggcgtataagactgtaa

SEQ ID NO: 25 - DNA, 2232 bp, Artificial Sequence
  transgenic construct/anti-CD28
ttaagaaaaaatacgggtagaagcagtttgagagctccgttcgccaccatggagtttgggctgagttgggtctttctggtcgctctgttccggggggtgcagtgtcaggtgcagctggtgcagtctggggccgaggtcaagaagcccggcgcctccgtgaaggtgtcttgcaaggccagcggctacaccttcacaagctactatatccactgggtgaggcaggcaccaggacagggcctggagtggatcggctgttaccctggcaacgtgaataccaactataatgagaagtttaaggaccgcgccacaatcctgacctgggatacaagctccaccgcctatatggagctgtccaggctgcgctctgacgatacagccgtgtacttctgcaccaggtcccactatggcctgcagtggaactttgacgtgtggggccagggcaccacagtgacagtgtctagcgcctctaccaagggccctagcgtgttccctctggccccatgctcccgctctacaagcgagtccaccgccgccctgggatgtctggtgaaggattacttccctgagccagtgaccgtgagctggaattccggcgccctgacaagcggagtgcacacctttccagccgtgctgcagtcctctggcctgtactccctgagctccgtggtgacagtgccttctagctccctgggcaccaagacatatacctgcaacgtggaccacaagccaagcaataccaaggtggataagcgggtggagagcaagtacggaccaccttgcccatcctgtccagcacctgagtttctgggaggaccttccgtgttcctgtttccacccaagccaaaggacacactgatgatctctagaacacccgaggtgacctgcgtggtggtggacgtgagccaggaggatcctgaggtgcagttcaactggtacgtggatggcgtggaggtgcacaatgccaagaccaagccacgggaggagcagtttaactccacatacagagtggtgtctgtgctgaccgtgctgcaccaggactggctgaacggcaaggagtataagtgcaaggtgtccaataagggcctgccctctagcatcgagaagacaatctctaaggcaaagggacagccaagggagcctcaggtgtacaccctgcctccaagccaggaggagatgacaaagaaccagtggtccctgacctgtctggtgaagggcttctatccttctgacatcgccgtggagtgggagagcaatggccagccagagaacaattacaagaccacaccccctgtgctggactctgatggcagcttctttctgtattctaggctgacagtggataagagccgctggcaggagggcaacgtgttttcttgcagcgtgatgcacgaggccctgcacaatcactacacccagaagtccctgtctctgagcctgggcaagcggagaaagaggggaagcggagagggaagaggctccctgctgacatgtggcgatgtggaggagaacccaggccccatgaagtatctgctgcccaccgcagccgccggcctgctgctgctggcagcccagccagccatggccgacatccagatgacccagagcccatcctctctgtccgcctctgtgggcgatagggtgacaatcacctgccacgcctcccagaacatctacgtgtggctgaattggtatcagcagaagccaggcaaggcccccaagctgctgatctacaaggcctccaatctgcacacaggagtgccttctcgcttcagcggatccggatctggaaccgactttacactgaccatcagctccctgcagccagaggatttcgccacctactattgccagcagggccagacatacccctatacctttggcggcggcacaaaggtggagatcaagaggaccgtggcagcaccatccgtgttcatctttccaccctctgacgagcagctgaagagcggaaccgcatccgtggtgtgcctgctgaacaatttctaccctagagaggccaaggtgcagtggaaggtggataacgccctgcagtccggcaattctcaggagagcgtgacagagcaggactccaaggattctacctatagcctgtctagcacactgaccctgagcaaggccgactacgagaagcacaaggtgtatgcctgtgaagtcacccaccaggggctgagttcaccttggacaaagagtttcaatcggggagagtgttgataa

SEQ ID NO: 26 - DNA, 2232 bp, Artificial Sequence
  transgenic construct/anti-CD33 (Gemtuzumab)
ttaagaaaaaatacgggtagaagcagtttgagagctccgttcgccaccatggagttcggcctgagttgggtgttcctggtcgccctgtttcggggggtccagtgtgaagtgcagctggtccagtccggggcagaagtcaagaagccaggcagctccgtgaaggtgtcttgcaaggccagcggctacaccatcacagactccaacatccactgggtgaggcaggcacctggacagtctctggagtggatcggctacatctatccatacaatggcggcaccgactataaccagaagttcaagaatagggccaccctgacagtggataaccccaccaatacagcctacatggagctgtctagcctgcgcagcgaggacacagccttttactattgcgtgaacggcaatccatggctggcctattggggccagggcacactggtgaccgtgtcctctgccagcaccaagggaccatccgtgttcccactggcaccttgctcccgctctacaagcgagtccaccgccgccctgggatgtctggtgaaggattacttccccgagcctgtgaccgtgtcctggaactctggcgccctgacaagcggagtgcacacctttccagccgtgctgcagagctccggcctgtattccctgtctagcgtggtgacagtgccctcctctagcctgggcaccaagacatacacctgcaacgtggaccacaagcctagcaataccaaggtggataagcgggtggagtccaagtatggaccaccttgcccaccatgtccagcaccagagtttctgggaggaccaagcgtgttcctgtttcctccaaagcctaaggacaccctgatgatcagcagaacaccagaggtgacctgcgtggtggtggacgtgtcccaggaggatcccgaggtgcagttcaactggtacgtggatggcgtggaggtgcacaatgccaagaccaagccccgggaggagcagtttaattctacatatagagtggtgagcgtgctgaccgtgctgcaccaggattggctgaacggcaaggagtacaagtgcaaggtgagcaataagggcctgccttcctctatcgagaagacaatctccaaggcaaagggacagccaagggagccacaggtgtataccctgcccccttctcaggaggagatgacaaagaaccaggtgagcctgacctgtctggtgaagggcttctacccctccgacatcgccgtggagtgggagtctaatggccagcctgagaacaattataagaccacaccacccgtgctggacagcgatggctccttctttctgtacagcaggctgacagtggataagtcccgctggcaggagggcaacgtgttttcttgcagcgtgatgcacgaggccctgcacaatcactatacccagaagtccctgtctctgagcctgggcaagcggagaaagaggggatccggagagggaaggggctctctgctgacatgtggcgacgtggaggagaaccctggcccaatgaagtacctgctgcctaccgcagccgccggcctgctgctgctggcagcccagccagccatggccgatatccagctgacacagtccccatctaccctgagcgcctccgtgggcgaccgggtgacaatcacctgcagagcctctgagagcctggataactatggcatccggttcctgacatggtttcagcagaagcctggcaaggccccaaagctgctgatgtacgcagcatccaatcagggatctggagtgcccagcagattctccggctctggcagcggcaccgagtttacactgaccatcagctccctgcagcctgacgatttcgccacatactattgccagcagaccaaggaggtgccatggagctttggccagggcacaaaggtggaggtgaagaggaccgtggcagcacctagcgtgttcatctttcctccaagcgacgagcagctgaagtccggcaccgcctctgtggtgtgcctgctgaacaatttctatccacgcgaggccaaggtgcagtggaaggtggataacgccctgcagtctggcaatagccaggagtccgtgacagagcaggactctaaggatagcacctactccctgtctagcacactgaccctgtccaaggccgactatgagaagcacaaggtgtacgcctgtgaggtcacccatcaggggctgtcatcaccagtcacaaagagtttcaatcggggggagtgctga

SEQ ID NO: 27 - DNA, 2250 bp, Artificial Sequence
  transgenic construct/anti-CD39
ttaagaaaaaatacgggtagaagcagtttgagagctccgttcgccaccatggagttcggcctgagttgggtgttcctggtcgccctgtttcggggggtccagtgccagatccagctggtgcagtccggccctgagctgaagaagccaggcgagacagtgaagatctcttgcaaggccagcggctacacattccggaactatggcatgaattgggtgaagcaggcaccaggcaagggcctgaagtggatgggctggatcaacacctacacaggcgagcctacctatgccgacgacttcaagggcaggttcgcctttagcctggccacctccgcctctacagcctacctgcagatctccaacctgaagaatgaggacaccgccacatatttctgcgcccgcaaggcctactatggcagcaattactattttgattactggggccagggcaccacactgaccgtgagctccgcctctacaaagggacctagcgtgttcccactggcaccatctagcaagagcacctccggaggaacagccgccctgggctgtctggtgaaggactacttccccgagcctgtgaccgtgtcctggaactctggcgccctgaccagcggagtgcacacatttccagccgtgctgcagtcctctggcctgtactccctgagctccgtggtgaccgtgccctctagctccctgggcacccagacatatatctgcaacgtgaatcacaagccttccaatacaaaggtggacaagagggtggagccaaagtcttgtgataagacccacacatgcccaccttgtccagcaccagagctgctgggaggaccatccgtgttcctgtttccacccaagcccaaggacaccctgatgatctctcgcacccctgaggtgacatgcgtggtggtggacgtgagccacgaggatccagaggtgaagttcaactggtacgtggatggcgtggaggtgcacaatgccaagaccaagcccagggaggagcagtaccagtccacctatcgcgtggtgtctgtgctgacagtgctgcaccaggactggctgaacggcaaggagtacaagtgcaaggtgtccaataaggccctgcctgccccaatcgagaagaccatctctaaggcaaagggacagcctagggagccacaggtgtacacactgcctccatctagagaggagatgaccaagaaccaggtgagcctgacatgtctggtgaagggcttctatcccagcgacatcgccgtggagtgggagtccaatggccagcctgagaacaattacaagaccacaccccctgtgctggacagcgatggctccttctttctgtattctaagctgaccgtggataagagccggtggcagcagggcaacgtgttttcttgcagcgtgatgcacgaggccctgcacaatcactacacccagaagtccctgtctctgagcccaggcaagaggagaaagaggggaagcggagagggaaggggctccctgctgacctgtggcgacgtggaggagaacccaggacctatgaagtatctgctgccaacagcagccgccggcctgctgctgctggcagcacagcccgccatggccgatatcgtgatgacccagagccacaagttcatgtccacatctgtgggcgacagggtgtccatcacctgcaaggcatcccaggacgtgagcacagcagtggcatggtaccagcagaagccaggacagagccctaagctgctgatctatagcgcctcctaccggtataccggcgtgcctgacagattcaccggctctggcagcggcacagatttcacctttacaatctccacagtgcaggccgaggacctggccgtgtactattgccagcagcactacaccacaccaccctatacctttggcggcggcacaaagctggagatcaagaggaccgtggcagcaccttccgtgttcatctttcctccatctgacgagcagctgaagagcggcacagcctccgtggtgtgcctgctgaacaacttctacccaagagaggccaaggtgcagtggaaggtggataacgccctgcagtctggcaatagccaggagtccgtgaccgagcaggactctaaggatagcacatattccctgtctagcaccctgacactgagcaaggccgattacgagaagcacaaggtgtatgcctgtgaagtgacccaccaggggctgtcctctccagtgaccaaatctttcaataggggcgaatgctgataa

SEQ ID NO: 28 - DNA, 2256 bp, Artificial Sequence
  transgenic construct/anti-CA 15-3
ttaagaaaaaatacgggtagaagcagtttgagagctccgttcgccaccatggagttcggcctgagttgggtcttcctggtcgccctgtttcggggggtccagtgtgaggtccagctgcagcagtccgggggagagagagccacccccggcgcctccgtgaagatgtcttgcaagacaagcggctacaccttcacaaactattggatgcactgggtgaagcagcggcccggacagggcctggagtggatcatcggctacatcaaccctagctccggctacacccagtataatcagaagtttaaggacaaggccaccctgacagccgataagtctagctccaccgcctatatccagctgtctagcctgacatctgaggacagcgccgtgtactattgctccacctactatggcgattacctgttcccatattggggacagggcaccctggtgacagtgtccgccgcatctacaaagggaccaagcgtgtttccactggcaccttcctctaagtctaccagcggaggaacagccgccctgggatgtctggtgaaggactacttccctgagccagtgaccgtgagctggaactccggcgccctgacctccggagtgcacacatttcctgccgtgctgcagagctccggcctgtacagcctgtctagcgtggtgaccgtgccatcctctagcctgggcacccagacatatatctgcaacgtgaatcacaagccttccaatacaaaggtggacaagcgggtggagccaaagtcttgtgataagacccacacatgcccaccttgtccagcacctgagctgctgggaggaccaagcgtgttcctgtttccacccaagcccaaggacaccctgatgatcagcagaacccccgaggtgacatgcgtggtggtggacgtgtcccacgaggatcctgaggtgaagttcaactggtacgtggatggcgtggaggtgcacaatgccaagaccaagcctcgggaggagcagtacaactccacctatagagtggtgtctgtgctgacagtgctgcaccaggactggctgaacggcaaggagtacaagtgcaaggtgtctaataaggccctgccagcccccatcgagaagaccatcagcaaggcaaagggacagccaagggagccacaggtgtacacactgcctccatctcgcgacgagctgaccaagaaccaggtgagcctgacatgtctggtgaagggcttctatccaagcgacatcgccgtggagtgggagtccaatggccagcccgagaacaattacaagaccacaccccctgtgctggactctgatggcagcttctttctgtattctaagctgaccgtggataagagccggtggcagcagggcaacgtgttttcctgctctgtgatgcacgaggccctgcacaatcactacacacagaagagcctgtccctgtctcccggcaagcggagaaagaggggaagcggagagggaagaggctccctgctgacctgtggcgacgtggaggagaaccctggcccaatgaagtatctgctgcctacagcagccgccggcctgctgctgctggcagcacagccagccatggccgatgtgctgatgacccagacaccactgtccctgccagtgtctctgggcgaccaggccagcatctcctgcaggtcctctcaggacatcgtgtacggcaacggcaatacctacctggagtggtatctgcagaagcccggccagtcccctaagctgctgatctataaggtgagcaataggttctccggcgtgccagaccgcttttctggcagcggctccggcaccgatttcacactgaagatctccagggtggaggcagaggacctgggcgtgtactattgcttccagggcagccacgtgccctacacctttggcggcggcacaaagctggagatcaagaggaggaccgtggcagcaccttccgtgttcatctttccaccctctgacgagcagctgaagtctggcacagccagcgtggtgtgcctgctgaacaacttctaccccagggaggccaaggtgcagtggaaggtggataacgccctgcagtccggcaattctcaggagagcgtgaccgagcaggactccaaggattctacatatagcctgagctccaccctgacactgagcaaggccgattacgagaagcacaaggtgtatgcctgtgaggtcacccaccagggactgagtagccccgtcaccaaatccttcaatagaggcgagtgttga

SEQ ID NO: 29 - DNA, 2262 bp, Artificial Sequence
  transgenic construct/ anti-CA 19-9
ttaagaaaaaatacgggtagaagcagtttgagagctccgttcgccaccatggagtttgggctgtcatgggtctttctggtcgctctgtttcggggggtgcagtgtcaggtgcagctggtcgagtcaggggggggaagtgtgcagcctggccggagcctgagactgagctgcgaggcatccggcttcacctttgaggcatacgcaatgcactgggtgcggcagccacctggcaagggcctggagtgggtgagctccatcaactggaattccggccggatcgcctacgccgactctgtgaagggcagattcaccatcagcagggataacgcccgcaattccctgtatctgcagatgaactctctgagactggaggacaccgccttttactattgcgccaaggacatccggagattcagcacaggcggcgccgagtttgagtactggggccagggcaccctggtgacagtgtctagcgccagcaccaagggaccatccgtgttccctctggcaccatcctctaagagcacctccggaggaacagccgccctgggctgtctggtgaaggactatttcccagagcccgtgacagtgtcctggaactctggcgccctgaccagcggagtgcacacatttcctgccgtgctgcagagctccggcctgtactccctgtctagcgtggtgaccgtgccatcctctagcctgggcacccagacatatatctgcaacgtgaatcacaagccatccaatacaaaggtggacaagagggtggagcccaagtcttgtgataagacccacacatgcccaccctgtcctgcaccagagctgctgggaggaccaagcgtgttcctgtttcctccaaagcctaaggacaccctgatgatcagccgcaccccagaggtgacatgcgtggtggtggacgtgtcccacgaggatcccgaggtgaagttcaactggtacgtggatggcgtggaggtgcacaatgccaagaccaagcccagggaggagcagtacaactctacctatcgcgtggtgagcgtgctgacagtgctgcaccaggactggctgaacggcaaggagtataagtgcaaggtgtctaataaggccctgcccgcccctatcgagaagaccatctccaaggcaaagggacagccaagggagcctcaggtgtacacactgcccccttctcgcgacgagctgaccaagaaccaggtgagcctgacatgtctggtgaagggcttctatcctagcgacatcgccgtggagtgggagtccaatggccagccagagaacaattacaagaccacaccacccgtgctggacagcgatggctccttctttctgtatagcaagctgaccgtggacaagtccaggtggcagcagggcaacgtgttttcttgcagcgtgatgcacgaggccctgcacaatcactacacacagaagtccctgtctctgagcccaggcaagaggaggaagaggggatccggagagggaagaggctctctgctgacctgtggcgatgtggaggagaatccaggccccatgaagtatctgctgcccacagcagccgccggcctgctgctgctggcagcacagcctgccatggcccagagcgtgctgacccagcctccatccgcctctggaaccccaggacagagggtgacaatcagctgctccggctcctctagcaacatcggcagcaatttcgtgtactggtatcagcagctgccaggaaccgcaccaaagctgctgatctacaggaacaatcagcggccctccggagtgccagacaggttttctggcagccgctccggcacatctgccagcctggccatctctggcctgcggagcgaggacgaggcagattactattgcgcagcatgggacgatagcctgggaggacactacgtgttcggaaccggaacaaaggtgaccgtgctgagaacagtggcagcaccaagcgtgttcatctttccccctagcgacgagcagctgaagtccggcaccgcctctgtggtgtgcctgctgaacaacttctaccctagagaggccaaggtgcagtggaaggtggataacgccctgcagtctggcaatagccaggagtccgtgaccgagcaggactctaaggatagcacatattccctgtcctctaccctgacactgtccaaggccgattacgagaagcacaaggtgtatgcctgtgaagtgacccaccagggactgagcagccccgtgaccaaatctttcaaccgaggcgaatgttga

SEQ ID NO: 30 - DNA, 2232 bp, Artificial Sequence
  transgenic construct/anti-CA 125
ttaagaaaaaatacgggtagaagcagtttgagagctccgttcgccaccatggagtttgggctgtcctgggtgttcctggtcgctctgtttaggggggtgcagtgtgaagtgcagctggtcgtcgagtccggggggggcctggtgcagccaggaggcagcctgaggctgtcctgcgcagcatccggctactctatcaccaacgactatgcctggaattgggtgagacaggcaccaggcaagggcctggagtgggtgggctacatctcttatagcggctacaccacatataaccccagcctgaagtccaggttcaccatctctcgcgataccagcaagaacacactgtacctgcagatgaattccctgagggcagaggacaccgccgtgtactattgcgccagatggacaagcggcctggattattggggccagggcaccctggtgacagtgagctccgccagcaccaagggcccttccgtgtttcccctggccccttctagcaagtccacctctggaggaacagccgccctgggctgtctggtgaaggactacttccctgagccagtgaccgtgtcttggaacagcggcgccctgacctccggagtgcacacatttcccgccgtgctgcagtcctctggcctgtacagcctgagctccgtggtgaccgtgccttctagctccctgggcacccagacatatatctgcaacgtgaatcacaagccatccaatacaaaggtggacaagcgggtggagcccaagtcttgtgataagacccacacatgcccaccttgtccagcacctgagctgctgggaggaccatccgtgttcctgtttccacccaagcctaaggacaccctgatgatcagcagaaccccagaggtgacatgcgtggtggtggacgtgtcccacgaggatcccgaggtgaagttcaactggtacgtggatggcgtggaggtgcacaatgccaagaccaagcccagggaggagcagtacaactctacctatcgcgtggtgagcgtgctgacagtgctgcaccaggactggctgaacggcaaggagtacaagtgcaaggtgtctaataaggccctgccagcccccatcgagaagaccatcagcaaggcaaagggacagccaagggagccacaggtgtacacactgcctccatctcgcgacgagctgaccaagaaccaggtgagcctgacatgtctggtgaagggcttctatcccagcgacatcgccgtggagtgggagtccaatggccagcctgagaacaattacaagaccacaccccctgtgctggactccgatggctctttctttctgtatagcaagctgaccgtggataagtcccggtggcagcagggcaacgtgtttagctgctccgtgatgcacgaggccctgcacaatcactacacacagaagtctctgagcctgtccccaggcaagaggagaaagaggggatccggcgagggcagaggctctctgctgacctgtggcgacgtggaggagaaccctggcccaatgaagtatctgctgcctacagcagccgccggcctgctgctgctggcagcacagcctgccatggccgacatccagatgacccagtccccatctagcctgtctgccagcgtgggcgacagggtgaccatcacatgcaaggccagcgatctgatccacaattggctggcctggtaccagcagaagcctggcaaggccccaaagctgctgatctatggagccacctctctggagacaggagtgccaagccgcttctccggatctggaagcggcaccgactttaccctgacaatctcctctctgcagcctgaggatttcgccacatactattgccagcagtactggaccacaccattcacctttggccagggcacaaaggtggagatcaagaggaccgtggccgccccttccgtgttcatctttccaccctctgacgagcagctgaagtccggcacagcctctgtggtgtgcctgctgaacaacttctacccacgcgaggccaaggtgcagtggaaggtggataacgccctgcagagcggcaattcccaggagtctgtgaccgagcaggacagcaaggattccacatattctctgagctccaccctgacactgagcaaggccgactacgagaagcacaaggtgtatgcctgtgaagtcacccaccaggggctgtcatcaccagtcactaagagtttcaatcggggggaatgctga

SEQ ID NO: 31 - DNA, 2238 bp, Artificial Sequence
  transgenic construct/anti-EGFR
ttaagaaaaaatacgggtagaagcagtttgagagctccgttcgccaccatggagttcgggctgagttgggtgttcctggtcgctctgtttcggggggtgcagtgtcaggtgcagctgaagcagtctgggcctgggctggtccagccctctcagagcctgtccatcacctgcacagtgtctggcttcagcctgaccaattacggagtgcactgggtgcggcagagccctggcaagggcctggagtggctgggcgtgatctggtccggaggcaatacagactataacacccccttcacctccagactgtctatcaataaggataactctaagagccaggtgttctttaagatgaatagcctgcagtccaacgacaccgccatctactattgcgccagggccctgacatactatgattacgagttcgcctattggggccagggcaccctggtgacagtgtctgccgccagcaccaagggccctagcgtgtttcccctggcccctagctccaagtccacctctggaggaacagccgccctgggctgtctggtgaaggactacttccctgagccagtgaccgtgtcctggaattctggcgccctgaccagcggcgtgcacacatttcccgccgtgctgcagtctagcggcctgtactccctgtcctctgtggtgaccgtgcctagctcctctctgggcacccagacatatatctgcaatgtgaaccacaagccatccaacacaaaggtggacaagaaggtggagcccaagtcttgtgataagacccacacatgcccaccttgtccagcacctgagctgctgggaggaccatccgtgttcctgtttccacccaagcctaaggacaccctgatgatctctagaaccccagaggtgacatgcgtggtggtggacgtgtcccacgaggatcccgaggtgaagttcaattggtacgtggatggcgtggaggtgcacaacgccaagaccaagccacgggaggagcagtacaactccacctatagagtggtgtctgtgctgacagtgctgcaccaggactggctgaatggcaaggagtacaagtgcaaggtgagcaacaaggccctgccagcccccatcgagaagaccatctccaaggcaaagggacagccaagggagccacaggtgtacacactgcctccatctcgcgacgagctgaccaagaatcaggtgagcctgacatgtctggtgaagggcttctatcctagcgacatcgcagtggagtgggagtccaacggacagccagagaacaattacaagaccacaccccctgtgctggacagcgatggctccttctttctgtattctaagctgaccgtggataagagccggtggcagcagggcaacgtgttcagctgctccgtgatgcacgaggccctgcacaaccactacacacagaagtctctgagcctgtcccctggcaagcggagaaagaggggaagcggagagggaaggggctccctgctgacctgtggcgacgtggaggagaatcctggcccaatgaagtatctgctgcccacagcagccgccggcctgctgctgctggcagcacagcctgccatggccgacatcctgctgacccagagccctgtgatcctgtctgtgagcccaggcgagcgggtgtccttctcttgcagagccagccagtccatcggcaccaatatccactggtaccagcagaggacaaacggcagccctcgcctgctgatcaagtatgcctctgagagcatctccggcatcccatccagattctctggcagcggctccggcaccgactttacactgtctatcaacagcgtggagtccgaggacatcgccgattactattgccagcagaacaataactggccaaccacattcggcgccggcaccaagctggagctgaagaggacagtggccgccccctccgtgttcatctttccaccctctgacgagcagctgaagtctggcaccgccagcgtggtgtgcctgctgaataacttttacccccgcgaggccaaggtgcagtggaaggtggataatgccctgcagtctggcaacagccaggagtccgtgaccgagcaggactctaaggatagcacatattccctgagctccaccctgacactgagcaaggccgattacgagaagcacaaggtgtatgcctgtgaagtcacccaccaggggctgtcctcacccgtcacaaaatctttcaataggggggagtgttga

SEQ ID NO: 32 - DNA, 1458 bp, Artificial Sequence
  transgenic construct/anti-nfP2X7
ttaagaaaaaatacgggtagaagcagtttgagagctccgttcgccaccatggagttcgggctgagttgggtgttcctggtcgctctgtttaggggggtgcagtgtgaagtgcagctgctggagtccggggggggcctggtccagccaggaggcagcctgaggctgtcctgcgcagcatctggcttcacctttaggaaccacgacatgggatgggtgcgccaggcacctggcaagggcctggagtgggtgagcgccatctctggaagcggaggatccacatactatgccaactctgtgaagggccggttcaccatcagcagagacaactccaagaatacactgtacctgcagatgaatagcctgcgggccgaggataccgccgtgtactattgcgccgagccaaagcccatggacacagagttcgattatagatccccaggcaccctggtgacagtgagctccgcctctaccaagggccccagcgtgtttcctctggccccatctagcaagtccacctctggaggaacagccgccctgggctgtctggtgaaggattacttccctgagccagtgacagtgtcttggaacagcggcgccctgacctctggagtgcacacatttcccgccgtgctgcagtcctctggcctgtactccctgagctccgtggtgaccgtgccttctagctccctgggcacccagacatatatctgcaacgtgaatcacaagccttctaatacaaaggtggacaagagggtggagccaaagagctgtgataagacccacacatgcccaccttgtccagcacctgagctgctgggaggaccaagcgtgttcctgtttccacccaagcccaaggacaccctgatgatctcccgcacccctgaggtgacatgcgtggtggtggacgtgtctcacgaggatccagaggtgaagtttaactggtacgtggatggcgtggaggtgcacaatgccaagaccaagccccgggaggagcagtacaacagcacctatagagtggtgtccgtgctgacagtgctgcaccaggactggctgaacggcaaggagtataagtgcaaggtgagcaataaggccctgccagcccccatcgagaagaccatctccaaggcaaagggacagccaagggagcctcaggtgtacacactgcctccaagccgcgacgagctgaccaagaaccaggtgtccctgacatgtctggtgaagggcttctatccctccgacatcgccgtggagtgggagtctaatggccagcctgagaacaattacaagaccacaccccctgtgctggactccgatggctctttctttctgtattctaagctgaccgtggataagagcagatggcagcagggcaacgtgttcagctgttccgtgatgcacgaagcactgcacaaccactacactcagaaatccctgtcactgtcacctggcaagtgataa

SEQ ID NO: 33 - DNA, 2232 bp, Artificial Sequence
  transgenic construct/anti-PSMA
ttaagaaaaaatacgggtagaagcagtttgagagctccgttcgccaccatggagtttggactgagttgggtgttcctggtcgccctgtttagaggcgtgcagtgtgaagtgcagctggtgcagagcggccctgaagtgaaaaagccaggcgccaccgtgaagatctcttgcaagacaagcggctacaccttcacagagtataccatccactgggtgaagcaggcaccaggcaagggcctggagtggatcggcaacatcaatcctaacaatggcggcaccacatacaaccagaagtttgaggacaaggccaccctgacagtggacaagagcaccgatacagcctatatggagctgagctccctgcggtccgaggacaccgccgtgtactattgcgccgccggctggaatttcgattactggggccagggcaccctgctgacagtgtctagcgccagcacaaagggaccatccgtgtttccactggcaccttcctctaagtccacctctggaggaacagccgccctgggctgtctggtgaaggattacttccctgagccagtgaccgtgtcctggaactctggcgccctgacctccggagtgcacacatttccagccgtgctgcagagctccggcctgtacagcctgtctagcgtggtgaccgtgccttcctctagcctgggcacccagacatatatctgcaacgtgaatcacaagccatccaatacaaaggtggacaagcgggtggagcccaagtcttgtgataagacccacacatgcccaccttgtccagcacctgagctgctgggaggaccatccgtgttcctgtttccacccaagcctaaggacaccctgatgatctctagaacccctgaggtgacatgcgtggtggtggacgtgagccacgaggatccagaggtgaagttcaactggtacgtggatggcgtggaggtgcacaatgccaagaccaagcctagggaggagcagtacaactctacctatcgcgtggtgagcgtgctgacagtgctgcaccaggactggctgaacggcaaggagtataagtgcaaggtgtccaataaggccctgccagcccccatcgagaagaccatcagcaaggcaaagggacagccaagggagccacaggtgtacacactgcctccatctagagacgagctgaccaagaaccaggtgagcctgacatgtctggtgaagggcttctatccatccgacatcgccgtggagtgggagtctaatggccagcccgagaacaattacaagaccacaccccctgtgctggacagcgatggctccttctttctgtatagcaagctgaccgtggacaagtccaggtggcagcagggcaacgtgtttagctgctccgtgatgcacgaggccctgcacaatcactacacacagaagtctctgagcctgtccccaggcaagaggagaaagaggggatccggagagggaaggggctctctgctgacctgtggcgatgtggaggagaaccctggcccaatgaagtatctgctgcctacagcagccgccggcctgctgctgctggcagcacagcctgccatggccgacatccagatgacccagtccccatcctctctgtctacaagcgtgggcgatcgcgtgaccctgacatgcaaggcatcccaggacgtgggaaccgcagtggattggtaccagcagaagcccggccctagcccaaagctgctgatctattgtgccagcacccggcacacaggcatcccatccagattctccggctctggcagcggcaccgactttaccctgacaatcatcagctccctgcagccagaggacttcgcagcagattactattgccagcagtacaatagctatcctctgacctttggcccaggcacaaaggtggacatcaagaggaccgtggccgcccctagcgtgttcatctttccacccagcgatgagcagctgaagtccggcacagcctctgtggtgtgcctgctgaacaacttctacccacgcgaggccaaggtgcagtggaaggtggacaacgccctgcagtctggcaatagccaggagtccgtgaccgagcaggactctaaggatagcacatattccctgtctagcaccctgacactgagcaaggccgattacgagaagcacaaggtgtatgcctgcgaggtcactcatcaggggctgtcatcaccagtcaccaaaagtttcaataggggggaatgttga

SEQ ID NO: 34 - DNA, 768 bp, Artificial Sequence
  transgenic construct/eGFP
ttaagaaaaaatacgggtagaattggagcggccgcattgtgccaagatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaga

SEQ ID NO: 35 - DNA, 17430 bp, Newcastle disease virus
  Mut HN(F277L)/M(G165W)/F(F117S)/F(F190L)/ F(L289A)/L(V757I)/L(F1551S)/L(R1700L)-Sofituzumab
accaaacagagaatccgtgagatacgataaaaggcgaaggagcaatcgaagtcgtacgggtagaaggtgtgaatctcgagtgcgagaccgaagcttaaactcgagagagccttctgccaacatgtcttccgtatttgacgagtatgaacagctcctcgcggctcagactcgccctaatggagctcacggaggaggagagaaggggagcaccttaaaagttgaagtcccggtattcactcttaacagtgacgacccagaagatagatggaactttgcagtattctgtcttaggattgctgttagcgaagatgccaacaaaccactcagacaaggtgctctcatatctctcttatgctcccattctcaagtgatgaggaaccatgtcgcccttgcagggaaacagaatgaggccacactggctgttcttgagatcgatggtttcaccaacagcgtgcctcaatttaacaacaggagtggagtgtctgaggagagagcacagagattcttgatgatagcagggtccctccctcgggcatgcagcaacggtaccccgttcatcacagctggggttgaagatgatgcaccagaagacatcactgatactctggaaagaatcatctctatccaggctcaagtatgggtcacggtagcaaaggccatgactgcatatgagacagcggatgagtcggaaacaagaagaatcaataagtatatgcagcaaggcagagtccaaaagaagtacatcctccaccccgtatgtaggagcgcaattcaactcacgattagacaatctctggcagtccgcattttcttggttagcgagcttaagagaggccgcaacacggcaggtggaagttccacttattatagcttggtaggggacatagactcatacatcaggaacaccgggcttactgcattcttcctgacactcaggtatggaattaacaccaagacatcagcccttgcactcagcagcctcgcaggcgatatccaaaaaatgaagcagctcatgcgtctatatcggatgaaaggagataatgcgccgtacatgacactgctcggtgacagtgaccagatgagctttgcaccggctgagtatgcacaactctactcttttgcaatgggtatggcatccgtcctagacaaaggaactggcaagtaccagttcgccagagactttatgagcacatcattctggagacttggagtagagtatgctcgggctcagggaagcagcatcaatgaggatatggctgccgagctaaagctgaccccagcggcaaggaggggcctggcagctgctgcccaacgagcgtccgaggagaccggcagcatggacattcccactcaacaagctggggtcctcacagggctcagcgacggaggccctcaagccccacaaggtggactgaacaggtcgcaagggcaaccggacgccggagatggggagacccaatttttagatttgatgagagcagtggcaaatagtatgagagaagcgccaaactctgtgcagaacaccactcagcaggagcctccatctactcctgggccatctcaagacaacgacaccgactggggatactgaccgacaacactcagcctgcctccatcgaattatctcgattcttctgcctgtaacctaacccctggtccacagtctcacacggccaaacccgcaaacgagcccccccctctctccccccccctccagccacacggctccaaccgcccaaaacaacacgggcacaactcgactcactcataatctacacagagccgaagatcttagaaaaaaatacgggtagaagagagacattcagagatcaggacaaatcatcagggtctccgctctcccctctacccagcagaccagagtgaagatggccaccttcacggatgcagagatcgacgatctatttgaaaccagtggaactgtcattgacagcataattacggcccaaggcaacccagtagagactgttggaaggagcgcaatcccacaaggcaaaactaaagcattgagtgcagcatgggagaaacatggaagcacccaaccgccggccggccaagacacccctgatcgaatggacagatcagacaaacaaccatccccacccgaacaggcaaccacacacaacagcccgccagccacatccaccgaccagccccccacccaggccgcaggcgaagccggtgacacacagctcaagactggagcaagcaactccctcctgtcgatgctcgacaagcttagcaataaatcgtccaatgctaaaaagggcccacggtcgagtccccaggaagggcaccaccaacctccgacccagcagcaagggagtcaacagagccgcggaaacagtcaggagagatcgcaaaaccaggtcaaggccgcctctggagaccagggcacagacgcgaacacagcatatcatggacaatggaaggagtcacaaccatcagctggtgcaatccctcatgctctccggtcagggcagagccaagccaatactcctgcatctgtggatcatgtccagctacctgtcgactttgtgcaggcgatgatgtctatgatggaggcgatatcacagaaggtaaataaagtcgactatcagctagaccttgtctcaaagcagacatcctccatccccatgatgcgatctgaaatccaacagctgaaaacatccgttgcggtcatggaagccaacctgggcatgatgaagattctggaccctggttgtgctaacgtttcatctttaagtgacctacgggcggtcgcccgctctcacccagttttagtttcaggccccggagacccatctccttatgtgacacaagggggtgaattgatactcaataaactctcgcaaccagtgcaacatccttctgagttgattaaacctgccacgataagcgggcctgatataggagtggagaaggacactgtccgtgcattgatcacctcacgcccaatgcatccgagctcttcagctaagctcctgagtaagctggatgcagccgggtcgatagaagaaattaggaaaatcaaacgccttgcgctgaatggctgatcactactacaacccgcaacgggttcccgtttatctattgtcacaaggaacccgccccgaagcttaagaaaaaatacgggtagaagcagtttgagagctccgttcgccaccatggagtttgggctgtcctgggtgttcctggtcgctctgtttaggggggtgcagtgtgaagtgcagctggtcgtcgagtccggggggggcctggtgcagccaggaggcagcctgaggctgtcctgcgcagcatccggctactctatcaccaacgactatgcctggaattgggtgagacaggcaccaggcaagggcctggagtgggtgggctacatctcttatagcggctacaccacatataaccccagcctgaagtccaggttcaccatctctcgcgataccagcaagaacacactgtacctgcagatgaattccctgagggcagaggacaccgccgtgtactattgcgccagatggacaagcggcctggattattggggccagggcaccctggtgacagtgagctccgccagcaccaagggcccttccgtgtttcccctggccccttctagcaagtccacctctggaggaacagccgccctgggctgtctggtgaaggactacttccctgagccagtgaccgtgtcttggaacagcggcgccctgacctccggagtgcacacatttcccgccgtgctgcagtcctctggcctgtacagcctgagctccgtggtgaccgtgccttctagctccctgggcacccagacatatatctgcaacgtgaatcacaagccatccaatacaaaggtggacaagcgggtggagcccaagtcttgtgataagacccacacatgcccaccttgtccagcacctgagctgctgggaggaccatccgtgttcctgtttccacccaagcctaaggacaccctgatgatcagcagaaccccagaggtgacatgcgtggtggtggacgtgtcccacgaggatcccgaggtgaagttcaactggtacgtggatggcgtggaggtgcacaatgccaagaccaagcccagggaggagcagtacaactctacctatcgcgtggtgagcgtgctgacagtgctgcaccaggactggctgaacggcaaggagtacaagtgcaaggtgtctaataaggccctgccagcccccatcgagaagaccatcagcaaggcaaagggacagccaagggagccacaggtgtacacactgcctccatctcgcgacgagctgaccaagaaccaggtgagcctgacatgtctggtgaagggcttctatcccagcgacatcgccgtggagtgggagtccaatggccagcctgagaacaattacaagaccacaccccctgtgctggactccgatggctctttctttctgtatagcaagctgaccgtggataagtcccggtggcagcagggcaacgtgtttagctgctccgtgatgcacgaggccctgcacaatcactacacacagaagtctctgagcctgtccccaggcaagaggagaaagaggggatccggcgagggcagaggctctctgctgacctgtggcgacgtggaggagaaccctggcccaatgaagtatctgctgcctacagcagccgccggcctgctgctgctggcagcacagcctgccatggccgacatccagatgacccagtccccatctagcctgtctgccagcgtgggcgacagggtgaccatcacatgcaaggccagcgatctgatccacaattggctggcctggtaccagcagaagcctggcaaggccccaaagctgctgatctatggagccacctctctggagacaggagtgccaagccgcttctccggatctggaagcggcaccgactttaccctgacaatctcctctctgcagcctgaggatttcgccacatactattgccagcagtactggaccacaccattcacctttggccagggcacaaaggtggagatcaagaggaccgtggccgccccttccgtgttcatctttccaccctctgacgagcagctgaagtccggcacagcctctgtggtgtgcctgctgaacaacttctacccacgcgaggccaaggtgcagtggaaggtggataacgccctgcagagcggcaattcccaggagtctgtgaccgagcaggacagcaaggattccacatattctctgagctccaccctgacactgagcaaggccgactacgagaagcacaaggtgtatgcctgtgaagtcacccaccaggggctgtcatcaccagtcactaagagtttcaatcggggggaatgctgagctgaccgagccctcctctataaacccaagattcaaggctccaagcgataaccttctcccgcctcttccatcccactgaatgatcgcgcagctgcaattaatctagcaacattaaagattaagaaaaaatacgggtagaatcggagtgcaccgattgtgccaagatggactcatctaggacaattgggttatactttgattctgcccttccttctagcaacctgttagcattcccgattgtcttacgagatgtaggagatggaaagaagcaaattactccgcaatataggatccggcgtcttgactcgtggacagacagtaaagaagactcggtattcatcactacctacggattcatcttccaggttgggaatgaagaagtcactgtcggcatgatcaatgataatcccaagcgagagttactctccgctgcgatgctctgcctaggaagcgtcccgaatgttggagatcctgttgagctggcaagggcctgcctcactatggtggtaacatgcaagaagagtgcaactaatactgagagaatggtcttctcagtagtgcaggcaccccgggtgctgcaaagctgtagggtcgtggcagacaaatactcgtcagtgaatgcagttaagcacgtgaaagcaccagagaagatcccttggagtgaaaccctagagtacaaggtgaattttgtctctttgactgtggtgccgaagaaggatgtctacaagatcccaaccgcagtattgaaggtatccggctcgagcctgtataatcttgcgctcaatgtcactattgatgtggaggtagacccaaagagcccgttagtcaagtccctttctaggtccgacagtggatactatgctaatctcttcttacatatcggacttatgtccactgtagataagaggggaaagaaagtgacatttgaccagctggagaggaagataaggagacttgatctatctgtcgggctcagtgatgtgctcggaccttctgtgcttgtgaaggcgagaggtgcacggactaggctgctggcacctttcttctctaacagcgggacagcctgctaccctatagcaaatgcctctcctcaggtagctaagatactctggagtcagaccgcgtgcctgcggagtgtaaaaatcattattcaagcgggcacccaacgcgctgtcgcagtgactgctgaccatgaggttacctctactaagatagagaagaggcataccatcgctaaatacaatcccttcaagaaataggccgtatccctgagactgcaattcacccgccttcccaaaacaccatgacaccagataatgatctgtcttgattacttacagttagtttccctgtctatcaaattagaaaaaacacgggtagaagagttcggatcccggccggcgcaccaaaagcgcaagatgggccccagatcttctaccaggatcccagtacctctaatgctgaccatacggatcacgctggcactgagttatgtccgtctgacaagttctcttgatggcaggcctcttgcagccgcagggatcgtggtaacaggggataaagcagttaacatatacacctcatcccagacagggtcaatcatagtcaagttactcccaaatatgcccaaggacaaagaggcatgtgcaaaagccccattggaggcttacaacaggacactgactactttgcttaccccccttggtgattctatccgcaggatacaagagtctgtgactacatccggaggaaggagacagagacgctctataggtgccattattggcagtgtagctctaggggttgcaacagctgcacagataacggcagcctcggctctgatacaagccaaccagaatgctgctaacatcctccggcttaaggagagcattgctgcaaccaatgaagctgtacacgaggtcactggcggattgtcacagttagcagtggcagttggaaagatgcagcaatttgttaatgaccagcttaataacacagctcaagagttggactgtataaaaattacacagcaggttggtgtagaactcaacttatacttaactgaattgactacagtgttcgggccacaaatcacttcccctgccttaactcagttgactgttcaggctctttacaatctggctggtggtaatgtagattacttgttgactaagttaggtgtagggaacaaccagctcagctcattgattggtagcggcttgatcaccggtaaccctattttttacgactcacagactcaactcttgggcatacaggtgactgcaccctcagtcgggaacctaaataatatgcgtgccacctacttggagaccttgtctgtaagcacaacaaagggatttgcctcagcacttgtcccaaaagtagtgacacaggtcggttctgtgatagaagagcttgacacctcatactgtatagaagctgatttggatttatattgtacaagaatagtgacattccctatgtcccctggtatttattcctgtttgagcggcaatacatcggcttgcatgtattcaaggactgaaggcgcacttactacaccatacatgactctcaaaggctcagttgttgccaattgccagatgacaacatgtagatgtgcagaccccccgggtatcatatcacaaaattatggagaggctgtgtctctaatagataagcactcatgcaatgtcgtatccttagacgggataactttgaggctcagtggggaatttgatgcaacttatcaaaagaatatctcaatattagattctcaagtactagtgacaggcaatctcgatatctcaactgagcttgggaatgtcaaccactcaataagtaatgctttggataagttagaggaaagcaacagcaaactagacaaagtcaatgtcaaactgaccagcacatctgctctcattacctatattgttttaactgtcatatctcttgttcttggtatgcttagcctggttctagcatgctatctgatgtacaagcaaaaagcgcaacgaaagaccttgttgtggcttggaaataataccctagatcagatgagagctactacaaaaatgtgaatgcagatgagaggcagaggtatccccaatagcaatctgtgtgtcaattctggcagcctgttaatcagaagaattaagaaaaaactaccggatgtaggtgaacaaaagggaatatacgggtagaacggcctgagaggccacccctcaatcgggagccaggccccactacgtccgctctaccgcatcaccaacagcagtcttcagtcatggacagcgcagttagccaagttgcgctagagaatgatagaagagaagcgaataatacatggcgcttggttttccggatcgcagccttacttttactggtaataaccttagccgtctctgcagtcgccctggcatatagtatggaggctagcacacctggcgaccttgtaagcataccgactgcgatctctagggcagaggaaaggattacatctgcactcggttccaatcaagatgtagtagataggatatacaagcaagtggcccttgagtctccattggcactgctaaataccgaatctataattatgaatgcaataacgtctctctcttatcaaatcaatggagccacaaataatagcgggtgtggggcacctgttcatgacccagattacatcggggggataggtcaagaacttattgtagatgatacgagtgacgtcacatcattttatccctctgcattccaagaacacctgaattttatcccggcgcccactacaggatcaggctgcactcggataccctcattcgacatgagtgctacccattactgttacactcacaatgtgatattgtctggctgcagagatcactcacactcacatcagtatttagcacttggtgtgcttcggacatctgcaacagggagggtattcttttctactctgcgttccatcaacttggatgacgcccaaaatcggaagtcttgcagtgtgagtgcaactcctttaggttgtgatatgctgtgctctaaaatcacagagactgaggaagaggattataaatcagttatccccacatcgatggtacatggaaggttagggcttgacggccaataccatgagaaggacctagacgtcacaacactatttagggactgggtggcaaattacccaggagtaggaggtgggtcttttattaacaatcgcgtatggttcccggtctacggagggctaaaacccagctcgcctagtgacactgcacaagaagggagatacgtaatatacaagcgatacaatgacacatgcccggatgagcaagattaccagattcggatggctaagtcatcgtataagcctaggcggtttggtggaaaacgcgtacagcaggccatcctatccatcaaggtgtcaacatccttgggtgaggacccggtgttgactgtaccgcctaatacggtcgcactcatgggggccgaaggcagagttctcacagtggggacatctcatttcttatatcagcgagggtcatcatacttctctcccgctttgttataccctatgacagtcaacaacaaaacagccactcttcataatccttatacattcaatgctttcactcggccaggtagtgtcccttgccaggcttcagcaagatgccccaactcatgtgttaccggagtctatactgatccatatcccttagtcttccataggaaccacaccttgcgaggggtattcggaacaatgcttgatgacgaacaagcaagactcaaccctgtatctgcagtatttgataacatatcccgcagtcgcataactcgggtaagttcaagcagtaccagggcagcatacacgacatcaacgtgttttaaagttgtcaagaccaataaaacctattgcctcagcattgcagaaatatccaataccctcttcggggaattcagaattgtccccttactagttgagattctcaaggatggtggggtttaagaagctaggaatggtcggttgagtcaaccgtgagaaggcggggaagatgatattgcatcatctatcttctgtaacaccaagaatcaaatcgaataccagtgcgagctcgaatcctacgctgccagtcagccataatcggctggtgctaatgtggttagcctgaatcttgtcgatagtcacttgattaagaaaaaatgtggacggtagtgaaatataaggcaaagcagctcacagaggacagcacgggtaggacatggcgggctccggacccgaaagggcagagcatcagattatcctaccagagtcacatctgtcctcaccattggtcaagcataaattgctttattactggaaattaactgggctaccgcttcctgacgaatgtgacttcgaccatcttattatcagccgacaatggaagaaggtacttgaatcggccacccctgacattgagagaatgataaaactagggcgggcagtacaccagactctcaaccacaattccaggataaccggagtactccatccccggtgtttagaggaactggctagtattgaggttcctgattcaaccaacaaatttcggaagatcgaaaaaaagatccagattcacaacacaaggtatggagaactgttcacaagattatgcacgcatgtagaaaagaaactattggggtcatcttggtctaacaatgtcccacggtcagaggaattcaacagcatccgtacagatccggcattttggtttcactcaaaatggtccacagccaaatttgcatggctccatataaaacaggtccagaggcatctgattgtagcagcaagaacaaggtccgcagtcaacaaattagtgacgctgacccataaggtaggccaaatctttgttactcctgagcttgtcattgtgacacatacagatgagaacaagttcacgtgtcttacccaggaacttgtgttgatgtatgcagatatgatggagggcagagatatggtcagcataatatcatccacggcggcacatcttaggagcttatcagagaaaattgatgatattctgcggttagtagatgctctggcaagagatttgggcaatcaagtctacgatgttgtagcactaatggagggattcgcatacggcgctgttcagctgcttgaaccgtcaggtacatttgcgggggatttcttcgcattcaacctgcaggagctcaaagatactctaaccggactcctccccaaggatatcgcagaatctgtgactcacgcaatcgcaaccatattctctggcttagaacaaaatcaagcagctgagatgttgtgcctgttgcgtctgtggggtcacccactactcgagtcccgtattgcagcaaaagcagttaggagtcaaatgtgcgcaccaaaaatggtagactttgatatgatcctccaggtattatctttctttaaaggaacaatcatcaatggatatagaaagaagaatgcaggcgtgtggccacgtgtcaagatagatacgatatacgggaaggtcatagggcagctacacgcagattctgcagagatttcacatgatgtcatgttgagggaatacaagagtttatctgcacttgaattcgagccatgtatagagtatgaccctgtcaccaatctgagcatgtttttaaaagacaaggcaatcgcacacccgaaagacaactggctcgcttcgtttaggcgaaaccttctctctgaggaccagaagaaacatgtaagagaggcaacctcaactaaccgcctcttgatagagttcttagagtcaaatgattttgatccatataaagagatggaatatctgacgacccttgagtacctaagagatgataatgtggcagtatcatactcactcaaagaaaaagaggtgaaagttaatgggcggattttcgcaaagctaacaaatagattaaggaattgtcaggtaatggcagaagggatcctagctgaccagattgcacctttcttccagggaaatggggtcattcaggatagcatatctttgactaaaagtatgctagcgatgagtcaactgtctttcaacagcaacaagaaacgtattactgactgcaaagaaagagtatcctcaaaccgcaatcatgatccgaagagcaagaatcgtcggagagttgccacttttgtaactaccgacctgcaaaagtattgtctcaattggagatatcagacagtcaagctgttcgctcatgccatcaatcagctgatgggcttacctcacttcttcgagtggattcatcttagactaatggatactacgatgtttgtaggggaccctttcaatcctccaagtgaccctacggactgtgatctatcaagagtcccaaatgatgacatatatattgtcagtgctaggggaggcattgagggattatgccagaagctatggacaatgatctcaattgctgcaatccagcttgctgcagcaagatcacattgtcgcgttgcctgtatggtacaaggcgacaatcaagtaatagctataacgagagaggtaagatcagatgactccccagagatggtgttaacacaattgcatcaagccagtgataatttcttcaaggaattgattcatgtcaatcatttgatcggccataatttgaaggatcgtgaaaccatcaggtcagacacgttcttcatatacagcaaacgaatattcaaagatggagcaatactcagtcaggtcctcaaaaactcatctaaattagtgctaatatcaggcgaccttagtgaaaacactgtaatgtcttgtgccaacattgcatctactgtagcacggttatgcgagaacgggcttcctaaggatttctgttattacttaaactacttaatgagttgcgtgcagacatacttcgattccgaattttccatcaccaacaactcgcaactcgattctaaccagtcgtggatagaggacatttcttttgtgcactcatatgtcctgacccctgctcaactggggggactgagtaaccttcaatactcaaggctctacacaaggaacatcggcgacccgggaaccactgctttcgcagagatcaagagattagaggcagtggggttactgagtcctagcattatgactaacatcttaactaggccgcctggaaatggagattgggccagtctgtgcaacgatccatactcctttaattttgagactgtcgcaagcccaaatattgtccttaagaaacatacacaaagagtcctatttgaaacttgctcgaatcccttattatctggagtgcacacagaggataatgaggcagaagagaaggcattggctgaattcttgctcaatcaagaaatgattcatccacgtgtcgcgcatgctatcatggaagcaagctctgtaggtaggagaaagcaaattcaggggcttgttgacacaacaaacaccgtgattaagattgcactgactaggaggccactcggcatcaagaggctgatgcggatagtcaattactcgagcatgcatgcaatgctatttagagatgatgttttctcgtctaatagatccaaccatcccttagtctcttctgatatgtgttctcagacactggcagactatgcacggaacagaagctggtcacctttgacagggggtagaaaaatactgggtgtatctaatcctgataccatagaacttgtagagggtgagatccttagtgtcagtggagggtgcactagatgtgacagtggggatgaacagtttacttggttccatcttccaagcaatatagagctaaacgatgacaccagcaagaatcctccaatgagagtgccatatctcgggtcaaagactcaagagaggagagccgcctcgcttgcgaaaatagctcatatgtcaccacacgtgaaggcggctctaagggcgtcatctgtgttaatctgggcttatggggacaacgaaataaactggactgctgcccttaagattgcaaggtctcggtgcaacataagctcagagtatcttcgactattgtcacccttgcctacagctgggaatctccaacatagattggacgacggcataactcagatgacattcacccctgcatctctatacagggtgtcaccttacattcatatatctaatgattctcaaaggctatttactgaagaaggagtcaaagaggggaatgtggtttatcagcaaatcatgctcttgggtttgtccttaattgagtcactcttcccaatgacaacaaccaagacatatgatgaaatcacattgcacctccacagtaaatttagctgctgtatcagggaagcacctgttgcagttcctttcgagctactcggggtggcaccggaactaagggcagtaacctcaaataagtttatgtatgatcctagccctgtatcagagggagactttgcgagacttgacttagctatctttaagagttatgaacttaatttagagtcatattccacaatagagctaatgaacgttctttcaatatctagtgggaagttgattggccagtctgtggtttcttatgatgaagatacctccataaagaatgacgctataatagtgtatgacaacacacggaattggatcagcgaagctcagaattcagatgtggtccgcctattcgagtatgcggcactcgaagtgctcctcgactgttcttatcaactctactatctgagagtaagaggcctaaacaatatcgtcctgtacatgagtgatttatacaagaatatgccaggaattctactctctaatattgcagccacaatatctcaccctgtcatccattcaaggttgaatgcagtaggtctggtcaaccatgacgggtcacaccaacttgcagacacagatttcattgaaatgtctgcaaagctgctagtctcttgcactcgacgcgtggtctcaggtttacatgcagggaataagtatgacctgctgtttccatctgtcttggatgataacctaagtgagaagatgcttcagttgatatcccaattatgctgtctgtatacggtgctctttgctacaacaagagaaatcccgaaaataagaggcttatctgcagaagagaaatgttcagtacttactgagtacctactgtcagatgctgtgaaaccgttacttgggtccgagcaagtgagctctatcatgtctcccaacatagttacgttcccagccaatctgtattacatgtctaggaagagccttaatttgatcagggagagagaggacagggatactatcttggcattgttgttccctcaagaaccgctgctcgagtttcctctggtacgagatattggtgctcgtgtaaaagatccatttaccctacaacctgcggcgtttttacaagagttagatttgagtgctccggcaaggtatgacgcattcacaatcagtcaggcgcattctgaacacatattgccaaacccagaggaagatcacttagtacgatacttgttcagaggaatagggactgcgtcctcctcttggtataaggcatcccatcttctttctgtacccgaggtcagatgtgcaaggcatgggaactccttatatttagcagaaggaagtggagccatcatgagtcttctcgaattgcatataccacacgaaactatctattacaatacacttttctcgaacgagatgaaccccccacagcgacatttcggaccaactccaacacagtttctgaattcggttgtttttaggaatttacaggcggaagtaccatgcaaggatggatttgtccaggagttccgtccgttatggagagagaatacagaagaaagcgatctgacctcagataaagcagtgggatatatcacatctgtagtgccgtacaggtctgtatcattgctgcattgtgacattgaaatccctccaggatctaatcaaagcttactagatcagctggctaccaatctgtctctgattgccatgcattctgtgaaggagggcggggtcgtgattatcaaagtactgtatgcaatgggatattacttccatctactcatgaacttgtttactccatgttccacgaaaggatatattctctctaatggctatgcctgtagaggggatatggagtgttacctgatatttgtcatgggctacctaggcgggcctacatttgtacatgaggtggtgaggatggcaaaaacgctagtacagcggcatggcacacttttgtctaagtcagatgagattacactgactaggttatttacttcacagcaacagcgtgtaacagacatcctatccagccctttaccgagactaatgaagtacttgagagagaatattgatactgcactgattgaagccgggggacagcctgtccgtccattctgtgcagagagtttagtgagcacactaacagacatgactcagacaacccagatcatcgccagccacattgatacagtcattcgatctgtgatctatatggaagcagagggtgatcttgctgacacagtattcttatttaccccttacaacctctctatggacgggaaaaagagaacatcacttaaacagtgcacaagacagatcttagaggtcacaatactgggtctcagagtcaaaaatctcaataaagtaggtgatgtaatcagcctagtactcagaggtatgatttctctggaggaccttctcccactgagaacctacttgaagtgtagtacctgccctaagtatttgaaggctgtcctaggtattaccaaactcaaagaaatgttcacagacgcctctttattatacttgactcgtgctcaacaaaaattctacatgaaaactataggcaatgcagtcaagggatactatagtaactgtgactcttaaaggcaaccacatatcaataggccctctttctagccgatcgtattcttgttgacttcattataccatattagaaaaaaattgaattccgaccctttaagactcgtattcggattcaaataattatctcagaaaaaaagtgcacgtagttgttcttgattatagtcccgtcattcaccaagtctttgtttggt

SEQ ID NO: 36 - protein, 571 aa, Newcastle disease virus
  HN protein
MDSAVSQVALENDRREANNTWRLVFRIAALLLLVITLAVSAVALAYSMEASTPGDLVSIPTAISRAEERITSALGSNQDVVDRIYKQVALESPLALLNTESIIMNAITSLSYQINGATNNSGCGAPVHDPDYIGGIGQELIVDDTSDVTSFYPSAFQEHLNFIPAPTTGSGCTRIPSFDMSATHYCYTHNVILSGCRDHSHSHQYLALGVLRTSATGRVFFSTLRSINLDDAQNRKSCSVSATPLGCDMLCSKITETEEEDYKSVIPTSMVHGRLGLDGQYHEKDLDVTTLFRDWVANYPGVGGGSFINNRVWFPVYGGLKPSSPSDTAQEGRYVIYKRYNDTCPDEQDYQIRMAKSSYKPRRFGGKRVQQAILSIKVSTSLGEDPVLTVPPNTVALMGAEGRVLTVGTSHFLYQRGSSYFSPALLYPMTVNNKTATLHNPYTFNAFTRPGSVPCQASARCPNSCVTGVYTDPYPLVFHRNHTLRGVFGTMLDDEQARLNPVSAVFDNISRSRITRVSSSSTRAAYTTSTCFKVVKTNKTYCLSIAEISNTLFGEFRIVPLLVEILKDGGV

SEQ ID NO: 37 - protein, 364 aa, Newcastle disease virus
  M protein
MDSSRTIGLYFDSALPSSNLLAFPIVLRDVGDGKKQITPQYRIRRLDSWTDSKEDSVFITTYGFIFQVGNEEVTVGMINDNPKRELLSAAMLCLGSVPNVGDPVELARACLTMVVTCKKSATNTERMVFSVVQAPRVLQSCRVVADKYSSVNAVKHVKAPEKIPWSETLEYKVNFVSLTVVPKKDVYKIPTAVLKVSGSSLYNLALNVTIDVEVDPKSPLVKSLSRSDSGYYANLFLHIGLMSTVDKRGKKVTFDQLERKIRRLDLSVGLSDVLGPSVLVKARGARTRLLAPFFSNSGTACYPIANASPQVAKILWSQTACLRSVKIIIQAGTQRAVAVTADHEVTSTKIEKRHTIAKYNPFKK

SEQ ID NO: 38 - protein, 553 aa, Newcastle disease virus
  F protein
  misc_feature (190)..(190): Xaa can be Phe or Leu
  misc_feature (289)..(289): Xaa can be Leu or Ala
MGPRSSTRIPVPLMLTIRITLALSYVRLTSSLDGRPLAAAGIVVTGDKAVNIYTSSQTGSIIVKLLPNMPKDKEACAKAPLEAYNRTLTTLLTPLGDSIRRIQESVTTSGGRRQRRSIGAIIGSVALGVATAAQITAASALIQANQNAANILRLKESIAATNEAVHEVTGGLSQLAVAVGKMQQFVNDQXNNTAQELDCIKITQQVGVELNLYLTELTTVFGPQITSPALTQLTVQALYNLAGGNVDYLLTKLGVGNNQLSSLIGSGLITGNPIFYDSQTQLLGIQVTXPSVGNLNNMRATYLETLSVSTTKGFASALVPKVVTQVGSVIEELDTSYCIEADLDLYCTRIVTFPMSPGIYSCLSGNTSACMYSRTEGALTTPYMTLKGSVVANCQMTTCRCADPPGIISQNYGEAVSLIDKHSCNVVSLDGITLRLSGEFDATYQKNISILDSQVLVTGNLDISTELGNVNHSISNALDKLEESNSKLDKVNVKLTSTSALITYIVLTVISLVLGMLSLVLACYLMYKQKAQRKTLLWLGNNTLDQMRATTKM

SEQ ID NO: 39 - protein, 2204 aa, Newcastle disease virus
  L protein
  misc_feature (1717)..(1717): Xaa can be Tyr or His
  misc_feature (1910)..(1910): Xaa can be Glu or Lys
MAGSGPERAEHQIILPESHLSSPLVKHKLLYYWKLTGLPLPDECDFDHLIISRQWKKVLESATPDIERMIKLGRAVHQTLNHNSRITGVLHPRCLEELASIEVPDSTNKFRKIEKKIQIHNTRYGELFTRLCTHVEKKLLGSSWSNNVPRSEEFNSIRTDPAFWFHSKWSTAKFAWLHIKQVQRHLIVAARTRSAVNKLVTLTHKVGQIFVTPELVIVTHTDENKFTCLTQELVLMYADMMEGRDMVSIISSTAAHLRSLSEKIDDILRLVDALARDLGNQVYDVVALMEGFAYGAVQLLEPSGTFAGDFFAFNLQELKDTLTGLLPKDIAESVTHAIATIFSGLEQNQAAEMLCLLRLWGHPLLESRIAAKAVRSQMCAPKMVDFDMILQVLSFFKGTIINGYRKKNAGVWPRVKIDTIYGKVIGQLHADSAEISHDVMLREYKSLSALEFEPCIEYDPVTNLSMFLKDKAIAHPKDNWLASFRRNLLSEDQKKHVREATSTNRLLIEFLESNDFDPYKEMEYLTTLEYLRDDNVAVSYSLKEKEVKVNGRIFAKLTNRLRNCQVMAEGILADQIAPFFQGNGVIQDSISLTKSMLAMSQLSFNSNKKRITDCKERVSSNRNHDPKSKNRRRVATFVTTDLQKYCLNWRYQTVKLFAHAINQLMGLPHFFEWIHLRLMDTTMFVGDPFNPPSDPTDCDLSRVPNDDIYIVSARGGIEGLCQKLWTMISIAAIQLAAARSHCRVACMVQGDNQVIAITREVRSDDSPEMVLTQLHQASDNFFKELIHVNHLIGHNLKDRETIRSDTFFIYSKRIFKDGAILSQVLKNSSKLVLISGDLSENTVMSCANIASTVARLCENGLPKDFCYYLNYLMSCVQTYFDSEFSITNNSQLDSNQSWIEDISFVHSYVLTPAQLGGLSNLQYSRLYTRNIGDPGTTAFAEIKRLEAVGLLSPSIMTNILTRPPGNGDWASLCNDPYSFNFETVASPNIVLKKHTQRVLFETCSNPLLSGVHTEDNEAEEKALAEFLLNQEMIHPRVAHAIMEASSVGRRKQIQGLVDTTNTVIKIALTRRPLGIKRLMRIVNYSSMHAMLFRDDVFSSNRSNHPLVSSDMCSQTLADYARNRSWSPLTGGRKILGVSNPDTIELVEGEILSVSGGCTRCDSGDEQFTWFHLPSNIELNDDTSKNPPMRVPYLGSKTQERRAASLAKIAHMSPHVKAALRASSVLIWAYGDNEINWTAALKIARSRCNISSEYLRLLSPLPTAGNLQHRLDDGITQMTFTPASLYRVSPYIHISNDSQRLFTEEGVKEGNVVYQQIMLLGLSLIESLFPMTTTKTYDEITLHLHSKFSCCIREAPVAVPFELLGVAPELRAVTSNKFMYDPSPVSEGDFARLDLAIFKSYELNLESYSTIELMNVLSISSGKLIGQSVVSYDEDTSIKNDAIIVYDNTRNWISEAQNSDVVRLFEYAALEVLLDCSYQLYYLRVRGLNNIVLYMSDLYKNMPGILLSNIAATISHPVIHSRLNAVGLVNHDGSHQLADTDFIEMSAKLLVSCTRRVVSGLHAGNKYDLLFPSVLDDNLSEKMLQLISQLCCLYTVLFATTREIPKIRGLSAEEKCSVLTEYLLSDAVKPLLGSEQVSSIMSPNIVTFPANLYYMSRKSLNLIREREDRDTILALLFPQEPLLEFPLVRDIGARVKDPFTLQPAAFLQELDLSAPARXDAFTISQAHSEHILPNPEEDHLVRYLFRGIGTASSSWYKASHLLSVPEVRCARHGNSLYLAEGSGAIMSLLELHIPHETIYYNTLFSNEMNPPQRHFGPTPTQFLNSVVFRNLQAEVPCKDGFVQEFRPLWRENTEESDLTSDKAVGYITSVVPYRSVSLLHCDIEIPPGSNQSLLDQLATNLSLIAMHSVKXGGVVIIKVLYAMGYYFHLLMNLFTPCSTKGYILSNGYACRGDMECYLIFVMGYLGGPTFVHEVVRMAKTLVQRHGTLLSKSDEITLTRLFTSQQQRVTDILSSPLPRLMKYLRENIDTALIEAGGQPVRPFCAESLVSTLTDMTQTTQIIASHIDTVIRSVIYMEAEGDLADTVFLFTPYNLSMDGKKRTSLKQCTRQILEVTILGLRVKNLNKVGDVISLVLRGMISLEDLLPLRTYLKCSTCPKYLKAVLGITKLKEMFTDASLLYLTRAQQKFYMKTIGNAVKGYYSNCDS

SEQ ID NO: 40 - DNA, 36 bp, Artificial Sequence
  Primer
acgactcactataggaccaaacagagaatccgtgag

SEQ ID NO: 41 - DNA, 35 bp, Artificial Sequence
  Primer
ccgggaagatccagggcactcttcttgcatgttac

SEQ ID NO: 42 - DNA, 35 bp, Artificial Sequence
  Primer
gggcctgcctcactatggtggtaacatgcaagaag

SEQ ID NO: 43 - DNA, 35 bp, Artificial Sequence
  Primer
tgcatgttaccaccaatgtgtcattgtatcgcttg

SEQ ID NO: 44 - DNA, 37 bp, Artificial Sequence
  Primer
caagaagggagatacgtaatatacaagcgatacaatg

SEQ ID NO: 45 - DNA, 35 bp, Artificial Sequence
  Primer
tcgcttgtatattacttgttgtagcaaagagcacc

SEQ ID NO: 46 - DNA, 38 bp, Artificial Sequence
  Primer
ggcctggatcttcccattatgctgtctgtatacggtgc

SEQ ID NO: 47 - DNA, 38 bp, Artificial Sequence
  Primer
atgccatgccgacccaccaaacaaagacttggtgaatg

SEQ ID NO: 48 - DNA, 24 bp, Artificial Sequence
  Primer
cctatagtgagtcgtattaatttc

SEQ ID NO: 49 - DNA, 20 bp, Artificial Sequence
  Primer
cctggatcttcccgggtcgg

SEQ ID NO: 50 - DNA, 22 bp, Artificial Sequence
  Primer
gggtcggcatggcatctccacc

SEQ ID NO: 51 - DNA, 22 bp, Artificial Sequence
  Primer
gggaagatccaggcctatagtg

SEQ ID NO: 52 - DNA, 35 bp, Artificial Sequence
  Primer
ctctagagtcgacccttctgccaacatgtcttccg

SEQ ID NO: 53 - DNA, 35 bp, Artificial Sequence
  Primer
gggaagcggccgcccgtcggtcagtatccccagtc

SEQ ID NO: 54 - DNA, 37 bp, Artificial Sequence
  Primer
ctctagagtcgaccccagagtgaagatggccaccttc

SEQ ID NO: 55 - DNA, 35 bp, Artificial Sequence
  Primer
gggaagcggccgcccgtagtagtgatcagccattc

SEQ ID NO: 56 - DNA, 35 bp, Artificial Sequence
  Primer
ctctagagtcgacccgggtaggacatggcgggctc

SEQ ID NO: 57 - DNA, 37 bp, Artificial Sequence
  Primer
gggaagcggccgccctgcctttaagagtcacagttac

SEQ ID NO: 58 - RNA, 3854 nt, Artificial Sequence
  Reovirus type 3 L1
gctacacgttccacgacaatgtcatccatgatactgactcagtttggaccgttcattgagagcatttcaggtatcactgatcaatcgaatgacgtgtttgaagatgcagcaaaagcattctctatgtttactcgcagcgatgtctacaaggcgctggatgaaatacctttctctgatgatgcgatgcttccaatccctccaactatatatacgaaaccatctcacgattcatattattacattgatgctctaaaccgtgtgcgtcgcaaaacatatcagggccctgatgacgtgtacgtacctaattgttctattgttgaattgctggagccacatgagactctgacatcttatgggcggttgtccgaggccatcgagaatcgtgccaaggatggggacagccaagccagaatcgccacaacgtatggtagaatcgctgaatctcaagctcgacagattaaggctccattggagaagtttgtgttggcactattagtggccgaagcaggggggtctttatatgatccagttttgcagaagtatgatgagattccagatctatcgcataattgccctttatggtgttttagagagatctgtcgtcacatatctggtccattaccagatcgggcaccttatctttacttatctgcaggggttttctggttaatgtcaccacgaatgacgtctgcaatccctccgctactatccgatcttgttaatttagctattttgcaacaaactgcgggtttagatccatcattagtgaaattgggagtacagatatgccttcatgcagcagctagctcaagttatgcatggtttatcttaaagactaagtctatttttcctcaaaacacgttgcacagtatgtatgaatctctagaagggggatactgtcctaatcttgaatggttagagcctagatcagactataagttcatgtacatgggagtcatgccattgtccgctaagtatgctaggtcggcgccgtccaatgataagaaagcgcgggaacttggcgagaaatatggactgagctcagtcgtcggtgagcttcgtaaacggacaaagacgtatgttaaacatgactttgcttcagtgaggtacattcgtgacgctatggcatgtactagcggtattttcttggtaagaacacccaccgaaacggtattgcaagaatatacgcagagtccggagattaaggttcccattccccagaaagactggacaggcccaataggtgaaatcagaattctaaaagatacaacaagttccatcgcgcgttacttatatagaacatggtacttggcagcggcgagaatggcggctcaaccacgtacgtgggatccattgtttcaagcgattatgagatctcaatacgtgacagctaggggtggatctggcgcagcactccgcgaatctttgtatgcaatcaatgtgtcgttacctgatttcaagggcttaccagtgaaggcagcaactaagatattccaggcggcacaattagcgaacttgccgttctcccacacatcagtggctatactagctgacacttcaatgggattgcgaaatcaggtgcagaggcggccacgatccattatgccattaaatgtgccccagcagcaggtttcggcgccccatacattgacagcggattacattaactaccacatgaatctatcaaccacgtctggtagtgcggtcattgagaaggtgattcctttaggtgtatacgcttcgagccctcctaaccagtcgatcaacattgacatatctgcgtgtgacgctagtattacttgggatttctttctgtcagtgattatggcggctatacacgaaggtgtcgctagtagctccattggaaaaccatttatgggggttcctgcatccattgtaaatgatgagtctgtcgttggagtgagagctgctaggccgatatcgggaatgcagaacatgattcagcatctatcgaaactatataaacgtggattttcatatagagtaaacgattctttttctccaggtaacgattttactcatatgactaccactttcccgtcaggttcaacagccacctctactgagcatactgctaataatagtacgatgatggaaactttcctgacagtatggggacccgaacatactgacgaccctgacgtcttacgtttaatgaagtctttaactattcaaaggaattacgtatgtcaaggtgatgatggattaatgattatcgatgggactactgctggtaaggtgaacagtgaaactattcagaagatgctagaattaatctcaaaatatggtgaggaattcggatggaaatatgacatagcgtacgatgggactgccgaatacttaaagctatacttcatatttggctgtcgaattccaaatcttagtcgccatccaatcgtggggaaagaacgggcgaattcttcagcagaggagccatggccagcaattctagatcagattatgggtgtcttctttaatggtgttcatgatgggttacagtggcagcggtggatacgttattcatgggctctatgctgtgctttctcacgtcaaagaacaatgattggtgagagcgtgggttaccttcaatatcctatgtggtcttttgtctactggggattaccactggttaaagcgtttgggtcagacccatggatattttcttggtacatgcctactggagatctgggaatgtatagttggattagcttgatacgccctctgatgacaagatggatggtggctaatggttacgtaactgacagatgctcacccgtattcgggaacgcagattatcgcaggtgtttcaatgaacttaaactatatcaaggttattatatggcacaattgcccaggaatcctaagaagtctggacgagcggcccctcgggaggtaagagaacaattcactcaggcattatccgactatctaatgcaaaatccagagctgaagtcacgtgtgctacgtggtcgtagtgagtgggagaaatatggagcggggataattcacaatcctccgtcattattcgatgtgccccataaatggtatcagggtgcgcaagaggcagcaatcgctacgagagaagagctggcagaaatggatgagacattaatgcgcgctcgaaggcacagctattcgagcttttcaaagttattagaggcgtatctgctcgtgaaatggcgaatgtgcgaggcccgcgaaccgtcggttgatttgcgattaccattatgtgcgggtattgacccattaaactcagatccttttctcaagatggtaagcgttggaccaatgctccagagtacgagaaagtactttgctcagacactattcatggcaaagacggtgtcgggtcttgacgttaacgcgattgatagcgcgttattacgactgcgaacattaggtgctgataagaaagcattaacggcgcagttattaatggtggggcttcaggagtcagaagcggacgcattggccgggaagataatgctacaggatgtgaatactgtgcaattagccagagtggttaacttagctgtgccagatacttggatgtcgttagactttgactctatgttcaaacaccacgtcaagctgcttcccaaagatggacgtcatctaaatactgatattcctcctcgaatgggatggttacgggccattttacgattcttaggtgccggaatggtaatgactgcgactggagttgctgtcgacatctatctggaggatatacatggcggtggtcggtcacttggacagagattcatgacttggatgcgacaggaaggacggtcagcgtgagtctaccatgggtcgtggtgcgtcaactcatc

SEQ ID NO: 59 - RNA, 3916 nt, Artificial Sequence
  Reovirus type 3 L2
gctaaatggcgcgatggcgaacgtttggggggtgagacttgcagactcgttatcttcacccactattgagacacgaacgcgtcagtataccttacacgatctttgctcagacctagatgctaatccggggagggaaccgtggaaacctctgcgtaatcagcgtactaataatattgtggctgtgcaattattcagaccattgcagggtttagttttagatacccagctttatggatttccaggagcatttgatgactgggagcgattcatgagagagaagctgcgtgtgctaaagtatgaagtattgcgcatctatccaatcagcaactatagcaatgaacatgtcaacgtcttcgtggccaatgctttggtgggcgctttcctgtcgaatcaagctttctatgacctgctaccgttgttgataattaatgacactatgattggtgatctacttggcacgggggcatcgctatcacagttctttcaatctcatggagatgtgctggaagtcgcagctggtcgtaagtatctgcagatggaaaactactccaacgatgacgatgatcctccattatttgcgaaagacctgtcagattatgctaaagcattctacagtgacacatatgaagtgttggacaggttcttttggacgcatgactcttcagcgggggtcttagtgcattatgataagccaacgaatggtcatcactatctgctgggtactttgactcagatggtcagtgcacctccttatattattaacgctactgacgcaatgttgcttgaatcctgtctagaacagttctcagctaatgtgcgtgcgagacctgcgcaacccgttacacgcttagaccaatgctatcatttaagatggggagcacaatatgtaggagaagattcactgacatatcggttgggggtgttatccttgctggctaccaatggatatcaattagctagaccgattccaagacagttgacgaatcgatggttgtcgagctttgtgagtcaaattatgtctgacggcgtcaacgagactccactgtggccccaagaaaggtatgtgcagatcgcttatgattcaccatccgttgttgatggggctacgcaatatggctatgtcaggaagaatcaactcagactcggcatgagaatatcggcgctgcaatcgctgagtgatacgccctcgccggtacagtggcttccacaatacaccatcgaccaggcagcgatggacgaaggcgatctgatggttagtcggcttacgcaactcccgttacgtcctgattatggtaatatctgggtcggcgatgcgctatcctattatgtggactacaatcggagtcatcgagtcgtgctttcatcggaacttcctcagcttccggacacatattttgatggcgatgaacagtatgggcgcagcctgttctcactagctcgtaagattggtgaccgctcgttagtgaaagatacggctgtcttgaagcacgcttaccaagccatcgatccaaatactggtaaggagtatctgagatctgggcaatctgtcgcatattttggtgcatcagcgggtcattctggtgccgaccagccgttagtcatagagccctggattcaagggaaaatcagtggtgtgccgccaccctcctcagtgcgacagttcggctatgatgttgcccgtggcgcgatcgtcgatctggcgagaccatttccttctggagattatcaatttgtctattcggatgttgaccaggtggtcgatggccatgacgatctgagtatatcatctggactggtggagagccttttgtcttcatgcatgcacgccacagcacccgggggctcatttgttgttaagataaattttccgactagacccgtatggcactacatcgaacagaagatcttgcccaatattacgtcatacatgttgatcaagcctttcgtcaccaacaacgtcgaattgttcttcgtcgctttcggtgtgcatcaacactcatcacttacttggacatctggagtgtacttcttcttggtggaccatttttatcgttatgagactttatctacgatctcacgacaattgccgtcttttgggtatgttgatgatgggtcttccgtgactggtatcgagacaattagtattgagaaccctggcttctcgaatatgacccaggccgctcgcattggtatctcaggattgtgtgctaatgtaggtaacgcgcgtaagtccattgccatttacgaatcccatggggccagagtattaactatcacatcaaggagatctccggcatcagctagaagaaagtctaggttgcgatatttgccattaatagaccctaggtcgttagaggtacaggcgcgcactattctgccagctgatccagtgttatttgaaaacgtgagcggagcgtcaccccatgtttgtctgacaatgatgtacaacttcgaagtgtcgtcagcggtatatgatggagacgttgtgctagatcttgggacgggaccagaggctaaaatccttgaactgatacccgcaacctctccagtcacatgcgtggacatacggcctacagcgcagcctagtggatgttggaacgttcgtaccacgttccttgagttagattatttgagcgatggatggatcactggggtgcgtggggacatagttacttgtatgttatctttgggggccgctgccgctggaaaatcaatgacttttgacgctgcgtttcagcaattaatcaaagtattatccaagagtacggctaatgttgtgctggtgcaggttaactgccctacagacgtggtgaggagcattaagggctacctagagatagattcgactaacaagaggtataggttccccaaatttggtcgagacgagccgtactctgacatggatgcgctggagaaaatatgtcgtaccgcctggccaaactgctcaattacctgggttccattgtcatacgacttgcggtggactagactggcattattagagtccacgacattgagtagcgcgtcgattagaattgctgagctgatgtataaatacatgcctattatgaggattgatattcatggactacccatggaaaagcgaggtaacttcatagtggggcagaactgctcattagtaatccctggttttaatgcgcaggatgtctttaactgttatttcaattccgccctcgctttctcgactgaagatgtcaatgctgcgatgattccccaagtgtctgcgcagtttgatgcgactaagggtgagtggacgttggatatggtcttatccgacgcaggaatctataccatgcaggctctagtgggatctaatgctaatccagtctctttgggttcctttgtagttgattctccagatgtagatataactgacgcttggccagctcagttagactttacgatcgcgggaactgatgtcgatataacagttaatccttattaccgtctgatgacctttgtaaggatcgatggacagtggcagattgccaatccagacaaatttcaattcttttcgtcggcgtctgggacgttagtgatgaacgtcaaattagatatcgcagataaatatctactatactatatacgagatgtccagtctcgagatgttggcttttacattcagcatccacttcaacttttgaatacgatcacattgccaaccaacgaggacctttttctgagcgcacctgacatgcgagagtgggcagttaaggaaagcggtaacacgatatgtatactcaatagtcaagggtttgtgctacctcaagattgggatgtgttaacagataccataagttggtccccatcgatacccacatacattgtgccaccgggtgattataccttgactcctctgtaactcactgtccctcgtgagcgcgcctaattcatc

SEQ ID NO: 60 - RNA, 3901 nt, Artificial Sequence
  Reovirus type 3 L3
gctaatcgtcaggatgaagcggattccaaggaagacaaagggcaaatccagcggaaagggcaatgactcaacagagagagcggacgatggctcgagccaattaagagacaagcaaaacaataaggctggccccgccactacggagcctggcacatccaaccgagagcaatacaaagctcgaccaggtattgcatctgtgcagagggccactgaaagtgcagaaatgcccatgaagaataatgacgaagggacgccagataagaaaggaaatactaagggcgacctagttaatgagcatagtgaggctaaagacgaggcggatgaagcgacgaagaagcaggcaaaggatacagacaaaagtaaagcgcaagtcacatattcagacactggtatcaataatgctaatgaactgtcaagatctgggaatgtggataatgagggtggaagtaatcagaagccgatgtctaccagaatagctgaggcaacgtctgctatagtgtcgaaacatcctgcgcgtgttgggctgccacctaccgctagcagtggtcatgggtatcagtgccatgtctgttctgcagtcctgtttagtcctttagacctagatgcccacgtcgcctcacatggtttgcatggtaacatgacattaacatcgagtgatatccagcgacatataactgagttcatcagctcatggcaaaatcatcctattgttcaagtttcggctgatgtcgaaaataagaaaactgctcaattgcttcacgctgacactcctcgactcgtcacttgggatgctggtttgtgtacttcattcaaaatcgtcccgattgtgccagctcaggtgccgcaggatgtactggcctatacgtttttcacctcttcatacgctatccaatcgccgtttccagaggcggcagtgtctaggattgtggtgcatacgagatgggcatctaatgttgactttgaccgagactcgtctgtcatcatggcgccacctacagaaaacaatatccatttgtttaaacagttactaaatactgaaaccctgtctgtaaggggggctaatccgctaatgttcagggcgaatgtgttgcatatgttgctagagttcgtattagataacttgtatctgaacagacatacgggattctctcaagaccacacgccatttactgagggtgctaatttgcgttcacttcctggccccgatgctgagaaatggtactcgattatgtatccaacgcgcatgggaacgccgaatgtatccaaaatatgtaatttcgtcgcctcttgtgtgcgaaatcgggttggacggtttgatcgagcacagatgatgaacggagctatgtcagagtgggtggatgtcttcgagacttcagacgcgctaaccgtctccattcgaggtcgatggatggctagactagctcgcatgaacataaatccaacagagatcgaatgggcattgactgaatgtgcacaaggatatgtgactgtcacaagtccttacgctcctagcgtaaatagattgatgccctatcgtatctccaacgctgagcggcaaatatcacagataatcaggatcatgaacattggcaataacgcgacggtgatacaacctgttctgcaagatatttcggtactccttcaacgcatatcaccactccaaatagatccaactattatttccaacactatgtcaacagtctcggagtctactactcagaccctcagccccgcgtcctcaattttgggtaaactacgaccaagcaactcagatttttctagttttagagtcgcgttggctggatggctttataatggggttgtgacgacggtgattgatgatagttcatatccaaaagacggcggcagcgtgacctcacttgaaaatctgtgggatttcttcatccttgcgcttgctctaccactgacaactgacccctgtgcacctgtgaaagcattcatgaccctagccaacatgatggttggtttcgagacaatccctatggataatcagatctatactcaatcgagacgcgcgagtgctttctcaacgcctcacacgtggccacgatgctttatgaacatccagttaatttctccaatcgacgctcccatcttgcgacagtgggctgaaattattcatagatactggcctaacccttcacagattcgttatggtgcaccgaacgttttcggctcggcaaatttgttcactccacctgaggtgctgttattgccaatcgatcatcaaccagctaatgtaacaacgccaacgctggacttcaccaatgagttaactaattggcgcgctcgtgtctgtgagcttatgaagaatctcgttgataaccaaagatatcaacctggatggacacaaagtctagtctcgtcaatgcgcggaacgctagacaaattgaagttgattaaatcgatgacaccaatgtatctgcaacagctggctccggtagagttagcagtgatagctcccatgttgccttttccacctttccaggtgccatacgtccgtctcgatcgtgacagagttccaacaatggttggagtaacacgacagtcacgagatactattactcagccggcgctatcgctgtcgacaaccaatactactgttggcgtgccactagctctagacgcgagggctatcaccgttgcgctgttgtcagggaaatatccgccggatttggtgacaaatgtatggtacgctgatgccatttacccaatgtatgcagacacggaggtgttctctaatcttcagagagacatgattacctgcgaggccgtgcagacattagtgactctggtggcgcaaatatcagagacccagtatcctgtagataggtatcttgattggatcccatcactgagagcatcggcggcgacggcggcgacatttgctgagtgggttaatacttcaatgaagacggcgtttgatttgtctgatatgctgttagagcctctcctaagcggtgatccgaggatgactcaactagcgattcagtatcagcagtacaatggcagaacgtttaatatcatacctgaaatgccaggttcagtaattgctgactgcgttcaattaacagcagaagtctttaatcacgaatataacctgtttgggattgcgcggggtgatatcatcattggccgtgttcagtcgacacatttgtggtcaccgctggctcctccacctgacctggtgtttgatcgtgatacccctggtgttcacatcttcggacgagattgccgtatatcgtttggaatgaatggcgccgcgccaatgattagagatgagactggactgatggtgccttttgaaggaaattggattttcccactggcgctttggcaaatgaatacacgatattttaatcaacagttcgacgcgtggattaagacaggagagttgcgaatccgcattgagatgggcgcgtatccatatatgttgcattactatgatccacgtcagtacgctaatgcatggaatttaacatccgcctggcttgaagaaattacgccgacgagcatcccatccgtgcctttcatggtgcccatttcaagtgatcatgacatttcctctgccccagctgtccaatatatcatttcaactgaatataatgatcggtctctgttctgcactaactcatcatctccccaaaccatcgctggaccagacaaacacattccagttgagagatataacattctgaccaaccccgacgctccacccacgcagatacaactgcctgaagtcgttgacttgtacaacgtcgtcacacgctatgcgtatgagactccgcctattaccgctgttgttatgggtgttccttgatcctcatcctcccaacaggtgctagagcattgcgctcaatgctagttgggccgattcatc

SEQ ID NO: 61 - RNA, 2304 nt, Artificial Sequence
  Reovirus type 3 M1
gctattcgcggtcatggcttacatcgcagttcctgcggtggtggattcacgttcgagtgaggctattggactgctagaatcgtttggagtagacgctggggctgacgcgaatgacgtttcatatcaagatcatgactatgtgttggatcagttacagtacatgttagatggatatgaggctggtgacgttatcgatgcactcgtccacaagaattggttacatcactctgtctattgcttgttgccacccaaaagtcaactattagagtattggaaaagtaatccttcagcgataccggacaacgttgatcgtcggcttcgtaaacgactaatgctaaagaaagatctcaggaaagatgatgaatacaatcagctagcgcgtgctttcaagatatcggatgtctacgcacctctcatctcatccacgacgtcaccgatgacaatgatacagaacttgaatcaaggcgagatcgtgtacaccacgacggacagggtaataggggctagaatcttgttatatgctcctagaaagtactatgcgtcaactctgtcatttactatgactaagtgcatcattccgtttggtaaagaggtgggtcgtgttcctcactctcgatttaatgttggcacatttccgtcaattgctaccccgaaatgttttgtcatgagtggggttgatattgagtccatcccaaatgaatttatcaagttgttttaccagcgcgtcaagagtgttcacgctaacatactaaatgacatatctcctcagatcgtctctgacatgataaacagaaagcgtctgcgcgttcatactccatcagatcgtcgagccgcgcagttgatgcatttgccttaccatgttaaacgaggagcgtctcacgtcgacgtttacaaggtggatgttgtagacatgttgttcgaggtagtggatgtggccgatgggttgcgcaacgtatctaggaaactaactatgcataccgttccggtatgtattcttgaaatgttgggtattgagattgcggactattgcattcgtcaagaggatggaatgctcacagattggttcctacttttaaccatgctatctgatggcttgactgatagaaggacgcattgtcaatacttgattaatccgtcaagtgtgcctcctgatgtgatacttaacatctcaattactggatttataaatagacatacaatcgatgtcatgcctgacatatatgacttcgttaaacccattggcgctgtgctgcctaagggatcatttaaatcaacaattatgagagttcttgattcaatatcaatattaggaatccaaatcatgccgcgcgcgcatgtagttgactcagatgaggtgggcgagcaaatggagcctacgtttgagcaggcggttatggagatatacaaagggattgctggcgttgactcgctggatgatctcatcaagtgggtgctgaactcggatctcattccgcatgatgacaggcttggtcaattatttcaagcgtttttgcctctcgcaaaggacttattagctccaatggccagaaagttttatgataactcaatgagtgagggtagattgctaacattcgctcatgccgacagtgagttgctgaacgcaaattattttggtcatttattgcgactaaaaataccatatattacagaggttaatctgatgattcgcaagaatcgtgagggtggagagctatttcagcttgtgttatcttatctatataaaatgtatgctactagcgcgcagcctaaatggtttggatcattattgcgattgttaatatgtccctggttacatatggagaaattaataggagaagcagacccggcatctacgtcggctgaaattgggtggcatatccctcgtgaacagctgatgcaagatggatggtgtggatgtgaagacggattcattccctatgttagcatacgtgcgccaagactggttatagaggagttgatggagaagaactggggccaatatcatgcccaagttattgtcactgatcagcttgtcgtaggcgaaccgcggagggtatctgctaaggctgtgatcaagggtaaccacttaccagttaagttagtttcacgatttgcatgtttcacattgacggcgaagtatgagatgaggctttcgtgcggccatagcactggacgtggagctgcatacagtgcgagactagctttccgatctgacttggcgtgatccgtgacatgcgtagtgtgacacctgctcctaggtcaatgggggtagggggcgggctaagactacgtacgcgcttcatc

SEQ ID NO: 62 - RNA, 2203 nt, Artificial Sequence
  Reovirus type 3 M2
gctaatctgctgaccgttactctgcaaagatggggaacgcttcctctatcgttcagacgatcaacgtcactggagatggcaatgtatttaaaccatcagctgaaacttcatctaccgctgtaccatcgttaagcttatcacctggaatgctgaatcccggaggggtaccatggattgctgttggagatgagacatctgtgacttcaccaggcgcattacgtcgaatgacgtcaaaggacatcccggaaacggcaataatcaacacagacaattcatcaggcgccgtgccaagcgaatcagccttggtgccctacatcgatgagccgctggtagtggttacagagcatgctattaccaacttcaccaaagctgagatggcacttgaattcaatcgtgagttccttgacaagatgcgtgtgctgtcagtgtcaccaaaatattcggatcttctgacctatgttgactgctacgtcggtgtgtctgctcgtcaggctttaaacaattttcagaaacaagtgcctgtgattacacctactaggcagacgatgtatgtcgactcgatacaagcggccttgaaagctttagaaaagtgggagattgatctgagagtggctcaaacgttgctgcctacgaacgttccgattggagaagtctcttgtccaatgcagtcggtagtgaaactgctggatgatcagctgccagatgacagcctgatacggaggtatcccaaggaagccgccgtcgctttggctaaacgaaacgggggaatacaatggatggacgtatcagaaggcaccgtgatgaacgaggctgtcaacgctgttgcagctagtgcactggcaccttcagcatcagccccacccttagaagagaagtcaaagttaaccgaacaagcgatggatctcgtgaccgcggctgagcctgagataattgcctcactcgtgccagttcccgcacccgtgtttgccataccacctaaaccagcagattataatgtgcgtactctgaggatcgacgaggccacttggctgcgaatgattccaaaatcaatgaacacaccttttcaaatccaggtgactgataacacaggaactaattggcatctcaatttgaggggggggactcgtgtagtgaatctggaccaaatcgctccgatgcggtttgtattagatctagggggaaagagttataaagagacgagctgggatccaaacggcaagaaggtcggattcatcgtttttcaatcgaagataccattcgaactttggactgctgcttcacagatcggtcaagccacggtggttaactatgtccaactatacgctgaagacagctcatttaccgcgcagtctatcattgctactacctctttggcttataactatgagcctgagcagttgaataagactgaccctgagatgaattattatcttttggcgacctttatagactcagccgctataacgccaacgaatatgacacagcctgatgtttgggatgccttgctgacgatgtccccactatcagctggcgaggtgacagtgaagggtgcggtagtgagtgaagtagtccctgcagacttgataggtagctacactccagaatccctaaacgcctcacttccgaatgatgctgctagatgcatgatcgatagagcttcgaagatagccgaagcaatcaagattgatgatgatgctggaccagatgaatattccccaaactctgtaccaattcaaggtcagcttgctatctcgcaactcgaaactggatatggtgtgcgaatattcaaccctaaagggatcctttctaaaattgcatctagggcaatgcaggctttcattggtgacccgagcacaatcatcacgcaggcggcgccagtgttatcagacaagaataattggattgcattggcacagggagtgaaaactagtctgcgtactaaaagtctatcagcgggagtgaagactgcagtgagtaagctgagctcatctgagtctatccagaattggactcaaggattcttggataaagtgtcagcgcattttccagcaccaaagcccgattgtccgactagcggagatagtggtgaatcgtctaatcgccgagtgaagcgcgactcatacgcaggagtggtcaaacgtgggtacacacgttaggccgctcgccctggtgacgcggggttaagggatgcaggcaaatcatc

SEQ ID NO: 63 - RNA, 2241 nt, Artificial Sequence
  Reovirus type 3 M3
gctaaagtgaccgtggtcatggcttcattcaagggattctccgccaacactgttccagtttctaaggccaagcgtgacatatcatctcttgccgctactcctggacttcgttcacaatccttcactccgtctgtggatatgtctcaatcgcgtgaattcctcacaaaggcaattgagcaagggtccatgtctataccttatcagcatgtgaatgtaccgaaagttgatcgtaaagttgttagcctggtagtgcgacctttctcttcaggtgctttctctatctctggagtgatttcgccagcccatgcctatctactagagtgtctaccccagcttgagcaggcgatggcttttgttgcttcacctgagtctttccaggcttccgacgtcgcgaagcgctttgccataaagccaggtatgagcctccaggatgccatcactgcctttattaactttgtgtccgcgatgctgaaaatgacggtgactcgtcaaaactttgacgttattgtggctgagatcgagaggcttgcttcaaccagcgtgtccgtcaggactgaagaagcgaaggttgctgatgaggagctaatgctattcgggttagatcatagagggccacagcagctggatgtttctgacgctaaagggataatgaaggctgctgatattcagacaactcatgatgtccatttggcaccaggcgttggtaatattgatcctgaaatctataacgaggggcggttcatgttcatgcagcacaagccacttgcggcggatcaatcgtatttcaccttggagactgcggattatttcaagatttatccaacatacgatgaacatgatggcaggatggctgaccaaaagcagtcgggattgatactgtgtactaaggacgaggtattggctgagcaaactatatttaaactggacgcccctgatgacaagactgttcatctgttggatcgcgatgacgaccacgttgttgccagatttactaaggtatttatagaggacgtggctcccgggcatcatgctgctcaaagatcgggacaacgctctgtgcttgatgacctatatgcgaatacgcaagtgatttccattacttctgctgctttaaagtgggtggtcaagcacggcgtatctgatggaatcgtgaacaggaagaatgtcaaagtgtgtgttggttttgaccccctgtacaccttgtctacacataacggggtgtccttatgtgccctgctgatggacgaaaaactctctgtgctgaacagtgcgtgtcgtatgacgttacgctcactcatgaagaccggacgcgacgttgatgcacacagagcttttcagcgagtcctctctcaaggatacacatcgctaatgtgctactatcatccttcacggaagttggcatatggtgaggtgctctttctagaacgatccaatgacgtgacagatgggatcaagcttcagttggacgcatctagacagtgtcatgaatgtcctgtgttgcagcagaaagtggttgagttagagaaacagattattatgcagaagtcaatccagtcagaccctaccccagtggcgctgcaaccattgttgtctcagttgcgtgagttgtctagtgaagttactaggctacagatggagttgagtcgagctcagtccctgaatgctcagttggaggcggatgtcaagtcagctcaatcatgtagcttggatatgtatctgagacaccacacttgcattaatggtcatgctaaagaagatgaattgcttgacgctgtgcgtgtcgcgccggatgtgaggagagaaatcatggaaaagaggagtgaagtgagacaaggttggtgcgaacgtatttctaaggaagcagctgccaaatgtcaaactgttattgatgacctgactttgatgaatggaaagcaagcacaagagataacagaattacgtgattcggctgaaaaatatgagaaacagattgcagagctggtgagtaccatcacccaaaaccagataacgtatcagcaagagctacaagccttggtagcgaaaaatgtggaattggacgcgttgaatcagcgtcaggctaagtctttgcgtattactccctctcttctatcagccactcctaccgattcagttgatggtgttgctgacttaattgatttctctgttccaactgatgagttgtaaataatccgtgatgcagtgttgccctaatcccttaagccttcccgacccccattcatc

SEQ ID NO: 64 - RNA, 1416 nt, Artificial Sequence
  Reovirus type 3 S1
gctattggtcggatggatcctcgcctacgtgaagaagtagtacggctgataatcgcattaacgagtgataatggagtatcactgtcaaaagggcttgaatcaagggtctcggcgctcgagaagacgtctcaaatacactctgatactatcctccggatcacccagggactcgatgatgcaaacaaacgaatcatcgctcttgagcaaagtcgggatgacttggttgcatcagtcagtgatgctcaacttgcaatctccagattggaaagctctatcggagccctccaaacagttgtcaatggacttgattcgagtgttacccagttgggtgctcgagtgggacaacttgagacaggacttgcagagctacgcgttgatcacgacaatctcgttgcgagagtggatactgcagaacgtaacattggatcattgaccactgagctatcaactctgacgttacgagtaacatccatacaagcggatttcgaatctaggatatccacattagagcgcacggcggtcactagcgcgggagctcccctctcaatccgtaataaccgtatgaccatgggattaaatgatggactcacgttgtcagggaataatctcgccatccgattgccaggaaatacgggtctgaatattcaaaatggtggacttcagtttcgatttaatactgatcaattccagatagttaataataacttgactctcaagacgactgtgtttgattctatcaactcaaggataggcgcaactgagcaaagttacgtggcgtcggcagtgactcccttgagattaaacagtagcacgaaggtgctggatatgctaatagacagttcaacacttgaaattaattctagtggacagctaactgttagatcgacatccccgaatttgaggtatccgatagctgatgttagcggcggtatcggaatgagtccaaattataggtttaggcagagcatgtggataggaattgtctcctattctggtagtgggctgaattggagggtacaggtgaactccgacatttttattgtagatgattacatacatatatgtcttccagcttttgacggtttctctatagctgacggtggagatctatcgttgaactttgttaccggattgttaccaccgttacttacaggagacactgagcccgcttttcataatgacgtggtcacatatggagcacagactgtagctatagggttgtcgtcgggtggtgcgcctcagtatatgagtaagaatctgtgggtggagcagtggcaggatggagtacttcggttacgtgttgaggggggtggctcaattacgcactcaaacagtaagtggcctgccatgaccgtttcgtacccgcgtagtttcacgtgaggatcagaccaccccgcggcactggggcatttcatc

SEQ ID NO: 65 - RNA, 1331 nt, Artificial Sequence
  Reovirus type 3 S2
gctattcgctggtcagttatggctcgcgctgcgttcctattcaagactgttgggtttggtggtctgcaaaatgtgccaattaacgacgaactatcttcacatctactccgagctggtaattcaccatggcagttaacacagtttttagactggataagccttgggaggggtttagctacatcggctctcgttccgacggctgggtcaagatactatcaaatgagttgccttctaagtggcactctccagattccgttccgtcctaaccaccgatggggagacattaggttcttacgcttagtgtggtcagctcctactctcgatggattagtcgtagctccaccacaagttttggctcagcccgctttgcaagcacaggcagatcgagtgtacgactgcgatgattatccatttctagcgcgtgatccaagattcaaacatcgggtgtatcagcaattgagtgctgtaactctacttaacttgacaggttttggcccgatttcctacgttcgagtggatgaagatatgtggagtggagatgtgaaccagcttctcatgaactatttcgggcacacgtttgcagagattgcatacacattgtgtcaagcctcggctaataggccttgggaatatgacggtacatatgctaggatgactcagattgtgttatccttgttctggctatcgtatgtcggtgtaattcatcagcagaatacgtatcggacattctattttcagtgtaatcggcgaggtgacgccgctgaggtgtggattctttcttgttcgttgaaccattccgcacaaattagaccgggtaatcgtagcttattcgttatgccaactagcccagattggaacatggacgtcaatttgatcctgagttcaacgttgacggggtgtttgtgttcgggttcacagctgccactgattgacaataattcagtacctgcagtgtcgcgtaacatccatggctggactggtagagctggtaaccaattgcatgggttccaggtgagacgaatggtgactgaattttgtgacaggttgagacgcgatggtgtcatgacccaagctcagcagaatcaagttgaagcgttggcagatcagactcaacagtttaagagggacaagctcgaaacgtgggcgagagaagacgatcaatataatcaggctcatcccaactccacaatgttccgtacgaaaccatttacgaatgcgcaatggggacgaggtaatacgggggcgactagtgccgcgattgcagcccttatctgatcgtcttggagtgagggggtccccccacacacctcacgactgaccacacattcatc

SEQ ID NO: 66 - RNA, 1198 nt, Artificial Sequence
  Reovirus type 3 S3
gctaaagtcacgcctgtcgtcgtcactatggcttcctcactcagagctgcgatctccaagatcaagagggatgacgtcggtcagcaagtttgtcctaattatgtcatgctgcggtcctctgtcacaacaaaggtggtacgaaatgtggttgagtatcaaattcgtacgggcggattcttttcgtgcttagctatgctaaggccactccagtacgctaagcgtgagcgtttgcttggtcagaggaatctggaacgtatatcgactagggatatccttcagactcgtgatttacactcactatgtatgccaactcctgatgcgccaatgtctaatcatcaagcatccaccatgagagagctgatttgcagttacttcaaggtcgatcatgcggatgggttgaaatatatacccatggatgagagatactctccgtcatcacttgccagattgtttaccatgggcatggctgggctgcacattaccactgagccatcttataagcgtgttccgattatgcacttagctgcggacttggactgtatgacgctggctctaccttacatgattacgcttgatggtgatactgtggttcctgtcgctccaacactgtcagcggaacagcttctggacgacggactcaaaggattagcatgcatggatatctcctatggatgtgaggtggacgcgaatagccggccggctggtgatcagagtatggactcttcacgctgcatcaacgagttgtattgcgaggagacagcagaagccatctgtgtgcttaagacatgccttgtgttaaattgcatgcagtttaaacttgagatggatgacctagcacataacgctgctgagctggacaagatacagatgatgatacccttcagtgagcgtgtttttaggatggcctcgtcctttgcgactattgatgcccagtgttttaggttttgcgtgatgatgaaggataaaaatctgaaaatagatatgcgtgaaacgacgagactgtggactcgttcagcatcagatgattctgtggccacgtcatctttaagtatttccctggaccggggtcgatgggtggcggctgacgccagtgatgctagactgctggtttttccgattcgcgtgtaatgggtgagtgagctgatgtggtcgccaagacatgtgccggtgtcttggtggtgggtgacgcctaatcatc

SEQ ID NO: 67 - RNA, 1196 nt, Artificial Sequence
  Reovirus type 3 S4
gctatttttgcctcttcccagacgttgtcgcaatggaggtgtgcttgcccaacggtcatcaggtcgtggacttgattaacaacgcttttgaaggtcgtgtatcaatctacagcgcgcaagagggatgggacaaaacaatctcagcacagccagatatgatggtatgtggtggcgccgtcgtttgcatgcattgtctaggtgttgttggatctctacaacgcaagctgaagcatttgcctcaccatagatgtaatcaacagatccgtcatcaggattacgtcgatgtacagttcgcagaccgtgttactgctcactggaagcggggtatgctgtccttcgttgcgcagatgcacgagatgatgaatgacgtgtcgccagatgacctggatcgtgtgcgtactgagggaggttcactagtggagctgaactggcttcaggttgacccaaattcaatgtttagatcaatacactcaagttggacagatcctttgcaggtggtggacgaccttgacactaagctggatcagtactggacagccttaaacctgatgatcgactcatccgacttgatacccaactttatgatgagagacccatcacacgcgttcaatggtgtgaaactggagggagatgctcgtcaaacccaattctccaggacttttgattcgagatcgagtttggaatggggtgtgatggtttatgattactctgagctggagcatgatccatcgaatggccgtgcttacagaaaggaattggtgacgccagctcgagatttcggtcactttggattatcccattattctagggcgactaccccaatccttggaaagatgccggccgtattctcaggaatgttgactgggaactgtaaaatgtatccattcattaaaggaacggctaagctgaagacagtgcgcaagctagtggaggcagtcaatcatgcttggggtgtcgagaagattagatatgctcttgggccaggtggcatgacgggatggtacaataggactatgcaacaggcccccattgtgctaactcctgctgctctcacaatgttcccagataccatcaagtttggggatttgaattatccagtgatgattggcgatccgatgattcttggctaaacacccccatcttcacagcgccgggcttgaccaacctggtgtgacgtgggacaggcttcattcatc

(19) United States
(12) Patent Application Publication
(10) Pub. No.: US 2023/0147975 A1
(43) Pub. Date: May 11, 2023

(54) PHARMACEUTICAL FORMULATION COMPRISING A COMBINATION OF RECOMBINANT NEWCASTLE DISEASE VIRUSES FOR THE TREATMENT OF CANCER

(71) Applicants: Jan Merlin Jonas Aietos Thaller, Markt Berolzheim (DE); Tristan Wolfram Thaller, Weßling (DE); Milena Mechthild Weis, Hallstadt (DE); Gudrun Kvetoslava Thaller, Freiburg (DE); Linda Libuska Nerlich, München (DE); Svetlana Thaller-Honold, Köln (DE)

(72) Inventor: Arno THALLER, Markt Berolzheim (DE)

(21) Appl. No.: 18/017,878

(22) PCT Filed: Jul. 21, 2021
(86) PCT No.: PCT/EP2021/070336

§ 371 Date: Jan. 25, 2023

(30) Foreign Application Priority Data
Jul. 27, 2020 (EP) 20187938.4

(51) Int. Cl. A61K 35/768; C12N 15/86

(52) U.S. Cl. CPC A61K 35/768; C12N 15/86; A61K 2039/505

(57) ABSTRACT

The invention relates to a pharmaceutical formulation comprising at least three recombinant transgene expressing Newcastle Disease Virus (NDV) strains, which have been demonstrated to possess significant oncolytic activity against mammalian cancers and an improved safety profile, a non-recombinant NDV strain, a reovirus type-3 and optionally a vaccinia virus. At least one of the recombinant NDV strains comprises in its viral genome a nucleic acid sequence comprising at least one foreign gene, the at least one foreign gene encoding a checkpoint modulator, and at least one of the recombinant NDV strains comprises in its viral genome a nucleic acid sequence comprising at least one foreign gene, the at least one foreign gene encoding an angiogenesis inhibitor. The viral genome of each of the at least three recombinant NDV strains comprises a mutation in the HN gene, said mutation allowing replication of said rgNDV in a cancer cell to a higher level than replication of an otherwise identical NDV not having said mutation in the HN gene. The pharmaceutical formulation provides an improved treatment of cancer, because instead of a monotherapy, a mixture of oncolytic viruses is applied.

The present invention is in the field of compositions and treatments for cancer. In particular, the present invention generally relates to compositions comprising at least three different recombinant Newcastle Disease Virus strains, which have been demonstrated to possess significant oncolytic activity against mammalian cancers, especially selected from the specific cancers as described herein and/or mentioned in the claims, and which provide improved viral safety, at least one non-recombinant NDV strain, thereby improving the efficiency of the therapeutic agent in the treatment of cancer, a reovirus type 3, beneficially modulating the immunotherapeutic aspect of the cancer treatment and optionally a vaccinia virus. By combining the NDV strains with reovirus type 3 the therapeutic potency can be markedly increased. These recombinant strains have been obtained through the use of genetic engineering, including the transfer of genes across species boundaries, so that they are carrying transgenes useful in the treatment of cancer. The compositions of the present invention provide novel and improved oncolytic agents and are intended for administration to subjects having such health conditions.

### BACKGROUND

NDV is known as an oncolytic virus that is a virus for use in oncological treatment, preferably in the treatment of human subjects in need thereof. A number of RNA viruses, including NDV, reovirus, measles virus, and vesicular stomatitis virus (VSV), are members of this novel class of viruses being exploited as potential oncolytic agents. These oncolytic viruses are characterized in inherently replicating selectively within and killing tumor cells while leaving non-tumor cells unharmed. Accordingly these oncolytic viruses offer an attractive tool for cancer therapy.

NDV derives its name from the site of the original outbreak in chickens at a farm near Newcastle-upon-Tyne in England in 1926. The virus is an economically important pathogen in multiple avian species and it is endemic in many countries. NDV is a member of the Avulavirus genus in the Paramyxoviridae family and is a non-segmented, negative-strand RNA virus, whose natural host range is limited to avian species; however, it is known to enter cells by binding to sialic acid residues present on a wide range of human and rodent cancer cells. The oncolytic property of NDV, i.e. to selectively replicate in and destroy tumor cells, while sparing normal cells, is believed to be based in part on defective antiviral responses in tumor cells. Normal cells, which are competent in launching an efficient antiviral response rapidly after infection, are able to inhibit viral replication before viral-mediated cell damage can be initiated. The sensitivity of NDV to interferon (IFN), coupled with defective IFN signaling pathways in tumor cells, provides one plausible mechanism whereby NDV can replicate exclusively within neoplastic tissue. The selective replication of NDV in human tumor cells may also be caused by several other mechanisms, including defects in activation of anti-viral signaling pathways, and activation of Ras signaling and/or expression of Rac1 (Schirrmacher, 2015, Expert Opin. Biol. Ther. 15:17 57-71).

Due to its tumor-specificity NDV is already under investigation as a safe clinical oncolytic virotherapy agent (Cassel et al., 1965, Cancer 1:863-888; Steiner et al., 2004, J. Clin. Oncol. 22:4272-4281; Schulze et al., 2009, Cancer Immunol. Immunother. 58:61-69; Csatary et al., 1999, Anticancer Res. 19:635-638; Pecora et al., 2002, J. Clin. Oncol. 20:2251-2266; Lorence et al., 2003, Curr. Opin. Mol. Ther. 5:618-624; Freeman et al., 2006, Mol. Ther. 13:221-228). The safety margin of NDV is a function of its inability to replicate in and kill normal human cells.

Typically, an oncolytic NDV strain is defined as an NDV for use in oncological virotherapy, preferably in the treatment of human subjects in need thereof. Multiple preclinical model studies have shown significant anti-tumor activity of natural and recombinant oncolytic NDV strains after varying treatment modalities. Promising results were noted in preclinical models of glioblastoma multiforme, anaplastic astrocytoma, leukemia, lymphoma, melanoma, neuroblastoma, osteosarcoma, rhabdomyosarcoma, Ewing's sarcoma, fibrosarcoma, pheochromocytoma, colon carcinoma, lung carcinoma, prostate carcinoma, breast carcinoma, ovary carcinoma, gastric carcinoma, mesothelioma, renal cell carcinoma, and head and neck carcinoma. One typically useful oncolytic NDV that has been extensively explored for use in the treatment of humans, and for which an advantageous safety and efficacy profile has been established in human trials, is a strain named MTH-68/H (Csatary L K, et al., J Neurooncol. 2004 March-April; 67(1-2):83-93). MTH-68/H therapy has been employed in a range of different tumors with success. MTH-68/H has been developed into a highly purified, lyophilized product, containing live, replication competent viral particles, grown to standardized titers. A Phase II clinical trial in humans was completed where the inhalatory mode of administration was used on patients suffering from a variety of advanced malignancies no longer responding to conventional cancer therapies. The study demonstrated relative efficacy, an overall improved quality of life, and a relatively benign side effect profile (Csatary L K, et al., Cancer detection and Prevention, 1993, 17(6):619-627). Success of treatment with MTH-68/H has also been shown in glioblastomas and anaplastic astrocytomas. These patients regularly received the MTH-68/H viral therapy for a number of years without interruption as a form of monotherapy once the classical modalities failed. The MRI and CT results have revealed an objective decrease in size of the tumors, in some cases the near total disappearance of the tumor mass.

As reviewed by Zamarin and Palese (Future Microbiol. 2012 March; 7(3): 347-367), when compared to other oncolytic agents in development such as poxvirus, HSV-1, adenovirus, measles, and reovirus vectors, NDV as an oncolytic virus exhibits several advantages over other viruses for use in oncological treatment. The first advantage is that NDV is an avian pathogen. This avoids the problems of pre-existing immunity that can neutralize virus infectivity and pathogenicity of the virus in humans. To attest for viral safety, administration of virulent strains to humans has been shown to result in only mild to moderate adverse effects, with mild conjunctivitis, laryngitis, and flu-like symptoms as the only reported side effects. The second advantage offered by using NDV as an oncolytic agent is that NDV, similarly to other oncolytic viruses, possesses strong immunostimulatory properties that are the basis for oncolytic therapy being considered an immunotherapy. These properties include the induction of type I IFN and chemokines, upregulation of MHC and cell adhesion molecules, and facilitation of adhesion of lymphocytes and antigen-presenting cells (APCs) through expression of viral glycoproteins on the surface of infected cells. These properties have been shown to generate effective anti-tumor immune responses, which may persist long after clearance of the primary viral infection. A third advantage is that the NDV genome has the plasticity to enable the incorporation and stable expression of foreign genes of relatively large size. Since these viruses replicate in the cytoplasm of the cell and not in the nucleus, an unintended integration of the foreign gene into the host genome is avoided. Such an unintended integration of foreign genes carried by an oncolytic agent into the host genome could be a safety problem with some DNA oncolytic vectors. Moreover, the absence of homologous RNA recombination ensures that foreign genes incorporated in and expressed from the NDV genome are stable for many serial passages in cell culture and in tumor cells. Lastly, the ubiquitous nature of the NDV receptor allows for utilization of the virus against a wide variety of tumor types. The specificity of NDV for cancer cells due to their defects in antiviral pathways ensures viral safety.

Similar to other paramyxoviruses in its family, the 15186, 15192 or 15198 nucleotide (nt)-long negative single-strand RNA genome of NDV encodes six genes including the nucleocapsid protein (NP), phosphoprotein (P), matrix protein (M), fusion protein (F), hemagglutinin-neuraminidase (HN), and RNA-dependent RNA polymerase (L) (Lamb R, Paramyxoviridae: the viruses and their replication. In: Knipe D, editor. Fields Virology, Lippincott Williams & Wilkins; 2007, pp. 1449-1496). The genes are separated by junction sequences that consist of three elements, known as gene start (GS), intergenic (IG), and gene-end (GE) motifs, which regulate mRNA transcription. In the P gene, a unique RNA editing mechanism adds non-templated G residues, resulting in the expression of V and W proteins that are colinear to the P protein in the amino-terminal end. The genomic RNA is bound in a ribonucleotide protein complex (RNP) consisting of NP, P, and L proteins and is surrounded by a lipid envelope containing three virus glycoprotein spikes, HN, M and F. An important factor that influences the extent of virulence of NDV in birds is the cleavage site of the F protein.

Pathogenic classification and virulence of NDV strains in birds generally correlates with their oncolytic properties in human cancer cells. Velogenic (high virulence) strains produce severe respiratory and nervous system signs, spread rapidly through chicken flocks, and can cause up to 90% mortality. Mesogenic (intermediate virulence) strains cause coughing, affect egg production and quality, and can result in up to 10% mortality. Lentogenic (low virulence) strains produce only mild symptoms with little if any mortality. Correspondingly, velogenic strains can efficiently carry out multicycle replication in multiple human cancer cells with effective and efficient cell lysis, lentogenic strains are more attenuated due to lack of activation of the F0 protein, and mesogenic strains convey intermediate effects. On the basis of these observations in human cancers, NDV strains have been classified as either lytic or non-lytic, with velogenic and mesogenic viruses being lytic (high and low respectively), and lentogenic viruses in general being non-lytic. Early studies demonstrated that the lytic abilities of lentogenic NDV strains could be enhanced by the introduction of a polybasic cleavage site into their F proteins (Peeters et al, J. Virol. 1999; 73:5001-5009). Other NDV proteins including NP, P, V, HN, and L have also been shown to be implicated in virulence in birds. A deletion (18 nt) introduced in the NP gene (Mebatsion T et al., J Virol. 2002 October; 76 (20):10138-46) resulted in the deletion of NP residues 443-460. The resulting mutant NDV propagated in embryonated specific-pathogen-free (SPF) chicken eggs to a level fully comparable to that of the parent virus. In addition, a B-cell epitope of the S2 glycoprotein of murine hepatitis virus (MHV) was inserted in-frame. Recombinant NDV viruses properly expressing the introduced MHV epitope were successfully generated, demonstrating that the NP can be used as the insertion point in the NDV genome to insert foreign or transgenic sequences to be co-expressed with NDV during virus infection.

Recently, two studies showed the role of the polymerase complex in viral pathogenesis, with L protein contributing to pathogenicity of the mesogenic Beaudette C strain, and NP, P, and L proteins contributing to pathogenicity of the velogenic Herts strain (Rout & Samal, 2008, J Virol. 82:7828-7836; Dortmans et al., 2010, J Virol. 84:10113-10120). In addition, several studies noted the contribution of the HN protein to virulence in birds (Römer-Oberdörfer et al., 2006, Avian Dis. 50:259-263; Paldurai et al., 2014, J Virol. 88: 8579-8596.) The HN (hemagglutinin-neuraminidase) protein of NDV is a multifunctional protein with receptor-recognition, hemagglutinin (HA) and receptor-destroying neuraminidase (NA) activities associated with the virus. HN is thought to possess both the receptor recognition of sialic acid at the termini of host glycoconjugates and the neuraminidase activity to hydrolyze sialic acid from progeny virion particles in order to prevent viral self-aggregation. It also recognizes sialic acid-containing receptors on cell surfaces and promotes the fusion activity of the F protein, thereby allowing the virus to penetrate the cell surface. Thus, the HN protein plays a critical role in viral infection of birds.

NDV strains having a mutation in the HN gene resulting in an increased replication capability in a cancer cell are disclosed in WO 2019/197275 A1 and WO 2020/043835 A1. These NDV strains are also capable of expressing a foreign gene.

As discussed above, the viral F protein of NDV is responsible for viral fusion with the cell membrane and for viral spread from host cell to host cell via formation of syncytia. The presence of the multi-basic amino acid cleavage site in the F protein enables protein cleavage and activation by a broad range or proteases and is known to be a determinant of virulence in velogenic NDV strains. In order to increase the oncolytic potency of a highly attenuated lentogenic Hitchner B1 NDV strain, a polybasic cleavage site was introduced into the F protein to generate rNDV/F3aa with mutated F protein. Altomonte et al. (Mol Ther. 2010 18:275-84) reported an oncolytic NDV vector virus with the mutated F-protein rNDV/F(L289A), harboring an L289A mutation within the F protein, which is required for virus entry and cell-cell fusion.

A recombinant NDV vector virus can be provided by a reverse genetics (rg) system, which has been known since 1999 (Peeters et al., 1999, J. Virol. 73:5001-5009). This system allows desired modification and engineering of NDV genomes. Indeed, various reports have shown the successful use of recombinant NDV vectors engineered to express various transgenes, i.e. foreign genes incorporated in the genome of the virus, with the goal of improving viral oncolytic efficacy (Vigil et al. Cancer Res. 2007, 67:8285-8292; Janke et al., 2007, Gene Ther. 14:1639-1649).

Due to compelling preclinical data implicating oncolytic NDV as an ideal candidate for cancer therapy, phase I and II clinical trials have been initiated. Several trials have shown that NDV is a safe and potentially therapeutically useful therapeutic agent, with no reports of significant adverse effects in patients beyond conjunctivitis or mild flu-like symptoms (Csatary et al., 1999, Anticancer Res. 19:635-638; Pecora et al., 2002, J. Clin. Oncol. 20:2251-2266; Lorence et al., 2003, Curr. Opin. Mol. Ther. 5, 618-624; Freeman et al., 2006, Mol. Ther. 13:221-228). Although the results of these clinical trials have been encouraging, the extent of clinical responses has not been strong and robust enough to consider bringing one of these initial NDV strains into advanced clinical development. Thus, strategies for improving therapeutic effectiveness while maintaining an acceptable safety profile of oncolytic NDV strains are needed. Thus there remains a clear need for improved NDV based agents for the treatment of cancer and/or improvement in therapeutic outcome when exploiting the oncolytic potential of NDV in the treatment of cancer.

### DESCRIPTION OF THE PRESENT INVENTION

The present invention has solved the above needs by providing a pharmaceutical composition enabling a therapeutic approach in which, instead of a monotherapy, a combination of oncolytic viruses providing different effects is used. The combination therapy with the NDV strains markedly increases the therapeutic potency. This applies in particular in a combination therapy of reovirus, non-recombinant NDV strains and recombinant NDV strains encoding immunotherapeutic agents, preferably the one or more checkpoint modulators and angiogenesis inhibitors, and the virulence factor and/or the other foreign genes as specified supra.

Thus, in one aspect the present invention relates to a pharmaceutical composition comprising: a) at least three recombinant Newcastle Disease Virus (NDV) strains without or preferably in a suitable carrier medium selected from the group comprising:
at least one of the recombinant NDV strains comprises in its viral genome a nucleic acid sequence comprising at least one foreign gene, the at least one foreign gene encoding a checkpoint modulator selected from the group consisting of an antibody, preferably a monoclonal antibody, directed to the surface protein CTLA-4 or an antigen-binding part directed to the surface protein CTLA-4 (anti-CTLA-4), preferably Ipilimumab, an antigen-binding part of Ipilimumab, a variant of Ipilimumab or a variant of an antigen-binding part of Ipilimumab, and/or
   an antibody, preferably a monoclonal antibody, directed to protein PD-1 or an antigen-binding part directed to protein PD-1 (anti-PD-1), preferably Nivolumab, an antigen-binding part of Nivolumab, a variant of Nivolumab or a variant of an antigen-binding part of Nivolumab, and/or
   an antibody, preferably a monoclonal antibody, directed to protein PD-L1 or an antigen-binding part directed to protein PD-L1 (anti-PD-L1), preferably Atezolizumab, an antigen-binding part of Atezolizumab, a variant of Atezolizumab or a variant of an antigen-binding part of Atezolizumab, and/or
   at least one recombinant NDV strain comprising in its viral genome a nucleic acid sequence comprising at least one foreign gene encoding a protein with the ability to selectively induce apoptosis in human tumor cells, but not in normal human cells, or a part thereof with the ability to selectively induce apoptosis in human tumor cells, but not in normal human cells, preferably a gene encoding Apoptin, a part of Apoptin, a variant of Apoptin or a variant of a part of Apoptin,
at least one of the recombinant NDV strains comprises in its viral genome a nucleic acid sequence comprising at least one foreign gene, the at least one foreign gene encoding an angiogenesis inhibitor selected from the group consisting of an antibody, preferably a monoclonal antibody, or an antigen-binding part thereof directed to the growth factor protein VEGF-A (anti-VEGF-A), preferably Bevacizumab, an antigen-binding part of Bevacizumab, a variant of Bevacizumab or a variant of an antigen-binding part of Bevacizumab, and/or
an antibody, preferably a monoclonal antibody, or an antigen-binding part thereof directed to VEGFR (anti-VEGFR), preferably Ramucirumab, an antigen-binding part of Ramucirumab, a variant of Ramucirumab or a variant of an antigen-binding part of Ramucirumab, and
at least one recombinant NDV strain comprising in its viral genome a nucleic acid sequence comprising at least one foreign gene, the at least one foreign gene encoding a virulence factor, wherein the virulence factor is selected from a protein with the ability to modulate the virus replication cycle, or a part thereof with the ability to modulate the virus replication cycle, more preferably a gene encoding the non-structural protein NS1 of influenza A virus, a part of the non-structural protein NS1 of influenza A virus, a variant of the non-structural protein NS1 of influenza A virus or a variant of a part of the non-structural protein NS1 of influenza A virus, and/or
a protein that reduces or inhibits IFN expression such as an IFN-beta receptor, or a part thereof that reduces or inhibits IFN expression such as an IFN-beta receptor, preferably a gene encoding the viral protein B18R from vaccinia virus, a part of the viral protein B18R from vaccinia virus, a variant of the viral protein B18R from vaccinia virus or a variant of a part of the viral protein B18R from vaccinia virus,
and
wherein the viral genome of each of the at least three recombinant NDV strains comprises a nucleic acid comprising a nucleic acid sequence encoding a hemagglutinin-neuramidase protein (HN protein) with an amino acid substitution at position 277 to an amino acid with a hydrophobic side chain other than phenylalanine, preferably where phenylalanine (F) at position 277 is substituted to leucine (L)
b) at least one non-recombinant NDV strain, wherein the viral genome of the at least one non-recombinant NDV strain comprises a nucleic acid comprising a nucleic acid sequence encoding a hemagglutinin-neuramidase protein (HN protein) with an amino acid substitution at position 277 to an amino acid with a hydrophobic side chain other than phenylalanine, preferably where phenylalanine (F) at position 277 is substituted to leucine (L),

c) a reovirus type 3, and

d) optionally a vaccinia virus.

As used herein, checkpoint modulators are intended to mean substances or agents that target and influence “checkpoints”. The checkpoint modulator can be a checkpoint activator or a checkpoint inhibitor. An activator activates “checkpoints”, while an inhibitor blocks “checkpoints”. These “checkpoints” are proteins made by some types of immune system cells, such as T cells, and some cancer cells. They need to be activated or inactivated to start an immune response and thus also play an important role in the regulation of the immune response. Cancer cells sometimes find ways to use these checkpoints to avoid being attacked by the immune system. Thus, when these checkpoints are blocked, for example, cells of the immune system can kill cancer cells better. Examples of checkpoint proteins found on cells of the immune system or cancer cells include, but are not limited to, PD-1, PD-L1, CTLA-4, B7-1 and B7-2. The checkpoint proteins B7-1 and B7-2 can be found on antigen-presenting cells and CTLA-4 on T cells.

As used herein, angiogenesis inhibitors are intended to mean agents or substances that keep new blood vessels from forming. In the process of angiogenesis, i.e. the formation of new blood vessels, inter alia the vascular endothelial growth factor (VEGF) and other endothelial growth factors as well as receptors, such as the VEGF receptors (VEGFR) are involved. The VEGF receptors are receptors for VEGF. VEGF is a well-characterised signal protein which stimulates angiogenesis. There are three main subtypes of VEGFR, numbered 1, 2 and 3. Also, they may be membrane-bound (mbVEGFR) or soluble (sVEGFR), depending on alternative splicing. When VEGF and other endothelial growth factors bind to their receptors on endothelial cells, signals within these cells are initiated that promote the growth and survival of new blood vessels. Angiogenesis inhibitors interfere in several ways with various steps in blood vessel growth. Some are antibodies, preferably monoclonal antibodies that specifically recognize and bind to VEGF, so that VEGF is unable to activate the VEGFR. Other angiogenesis inhibitors may bind to VEGF and/or its receptor as well as to other receptors on the surface of endothelial cells or to other proteins in the downstream signalling pathways, blocking their activities. Angiogenesis inhibitors may also be immunomodulatory drugs that also have antiangiogenic properties. In cancer treatment, angiogenesis inhibitors can prevent the growth of new blood vessels that tumours need to grow, which in consequence prevents or slows the growth of cancer by starving it of its needed blood supply.

The NDV strains comprised in the pharmaceutical according to the present invention comprise a mutation in the viral HN gene, i.e. the nucleic acid sequence encoding the hemagglutinin-neuraminidase protein (HN protein), leading to a change in the amino acid sequence of the HN protein and providing the NDV with an increased replication capability in a cancer cell, preferably in a human cancer cell, as compared to an otherwise identical NDV not having the said mutation in the HN gene.

Preferably the mutation in the HN gene results in an unexpected beneficially increased replication capability of the NDV strain in cancer cells, preferably in human cancer cells, and more preferably results in a replication of said oncolytic NDV in a human cancer cell which is at least 2-fold higher, more preferably 2- to 10-fold higher, still more preferably 5- to 10-fold higher than an NDV strain not having said mutation in its viral HN gene. Preferably, said NDV strains used as parent strain for inserting the mutation in the HN gene, and optionally further mutations in other viral genes as described below, comprise or are oncolytic NDVs already having an advantageous safety and efficacy profile in humans, such as the NDV strain MTH-68/H or are derived from the NDV strain MTH-68/H. The nucleic acid sequence of the NDV strain MTH-68/H is shown in SEQ ID No. 1 of the sequence listing.

According to the invention the NDV strains comprise a mutated HN gene and the encoded hemagglutinin-neuramidase (HN protein) with an amino acid substitution at position 277 to an amino acid with a hydrophobic side chain other than phenylalanine. Preferably is the amino acid phenylalanine (F) substituted to leucine (L) at amino acid position 277. Herein an oncolytic NDV having such a mutation is also identified as NDV^{F277L} to discern it from a parent NDV having a phenylalanine at amino acid position 277 of the HN gene.

The mutation in the HN gene is also identified as HN(F277L), wherein “HN” identifies the gene/nucleic acid sequence of the viral genome (i.e. in this example the HN gene), “277” identifies the amino acid position in the respective gene product (i.e. in this example the amino acid at position 277 of the HN protein), “F” or in general the letter before the number, identifies the amino acid originally present in the respective wild type sequence, and “L” or in general the letter following the number identifies the amino acid present in the mutated NDV strain. That is the amino acid at amino acid position 277 of the HN protein is “L”, which stands for leucine. A respective nomenclature is used for all other mutations described in the following.

In describing a protein or peptide formulation, structure and function herein, reference is made to amino acids. In the present specification, amino acid residues are expressed by using the following abbreviations. Also, unless explicitly otherwise indicated, the amino acid sequences of peptides and proteins are identified from N-terminal to C-terminal (left terminal to right terminal), the N-terminal being identified as a first residue. Amino acids are designated by their 3-letter abbreviation, 1-letter abbreviation, or full name, as follows. Ala: A: alanine; Asp: D: aspartic acid; Glu: E: glutamic acid; Phe: F: phenylalanine; Gly: G: glycine; His: H: histidine; Ile: I: isoleucine; Lys: K: lysine; Leu: L: leucine; Met: M: methionine; Asn: N: asparagine; Pro: P: proline; Gln: Q: glutamine; Arg: R: arginine; Ser: S: serine; Thr: T: threonine; Val: V: valine; Trp: W: tryptophan; Tyr: Y: tyrosine; Cys: C: cysteine.

Within the meaning of the present invention the term “Newcastle disease virus” or “NDV” includes all known velogenic, mesogenic and lentogenic NDV strains. According to the present invention pharmaceutical compositions comprising mesogenic and/or lentogenic strains are preferred. Still more preferred are the NDVs, being recombinant or not, the NDV strain MTH-68/H or they are derived from NDV strain MTH-68/H. The nucleic acid sequence of the NDV strain MTH-68/H is shown in SEQ ID No. 1 of the sequence listing.

Within the meaning of the present invention “derived from NDV strain MTH-68/H” is intended to mean that the derived NDV strain is encoded by a viral genome or comprises a viral genome having a nucleic acid sequence with a sequence identity of at least 70%, including for example 75%, 76%, 77%, 78%, 79%, 80%, 81%, 82%, 83%, 84%, 85, 86%, 87%, 88%, 89%, 90%, 91%, 92%, 93%, 94%, 95%, 96%, 97%, 98%, preferably of at least 75%, more preferably of at least 90%, more preferred of at least 95% to the nucleic acid sequence of the NDV strain MTH-68/H. In one embodiment the sequence identity is at least 99% or 100%. Any inserted sequences, such as cloning sites and/or transgenic constructs comprising nucleic acid sequences encoding one or more foreign genes comprised in the recombinant NDV strains, are not considered in the sequence alignment, and the reference sequence for the sequence alignment is that of SEQ ID No. 1 of the sequence listing. Said in other words a parent NDV strain for obtaining recombinant and/or mutated NDVs as comprised in the pharmaceutical formulation of the present invention can have a sequence as set forth in SEQ ID No. 1 of the sequence listing or be a variant of that sequence, encoded by a viral genome or comprises a viral genome having a nucleic acid sequence with a sequence identity of at least 70%, including for example 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, preferably of at least 75%, more preferably of at least 90%, more preferred of at least 95% or at least 99% to the nucleic acid sequence as set forth in SEQ ID No. 1 of the sequence listing.

A sequence variation is for example the substitution, insertion or deletion of at least one nucleotide, preferably the substitution, insertion or deletion of up to 20 nucleotides, still more preferably of up to 15 nucleotides. Any combination of substitution, insertion and deletion is also comprised by that definition, provided that the final construct possesses the desired characteristics. The variation may result in at least one amino acid substitution, wherein each amino acid substitution can be a conservative or a non-conservative amino acid substitution.

A NDV strain derived from NDV strain MTH-68/H having a mutation in the HN gene product or HN protein at position 277, wherein the amino acid phenylalanine (F) is substituted to leucine (L) at position 277, is referred to herein also as NDV-Mut HN(F277L).

It has been surprisingly shown that the mutation in the HN gene results in a beneficially improved replication capability of the respective strain as compared to an otherwise identical NDV not having said mutation in the HN gene. The beneficially increased replication rate of the NDV not only results in higher number of virus particles capable of infecting and destroying cancer cells, but also results in a higher expression of a foreign gene carried by the respective recombinant NDV strains. As explained earlier, the respective gene products of the foreign gene results in an improved immunological response and thus an improved destruction of cancer cells.

The foreign gene or part or the respective variant is/are expressed in the recombinant NDV strain, when the virus is replicating in a suitable host or host cell. An expression of a gene means that the nucleic acid information encoded by the gene is translated in a respective amino acid sequence, which is the primary sequence for building up the respective protein or gene product. By way of example, the gene product of the HN gene is the HN protein. Using an NDV virus as a carrier for a foreign gene provides the advantage that the foreign gene is targeted to and expressed in tumor cells, so that the gene product is present in the micro-environment of the tumor cells only. This helps to increase the efficiency of the therapy and to avoid any undesired side effects, which may incur when the gene product, i.e. the protein, is administered to a patient in need thereof.

The pharmaceutical formulation, also referred to as pharmaceutical composition or composition or formulation herein, may also comprise a carrier medium comprising or consisting of at least one pharmaceutically acceptable carrier medium. The carrier medium may comprise pharmaceutically acceptable substances and/or liquids suitable for injection and/or stabilization of the oncolytic viruses and/or for improving their delivery to cancer cells. The solvent can be an aqueous carrier, for example sterile water for injection or sterile buffered physiological saline or another, preferably isotonic buffer system having a pH in the range of 7.2 to 7.6, or a sterile culture medium or any mixture thereof. The composition may also comprise one or more additional pharmaceutically acceptable additives, such as adjuvants or excipients.

The checkpoint modulator or the checkpoint modulators encoded by one or more of the recombinant NDV strains can be selected from:
An antibody, preferably a monoclonal antibody, directed to the surface protein CTLA-4 or an antigen-binding part directed to the surface protein CTLA-4 (anti-CTLA-4). In one embodiment the antibody is Ipilimumab, an antigen-binding part of Ipilimumab, a variant of Ipilimumab or a variant of an antigen-binding part of Ipilimumab.
The surface protein CTLA-4 acts as a type of “off switch” to keep the immune system in check. Attaching an antibody, preferably a monoclonal antibody, to CTLA-4 stops it from working by blocking the binding of B7-1/B7-2 to CTLA-4 and can boost the immune response against cancer cells by allowing the T cells to be active.
An antibody, preferably a monoclonal antibody, directed to checkpoint inhibition protein PD-1 (programmed cell death protein 1, also known as CD279) or an antigen-binding part directed to protein PD-1 (anti-PD-1). They work by blocking a signal that prevents activated T cells from attacking the cancer upon expression in rNDV-expresser cells, thus allowing the immune system to clear the cancer. In one embodiment the antibody is Nivolumab, an antigen-binding part of Nivolumab, a variant of Nivolumab or a variant of an antigen-binding part of Nivolumab.
An antibody, preferably a monoclonal antibody, directed to protein PD-L1 or an antigen-binding part directed to protein PD-L1 (anti-PD-L1). In one embodiment the antibody is Atezolizumab, an antigen-binding part of Atezolizumab, a variant of Atezolizumab or a variant of an antigen-binding part of Atezolizumab.
The checkpoint protein PD-1 normally acts as a type of “off switch” that helps keep the T cells from attacking other cells in the body, when it attaches to PD-L1, which can be present on cancer cells in large amounts. Antibodies, preferably monoclonal antibodies, that target either PD-1 or PD-L1 can block this binding, which in consequence can boost the immune response against cancer cells.
An antibody, preferably a monoclonal antibody, directed to killer-cell immunoglobulin-like receptors (KIRs), preferably KIR2DL1/2L3, or an antigen-binding part directed to killer-cell immunoglobulin-like receptors (KIRs), preferably KIR2DL1/2L3, (anti-KIR). They work by regulating the killing function of the cells expressing the receptors. In one embodiment the antibody is Lirilumab, an antigen-binding part of Lirilumab, a variant of Lirilumab or a variant of an antigen-binding part of Lirilumab.
An antibody, preferably a monoclonal antibody, directed to and inhibiting LAG-3 or an antigen-binding part directed to and inhibiting LAG-3 (anti-LAG-3). LAG-3 is a protein, which is present on the surface of T-cells. When the antibody or its antigen-binding part is bound to LAG-3 on T-cells, the T-cells are stimulated to attack cancer cells. In one embodiment the antibody is Relatlimab, an antigen-binding part of Relatlimab, a variant of Relatlimab or a variant of an antigen-binding part of Relatlimab. When bound to LAG-3 on T-cells, the T-cells are stimulated to attack cancer cells.
An antibody, preferably a monoclonal antibody, directed to NKG2A or an antigen-binding part directed to NKG2A (anti-NKG2A). Binding of the antibody to NKG2a prevents the binding of NKG2A to its ligand human leukocyte antigen-E (HLA-E), which induces an immune response against the cancer cells leading to their destruction. In one embodiment the antibody is Monalizumab, an antigen-binding part of Monalizumab, a variant of Monalizumab or a variant of an antigen-binding part of Monalizumab.
An antibody, preferably a monoclonal antibody, directed to and activating the glucocorticoid-induced TNF-superfamily receptor (GITR), thereby blocking the interaction of GITR with its ligand, or an antigen-binding part directed to the glucocorticoid-induced TNF-superfamily receptor (GITR) (anti-GITR). In one embodiment the antibody is TRX518, an antigen-binding part of TRX518, a variant of TRX518 or a variant of an antigen-binding part of TRX518.
An antibody, preferably a monoclonal antibody, directed to and activating the protein OX40 (also known as CD134 or TNFRSF4), thereby increasing the number of T-cells, or an antigen-binding part directed to and activating the protein OX40 (anti-OX40). In one embodiment the antibody is BMS 986178, an antigen-binding part of BMS 986178, a variant of BMS 986178 or a variant of an antigen-binding part of BMS 986178.
Any combination of these antibodies, antigen-binding parts or variants of these antibodies or antigen-binding parts thereof.

The antibodies as mentioned above can be monoclonal antibodies, still more preferred monoclonal humanized antibodies, in a preferred embodiment.

The nucleic acid sequence comprising a nucleic acid sequence encoding at least one checkpoint modulator, also referred to as “transgenic construct” or “inserted sequence” herein, can be or comprises a nucleic acid according to SEQ ID No. 9 to SEQ ID No. 16 or parts thereof. In another embodiment the said transgenic construct can have a sequence identity of at least 70%, including for example 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, more preferably of at least 75%, more preferred of at least 90%, still more preferred of at least 95% to the respective sequence of any one of SEQ ID No. 9 to SEQ ID No. 16. The sequences given by these SEQ ID numbers are as follows:
SEQ ID No. 9: Nucleic acid sequence of a transgenic construct comprising a nucleic acid sequence encoding an anti-CTLA-4 antibody;
SEQ ID No. 10: Nucleic acid sequence of a transgenic construct comprising a nucleic acid sequence encoding Nivolumab;
SEQ ID No. 11: Nucleic acid sequence of a transgenic construct comprising a nucleic acid sequence encoding the anti-PD-L1 antibody Atezolizumab;
SEQ ID No. 12: Nucleic acid sequence of a transgenic construct comprising a nucleic acid sequence encoding an anti-KIR antibody;
SEQ ID No. 13: Nucleic acid sequence of a transgenic construct comprising a nucleic acid sequence encoding an anti-LAG-3 antibody;
SEQ ID No. 14: Nucleic acid sequence of a transgenic construct comprising a nucleic acid sequence encoding an anti-NKG2A antibody;
SEQ ID No. 15: Nucleic acid sequence of a transgenic construct comprising a nucleic acid sequence encoding an anti-GITR antibody;
SEQ ID No. 16: Nucleic acid sequence of a transgenic construct comprising a nucleic acid sequence encoding an anti-OX40 antibody.

In one embodiment the recombinant NDV can encode any one of the checkpoint modulators as mentioned before. In another embodiment of the present invention a recombinant NDV can encode in its viral genome at least two checkpoint modulators or parts or variants thereof, which checkpoint modulators are selected from the above mentioned group. Still more preferred the sequences comprising a nucleic acid sequence encoding a checkpoint modulator can be any sequence according to SEQ ID No. 9 to SEQ ID No. 16 of the sequence listing or a variant thereof.

A “variant” of a starting nucleic acid is a nucleic acid that comprises a nucleic acid sequence different from that of the starting nucleic acid. Generally, a variant will possess at least 75% sequence identity, including for example 75%, 76%, 77%, 78%, 79%, 80%, 81%, 82%, 83%, 84%, 85%, 86%, 87%, 88%, 89%, 90%, 91%, 92%, 93%, 94%, 95%, 960%, 97%, 98%, 99%, preferably at least 90% sequence identity, more preferably at least 95% sequence identity, and most preferably at least 98% sequence identity with the native nucleic acid. Variants of a nucleic acid may be prepared by introducing appropriate nucleotide changes into the nucleic acid. Such variants include, for example, deletions from, and/or insertions into and/or substitutions of residues within the nucleic acid sequence of the gene of interest. Any combination of deletion, insertion, and substitution is possibly made to arrive at the final construct, provided that the final construct possesses the desired characteristics. Methods for generating nucleic acid sequence variants are known to the skilled person.

As used herein, a “variant” of a starting polypeptide or protein, e.g. an antibody or another protein, is a polypeptide or protein that comprises an amino acid sequence different from that of the starting polypeptide or protein. Generally, a variant will possess at least 75% sequence identity, including for example 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, preferably at least 90% sequence identity, more preferably at least 95% sequence identity, and most preferably at least 98% sequence identity with the native polypeptide or protein. Variants of a polypeptide or a protein may be prepared by introducing appropriate nucleotide changes into the nucleic acid encoding the polypeptide or protein. Such variants include, for example, deletions from, and/or insertions into and/or substitutions of residues within the amino acid sequence of the polypeptide/protein of interest. Any combination of deletion, insertion, and substitution is possibly made to arrive at the final construct, provided that the final construct possesses the desired characteristics. The amino acid changes also may alter post-translational processes of the polypeptide, such as changing the number or position of glycosylation sites. Methods for generating amino acid sequence variants of polypeptides are known to the skilled person.

Percentage sequence identity is determined after the best alignment of the sequence of interest with the respective reference sequence. The best alignment of the sequences may for example be produced by means of the Fitch et al., Proc. Natl. Acad. Sci. USA 80:1382-1386 (1983), version of the algorithm described by Needleman et al., J. Mol. Biol. 48:443-453 (1970), after aligning the sequences to provide for best homology, by means of the similarity search method of Pearson and Lipman, Proc. Natl Acad. Sci. USA 85, 2444 (1988), or by means of computer programs which use these algorithms (in particular BLASTN in Wisconsin Genetics Software Package, Genetics Computer Group, 575 Science Drive, Madison, Wis.). The percentage identity is calculated by determining the number of identical positions between the two sequences being compared, dividing this number by the number of positions compared and multiplying the result obtained by 100 so as to obtain the percentage identity between these two sequences. Preferably, sequence identity of a nucleotide sequence is determined using BLASTN, preferably BLASTN in standard settings as provided by the website of the U.S. National Library of Medicine “https://blast.ncbi.nlm.nih.gov”. Also preferably, sequence identity of an amino acid sequence is determined using BLASTP, preferably BLASTP in standard settings as provided by the website of the U.S. National Library of Medicine “https://blast.ncbi.nlm.nih.gov”. It is further preferred that the sequence identity is calculated over the entire length of the respective sequence.

As used herein, the term “part” means a fragment of a starting nucleic acid or amino acid sequence, which fragment is a made up of a consecutive sequence of the nucleotides or amino acids of the starting sequence. The part or fragment preferably comprises at least 10, more preferably at least 20 and preferably up to 500 nucleotides or amino acids. In any case a part according to the present invention is a functional part. That is nucleic acid or protein encoded by that nucleic acid must provide the desired function, e.g. an antigen-binding function or expression inhibiting function as specified herein, of the starting gene or protein.

The pharmaceutical formulation according to the present invention can comprise one NDV strain comprising in its viral genome a nucleic acid sequence comprising at least one foreign gene, the at least one foreign gene encoding a checkpoint modulator, preferably a checkpoint modulator as specified above, or the pharmaceutical formulation according to the present invention may comprise a combination of recombinant NDV strains each strain comprising a nucleic acid sequence encoding as a foreign gene another checkpoint modulator, preferably another checkpoint modulator as specified above.

The pharmaceutical formulation according to the present invention can for example comprise at least three recombinant NDV strains each strain comprising a nucleic acid sequence encoding as a foreign gene another checkpoint modulator, preferably another checkpoint modulator as specified above, for example in SEQ ID No. 9 to 16 or a variant thereof. Alternatively the pharmaceutical composition according to the present invention may comprise at least 4, 5, 6 or 7 recombinant NDV strains each strain comprising a nucleic acid sequence encoding as a foreign gene another checkpoint modulator, preferably another checkpoint modulator as specified above. Preferably the pharmaceutical formulation of the present invention does not comprise more than 10 different recombinant NDV strains each strain comprising a nucleic acid sequence encoding as a foreign gene another checkpoint modulator, preferably another checkpoint modulator as specified above.

A preferred combination of these different recombinant NDV strains is a combination of at least one recombinant NDV strain comprising in its viral genome a nucleic acid sequence comprising at least one foreign gene encoding an antibody, preferably a monoclonal antibody, directed to protein PD-1 or an antigen-binding part directed to protein PD-1 (anti-PD-1), and at least one recombinant NDV strain comprising in its viral genome a nucleic acid sequence comprising at least one foreign gene encoding an antibody, preferably a monoclonal antibody, directed to protein PD-L1 or an antigen-binding part directed to protein PD-L1 (anti-PD-L1), and at least one recombinant NDV strain comprising in its viral genome a nucleic acid sequence comprising at least one foreign gene encoding an antibody, preferably a monoclonal antibody, directed to the surface protein CTLA-4 or an antigen-binding part directed to the surface protein CTLA-4 (anti-CTLA-4). The anti-PD-1 antibody can be Nivolumab, for example as shown in SEQ ID No. 10, an antigen-binding part of Nivolumab, a variant of Nivolumab or a variant of an antigen-binding part of Nivolumab, the anti-PD-L1 antibody can be Atezolizumab, for example as shown in SEQ ID No. 11, an antigen-binding part of Atezolizumab, a variant of Atezolizumab or a variant of an antigen-binding part of Atezolizumab, and/or the anti-CTLA-4 antibody can be Ipilimumab, for example as shown in SEQ ID No. 9, an antigen-binding part of Ipilimumab, a variant of Ipilimumab or a variant of an antigen-binding part of Ipilimumab.

Even though PD-1- and PD-L1 interact with each other, it has been surprisingly found out by the present inventor that the combined use of an anti-PD-1 antibody and an anti-PD-L1 antibody improves the respective checkpoint inhibition and thus enables the immune system to kill more tumor cells.

Also it has been surprisingly found out by the present inventor that the combinatorial use of an anti-PD-1 antibody or an anti-PD-L1 antibody with an anti-CTLA-4 antibody has a synergistic effect resulting in an enhanced capability of the immune system to attack and kill tumor cells.

The angiogenesis inhibitor or the angiogenesis inhibitors encoded by one or more of the recombinant NDV strains can be selected from:
An antibody, preferably a monoclonal antibody, directed to the growth factor protein VEGF-A or an antigen-binding part thereof directed to the growth factor protein VEGF-A (anti-VEGF-A), such as Bevacizumab or a variant or an antigen-binding part thereof. VEGF-A is a growth factor protein that stimulates angiogenesis in a variety of diseases, especially in cancer. Thus the antibody is capable of blocking angiogenesis. In one embodiment the antibody is Bevacizumab, an antigen-binding part of Bevacizumab, a variant of Bevacizumab or a variant of an antigen-binding part of Bevacizumab.
An antibody, preferably an antagonistic antibody directed to VEGFR, or an antigen-binding part thereof directed to VEGFR (anti-VEGFR). The vascular endothelial growth factor (VEGF) is involved in angiogenesis. The natural ligands comprise VEGF-A, VEGF-C and VEGF-D. Antagonistic antibodies targeting VEGF lead to inhibition of VEGF-mediated tumor angiogenesis. In one embodiment the antibody is Ramucirumab, an antigen-binding part of Ramucirumab, a variant of Ramucirumab or a variant of an antigen-binding part of Ramucirumab.
Any combination of these antibodies, antigen-binding parts or variants of these antibodies or antigen-binding parts thereof.

The antibodies as mentioned above can be monoclonal antibodies, still more preferred monoclonal humanized antibodies, in a preferred embodiment.

As used herein, an agonist is intended to mean a substance that activates signal transduction in the associated cell by occupying a receptor. Chemical compounds that bind to a receptor, but do not activate it, and thus block and inhibit it, are called antagonists.

The antibodies as mentioned above can be monoclonal antibodies, still more preferred monoclonal humanized antibodies, in a preferred embodiment.

The nucleic acid sequence comprising a nucleic acid sequence encoding at least one angiogenesis inhibitor, also referred to as “transgenic construct” or “inserted sequence” herein, can be or comprises a nucleic acid according to SEQ ID No. 17 or SEQ ID No. 18 or parts thereof. In another embodiment the transgenic construct can have a sequence identity of at least 70%, including for example 75%, 76%, 77%, 78%, 79%, 80%, 81%, 82%, 83%, 84%, 85%, 86%, 87%, 88%, 89%, 90%, 91%, 92%, 93%, 94% 95%, 96%, 97%, 98%, 99%, more preferably of at least 75%, more preferred of at least 90%, still more preferred of at least 95% to the respective sequence of SEQ ID No. 17 or SEQ ID No. 18. The sequences given by these SEQ ID numbers are as follows:
SEQ ID No. 17: Nucleic acid sequence of a transgenic construct comprising a nucleic acid sequence encoding an anti-VEGF-A antibody;
SEQ ID No. 18: Nucleic acid sequence of a transgenic construct comprising a nucleic acid sequence encoding an anti-VEGFR antibody;

In one embodiment the recombinant NDV can encode any one of the angiogenesis inhibitors as mentioned before. In another embodiment of the present invention a recombinant NDV can encode in its viral genome at least two angiogenesis inhibitors or parts or variants thereof, which angiogenesis inhibitors are selected from the above ones. Still more preferred the sequences comprising a nucleic acid sequence encoding an angiogenesis inhibitor can be any sequence according to SEQ ID No. 17 or 18 of the sequence listing or a variant thereof.

The pharmaceutical formulation according to the present invention can comprise one NDV strain comprising in its viral genome a nucleic acid sequence comprising at least one foreign gene, the at least one foreign gene encoding an angiogenesis inhibitor, preferably a angiogenesis inhibitor as specified above, for example in SEQ ID No. 17 or 18 or a variant thereof. For example, the pharmaceutical formulation can comprise a recombinant NDV strain encoding an anti-VEGF-A antibody. The anti-VEGF-A antibody can be Bevacizumab, for example as shown in SEQ ID No. 17, an antigen-binding part of Bevacizumab, a variant of Bevacizumab or a variant of an antigen-binding part of Bevacizumab.

The pharmaceutical formulation according to the present invention may alternatively comprise a combination of recombinant NDV strains each strain comprising a nucleic acid sequence encoding as a foreign gene another angiogenesis inhibitor, preferably another angiogenesis inhibitor as specified above. The pharmaceutical formulation according to the present invention can for example comprise at least three recombinant NDV strains each strain comprising a nucleic acid sequence encoding as a foreign gene another angiogenesis inhibitor, preferably another angiogenesis inhibitor as specified above, for example in SEQ ID No. 17 or 18 or variants thereof. Alternatively the pharmaceutical composition according to the present invention may comprise at least 4, 5, 6 or 7 recombinant NDV strains each strain comprising a nucleic acid sequence encoding as a foreign gene another angiogenesis inhibitor, preferably another angiogenesis inhibitor including those as specified above. Preferably the pharmaceutical formulation of the present invention does not comprise more than 10 different recombinant NDV strains, each strain comprising a nucleic acid sequence encoding as a foreign gene another angiogenesis inhibitor, preferably another angiogenesis inhibitor as specified above.

A preferred combination of these different recombinant NDV strains is a combination of at least one recombinant NDV strain comprising in its viral genome a nucleic acid sequence comprising at least one foreign gene encoding an antibody, preferably a monoclonal antibody, directed to the growth factor protein VEGF-A or an antigen-binding part thereof directed to the growth factor protein VEGF-A (anti-VEGF-A) and at least one recombinant NDV strain comprising in its viral genome a nucleic acid sequence comprising at least one foreign gene encoding an antibody, preferably a monoclonal antibody, directed to VEGFR, or an antigen-binding part thereof directed to VEGFR (anti-VEGFR). The anti-VEGF-A antibody can be Bevacizumab, for example as shown in SEQ ID No. 17, an antigen-binding part of Bevacizumab, a variant of Bevacizumab or a variant of an antigen-binding part of Bevacizumab, and/or the anti-VEGFR antibody can be Ramucirumab, for example as shown in SEQ ID No. 18, an antigen-binding part of Ramucirumab, a variant of Ramucirumab or a variant of an antigen-binding part of Ramucirumab.

Also it has been surprisingly found out by the present inventor that the combinatorial use of an anti-VEGF-A antibody with an anti-VEGFR antibody has a synergistic effect resulting in an enhanced capability of the immune system to attack and kill tumor cells.

The pharmaceutical formulation according to the present invention further comprises at least one recombinant NDV strain comprising in its viral genome a nucleic acid sequence comprising at least one foreign gene, the at least one foreign gene encoding a virulence factor. The virulence factor is preferably a protein with the ability to modulate the virus replication cycle, or a part thereof with the ability to modulate the virus replication cycle. Alternatively or in addition, the virulence factor is a protein, or a part thereof, with the ability to achieve an inhibition of the host's immune response.

The virulence factor or the virulence factors encoded by one or more of the recombinant NDV strains can be selected from:
The non-structural protein NS1 of influenza A virus, a part of the non-structural protein NS1 of influenza A virus, a variant of the non-structural protein NS1 of influenza A virus or a variant of a part of the non-structural protein NS1 of influenza.
A protein that reduces or inhibits IFN expression such as an IFN-beta receptor, or a part thereof that reduces or inhibits IFN expression such as an IFN-beta receptor. In one embodiment the protein is the encoded viral protein B18R from vaccinia virus, a part of the viral protein B18R from vaccinia virus, or a variant of the viral protein B18R from vaccinia virus or a variant of a part of the viral protein B18R from vaccinia virus. B18R is a homologue of the human IFN-β receptor. B18R acts as a decoy for IFN-β, thereby inhibiting activation by cell signaling of the antiviral host response in native cells, resulting in increased lytic activity in cancer cells.
A combination of these proteins, parts or variants of these proteins.

The nucleic acid sequence comprising a nucleic acid sequence encoding at least one virulence factor, also referred to as “transgenic construct” or “inserted sequence” herein, can be or comprises a nucleic acid according to SEQ ID No. 19 or SEQ ID No. 20 or parts thereof. In another embodiment the transgenic construct can have a sequence identity of at least 70%, including for example 75%, 76%, 77%, 78%, 79%, 80%, 81%, 82%, 83%, 84%, 85%, 86%, 87%, 88%, 89%, 90%, 91%, 92%, 93%, 94% 95%, 96%, 97%, 98%, 99%, more preferably of at least 75%, more preferred of at least 90%, still more preferred of at least 95% to the respective sequence of SEQ ID No. 19 or SEQ ID No. 20.

The sequence given by these SEQ ID number is as follows:
SEQ ID No. 19: Nucleic acid sequence of a transgenic construct comprising a nucleic acid sequence encoding a non-structural protein NS1;
SEQ ID No. 20: Nucleic acid sequence of a transgenic construct comprising a nucleic acid sequence encoding B18R.

In one embodiment the recombinant NDV can encode any one of the virulence factors as mentioned before. In another embodiment of the present invention a recombinant NDV can encode in its viral genome at least two virulence factors or parts or variants thereof, which virulence factors are selected from the above ones. Still more preferred the sequences comprising a nucleic acid sequence encoding a virulence factor can be any sequence according to SEQ ID No. 19 or 20 of the sequence listing or a variant thereof.

The pharmaceutical formulation according to the present invention comprises one NDV strain comprising in its viral genome a nucleic acid sequence comprising at least one foreign gene, the at least one foreign gene encoding a virulence factor, preferably a virulence factor as specified above, for example in SEQ ID No. 19 or SEQ ID No. 20 or a variant thereof. For example, the pharmaceutical formulation can comprise a recombinant NDV strain encoding a non-structural protein NS1.

The pharmaceutical formulation according to the present invention may alternatively comprise a combination of recombinant NDV strains each strain comprising a nucleic acid sequence encoding as a foreign gene another virulence factor, preferably another virulence factor as specified above. The pharmaceutical formulation according to the present invention can for example comprise at least three recombinant NDV strains each strain comprising a nucleic acid sequence encoding as a foreign gene another virulence factor, preferably another virulence factor as specified above, for example in SEQ ID No. 19 or SEQ ID No. 20 or variants thereof. Alternatively the pharmaceutical composition according to the present invention may comprise at least 4, 5, 6 or 7 recombinant NDV strains each strain comprising a nucleic acid sequence encoding as a foreign gene another virulence factor, preferably another virulence factor including those as specified above. Preferably the pharmaceutical formulation of the present invention does not comprise more than 10 different recombinant NDV strains each strain comprising a nucleic acid sequence encoding as a foreign gene another virulence factor, preferably another virulence factor as specified above.

A preferred combination of these different recombinant NDV strains is a combination of at least one recombinant NDV strain comprising in its viral genome a nucleic acid sequence comprising at least one foreign gene encoding the non-structural protein NS1 of influenza A virus, for example as shown in SEQ ID No. 19, a part of the non-structural protein NS1 of influenza A virus, a variant of the non-structural protein NS1 of influenza A virus or a variant of a part of the non-structural protein NS1 of influenza A virus, and at least one recombinant NDV strain comprising in its viral genome a nucleic acid sequence comprising at least one foreign gene encoding B18R, for example as shown in SEQ ID No. 20, or a variant of B18R or a variant of a part of B18R.

It has been surprisingly found out by the present inventor that the combinatorial use of NS1 and B18R improves the effectiveness of the viruses and enable them to inhibit an interferon response.

Even though interleukin-12 is a potent immunostimulatory cytokine that activates the innate and adaptive cellular immune system and improves the ability of T cells to enter tumor cells, in an embodiment of the present invention the pharmaceutical formulation does not comprise a recombinant NDV strain comprising in its viral genome a nucleic acid comprising a nucleic acid sequence encoding interleukin-12 (IL-12), a part of interleukin-12, a variant of interleukin-12 or a variant of a part of interleukin-12.

The interleukin-12 can be a human interleukin 12 (hIL-12) protein, or a part or a variant thereof. Also the interleukin-12 may be a non-secreting interleukin-12, also referred to as (ns)IL-12 herein, or a part or a variant thereof. The non-secreting interleukin-12 can be a non-secreting human interleukin-12 ((ns)hIL-12), a part of (ns)hIL-12, a variant of (ns)hIL-12 or a variant of a part of (ns)hIL-12. As used herein “(ns)” is intended to mean “non-secreting”.

That is, the fact that in one embodiment the pharmaceutical formulation does not comprise a recombinant NDV strain encoding IL-12, does not mean that the present invention excludes formulations comprising a recombinant NDV strain encoding IL-12. Formulations comprising one or more recombinant NDV strain encoding IL-12, for example an IL-12 as further detailed below, e.g. with reference to SEQ ID No. 21 and SEQ ID No. 22, are also encompassed by the present invention. Also, does the fact that in one embodiment the pharmaceutical formulation does not comprise a recombinant NDV strain encoding IL-12, not mean that in the treatment of cancer the claimed pharmaceutical formulation cannot be administered in a combination with another pharmaceutical formulation comprising a recombinant NDV strain, based on a NDV strain as described herein, and comprising as a foreign gene a nucleic acid sequence encoding interleukin-12 (IL-12) or a part of interleukin-12 or a variant of interleukin-12 or a variant of a part of interleukin-12. Preferably, the foreign gene encodes a human interleukin 12 (hIL-12) protein, or a part thereof. The encoded protein can also be (ns)hIL-12, a part of (ns)hIL-12, a variant of (ns)hIL-12 or a variant of a part of (ns)hIL-12. A separate administration of interleukin, which can be given at a different time point as the pharmaceutical formulation according to the present invention, offers an additional tool in the treatment of cancer, but helps to avoid undesired side effects, which would be obtained when including IL-12 into the pharmaceutical formulation of the present invention.

The interleukin, either provided by the pharmaceutical formulation of this invention or separately administered, can be provided by at least one recombinant NDV strain comprising in its viral genome a nucleic acid sequence comprising at least one foreign gene, the at least one foreign gene encoding an interleukin-12 (IL-12) protein or a part of interleukin-12 or a variant of interleukin-12 or a variant of a part of interleukin-12.

The interleukin-12 can be a human interleukin 12 (hIL-12) protein, or a part thereof. In one embodiment the protein is (ns)hIL-12, a part of (ns)hIL-12, a variant of (ns)hIL-12 or a variant of a part of (ns)hIL-12. That is the interleukin is preferred to be a non-secreting human interleukin 12. A non-secreting interleukin may be advantageous over a normal interleukin, as it may prevent that there is an uncontrolled immune response, for example in the form of a massive cytokine secretion, triggered by administration of the recombinant NDV strain.

The nucleic acid sequence comprising a nucleic acid sequence encoding an interleukin-12 can be or comprises a nucleic acid according to SEQ ID No. 21 or SEQ ID No. 22 or parts thereof. In another embodiment the transgenic construct can have a sequence identity of at least 70%, including for example 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, more preferably of at least 75%, more preferred of at least 90%, still more preferred of at least 95% to the respective sequence of SEQ ID No. 21 or SEQ ID No. 22. The sequence given by these SEQ ID number is as follows:
SEQ ID No. 21: Nucleic acid sequence of a transgenic construct comprising a nucleic acid sequence encoding interleukin 12 (IL-12).
SEQ ID No. 22: Nucleic acid sequence of a transgenic construct comprising a nucleic acid sequence encoding (ns)hIL-12.

The pharmaceutical formulation according to the present invention further comprises at least one recombinant NDV strain comprising in its viral genome a nucleic acid sequence comprising at least one foreign gene, the at least one foreign gene being selected from the group of additional foreign genes consisting of:
A gene encoding the protein CD40 (cluster of differentiation 40), a part of CD40, a variant of CD40 or a variant of a part of CD40.
A gene encoding a membrane protein which is the receptor for the protein CD28, and which membrane protein is involved in the costimulatory signal essential for T-lymphocyte activation or a part of the membrane protein which is the receptor for the protein CD28, and which membrane protein is involved in the costimulatory signal essential for T-lymphocyte activation. In one embodiment the gene encoding the membrane protein CD80 (cluster of differentiation 80), a part of CD80, a variant of CD80 or a variant of a part of CD80,
A gene encoding a protein with the ability to selectively induce apoptosis in human tumor cells, but not in normal human cells, or a part thereof with the ability to selectively induce apoptosis in human tumor cells, but not in normal human cells. In one embodiment the protein is Apoptin (VP3 from chicken anemia virus), a part of Apoptin, a variant of Apoptin or a variant of a part of Apoptin.
A gene encoding an antibody, preferably an agonistic antibody, directed to CD28 or an antigen-binding part directed to CD28 (anti-CD28). CD28 (cluster of differentiation 28) is one of the proteins expressed on T cells that provide costimulatory signals required for T cell activation and survival. T cell stimulation through CD28 in addition to the T-cell receptor (TCR) can provide a potent signal for the production of various interleukins. In one embodiment the encoded antibody is Theralizumab, an antigen-binding part of Theralizumab, a variant of Theralizumab or a variant of an antigen-binding part of Theralizumab.
A gene encoding an antibody directed to CD33 or an antigen-binding part directed to CD33 (anti-CD33). In one embodiment the encoded antibody is Gemtuzumab, an antigen-binding part of Gemtuzumab, a variant of Gemtuzumab or a variant of an antigen-binding part of Gemtuzumab. That antibody or its antigen-binding part may preferably be used for the treatment of leukemia, for example myeloid leukemia.
A gene encoding an antibody, preferably an agonistic antibody, directed to CD39 or an antigen-binding part directed to CD39 (anti-CD39). CD39 (ecto-nucleoside triphosphate diphosphohydrolase 1, E-NTPDase1) converts ATP into AMP and plays an important role in the regulation of the ATP concentration. ATP is considered as an inhibitor of tumor cell proliferation, as it has a cytotoxic effect on tumors. Thus, the antibody or its antigen-binding part is used to activate an anti-tumor T cell response by inhibiting the conversion of the cytotoxic ATP.
A gene encoding an antibody, preferably an agonistic antibody, directed to CD40 or an antigen-binding part directed to CD40 (anti-CD40). CD40 (cluster of differentiation 40) is a costimulatory protein found on antigen-presenting cells and is required for their activation. Thus, the antibody or its antigen-binding part is used to activate an anti-tumor T cell response via activation of the antigen-presenting cells.
A gene encoding an antibody, preferably an agonistic antibody, directed to CD80 or an antigen-binding part directed to CD80 (anti-CD80). CD80 (cluster of differentiation 80) is a membrane protein which is the receptor for the proteins CD28 and CTLA-4, and which membrane protein is involved in the costimulatory signal essential for T-lymphocyte activation. Thus, the antibody or its antigen-binding part is used to activate T-lymphocytes.
A gene encoding an antibody directed to CA 15-3 or an antigen-binding part directed to CA 15-3 (anti-CA 15-3). CA 15-3 is also known as the human gene MUC1.
A gene encoding an antibody directed to CA 19-9 or an antigen-binding part directed to CA 19-9 (anti-CA 19-9). CA 19-9 is a muzin which corresponds to the sialylated Lewisa blood group antigen.
A gene encoding an antibody directed to CA 125 or an antigen-binding part directed to CA 125 (anti-CA 125). CA 125 is also known as the human gene MUC16. An antibody or its antigen-binding part targeting the MUC16 protein results in a G2/M-phase growth arrest and tumour cell apoptosis. In one embodiment the encoded antibody is Sofituzumab, an antigen-binding part of Sofituzumab, a variant of Sofituzumab or a variant of an antigen-binding part of Sofituzumab. That antibody or its antigen-binding part may preferably be used for the treatment of ovarian cancer.
A gene encoding an antibody, preferably an antagonistic antibody, directed to EGFR or an antigen-binding part directed to EGFR (anti-EGFR). The epidermal growth factor receptor (EGFR) is a transmembrane protein. Permanent activation of the receptor results in an uncontrolled cell proliferation and prevents controlled cell death (apoptosis), which contributes to the malignant transformation of cells. The antagonistic antibody is directed against the extracellular domain of the EGFR and leads to an inhibition of ligand binding. In one embodiment the encoded antibody is Cetuximab, an antigen-binding part of Cetuximab, a variant of Cetuximab or a variant of an antigen-binding part of Cetuximab. That antibody or its antigen-binding part may preferably be used for the treatment of colorectal carcinoma, squamous cell carcinoma and/or lung carcinoma.
A gene encoding an antibody, preferably an antagonistic antibody, directed to HER2 or an antigen-binding part directed to HER2 (anti-HER2). The antibody or its antigen-binding part is targeting the extracellular part of the HER2 receptor, which is overexpressed in certain cancers, and is intended to interrupt the growth-promoting signals. In one embodiment the encoded antibody is Trastuzumab, an antigen-binding part of Trastuzumab, a variant of Trastuzumab or a variant of an antigen-binding part of Trastuzumab. That antibody or its antigen-binding part may preferably be used for the treatment of breast cancer and/or stomach cancer.
A gene encoding an antibody directed to nfP2X₇ or an antigen-binding part directed to nfP2X₇ (anti-nfP2X₇). The non-functioning P2X₇ receptor is a form of the ATP-gated P2X₇ receptor in which the E200 epitope is exposed for antibody binding. The nfP2X₇ is able to form a Ca²⁺ channel but not an apoptotic pore and is found on every type of cancer cell but no normal cells. In one embodiment the encoded antibody is BIL=3s, an antigen-binding part of BIL=3s, a variant of BIL=3s or a variant of an antigen-binding part of BIL=3s.
A gene encoding an antibody directed to PSMA or an antigen-binding part directed to PSMA (anti-PSMA). The prostate specific membrane antigen (PSMA) is a cell surface peptidase highly expressed by malignant prostate epithelial cells and vascular endothelial cells of numerous solid tumor malignancies. In one embodiment the encoded antibody is J591, an antigen-binding part of J591, a variant of J591 or a variant of an antigen-binding part of J591. That antibody or its antigen-binding part may preferably be used for the treatment of prostate cancer.
A gene encoding a protein that induces the process of apoptosis by binding to a death receptor, such as death receptor DR4 and/or death receptor DR5, or a part thereof that induces the process of apoptosis by binding to a death receptor, such as death receptor DR4 and/or death receptor DR5. In one embodiment the encoded protein is TRAIL, a part of TRAIL, a variant of TRAIL or a variant of a part of TRAIL.
A gene encoding a green fluorescent protein, which can be used for the localization and monitoring of cells, in particular cancer cells, or a part of a green fluorescent protein. In one embodiment the encoded protein is enhanced green fluorescent protein (eGFP), a part of enhanced green fluorescent protein (eGFP), a variant of enhanced green fluorescent protein (eGFP) or a variant of a part of enhanced green fluorescent protein (eGFP).
Any combinations of these genes, parts or variants.

The antibodies as mentioned above can be monoclonal antibodies, still more preferred monoclonal humanized antibodies, in a preferred embodiment.

The nucleic acid sequence comprising a nucleic acid sequence encoding at least one foreign gene, also referred to as “transgenic construct” or “inserted sequence” herein, can be or comprises a nucleic acid according to SEQ ID No. 23 to SEQ ID No. 34 or parts thereof. In another embodiment the transgenic construct can have a sequence identity of at least 70%, including for example 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 9900, more preferably of at least 75%, more preferred of at least 90%, still more preferred of at least 95% to the respective sequence of any one of SEQ ID No. 23 to 34. The sequences given by these SEQ ID numbers are as follows:
SEQ ID No. 23: Nucleic acid sequence of a transgenic construct comprising a nucleic acid sequence encoding a CD80 protein;
SEQ ID No. 24: Nucleic acid sequence of a transgenic construct comprising a nucleic acid sequence encoding Apoptin;
SEQ ID No. 25: Nucleic acid sequence of a transgenic construct comprising a nucleic acid sequence encoding an anti-CD28 antibody;
SEQ ID No. 26: Nucleic acid sequence of a transgenic construct comprising a nucleic acid sequence encoding an anti-CD33 antibody;
SEQ ID No. 27: Nucleic acid sequence of a transgenic construct comprising a nucleic acid sequence encoding an anti-CD39 antibody;
SEQ ID No. 28: Nucleic acid sequence of a transgenic construct comprising a nucleic acid sequence encoding an anti-CA 15-3 antibody;
SEQ ID No. 29: Nucleic acid sequence of a transgenic construct comprising a nucleic acid sequence encoding an anti-CA 19-9 antibody;
SEQ ID No. 30: Nucleic acid sequence of a transgenic construct comprising a nucleic acid sequence encoding an anti-CA 125 antibody;
SEQ ID No. 31: Nucleic acid sequence of a transgenic construct comprising a nucleic acid sequence encoding an anti-EGFR antibody;
SEQ ID No. 32: Nucleic acid sequence of a transgenic construct comprising a nucleic acid sequence encoding an anti-nfP2X₇ antibody;
SEQ ID No. 33: Nucleic acid sequence of a transgenic construct comprising a nucleic acid sequence encoding an anti-PSMA antibody;
SEQ ID No. 34: Nucleic acid sequence of a transgenic construct comprising a nucleic acid sequence encoding eGFP.

In one embodiment the recombinant NDV can comprise in its viral genome any one of the above mentioned additional foreign genes or parts or variants thereof. In another embodiment of the present invention a recombinant NDV can comprise in its viral genome at least two of these additional foreign genes or parts or variants thereof. Still more preferred the sequences comprising a nucleic acid sequence comprising an additional foreign gene can be any sequence according to SEQ ID No. 23 to 34 of the sequence listing or a variant thereof.

The pharmaceutical formulation according to the present invention can comprise one NDV strain comprising in its viral genome a nucleic acid sequence comprising at least one foreign gene, the at least one foreign gene being an additional foreign gene as specified above, or the pharmaceutical formulation according to the present invention may comprise a combination of recombinant NDV strains each strain comprising a nucleic acid sequence comprising as a foreign gene another additional foreign gene as specified above.

The pharmaceutical formulation according to the present invention can for example comprise at least three recombinant NDV strains each strain comprising a nucleic acid sequence comprising as a foreign gene another additional foreign gene as specified above. Alternatively the pharmaceutical composition according to the present invention may comprise at least 4, 5, 6 or 7 recombinant NDV strains each strain comprising a nucleic acid sequence comprising another additional foreign gene as specified above. Preferably the pharmaceutical formulation of the present invention does not comprise more than 10 different recombinant NDV strains each strain comprising a nucleic acid sequence comprising another additional foreign gene as specified above.

The one or more recombinant NDV strain comprising in its viral genome a nucleic acid sequence comprising at least one foreign gene, the at least one foreign gene being selected from the group of additional foreign genes as specified above, for example in SEQ ID No. 23 to SEQ ID No. 34 or a variant thereof, can be comprised in the pharmaceutical formulation for further improving its efficacy. Depending on the actual need or the particular cancer to be treated one or more recombinant NDV strain each encoding another additional foreign gene may be selected. The combinations are preferably such that the encoded gene products support each other in their mode of action.

The NDV strain used as viral backbone of a recombinant NDV strain, i.e. the NDV strain which is used for inserting in its viral genome a nucleic acid sequence comprising at least one foreign gene encoding a checkpoint modulator, preferably a checkpoint modulator being selected from the above specified group, for example with reference to SEQ ID No. 9 to SEQ ID No. 16, and/or encoding an angiogenesis inhibitor, preferably an angiogenesis inhibitor being selected from the above specified group, for example with reference to SEQ ID No. 17 or SEQ ID No. 18, and/or encoding a virulence factor, preferably a virulence factor being selected from the above specified ones, for example with reference to SEQ ID No. 19 or SEQ ID No. 20, and/or encoding another additional foreign gene being selected from the above specified group, for example with reference to SEQ ID No. 23 to SEQ ID No. 34, and/or an interleukin-12 as specified above, for example with reference to SEQ ID No. 21 or SEQ ID No. 22, can, beside the mutation in the HN gene as specified above, further comprise additional mutations in its viral genome, or said in other words can comprise preferred amino acids at specific positions of its viral proteins.

These amino acid substitutions are also applicable for non-recombinant NDV strains not encoding a foreign gene, which are comprised in the pharmaceutical formulation according to the present invention as a further oncolytic agent. These non-recombinant NDV strains comprise a nucleic acid comprising a nucleic acid sequence encoding a hemagglutinin-neuramidase protein (HN protein) with an amino acid substitution at position 277 as specified above, and may in addition also comprise any one of the amino acid substitutions as specified in the following. That is this teaching is applicable for recombinant and non-recombinant NDV strains which may be present in the claimed pharmaceutical formulation.

Thus, the NDV strains, being recombinant or not, comprised in the pharmaceutical formulation according to the present invention, can have a viral genome, wherein the viral genome of at least one or each of the recombinant or not recombinant NDV strains further comprising in addition to the above described mutation in the HN gene, at least one mutation in the F gene and the thus encoded fusion protein (F protein). The mutated F gene encodes a fusion protein with an amino acid substitution at amino acid position 117 to an amino acid with a hydroxylated side chain, preferably where phenylalanine (F) is substituted to serine (S) at position 117 of the F gene product (also referred to as F(F117S) in the following), and/or with an amino acid substitution at amino acid position 190 to an amino acid with an aliphatic amino acid side chain, preferably where phenylalanine (F) is substituted to leucine (L) at position 190 of the F gene product (also referred to as F(F190L) in the following). The combination of both mutations at amino acid position 117 and 190 of the F gene is preferred. Thus, the pharmaceutical formulation according to the present invention can comprise recombinant or not recombinant NDV strains having following mutations or amino acid substitutions in the F protein: F(F117S), F(F190L), and F(F117S)+F(F190L).

It has been surprisingly shown that the at least one mutation, preferably both mutations (also referred to as “F2” herein), in the F gene at position 117 and 190 of the amino acid sequence of the resulting F protein beneficially improves the oncolytic potential of the respective NDV strains by improving the safety profile of the viruses in humans.

With regard to the viral safety profile, this in particular means that the intracerebral pathogenicity index (ICPI) is reduced by the mutations in the F gene at position 117 and/or 190, preferably by F2. The ICPI value indicates the pathogenicity of a Newcastle disease virus. Preferably, the ICPI is reduced by at least 10%, more preferably by at least 20% and still more preferred by at least 30%, as compared to the identically determined ICPI value of a NDV strain not having the respective mutation(s) in the F gene. The ICPI can be determined according to the method set forth in World Organization for Animal Health (OIE) 2009, Chapter 2.3.14. Newcastle disease, pp. 576-589, in: Manual of Diagnostic Tests and Vaccines for Terrestrial Animals 2009, World Organization for Animal Health (OIE), Paris.

The NDV strains, being recombinant or not, comprised in the pharmaceutical formulation according to the present invention can have a viral genome, wherein the viral genome of at least one or each of the recombinant or not recombinant NDV strains further comprises in addition to the above described mutation in the HN gene and optionally in addition to the above described mutations in the F gene at amino acid position 117 and/or 190, at least one mutation in the F gene encoding an amino acid substitution at position 289 to an amino acid with an aliphatic side chain other than leucine (L), preferably where leucine (L) is substituted to alanine (A) at position 289 of the F protein. This mutation is capable of improving the oncolytic potential of the respective NDV strain. Preferably the amino acid at position 289 of the F protein is an amino acid with an aliphatic side chain other than leucine (L). More preferably leucine (L) is substituted to alanine (A) at position 289 of the F protein (also referred to as F(L289A) in the following). Thus, also the following mutations in the F protein are encompassed by the present invention: F(F117S)+F(L289A), F(F190L)+F(L289A), and F(F117S)+F(F190L)+F(L289A). I.e. the F(L289A) mutation can be comprised in combination with at least one further amino acid substitution at position 117 or 190. It has been surprisingly shown that the additional amino acid substitution at position 289 of the F protein in combination with at least one of the other mutations, preferably both mutations (also referred to as “F3” (=“F2”+L289A) herein) further improves the oncolytic potential of the NDV strain, i.e. the ability to infect and lyse cancer cells. That is these NDV strains show improved oncolytic power and improved safety. The amino acid substitutions at the respective positions are as specified supra.

The NDV strains, being recombinant or not, comprised in the pharmaceutical formulation according to the present invention can have a viral genome, wherein the viral genome of at least one or each of the recombinant or not recombinant NDV strains further comprises in addition to the above described mutations in the HN gene and optionally in addition to the above described mutations in the F gene at amino acid position 117 and/or 190 and/or 289, preferably the F2 or F3 substitutions, a nucleic acid sequence having a mutation in the M gene and the thus encoded matrix protein (M protein). Preferably the mutated M gene encodes a matrix protein (M protein) with an amino acid substitution at position 165 to an amino acid with an aromatic side chain. Still more preferred the amino acid glycine (G) at amino acid position 165 of the M gene is substituted to the amino acid tryptophane (W).

NDV strains comprising both a mutation in the HN gene and the M gene, as described supra, have been shown as being particularly suitable to infect cancer cells and to provide an enhanced replication rate within the cancer cells.

The NDV strains, being recombinant or not, comprised in the pharmaceutical formulation according to the present invention can comprise in addition to the above described mutation in the HN gene and optionally the mutations in the F gene at amino acid positions 117 and/or 190 and/or 289 as specified above, and/or optionally in addition to the above described mutations in the M gene, at least one mutation in the L gene and the thus encoded RNA-dependent RNA polymerase protein, also referred to as L protein or L gene product herein. Still more preferred the mutated L gene encodes a RNA-dependent RNA polymerase protein with an amino acid substitution at position 757 to an amino acid with an aliphatic amino acid side chain other than valine (V), wherein preferably valine (V) is substituted to isoleucine (I) at position 757 of the L gene product, and/or with an amino acid substitution at position 1551 to an amino acid with a hydroxylated side chain, wherein preferably phenylalanine (F) is substituted to serine (S) at position 1551 of the L gene product, and/or with an amino acid substitution at position 1700 to an amino acid with an aliphatic side chain, preferably wherein arginine (R) is substituted to leucine (L) at position 1700 of the L gene product. Preferably the NDV genome comprises a mutated L gene encoding a L protein with the amino acid substitution at position 757, position 1551 and position 1700. These NDV strains may in addition also comprise the above described mutation in the M gene.

The three mutations in the F gene at position 117, 190 and 289 of the amino acid sequence of the resulting F protein and the three mutations in the L gene at position 757, 1551 and 1700 of the amino acid sequence of the resulting L protein, either alone or in combination, have been shown to beneficially improve the oncolytic potential of the respective NDV strains and the safety profile of these viruses in humans. The combinations include any combination of the referenced mutations in the F gene and L gene, and the F protein and L protein, respectively. That is any combination from three to six mutations. Particularly preferred is a combination of three mutations at amino acid position 117, 190 and 289 of the F gene (also referred to as “F3” herein) and a combination of all six mutations, that is the mutations at positions 117, 190 and 289 of the F gene product and at positions 757, 1551 and 1700 of the L gene product (also referred to as “F3L3” herein). The respective amino acid substitutions are as specified supra. The F3 and F3L3 mutations result in an improved oncolytic potential and a reduced intracerebral pathogenicity index (ICPI). Preferably, the ICPI is reduced by at least 10%, more preferably by at least 20% and still more preferred by at least 30%, as compared to the identically determined ICPI value of a NDV strain not having the respective mutations in the F gene and the L gene.

In addition to the mutation at position 277 of the HN gene product, the optional mutations as specified above at position 165 of the M gene product, at positions 117 and/or 190 and/or 289 of F gene product, and at positions 757 and/or 1551 and/or 1700 of the L gene product, a NDV according to the present invention may also comprise at least one further mutation in the L gene and the encoded L protein having an amino acid substitution at position 1717 to an amino acid with aromatic side chain other than tyrosine (Y), preferably where tyrosine (Y) is substituted to histidine (H) at position 1717 of the L gene product, and/or at position 1910 to an amino acid with a basic side chain, preferably where glutamic acid (E) is substituted to lysine (K) at position 1910 of the L gene product.

Alternatively or in addition a NDV according to the present invention may comprise a mutation in the RNA-editing sequence of the P gene. More preferably, the mutation abolishes and/or decreases the expression of the V protein.

Table 1a shows a list of mutations for obtaining NDV variants with increased titer production and/or good or improved safety profile according to the present invention. The mutation in the HN gene is comprised in all NDV strains comprised in the pharmaceutical formulation according to the present invention, while the mentioned mutations in the M gene, F gene and L gene may or may not be present. If present, any possible combination of these mutations is encompassed by the present invention.

TABLE 1a

| Gene | Relevant amino acid |
|---|---|
| HN | F277L |
| M | G165W |
| F | F117S |
| F | F190L |
| F | L289A |
| L | V757I |
| L | F1551S |
| L | R1700L |
| L | Y1717H |
| L | E1910K |

Table 1b shows particularly preferred NDV variants with increased titer production and/or improved safety profile for use as a strain for obtaining a recombinant NDV being comprised in the pharmaceutical formulation or for direct use in the pharmaceutical formulation of the present invention. Also shown is the parent NDV strain MTH-68/H. “+” means that at the mentioned amino acid position the original (first mentioned) amino acid is substituted by the last mentioned amino acid, while “−” means that there is no amino acid substitution at the mentioned position, i.e. the first mentioned amino acid is present. For example and with regard to the HN gene “+” means that at amino acid position 277 phenylalanine (F) is substituted to leucine (L).

TABLE 1b

| | Relevant | | | | | | | | | |
|---|---|---|---|---|---|---|---|---|---|---|
| | amino | MTH- | | | | | | | | |
| Gene | acid | 68/H | | | | | | | | |
| HN | F277L | − | + | + | + | + | + | + | + | + |
| M | G165W | − | − | + | − | + | − | + | − | + |
| F | F117S | − | + | + | + | + | + | + | + | + |
| F | F190L | − | + | + | + | + | + | + | + | + |
| F | L289A | − | − | − | + | + | − | − | + | + |
| L | V757I | − | − | − | − | − | + | + | + | + |
| L | F1551S | − | − | − | − | − | + | + | + | + |
| L | R1700L | − | − | − | − | − | + | + | + | + |
| L | Y1717H | − | − | − | − | − | − | − | + | + |
| L | E1910K | − | − | − | − | − | − | − | + | + |

Table 1c shows all preferred mutations in the F gene. “+” means that at the mentioned amino acid position the original (first mentioned) amino acid is substituted by the last mentioned amino acid, while “−” means that there is no amino acid substitution at the mentioned position, i.e. the first mentioned amino acid is present.

TABLE 1c

| | Relevant | | | | | | |
|---|---|---|---|---|---|---|---|
| | amino | | | | | | |
| Gene | acid | 1 | 1 | 2 | 2 | 2 | 3 |
| F | F117S | + | − | + | + | − | + |
| F | F190L | − | + | + | − | + | + |
| F | L289A | − | − | − | + | + | + |

Table 1d shows preferred mutations in the L gene. “+” means that at the mentioned amino acid position the original (first mentioned) amino acid is substituted by the last mentioned amino acid, while “−” means that there is no amino acid substitution at the mentioned position, i.e. the first mentioned amino acid is present.

TABLE 1d

| | Relevant | | | | | | | | | | | | | | | | | | | | | |
|---|---|---|---|---|---|---|---|---|---|---|---|---|---|---|---|---|---|---|---|---|---|---|
| | amino | | | | | | | | | | | | | | | | | | | | | |
| Gene | acid | 1 | 1 | 1 | 2 | 2 | 2 | 3 | 2 | 2 | 3 | 3 | 3 | 3 | 4 | 3 | 3 | 4 | 4 | 4 | 4 | 5 |
| L | V757I | + | − | − | + | + | − | + | + | − | − | + | + | − | + | + | − | − | + | + | − | + |
| L | F1551S | − | + | − | + | − | + | + | − | + | − | + | − | + | + | − | + | − | + | − | + | + |
| L | R1700L | − | − | + | − | + | + | + | − | − | + | − | + | + | + | − | − | + | − | + | + | + |
| L | Y1717H | − | − | − | − | − | − | − | + | + | + | + | + | + | + | + | + | + | + | + | + | + |
| L | E1910K | − | − | − | − | − | − | − | − | − | − | − | − | − | − | + | + | + | + | + | + | + |

A NDV strain derived from NDV strain MTH-68/H having a mutation in the HN gene, wherein the amino acid phenylalanine (F) is substituted to leucine (L) at amino acid position 277, and having a mutation in the M gene, wherein the amino acid glycine (G) is substituted to the amino acid tryptophane (W) at position 165 of the M gene product, is herein referred to also as NDV-Mut HN(F277L)/M(G165W) or MutHu1 or NDV-NoThaBene-1.

A NDV strain derived from NDV strain MTH-68/H having a mutation in the HN gene, wherein the amino acid phenylalanine (F) is substituted to leucine (L) at amino acid position 277, having a mutation in the M gene, wherein the amino acid glycine (G) is substituted to the amino acid tryptophane (W) at position 165 of the M gene product, having two mutations in the F gene, wherein the amino acid phenylalanine (F) is substituted to the amino acid serine (W) at position 117, and the amino acid phenylalanine (F) is substituted to the amino acid leucine (L) at position 190, and having three mutations in the L gene, wherein the amino acid valine (V) is substituted to the amino acid isoleucine (I) at position 757, the amino acid phenylalanine (F) is substituted to the amino acid serine (S) at position 1551, and the amino acid arginine (R) is substituted to the amino acid leucine (L) at position 1700 of the L gene product is herein referred to also as NDV-Mut HN(F277L)/M(G165W)/F(F117S)/F(F190L)/L(V757I)/L(F1551S)/L(R1700L) or MutHu2a or NDV-NoThaBene-2a.

A NDV strain derived from NDV strain MTH-68/H having a mutation in the HN gene, wherein the amino acid phenylalanine (F) is substituted to leucine (L) at amino acid position 277, having a mutation in the M gene, wherein the amino acid glycine (G) is substituted to the amino acid tryptophane (W) at position 165 of the M gene product, having three mutations in the F gene, wherein the amino acid phenylalanine (F) is substituted to the amino acid serine (W) at position 117, the amino acid phenylalanine (F) is substituted to the amino acid leucine (L) at position 190, and the amino acid leucine (L) is substituted to the amino acid alanine (A) at position 289 of the F gene product, and having three mutations in the L gene, wherein the amino acid valine (V) is substituted to the amino acid isoleucine (I) at position 757, the amino acid phenylalanine (F) is substituted to the amino acid serine (S) at position 1551, and the amino acid arginine (R) is substituted to the amino acid leucine (L) at position 1700 of the L gene product is herein referred to also as NDV-Mut HN(F277L)/M(G165W)/F(F117S)/F(F190L)/F(L289A)/L(V757I)/L(F1551S)/L(R1700L) or MutHu2b or NDV-NoThaBene-2b.

According to the invention the NDV strains comprised in the pharmaceutical formulation of the present invention are selected from NDV-NoThaBene-1 and/or NDV-NoThaBene-2a and/or NDV-NoThaBene-2b. In case that the NDV strains are recombinant any one of NDV-NoThaBene-1 or NDV-NoThaBene-2a or NDV-NoThaBene-2b may be used as the viral backbone for obtaining the respective recombinant strain. The one or more foreign genes encoded by these recombinant strains are preferably those as specified above, for example as shown with reference to SEQ ID No. 9 to SEQ ID No. 16 for the checkpoint modulators, with reference to SEQ ID No. 17 and/or SEQ ID No. 18 for angiogenesis inhibitors, with reference to SEQ ID No. 19 and/or SEQ ID No. 20 for virulence factors, with reference to SEQ ID No. 21 and/or SEQ ID. No. 22 for interleukin-12, and with reference to SEQ ID No. 23 to SEQ ID No. 34 for the other foreign genes, or variants thereof.

According to the present invention the parent NDV strain used for obtaining the recombinant NDV strains as described above or non-recombinant NDV strains, which are comprised in the claimed pharmaceutical formulation, is encoded by and/or comprises the nucleic acid sequence according to SEQ ID No. 1 or parts thereof. Thus the viral backbone (i.e. the viral genome without a transgenic construct) of the recombinant NDV strain or the optionally comprised non-recombinant NDV strains of the pharmaceutical formulation according to the present invention can be encoded by and/or comprises the nucleic acids according to any one of SEQ ID No. 2 to 7 or parts thereof. According to another preferred embodiment the parent NDV strain for each of the recombinant or non-recombinant NDV strains of the claimed pharmaceutical formulation comprises a nucleic acid which is at least 70% identical, including for example 75%, 76%, 77%, 78%, 79%, 80%, 81%, 82%, 83%, 84%, 85, 86%, 87%, 88%, 89%, 90%, 91%, 92%, 93%, 94%, 95%, 96%, 97%, 98%, 9900, more preferably of at least 75%, more preferred of at least 90%, still more preferred of at least 95% to any one of a nucleic acid sequence according to SEQ ID No. 1 and/or the viral backbone of the recombinant NDV strain or the optionally comprised non-recombinant NDV strains of the pharmaceutical formulation according to the present invention comprises a nucleic acid which is at least 70% identical, including for example 75%, 76%, 77%, 78%, 79%, 80%, 81%, 82%, 83%, 84%, 85%, 86%, 87%, 88%, 89%, 90%, 91%, 920%, 93%, 94%, 95%, 96%, 97%, 98%, 99%, more preferably of at least 75% more preferred of at least 90%, still more preferred of at least 95% to any one of a nucleic acid sequence according to SEQ ID No. 2 to SEQ ID No. 7 of the sequence listing. The sequences given by these SEQ ID numbers are as follows:
SEQ ID No. 1: Nucleic acid (cDNA) sequence of the Newcastle disease virus (NDV) strain MTH-68/H genome;
SEQ ID No. 2: Nucleic acid (cDNA) sequence of Newcastle disease virus strain Mut HN(F277L)/M(G165W) genome having a mutation in the HN gene and a mutation in the M gene (=a preferred NDV-NoThaBene-1 genome).
SEQ ID No. 3: Nucleic acid (cDNA) sequence of Newcastle disease virus strain according to SEQ ID No. 2, but comprising in addition a 12 bp insert containing a AfeI restriction site between nucleotide positions 3134 and 3135 of SEQ ID No. 2.
SEQ ID No. 4: Nucleic acid (cDNA) sequence of Newcastle disease virus strain Mut HN(F277L)/M(G165W)/F(F117S)/F(F190L)/L(V757I)/L(F1551S)/L(R1700L) genome having a mutation in the HN gene, a mutation in the M gene, two mutations in the F gene (F2) and three mutations in the L gene (L3) (=a preferred NDV-NoThaBene-2a genome).
SEQ ID No. 5: Nucleic acid (cDNA) sequence of Newcastle disease virus strain according to SEQ ID No. 4, but comprising in addition a 12 bp insert containing a AfeI restriction site between nucleotide positions 3134 and 3135 of SEQ ID No. 4.
SEQ ID No. 6: Nucleic acid (cDNA) sequence of Newcastle disease virus strain Mut HN(F277L)/M(G165W)/F(F117S)/F(F190L)/F(L289A)/L(V757I)/L(F1551S)/L(R1700L) genome having a mutation in the HN gene, a mutation in the M gene, three mutations in the F gene (F3) and three mutations in the L gene (L3) (=a preferred NDV-NoThaBene-2b genome).
SEQ ID No. 7: Nucleic acid (cDNA) sequence of Newcastle disease virus strain according to SEQ ID No. 6, but comprising in addition a 12 bp insert containing a AfeI restriction site between nucleotide positions 3134 and 3135 of SEQ ID No. 6.

The nucleic acid sequence of SEQ ID No. 3 differs from the nucleic acid sequence of SEQ ID No. 2 therein that a 12 bp insert containing unique AfeI restriction site between the P and M genes (between nucleotide position 3134-3135 of SEQ ID No. 2) has been inserted. This 12 bp sequence is for inserting a transgenic construct between the AGC and the GCT sequence. In the same way the nucleic acid sequence of SEQ ID No. 5 differs from the nucleic acid sequence of SEQ ID No. 4 and the nucleic acid sequence of SEQ ID No. 7 differs from the nucleic acid sequence of SEQ ID No. 6. The sequence of the AfeI-site is AGCGCT. The nucleic acid sequence of this 12 bp insert is shown in SEQ ID No. 8 of the sequence listing.

By way of example the nucleic acid sequence of SEQ ID No. 35 of the sequence listing shows a transgenic or recombinant NDV strain according to the present invention. Shown is a nucleic acid (cDNA) sequence of Newcastle disease virus strain Mut HN(F277L)/M(G165W)/F(F117S)/F(F190L)/F(L289A)/L(V757I)/L(F1551S)/L(R1700L)-Sofituzumab genome created by reverse genetics as disclosed herein, wherein the nucleic acid comprises a mutation in the HN gene, a mutation in the M gene, three mutations in the F gene (F3) and three mutations in the L gene (L3), and additionally encodes Sofituzumab. The transgenic construct comprising the nucleic acid sequence encoding Sofituzumab is in this example inserted in the 12 nucleotide long sequence according to SEQ ID No. 8 of the sequence listing in the AfeI-site. Instead of the nucleic acid sequence comprising a nucleic acid sequence encoding anti-CA 125, preferably Sofituzumab, any other nucleic acid sequence according to any one of SEQ ID No. 9 to 29 and SEQ ID No. 31 to 34 can be inserted to obtain a preferred transgenic NDV strain according to the present invention. Also instead of the Newcastle disease virus strain Mut HN(F277L)/M(G165W)/F(F117S)/F(F190L)/F(L289A)/L(V757I)/L(F1551S)/L(R1700L) any other of the above mentioned NDV strains derived from MTH-68/H can be used as viral backbone. Other preferred NDV viral backbones are shown in SEQ ID No. 2 to 5 of the sequence listing.

A person skilled in the art is well aware of the fact that the nucleobase thymine in the nucleic acid sequence of DNA is replaced by the nucleobase uracil in the nucleic acid sequence of RNA.

In describing nucleic acid formulation, structure and function herein, reference is made to any of a group of polymeric nucleotides such as DNA or RNA. Nucleic acid is composed of either one or two chains of repeating units called nucleotides, which nucleotides consist of a nitrogen base (a purine or pyrimidine) attached to a sugar phosphate. In the present specification, nucleotide residues are identified by using the following abbreviations. Adenine residue: A; guanine residue: G; thymine residue: T; cytosine residue: C; uracil residue: U. Also, unless explicitly otherwise indicated, the nucleotide sequences of nucleic acid are identified from 5′-terminal to 3′-terminal (left to right terminal), the 5′-terminal being identified as a first residue.

As mentioned above a NDV strain derived from NDV strain MTH-68/H having the respective mutation in the HN gene product or HN protein at position 277 is referred to herein also as NDV-Mut HN(F277L). A NDV strain derived from NDV strain MTH-68/H having the respective mutations in the HN gene product or HN protein at position 277 and in the F gene resulting in a substitution of the amino acids at position 117 and/or 190 is referred to herein also as NDV-Mut HN(F277L)/F(F117S), NDV-Mut HN(F277L)/F(F190L) or NDV-Mut HN(F277L)/F(F117S)/(F190L). A NDV strain derived from NDV strain MTH-68/H having the respective mutations in the HN gene product or HN protein at position 277 and in the F gene resulting in a substitution of the amino acids at position 117 and/or 190 and 289 are referred to herein also as NDV-Mut HN(F277L)/F(F117S)/F(L289A), NDV-Mut HN(F277L)/F(F190L)/F(L289A) or NDV-Mut HN(F277L)/F(F117S)/(F190L)/F(L289A). The combination of both mutations at amino acid position 117 and 190 of the F gene product is particularly preferred.

In the same manner the respective mutations/amino acid substitutions in the M gene and the L gene are denoted in these shortcuts. As already detailed before the NDV strains, being recombinant or not, as comprised in the pharmaceutical formulation according to the present invention can in one embodiment additionally comprise a mutation in the M gene, preferably resulting in a substitution of the amino acid glycine (G) to the amino acid tryptophane (W) at position 165 of the M gene product. The recombinant and/or mutated NDV strains can comprise in addition to the above described mutation in the HN gene and the mutations in the F gene at amino acid positions 117 and/or 190 and/or 289 as specified above, at least one mutation in the L gene and the thus encoded RNA-dependent RNA polymerase protein at amino acid positions 757 and/or 1551 and/or 1700 as specified above. These recombinant NDV strains may in addition also comprise the above described mutation in the M gene. As it relates to preferred and more preferred embodiments of the substitutions at amino acid positions 117, 190 and 289 of the F gene, and amino acid positions 757, 1551 and 1700 of the L gene reference to above disclosure with regard to the novel NDV strains per se is made. The same preferred and more preferred embodiments or amino acids apply for the recombinant NDV strains.

The foreign gene or part or respective variant thereof carried by one of these strain is referred to as Atezolizumab, Bevacizumab, Lirilumab, Relatlimab, Monalizumab, TRX518, BMS 986178, CD40, CD80, (ns)hIL-12, eGFP, Nivolumab, Ipilimumab, IL-12, NS1, Apoptin, B18R, Theralizumab, Gemtuzumab, anti-CD39, anti-CD40, anti-CD80, anti-CA 15-3, anti CA 19-9, Sofituzumab, Cetuximab, Trastuzumab, BIL=3s, J591, Ramucircumab or TRAIL in this shortcut for the strain.

Within the shortcut for the recombinant NDV strains as used herein a reference to e.g. Atezolizumab is intended to comprise the complete antibody, antigen-binding parts, and variants of the antibody or of antigen-binding parts, but also encompasses the more general form of an anti-PD-L1-antibody and antigen-binding parts of this antibody. A reference to Bevacizumab is again intended to comprise the complete antibody, antigen-binding parts and variants of the antibody or antigen-binding parts, but also the more general form of an antibody to the growth factor protein VEGF-A, and antigen-binding parts of this antibody. A reference to Lirilumab is intended to comprise the complete antibody, antigen-binding parts, and variants of the antibody or of antigen-binding parts, but also encompasses the more general form of an anti-KIR-antibody and antigen-binding parts of this antibody. A reference to Relatlimab is intended to comprise the complete antibody, antigen-binding parts, and variants of the antibody or of antigen-binding parts, but also encompasses the more general form of an anti-LAG-3-antibody and antigen-binding parts of this antibody. A reference to Monalizumab is intended to comprise the complete antibody, antigen-binding parts, and variants of the antibody or of antigen-binding parts, but also encompasses the more general form of an anti-NKG2A-antibody and antigen-binding parts of this antibody. A reference to TRX518 is intended to comprise the complete antibody, antigen-binding parts, and variants of the antibody or of antigen-binding parts, but also encompasses the more general form of an anti-GITR-antibody and antigen-binding parts of this antibody. A reference to BMS 986178 is intended to comprise the complete antibody, antigen-binding parts, and variants of the antibody or of antigen-binding parts, but also encompasses the more general form of an anti-OX40-antibody and antigen-binding parts of this antibody. A reference to CD40 is intended to comprise the complete protein, parts and variants of the protein. A reference to CD80 is intended to comprise the complete protein, parts and variants of the protein, as well as the more general form of a membrane protein which is the receptor for the protein CD28, and which membrane protein is involved in the costimulatory signal essential for T-lymphocyte activation, or a respective part thereof. A reference to (ns)hIL-12 is intended to comprise the complete protein, parts and variants of the protein, as well as the more general form of a human interleukin 12 (hIL-12), or a respective part thereof. A reference to eGFP is intended to comprise the complete protein, parts and variants of the protein, as well as the more general form of a green fluorescent protein, or a respective part thereof. A reference to Nivolumab is intended to comprise the complete antibody, antigen-binding parts, and variants of the antibody or of antigen-binding parts, but also encompasses the more general form of an anti-PD-1-antibody and antigen-binding parts of this antibody. A reference to Ipilimumab is again intended to comprise the complete antibody, antigen-binding parts and variants of the antibody or antigen-binding parts, but also the more general form of an anti-CTLA-4-antibody, and antigen-binding parts of this antibody. A reference to IL-12 is intended to comprise the complete protein, parts and variants of the protein, as well as the more general form of a protein which improves the cellular immune response and the ability of T cells to enter tumor cells, or a respective part thereof. A reference to NS1 is intended to comprise the complete protein, parts and variants of the protein, as well as the more general form of a protein with the ability to modulate the virus replication cycle, or a respective part thereof. A reference to Apoptin is intended to comprise the complete protein, parts and variants of the protein, as well as the more general form of a protein with the ability to selectively induce apoptosis in human tumor cells, but not in normal human cells, or a respective part thereof. A reference to B18R in this shortcut is intended to comprise the complete protein, parts and variants of the protein, as well as the more general form of a protein that reduces or inhibits IFN expression such as an IFN-beta receptor, or a respective part thereof. A reference to Theralizumab is intended to comprise the complete antibody, antigen-binding parts, and variants of the antibody or of antigen-binding parts, but also encompasses the more general form of an anti-CD28-antibody and antigen-binding parts of this antibody. A reference to Gemtuzumab is intended to comprise the complete antibody, antigen-binding parts, and variants of the antibody or of antigen-binding parts, but also encompasses the more general form of an anti-CD33-antibody and antigen-binding parts of this antibody. A reference to anti-CD39 is intended to comprise the complete antibody, antigen-binding parts, and variants of the antibody or of antigen-binding parts. A reference to anti-CD40 is intended to comprise the complete antibody, antigen-binding parts, and variants of the antibody or of antigen-binding parts. A reference to anti-CD80 is intended to comprise the complete antibody, antigen-binding parts, and variants of the antibody or of antigen-binding parts. A reference to anti-CA 15-3 is intended to comprise the complete antibody, antigen-binding parts, and variants of the antibody or of antigen-binding parts according to SEQ ID. 22, but also encompasses the more general form of an anti-CA 15-3-antibody and antigen-binding parts of this antibody. A reference to anti-CA 19-9 is intended to comprise the complete antibody, antigen-binding parts, and variants of the antibody or of antigen-binding parts, but also encompasses the more general form of an anti-CA 19-9-antibody and antigen-binding parts of this antibody. A reference to Sofituzumab is intended to comprise the complete antibody, antigen-binding parts, and variants of the antibody or of antigen-binding parts, but also encompasses the more general form of an anti-CA 125-antibody and antigen-binding parts of this antibody. A reference to Cetuximab is intended to comprise the complete antibody, antigen-binding parts, and variants of the antibody or of antigen-binding parts, but also encompasses the more general form of an anti-EGFR-antibody and antigen-binding parts of this antibody. A reference to Trastuzumab is intended to comprise the complete antibody, antigen-binding parts, and variants of the antibody or of antigen-binding parts, but also encompasses the more general form of an anti-HER2-antibody and antigen-binding parts of this antibody. A reference to BIL=3s is intended to comprise the complete antibody, antigen-binding parts, and variants of the antibody or of antigen-binding parts, but also encompasses the more general form of an anti-nfP2X₇-antibody and antigen-binding parts of this antibody. A reference to J591 is intended to comprise the complete antibody, antigen-binding parts, and variants of the antibody or of antigen-binding parts, but also encompasses the more general form of an anti-PSMA-antibody and antigen-binding parts of this antibody. A reference to Ramucirumab is intended to comprise the complete antibody, antigen-binding parts, and variants of the antibody or of antigen-binding parts, but also encompasses the more general form of an anti-VEGFR-antibody and antigen-binding parts of this antibody. A reference to TRAIL is intended to comprise the complete protein, parts and variants of the protein, as well as the more general form of a protein with the ability to induce the process of apoptosis by binding to a death receptor, such as death receptor DR4 and/or death receptor DR5, or a respective part thereof. A reference to the foreign gene in this shortcut is also intended to comprise the sequence of the transgenic construct comprising the respective foreign gene as identified in any of the nucleic acid sequences according to SEQ ID No. 9 to 34 of the sequence listing or a variant thereof.

The following recombinant or non-recombinant NDV strains can be comprised in the pharmaceutical formulation according to the present invention in a preferred embodiment:
NDV-Mut HN(F277L)
NDV-Mut HN(F277L)-Atezolizumab
NDV-Mut HN(F277L-Bevacizumab
NDV-Mut HN(F277L)-Lirilumab
NDV-Mut HN(F277L)-Relatlimab
NDV-Mut HN(F277L)-Monalizumab
NDV-Mut HN(F277L)-TRX518
NDV-Mut HN(F277L)-BMS 986178
NDV-Mut HN(F277L)-CD40
NDV-Mut HN(F277L)-CD80
NDV-Mut HN(F277L)-eGFP
NDV-Mut HN(F277L)-Nivolumab
NDV-Mut HN(F277L)-Ipilimumab
NDV-Mut HN(F277L)-NS1
NDV-Mut HN(F277L)-Apoptin
NDV-Mut HN(F277L)-B18R
NDV-Mut HN(F277L)-Theralizumab
NDV-Mut HN(F277L)-Gemtuzumab
NDV-Mut HN(F277L)-anti-CD39
NDV-Mut HN(F277L)-anti-CD40
NDV-Mut HN(F277L)-anti-CD80
NDV-Mut HN(F277L)-anti-CA 15-3
NDV-Mut HN(F277L)-anti-CA 19-9
NDV-Mut HN(F277L)-Sofituzumab
NDV-Mut HN(F277L)-Cetuximab
NDV-Mut HN(F277L)-Trastuzumab
NDV-Mut HN(F277L)-BIL=3s
NDV-Mut HN(F277L)-J591
NDV-Mut HN(F277L)-Ramucircumab
NDV-Mut HN(F277L)-TRAIL
NDV-Mut HN(F277L)-IL-12
NDV-Mut HN(F277L)-(ns)hIL-12
NDV-Mut HN(F277L)/F(F117S)/F(F190L)
NDV-Mut HN(F277L)/F(F117S)/F(F190L)-Atezolizumab
NDV-Mut HN(F277L)/F(F117S)/F(F190L)-Bevacizumab
NDV-Mut HN(F277L)/F(F117S)/F(F190L)-Lirilumab
NDV-Mut HN(F277L)/F(F117S)/F(F190L)-Relatlimab
NDV-Mut HN(F277L)/F(F117S)/F(F190L)-Monalizumab
NDV-Mut HN(F277L)/F(F117S)/F(F190L)-TRX518
NDV-Mut HN(F277L)/F(F117S)/F(F190L)-BMS 986178
NDV-Mut HN(F277L)/F(F117S)/F(F190L)-CD40
NDV-Mut HN(F277L)/F(F117S)/F(F190L)-CD80
NDV-Mut HN(F277L)/F(F117S)/F(F190L)-eGFP
NDV-Mut HN(F277L)/F(F117S)/F(F190L)-Nivolumab
NDV-Mut HN(F277L)/F(F117S)/F(F190L)-Ipilimumab
NDV-Mut HN(F277L)/F(F117S)/F(F190L)-NS1
NDV-Mut HN(F277L)/F(F117S)/F(F190L)-Apoptin
NDV-Mut HN(F277L)/F(F117S)/F(F190L)-B18R
NDV-Mut HN(F277L)/F(F117S)/F(F190L)-Theralizumab
NDV-Mut HN(F277L)/F(F117S)/F(F190L)-Gemtuzumab
NDV-Mut HN(F277L)/F(F117S)/F(F190L)-anti-CD39
NDV-Mut HN(F277L)/F(F117S)/F(F190L)-anti-CD40
NDV-Mut HN(F277L)/F(F117S)/F(F190L)-anti-CD80
NDV-Mut HN(F277L)/F(F117S)/F(F190L)-anti-CA 15-3
NDV-Mut HN(F277L)/F(F117S)/F(F190L)-anti-CA 19-9
NDV-Mut HN(F277L)/F(F117S)/F(F190L)-Sofituzumab
NDV-Mut HN(F277L)/F(F117S)/F(F190L)-Cetuximab
NDV-Mut HN(F277L)/F(F117S)/F(F190L)-Trastuzumab
NDV-Mut HN(F277L)/F(F117S)/F(F190L)-BIL=3s
NDV-Mut HN(F277L)/F(F117S)/F(F190L)-J591
NDV-Mut HN(F277L)/F(F117S)/F(F190L)-Ramucircumab
NDV-Mut HN(F277L)/F(F117S)/F(F190L)-TRAIL
NDV-Mut HN(F277L)/F(F117S)/F(F190L)-IL-12
NDV-Mut HN(F277L)/F(F117S)/F(F190L)-(ns)hIL-12

Thus, according to one embodiment the NDV strain, which is derived from NDV strain MTH-68/H, has a mutation in the HN gene, preferably resulting in a substitution of the amino acid phenylalanine (F) to leucine (L) at position 277 of the HN gene product, and at least two mutation in the F gene, resulting in a substitution of the amino acid phenylalanine (F) to the amino acid serine (S) at position 117 of the F gene product and resulting in a substitution of the amino acid phenylalanine (F) to the amino acid leucine (L) at position 190 of the F gene product.

Preferred NDV strains as comprised in the pharmaceutical formulation of the present invention may comprise a further mutation in the F gene resulting in a substitution of the amino acid leucine (L) to the amino acid alanine (A) at position 289 of the F gene product.
NDV-Mut HN(F277L)/F(F117S)/F(F190L)/F(L289A)
NDV-Mut HN(F277L)/F(F117S)/F(F190L)/F(L289A)-Atezolizumab
NDV-Mut HN(F277L)/F(F117S)/F(F190L)/F(L289A)-Bevacizumab
NDV-Mut HN(F277L)/F(F117S)/F(F190L)/F(L289A)-Lirilumab
NDV-Mut HN(F277L)/F(F117S)/F(F190L)/F(L289A)-Relatlimab
NDV-Mut HN(F277L)/F(F117S)/F(F190L)/F(L289A)-Monalizumab
NDV-Mut HN(F277L)/F(F117S)/F(F190L)/F(L289A)-TRX518
NDV-Mut HN(F277L)/F(F117S)/F(F190L)/F(L289A)-BMS 986178
NDV-Mut HN(F277L)/F(F117S)/F(F190L)/F(L289A)-CD40
NDV-Mut HN(F277L)/F(F117S)/F(F190L)/F(L289A)-CD80
NDV-Mut HN(F277L)/F(F117S)/F(F190L)/F(L289A)-eGFP
NDV-Mut HN(F277L)/F(F117S)/F(F190L)/F(L289A)-Nivolumab
NDV-Mut HN(F277L)/F(F117S)/F(F190L)/F(L289A)-Ipilimumab
NDV-Mut HN(F277L)/F(F117S)/F(F190L)/F(L289A)-NS1
NDV-Mut HN(F277L)/F(F117S)/F(F190L)/F(L289A)-Apoptin
NDV-Mut HN(F277L)/F(F117S)/F(F190L)/F(L289A)-B18R
NDV-Mut HN(F277L)/F(F117S)/F(F190L)/F(L289A)-Theralizumab
NDV-Mut HN(F277L)/F(F117S)/F(F190L)/F(L289A)-Gemtuzumab
NDV-Mut HN(F277L)/F(F117S)/F(F190L)/F(L289A)-anti-CD39
NDV-Mut HN(F277L)/F(F117S)/F(F190L)/F(L289A)-anti-CD40
NDV-Mut HN(F277L)/F(F117S)/F(F190L)/F(L289A)-anti-CD80
NDV-Mut HN(F277L)/F(F117S)/F(F190L)/F(L289A)-anti-CA 15-3
NDV-Mut HN(F277L)/F(F117S)/F(F190L)/F(L289A)-anti CA 19-9
NDV-Mut HN(F277L)/F(F117S)/F(F190L)/F(L289A)-Sofituzumab
NDV-Mut HN(F277L)/F(F117S)/F(F190L)/F(L289A)-Cetuximab
NDV-Mut HN(F277L)/F(F117S)/F(F190L)/F(L289A)-Trastuzumab
NDV-Mut HN(F277L)/F(F117S)/F(F190L)/F(L289A)-BIL=3s
NDV-Mut HN(F277L)/F(F117S)/F(F190L)/F(L289A)-J591
NDV-Mut HN(F277L)/F(F117S)/F(F190L)/F(L289A)-Ramucircumab
NDV-Mut HN(F277L)/F(F117S)/F(F190L)/F(L289A)-TRAIL
NDV-Mut HN(F277L)/F(F117S)/F(F190L)/F(L289A)-IL-12
NDV-Mut HN(F277L)/F(F117S)/F(F190L)/F(L289A)-(ns)hIL-12

Still further any one of the following NDV strains can be comprised in the pharmaceutical formulation of the present invention:
NDV-Mut HN(F277L)/M(G165W)
NDV-Mut HN(F277L)/M(G165W)-Atezolizumab
NDV-Mut HN(F277L)/M(G165W)-Bevacizumab
NDV-Mut HN(F277L)/M(G165W)-Lirilumab
NDV-Mut HN(F277L)/M(G165W)-Relatlimab
NDV-Mut HN(F277L)/M(G165W)-Monalizumab
NDV-Mut HN(F277L)/M(G165W)-TRX518
NDV-Mut HN(F277L)/M(G165W)-BMS 986178
NDV-Mut HN(F277L)/M(G165W)-CD40
NDV-Mut HN(F277L)/M(G165W)-CD80
NDV-Mut HN(F277L)/M(G165W)-eGFP
NDV-Mut HN(F277L)/M(G165W)-Nivolumab
NDV-Mut HN(F277L)/M(G165W)-Ipilimumab
NDV-Mut HN(F277L)/M(G165W)-NS1
NDV-Mut HN(F277L)/M(G165W)-Apoptin
NDV-Mut HN(F277L)/M(G165W)-B18R
NDV-Mut HN(F277L)/M(G165W)-Theralizumab
NDV-Mut HN(F277L)/M(G165W)-Gemtuzumab
NDV-Mut HN(F277L)/M(G165W)-anti-CD39
NDV-Mut HN(F277L)/M(G165W)-anti-CD40
NDV-Mut HN(F277L)/M(G165W)-anti-CD80
NDV-Mut HN(F277L)/M(G165W)-anti-CA 15-3
NDV-Mut HN(F277L)/M(G165W)-anti CA 19-9
NDV-Mut HN(F277L)/M(G165W)-Sofituzumab
NDV-Mut HN(F277L)/M(G165W)-Cetuximab
NDV-Mut HN(F277L)/M(G165W)-Trastuzumab
NDV-Mut HN(F277L)/M(G165W)-BIL=3s
NDV-Mut HN(F277L)/M(G165W)-J591
NDV-Mut HN(F277L)/M(G165W)-Ramucircumab
NDV-Mut HN(F277L)/M(G165W)-TRAIL
NDV-Mut HN(F277L)/M(G165W)-IL-12
NDV-Mut HN(F277L)/M(G165W)-(ns)hIL-12
NDV-Mut HN(F277L)/M(G165W)/F(F117S)/F(F190L)/L(V757I)/L(F1551S)/L(R1700L)
NDV-Mut HN(F277L)/M(G165W)/F(F117S)/F(F190L)/L(V757I)/L(F1551S)/L(R1700L)-Atezolizumab
NDV-Mut HN(F277L)/M(G165W)/F(F117S)/F(F190L)/L(V757I)/L(F1551S)/L(R1700L)-Bevacizumab
NDV-Mut HN(F277L)/M(G165W)/F(F117S)/F(F190L)/L(V757I)/L(F1551S)/L(R1700L)-Lirilumab
NDV-Mut HN(F277L)/M(G165W)/F(F117S)/F(F190L)/L(V757I)/L(F1551S)/L(R1700L)-Relatlimab
NDV-Mut HN(F277L)/M(G165W)/F(F117S)/F(F190L)/L(V757I)/L(F1551S)/L(R1700L)-Monalizumab
NDV-Mut HN(F277L)/M(G165W)/F(F117S)/F(F190L)/L(V757I)/L(F1551S)/L(R1700L)-TRX518
NDV-Mut HN(F277L)/M(G165W)/F(F117S)/F(F190L)/L(V757I)/L(F1551S)/L(R1700L)-BMS 986178
NDV-Mut HN(F277L)/M(G165W)/F(F117S)/F(F190L)/L(V757I)/L(F1551S)/L(R1700L)-CD40
NDV-Mut HN(F277L)/M(G165W)/F(F117S)/F(F190L)/L(V757I)/L(F1551S)/L(R1700L)-CD80
NDV-Mut HN(F277L)/M(G165W)/F(F117S)/F(F190L)/L(V757I)/L(F1551S)/L(R1700L)-(ns)hIL-12
NDV-Mut HN(F277L)/M(G165W)/F(F117S)/F(F190L)/L(V757I)/L(F1551S)/L(R1700L)-eGFP
NDV-Mut HN(F277L)/M(G165W)/F(F117S)/F(F190L)/L(V757I)/L(F1551S)/L(R1700L)-Nivolumab
NDV-Mut HN(F277L)/M(G165W)/F(F117S)/F(F190L)/L(V757I)/L(F1551S)/L(R1700L)-Ipilimumab
NDV-Mut HN(F277L)/M(G165W)/F(F117S)/F(F190L)/L(V757I)/L(F1551S)/L(R1700L)-NS1
NDV-Mut HN(F277L)/M(G165W)/F(F117S)/F(F190L)/L(V757I)/L(F1551S)/L(R1700L)-Apoptin
NDV-Mut HN(F277L)/M(G165W)/F(F117S)/F(F190L)/L(V757I)/L(F1551S)/L(R1700L)-B18R
NDV-Mut HN(F277L)/M(G165W)/F(F117S)/F(F190L)/L(V757I)/L(F1551S)/L(R1700L)-Theralizumab
NDV-Mut HN(F277L)/M(G165W)/F(F117S)/F(F190L)/L(V757I)/L(F1551S)/L(R1700L)-Gemtuzumab
NDV-Mut HN(F277L)/M(G165W)/F(F117S)/F(F190L)/L(V757I)/L(F1551S)/L(R1700L)-anti-CD39
NDV-Mut HN(F277L)/M(G165W)/F(F117S)/F(F190L)/L(V757I)/L(F1551S)/L(R1700L)-anti-CD40
NDV-Mut HN(F277L)/M(G165W)/F(F117S)/F(F190L)/L(V757I)/L(F1551S)/L(R1700L)-anti-CD80
NDV-Mut HN(F277L)/M(G165W)/F(F117S)/F(F190L)/L(V757I)/L(F1551S)/L(R1700L)-anti-CA 15-3
NDV-Mut HN(F277L)/M(G165W)/F(F117S)/F(F190L)/L(V757I)/L(F1551S)/L(R1700L)-anti-CA 19-9
NDV-Mut HN(F277L)/M(G165W)/F(F117S)/F(F190L)/L(V757I)/L(F1551S)/L(R1700L)-Sofituzumab
NDV-Mut HN(F277L)/M(G165W)/F(F117S)/F(F190L)/L(V757I)/L(F1551S)/L(R1700L)-Cetuximab
NDV-Mut HN(F277L)/M(G165W)/F(F117S)/F(F190L)/L(V757I)/L(F1551S)/L(R1700L)-Trastuzumab
NDV-Mut HN(F277L)/M(G165W)/F(F117S)/F(F190L)/L(V757I)/L(F1551S)/L(R1700L)-BIL=3s
NDV-Mut HN(F277L)/M(G165W)/F(F117S)/F(F190L)/L(V757I)/L(F1551S)/L(R1700L)-J591
NDV-Mut HN(F277L)/M(G165W)/F(F117S)/F(F190L)/L(V757I)/L(F1551S)/L(R1700L)-Ramucircumab
NDV-Mut HN(F277L)/M(G165W)/F(F117S)/F(F190L)/L(V757I)/L(F1551S)/L(R1700L)-TRAIL
NDV-Mut HN(F277L)/M(G165W)/F(F117S)/F(F190L)/L(V757I)/L(F1551S)/L(R1700L)-IL-12
NDV-Mut HN(F277L)/M(G165W)/F(F117S)/F(F190L)/L(V757I)/L(F1551S)/L(R1700L)-(ns)hIL-12
NDV-Mut HN(F277L)/M(G165W)/F(F117S)/F(F190L)/F(L289A)/L(V757I)/L(F1551S)/L(R1700L)
NDV-Mut HN(F277L)/M(G165W)/F(F117S)/F(F190L)/F(L289A)/L(V757I)/L(F1551S)/L(R1700L)-Atezolizumab
NDV-Mut HN(F277L)/M(G165W)/F(F117S)/F(F190L)/F(L289A)/L(V757I)/L(F1551S)/L(R1700L)-Bevacizumab
NDV-Mut HN(F277L)/M(G165W)/F(F117S)/F(F190L)/F(L289A)/L(V757I)/L(F1551S)/L(R1700L)-Lirilumab
NDV-Mut HN(F277L)/M(G165W)/F(F117S)/F(F190L)/F(L289A)/L(V757I)/L(F1551S)/L(R1700L)-Relatlimab
NDV-Mut HN(F277L)/M(G165W)/F(F117S)/F(F190L)/F(L289A)/L(V757I)/L(F1551S)/L(R1700L)-Monalizumab
NDV-Mut HN(F277L)/M(G165W)/F(F117S)/F(F190L)/F(L289A)/L(V757I)/L(F1551S)/L(R1700L)-TRX518
NDV-Mut HN(F277L)/M(G165W)/F(F117S)/F(F190L)/F(L289A)/L(V757I)/L(F1551S)/L(R1700L)-BMS 986178
NDV-Mut HN(F277L)/M(G165W)/F(F117S)/F(F190L)/F(L289A)/L(V757I)/L(F1551S)/L(R1700L)-CD40
NDV-Mut HN(F277L)/M(G165W)/F(F117S)/F(F190L)/F(L289A)/L(V757I)/L(F1551S)/L(R1700L)-CD80
NDV-Mut HN(F277L)/M(G165W)/F(F117S)/F(F190L)/F(L289A)/L(V757I)/L(F1551S)/L(R1700L)-eGFP
NDV-Mut HN(F277L)/M(G165W)/F(F117S)/F(F190L)/F(L289A)/L(V757I)/L(F1551S)/L(R1700L)-Nivolumab
NDV-Mut HN(F277L)/M(G165W)/F(F117S)/F(F190L)/F(L289A)/L(V757I)/L(F1551S)/L(R1700L)-Ipilimumab
NDV-Mut HN(F277L)/M(G165W)/F(F117S)/F(F190L)/F(L289A)/L(V757I)/L(F1551S)/L(R1700L)-NS1
NDV-Mut HN(F277L)/M(G165W)/F(F117S)/F(F190L)/F(L289A)/L(V757I)/L(F1551S)/L(R1700L)-Apoptin
NDV-Mut HN(F277L)/M(G165W)/F(F117S)/F(F190L)/F(L289A)/L(V757I)/L(F1551S)/L(R1700L)-B18R
NDV-Mut HN(F277L)/M(G165W)/F(F117S)/F(F190L)/F(L289A)/L(V757I)/L(F1551S)/L(R1700L)-Theralizumab
NDV-Mut HN(F277L)/M(G165W)/F(F117S)/F(F190L)/F(L289A)/L(V757I)/L(F1551S)/L(R1700L)-Gemtuzumab
NDV-Mut HN(F277L)/M(G165W)/F(F117S)/F(F190L)/F(L289A)/L(V757I)/L(F1551S)/L(R1700L)-anti-CD39
NDV-Mut HN(F277L)/M(G165W)/F(F117S)/F(F190L)/F(L289A)/L(V757I)/L(F1551S)/L(R1700L)-anti-CD40
NDV-Mut HN(F277L)/M(G165W)/F(F117S)/F(F190L)/F(L289A)/L(V757I)/L(F1551S)/L(R1700L)-anti-CD80
NDV-Mut HN(F277L)/M(G165W)/F(F117S)/F(F190L)/F(L289A)/L(V757I)/L(F1551S)/L(R1700L)-anti-CA 15-3
NDV-Mut HN(F277L)/M(G165W)/F(F117S)/F(F190L)/F(L289A)/L(V757I)/L(F1551S)/L(R1700L)-anti-CA 19-9
NDV-Mut HN(F277L)/M(G165W)/F(F117S)/F(F190L)/F(L289A)/L(V757I)/L(F1551S)/L(R1700L)-Sofituzumab
NDV-Mut HN(F277L)/M(G165W)/F(F117S)/F(F190L)/F(L289A)/L(V757I)/L(F1551S)/L(R1700L)-Cetuximab
NDV-Mut HN(F277L)/M(G165W)/F(F117S)/F(F190L)/F(L289A)/L(V757I)/L(F1551S)/L(R1700L)-Trastuzumab
NDV-Mut HN(F277L)/M(G165W)/F(F117S)/F(F190L)/F(L289A)/L(V757I)/L(F1551S)/L(R1700L)-BIL=3s
NDV-Mut HN(F277L)/M(G165W)/F(F117S)/F(F190L)/F(L289A)/L(V757I)/L(F1551S)/L(R1700L)-J591
NDV-Mut HN(F277L)/M(G165W)/F(F117S)/F(F190L)/F(L289A)/L(V757I)/L(F1551S)/L(R1700L)-Ramucircumab
NDV-Mut HN(F277L)/M(G165W)/F(F117S)/F(F190L)/F(L289A)/L(V757I)/L(F1551S)/L(R1700L)-TRAIL
NDV-Mut HN(F277L)/M(G165W)/F(F117S)/F(F190L)/F(L289A)/L(V757I)/L(F1551S)/L(R1700L)-IL-12
NDV-Mut HN(F277L)/M(G165W)/F(F117S)/F(F190L)/F(L289A)/L(V757I)/L(F1551S)/L(R1700L)-(ns)hIL-12.

According to another embodiment of the present invention a recombinant NDV may also comprise at least one further mutation in the L gene and the encoded L protein having an amino acid substitution at position 1717 to an amino acid with aromatic side chain other than tyrosine (Y), preferably where tyrosine (Y) is substituted to histidine (H) at position 1717 of the L gene product, and/or at position 1910 to an amino acid with a basic side chain, preferably where glutamic acid (E) is substituted to lysine (K) at position 1910 of the L gene product. Alternatively or in addition a recombinant NDV according to the present invention may comprise a mutation in the RNA-editing sequence of the P gene. More preferably, the mutation abolishes and/or decreases the expression of the V protein. For further details reference to the above disclosure, for example tables 1a to 1d, concerning the novel NDV strain per se is made.

In a preferred embodiment of the present invention the viral HN gene product can have or can comprise an amino acid sequence as identified in SEQ ID No. 36, and/or the viral M gene product can have or comprise an amino acid sequence as identified in SEQ ID No. 37, and/or the viral F gene product can have or comprise an amino acid sequence as identified in SEQ ID No. 38 and/or the viral L gene product can have or comprise an amino acid sequence as identified in SEQ ID No. 39. Also encompassed by the present invention are formulations comprising NDV strains comprising variants of the gene products according to SEQ ID No. 36 to 39.

In the pharmaceutical formulation each of the recombinant NDV strains can be comprised in such an amount that per dose, preferably per 20 ml dose, an amount of 10⁷ to 10⁹, preferably of 5×10⁷ to 5×10⁸, virus particles of each of the recombinant NDV strains can be administered. Within the present invention virus quantification is done by the endpoint dilution assay and the values given are the fifty-percent tissue culture infective dose (TCID₅₀).

As already mentioned before, the pharmaceutical formulation according to the present invention further comprises at least one NDV strain not encoding a foreign gene. The viral genome of the at least one NDV strain comprises a nucleic acid comprising a nucleic acid sequence encoding a hemagglutinin-neuramidase protein (HN protein) with an amino acid substitution at position 277 to an amino acid with a hydrophobic side chain other than phenylalanine, preferably where phenylalanine (F) at position 277 is substituted to leucine (L). In addition the viral genome of the at least one NDV strain may comprise a nucleic acid comprising a nucleic acid sequence encoding a M protein and/or F protein and/or L protein as specified before.

The at least one non-recombinant NDV strain comprised in the pharmaceutical formulation according to the present invention can be derived from NDV strain MTH-68/H. Preferred non-recombinant NDV strains comprised in the pharmaceutical formulation according to the present invention can be selected from the group consisting of NDV-Mut HN(F277L), NDV-Mut HN(F277L)/M(G165W) (Nothabene-1), NDV-Mut HN(F277L)/F(F117S)/F(F190L), NDV-Mut HN(F277L)/M(G165W)/F(F117S)/F(F190L)/L(V757I)/L(F1551S)/L(R1700L) (Nothabene-2a), NDV-Mut HN(F277L)/F(F117S)/F(F190L)/F(L289A), NDV-Mut HN(F277L)/M(G165W)/F(F117S)/F(F190L)/F(L289A)/L(V757I)/L(F1551S)/L(R1700L) (Nothabene-2b) and any mixture of these strains. More preferably the non-recombinant NDV strain can have a nucleic acid sequence as set forth in SEQ ID No. 2 to 7 of the sequence listing or parts thereof. Still more preferably the non-recombinant NDV strain comprised in the claimed pharmaceutical formulation can be NDV-Mut HN(F277L)/M(G165W), i.e. Nothabene-1, or have or comprise a nucleic acid sequence according to SEQ ID No. 2 or SEQ ID No. 3 of the sequence listing or parts thereof. Alternatively the non-recombinant NDV strain comprised in the claimed pharmaceutical formulation can be NDV-Mut HN(F277L)/M(G165W)/F(F117S)/F(F190L)/L(V757I)/L(F1551S)/L(R1700L), i.e. Nothabene-2a, or have or comprise a nucleic acid sequence according to SEQ ID No. 4 or SEQ ID No. 5 of the sequence listing or parts thereof, or alternatively be NDV-Mut HN(F277L)/M(G165W)/F(F117S)/F(F190L)/F(L289A)/L(V757I)/L(F1551S)/L(R1700L), i.e. Nothabene-2b, or have or comprise a nucleic acid sequence according to SEQ ID No. 6 or SEQ ID No. 7 of the sequence listing or parts or variants thereof. As described above, these NDV strains provide different advantages and may be selected according to the particular need.

Also preferably the pharmaceutical formulation can comprise as non-recombinant NDV strains a combination of NDV-Mut HN(F277L)/M(G165W) (Nothabene-1), and/or NDV-Mut HN(F277L)/M(G165W)/F(F117S)/F(F190L)/L(V757I)/L(F1551S)/L(R1700L) (Nothabene-2a) and/or NDV-Mut HN(F277L)/M(G165W)/F(F117S)/F(F190L)/F(L289A)/L(V757I)/L(F1551S)/L(R1700L) (Nothabene-2b), for example a combination of Nothabene-1 and Nothabene-2a and Nothabene-2b. Also, the pharmaceutical formulation can comprise as non-recombinant NDV strains a combination of a NDV strains having or comprising a nucleic acid sequence according to SEQ ID No. 2 or SEQ ID No. 3 and/or a NDV strain having or comprising a nucleic acid sequence according to SEQ ID No. 4 or SEQ ID No. 5 and/or a NDV strain having or comprising a nucleic acid sequence according to SEQ ID No. 6 or SEQ ID No. 7 of the sequence listing, or respective parts or variants thereof. The three NDV-strains are preferably comprised in the same amounts in the pharmaceutical formulation.

The addition of the at least one non-recombinant NDV strain to the pharmaceutical formulation provides an additional oncolytic agent, thereby improving the efficiency of the therapeutic agent in the treatment of cancer. Without wishing to be bound to that theory, a combination of recombinant and non-recombinant NDV strains in the pharmaceutical formulation can be advantageous because, the recombinant strains can be somewhat slower in their oncolytic potential due to the “additional load” of the foreign gene compared to the respective non-recombinant strains. The non-recombinant strains may ensure that the oncolytic potential is available immediately and also over a longer period of time.

The pharmaceutical formulation according to the present invention further comprises particles of a reovirus and/or of a vaccinia virus.

Reoviruses are another type of oncolytic viruses having the capacity to directly kill cancer cells, which makes the reoviruses useful in cancer virotherapy. Three reovirus serotypes circulate in humans, serotype 1, serotype 2, and serotype 3. Provided its clinical safety, the at least one reovirus comprised in the pharmaceutical formulation of the present invention can be a reovirus of any of these serotypes according to SEQ ID Nos. 58-67 of the sequence listing, wherein the sequence consists of a total of 10 gene-segments (L1-3, M1-3 and S1-4). Preferably the one or more reovirus strains comprised in the pharmaceutical formulation is of serotype 3. This is because reoviruses of serotype 3 induce more apoptosis.

The sequence given by these SEQ ID numbers is as follows:
SEQ ID No. 58: Nucleic acid sequence of Reovirus type 3 comprising gene segment L1;
SEQ ID No. 59: Nucleic acid sequence of Reovirus type 3 comprising gene segment L2;
SEQ ID No. 60: Nucleic acid sequence of Reovirus type 3 comprising gene segment L3;
SEQ ID No. 61: Nucleic acid sequence of Reovirus type 3 comprising gene segment M1;
SEQ ID No. 62: Nucleic acid sequence of Reovirus type 3 comprising gene segment M2;
SEQ ID No. 63: Nucleic acid sequence of Reovirus type 3 comprising gene segment M3;
SEQ ID No. 64: Nucleic acid sequence of Reovirus type 3 comprising gene segment S1;
SEQ ID No. 65: Nucleic acid sequence of Reovirus type 3 comprising gene segment S2;
SEQ ID No. 66: Nucleic acid sequence of Reovirus type 3 comprising gene segment S3;
SEQ ID No. 67: Nucleic acid sequence of Reovirus type 3 comprising gene segment S4;

Even though the therapeutic potency of reovirus monotherapy can be limited, it has been surprisingly shown that in combination therapy with the NDV strains as specified supra the therapeutic potency is markedly increased. This applies in particular in a combination therapy of reovirus and recombinant NDV strains encoding immunotherapeutic agents, preferably the one or more checkpoint modulators and angiogenesis inhibitors, and the virulence factor and/or the other foreign genes as specified supra. Thus, the reovirus can beneficially modulate the immunotherapeutic aspect of the cancer treatment provided by the present invention. Still further the reovirus may sensitize cancer cells to chemotherapeutic drugs and radiation treatment.

Vaccinia viruses are another oncolytic agent, which can be used in oncolytic therapies for cancer. Safe and efficacious tumor-targeted strains are in general available to the skilled person. These strains may be genetically modified recombinant to increase their safety due to their tumor selectivity. An inherent characteristic of vaccinia virus is its short life cycle that takes place in its entirety in the cytoplasm eliminating the risk of genome integration.

Preferably the at least one vaccinia virus strain which is comprised in the pharmaceutical formulation is based on the strain MVA, i.e. Modified Vaccinia Ankara, but does not comprise four deletions as present in MVA.

A person skilled in the art knows how to select suitable reovirus and vaccinia strains in order to ensure patient safety.

The pharmaceutical formulation according to the present invention comprises virus particles of the reovirus type 3 in such an amount that per dose, preferably per 20 ml dose, an amount of 6×10⁷ to 6×10⁹, preferably of 1×10⁸ to 1×10⁹, each given as TCID₅₀ values, can be administered. In addition the pharmaceutical formulation can comprise virus particles of the vaccinia virus, if present at all, in such an amount that per dose, preferably per 20 ml dose, an of amount 4×10⁵ to 4×10⁷, preferably of 1×10⁶ to 1×10⁷, each given as TCID₅₀ values, can be administered.

Preferably the pharmaceutical formulation according to the present invention comprises between 3 to 20, more preferably between 4 to 16, still more preferably between 5 and 12 different oncolytic virus strains, which not all must be recombinant. If recombinant, the foreign genes comprised in these different virus strains are preferably selected from the foreign genes as specified before, for example with reference to any one of SEQ ID No. 9 to SEQ ID No. 34. The NDV strains comprised in these mixtures are preferably derived from NDV strain MTH-68/H. More preferably the NDV strains are selected from the group consisting of NDV-Mut HN(F277L), Nothabene-1, NDV-Mut HN(F277L)/F(F117S)/F(F190L), Nothabene-2a, NDV-Mut HN(F277L)/F(F117S)/F(F190L)/F(L289A) and Nothabene-2b. For example the NDV strains are Nothabene-1, preferably having a nucleic acid sequence as shown in SEQ ID No. 2 or SEQ ID No. 3 of the sequence listing, Nothabene-2a, preferably having a nucleic acid sequence as shown in SEQ ID No. 4 or SEQ ID No. 5 of the sequence listing, and/or Nothabene-2b, preferably having a nucleic acid sequence as shown in SEQ ID No. 6 or SEQ ID No. 7 of the sequence listing. Any one of these viral backbones may be used to insert the foreign genes for obtaining the respective recombinant NDV strains.

A preferred pharmaceutical formulation according to the present invention comprises the following different virus strains:
A recombinant Newcastle Disease Virus (NDV) strain comprising in its viral genome a nucleic acid sequence encoding an antibody, preferably a monoclonal antibody, or an antigen-binding part thereof directed to protein PD-L1 (anti-PD-L1). In one embodiment the antibody is Atezolizumab, an antigen-binding part of Atezolizumab, a variant of Atezolizumab or a variant of an antigen-binding part of Atezolizumab.
The viral backbone of the recombinant NDV strain is preferably selected from the group consisting of NDV-Mut HN(F277L), Nothabene-1, NDV-Mut HN(F277L)/F(F117S)/F(F190L), Nothabene-2a, NDV-Mut HN(F277L)/F(F117S)/F(F190L)/F(L289A) and Nothabene-2b. More preferably it is or comprises a nucleic acid sequence as shown in any one of SEQ ID No. 2 to SEQ ID No. 7 of the sequence listing or parts thereof.
A recombinant Newcastle Disease Virus (NDV) strain comprising in its viral genome a nucleic acid sequence encoding an antibody, preferably a monoclonal antibody, or an antigen-binding part thereof directed to the surface protein CTLA-4 (anti-CTLA-4). In one embodiment the antibody is Ipilimumab, an antigen-binding part of Ipilimumab, a variant of Ipilimumab or a variant of an antigen-binding part of Ipilimumab.
The viral backbone of the recombinant NDV strain is preferably selected from the group consisting of NDV-Mut HN(F277L), Nothabene-1, NDV-Mut HN(F277L)/F(F117S)/F(F190L), Nothabene-2a, NDV-Mut HN(F277L)/F(F117S)/F(F190L)/F(L289A) and Nothabene-2b. More preferably it is or comprises a nucleic acid sequence as shown in any one of SEQ ID No. 2 to SEQ ID No. 7 of the sequence listing or parts thereof.
A recombinant Newcastle Disease Virus (NDV) strain comprising in its viral genome a nucleic acid sequence encoding an antibody, preferably a monoclonal antibody, or an antigen-binding part thereof directed to protein PD-1 (anti-PD-1). In one embodiment the antibody is Nivolumab, an antigen-binding part of Nivolumab, a variant of Nivolumab or a variant of an antigen-binding part of Nivolumab.
The viral backbone of the recombinant NDV strain is preferably selected from the group consisting of NDV-Mut HN(F277L), Nothabene-1, NDV-Mut HN(F277L)/F(F117S)/F(F190L), Nothabene-2a, NDV-Mut HN(F277L)/F(F117S)/F(F190L)/F(L289A) and Nothabene-2b. More preferably it is or comprises a nucleic acid sequence as shown in any one of SEQ ID No. 2 to SEQ ID No. 7 of the sequence listing or parts thereof.
A recombinant Newcastle Disease Virus (NDV) strain comprising in its viral genome a nucleic acid sequence encoding an antibody, preferably a monoclonal antibody, or an antigen-binding part thereof directed to the growth factor protein VEGF-A (anti-VEGF-A). In one embodiment the antibody is Bevacizumab, an antigen-binding part of Bevacizumab, a variant of Bevacizumab or a variant of an antigen-binding part of Bevacizumab.
The viral backbone of the recombinant NDV strain is preferably selected from the group consisting of NDV-Mut HN(F277L), Nothabene-1, NDV-Mut HN(F277L)/F(F117S)/F(F190L), Nothabene-2a, NDV-Mut HN(F277L)/F(F117S)/F(F190L)/F(L289A) and Nothabene-2b. More preferably it is or comprises a nucleic acid sequence as shown in any one of SEQ ID No. 2 to SEQ ID No. 7 of the sequence listing or parts thereof.
A recombinant Newcastle Disease Virus (NDV) strain comprising in its viral genome a nucleic acid sequence encoding the non-structural protein NS1 of influenza A virus, a part of the non-structural protein NS1 of influenza A virus, a variant of the non-structural protein NS1 of influenza A virus or a variant of a part of the non-structural protein NS1 of influenza A virus.

The viral backbone of the recombinant NDV strain is preferably selected from the group consisting of NDV-Mut HN(F277L), Nothabene-1, NDV-Mut HN(F277L)/F(F117S)/F(F190L), Nothabene-2a, NDV-Mut HN(F277L)/F(F117S)/F(F190L)/F(L289A) and Nothabene-2b. More preferably it is or comprises a nucleic acid sequence as shown in any one of SEQ ID No. 2 to SEQ ID No. 7 of the sequence listing or parts thereof.
At least one non-recombinant Newcastle Disease Virus (NDV). The viral genome of the at least one NDV strain comprises a nucleic acid comprising a nucleic acid sequence encoding a hemagglutinin-neuramidase protein (HN protein) with an amino acid substitution at position 277 to an amino acid with a hydrophobic side chain other than phenylalanine, preferably where phenylalanine (F) at position 277 is substituted to leucine (L). In addition the viral genome of the at least one NDV strain may comprise a nucleic acid comprising a nucleic acid sequence encoding a M protein and/or F protein and/or L protein as specified before. The at least one NDV strain is preferably selected from the group consisting of Nothabene-1, preferably having a nucleic acid sequence as shown in SEQ ID No. 2 or SEQ ID No. 3 of the sequence listing, Nothabene-2a, preferably having a nucleic acid sequence as shown in SEQ ID No. 4 or SEQ ID No. 5 of the sequence listing, and Nothabene-2b, preferably having a nucleic acid sequence as shown in SEQ ID No. 6 or SEQ ID No. 7 of the sequence listing. Still more preferably the NDV strain is Nothabene-1, preferably having a nucleic acid sequence as shown in SEQ ID No. 2 or SEQ ID No. 3 of the sequence listing. Also preferred is a combination of Nothabene-1 and/or Nothabene-2a and/or Nothabene-2b or a combination of NDV strains having or comprising a nucleic acid sequence according to SEQ ID No. 2 or SEQ ID No. 3 and SEQ ID No. 4 or SEQ ID No. 5 and SEQ ID No. 6 or SEQ ID No. 7 of the sequence listing or parts thereof.
A reovirus serotype 3.
Optionally a vaccinia virus.

This combination of oncolytic viruses has been surprisingly shown to provide an improved oncolytic and immunologic activity in the treatment of cancer. In particular the NDV strains provide on the one hand an improved replication capacity of the NDV particles in cancer cells and a good or even an improved safety profile, preferably in humans, which is associated with increased cancer cell lysis and increased anti-cancer activity, because the mutated and optionally recombinant NDV strains are still selective for cancer cells. The low to zero activity towards normal cells is associated with an increased therapeutic window or safety margin, as is commonly determined for cancer therapeutics. Beside the virological activity of the oncolytic viruses, the recombinant mutated NDV strains are in addition beneficial for the treatment of cancer from an immunological point of view. This is because the foreign gene carried by NDV strains of the present invention beneficially influences the ability of the immune system of the subject to be treated to attack and destroy cancer cells. Advantageously the foreign gene is expressed inside the tumor to be treated, that is directly in the side of need.

Depending on the actual need or the particular cancer to be treated, preferred combinations may be supplemented with one or more additional recombinant NDV strains, wherein each of these strains can encode any one of the above specified additional foreign genes, for example as specified with reference to SEQ ID No. 23 to SEQ ID No. 34 of the sequence listing or variants thereof. The combinations are preferably such that the encoded gene products support each other in their mode of action.

The invention also relates to a method of treating a subject suffering from cancer by administering a pharmaceutical composition and/or the use of the pharmaceutical composition according to the present invention in the field of medicine.

Thus, in a further aspect the present invention is concerned with the medical use of the pharmaceutical formulation of the present invention. In this regard the invention provides the above described pharmaceutical formulation for use in oncological treatment (this term is used herein also replaceable with “for use as a medicament, especially in a method of treatment of cancer”; or the use of said oncolytic NDV in the preparation of a pharmaceutical formulation for use in said method of treatment) in humans (or more generically animals, such as mammalian animals).

In this regard the present invention is first concerned with a pharmaceutical formulation according to the present invention for use in medicine. The pharmaceutical formulation is formulated as specified above. For all embodiments, be it preferred or not, reference to the above disclosure is made. All these pharmaceutical formulation can be used in the field of medicine.

Secondly, the present invention is more particularly concerned with a pharmaceutical formulation according to the present invention for use in a method of treating cancer in a subject considered in need thereof. In a preferred embodiment of the present invention the subject to be treated is a mammal, more preferably a mammalian animal or a human subject. Still more preferred the pharmaceutical formulation according to the present invention may be used for the treatment of adults and/or children, preferably human adults and/or human children.

The health condition which may be treated is preferably an indication selected from the group consisting of brain tumors, bone tumors, soft tissue tumors, gynecological tumors, gastrointestinal tumors, prostate tumors, lung tumors, ear, nose, throat tumors, tongue tumors, and skin tumors.

Accordingly the pharmaceutical formulation of the present invention is preferably used in a method for treating one or more indications selected from the group consisting of brain tumors, like glioblastoma, bone tumors, like osteosarcoma and/or Ewing's sarcoma, soft tissue tumors, like rhabdomyosarcoma, gynecological tumors, like breast cancer, ovary cancer and/or cervix cancer, gastrointestinal tumors, like esophageal tumors, stomach tumors, colon tumors, pancreas tumors, prostate tumors, lung tumors, ear, nose, throat tumors, tongue tumors, and skin tumors, like melanoma.

The pharmaceutical formulation can in general be administered in any mode suitable for the treatment of the respective cancer. The pharmaceutical formulation is preferably administered intratumoral or intraarterial.

For the indications mentioned herein, the appropriate dosage will, of course, vary depending upon, for example, the particular NDV strains to be employed, the mode of administration and the nature and severity of the condition being treated. In general, the pharmaceutical formulation can be administered preferably 1 to 4 times per month.

The pharmaceutical formulation used in the method for treating cancer may in one embodiment not comprise a recombinant NDV strain comprising in its viral genome a nucleic acid comprising a nucleic acid sequence encoding interleukin-12 (IL-12), a part of interleukin-12, a variant of interleukin-12 or a variant of a part of interleukin-12. This does however not exclude that pharmaceutical formulation according to this preferred embodiment is used in a combinatorial therapy with another pharmaceutical formulation comprising a recombinant NDV strain having a viral genome comprising a nucleic acid sequence encoding interleukin-12 (IL-12) or a part of interleukin-12 or a variant of interleukin-12 or a variant of a part of interleukin-12. Preferably, the foreign gene encodes a human interleukin 12 (hIL-12) protein, or a part thereof. The encoded protein can also be (ns)hIL-12, a part of (ns)hIL-12, a variant of (ns)hIL-12 or a variant of a part of (ns)hIL-12.

Preferred NDV strains which can be comprised in the pharmaceutical formulation according to the present invention or in another pharmaceutical formulation are preferably selected from the group consisting of NDV-Mut HN(F277L)-IL-12, Nothabene-1-IL-12, NDV-Mut HN(F277L)/F(F117S)/F(F190L)-IL-12, Nothabene-2a-IL-12, NDV-Mut HN(F277L)/F(F117S)/F(F190L)/F(L289A)-IL-12, Nothabene-2b-IL-12, NDV-Mut HN(F277L)-hIL-12, Nothabene-1-hIL-12, NDV-Mut HN(F277L)/F(F117S)/F(F190L)-hIL-12, Nothabene-2a-hIL-12, NDV-Mut HN(F277L)/F(F117S)/F(F190L)/F(L289A)-hIL-12, Nothabene-2b-hIL-12, NDV-Mut HN(F277L)-(ns)hIL-12, Nothabene-1-(ns)hIL-12, NDV-Mut HN(F277L)/F(F117S)/F(F190L)-(ns)hIL-12, Nothabene-2a-(ns)hIL-12, NDV-Mut HN(F277L)/F(F117S)/F(F190L)/F(L289A)-(ns)hIL-12, Nothabene-2b-(ns)hIL-12, and any mixtures of these strains. More preferably the viral backbone of that recombinant NDV strain is or comprises a nucleic acid sequence as shown in any one of SEQ ID No. 2 to 7 of the sequence listing or parts thereof.

Administration of interleukin-12 offers a further tool in the treatment of cancer. If the interleukin-12 is not provided by the pharmaceutical formulation according to the present invention, the pharmaceutical formulation according to the present invention and the interleukin-12, or a pharmaceutical formulation comprising interleukin-12, are preferably used sequentially, i.e. at different time points. This helps to avoid undesired side effects, which would be obtained when including IL-12 into the pharmaceutical formulation of the present invention.

Also within the scope of the present invention is the use of the pharmaceutical composition according to the present invention in combination with one or more other therapies suitable for the treatment of cancer. In a preferred embodiment the pharmaceutical formulation is used in a method for treating cancer, especially a cancer selected from the specific cancers as described and/or mentioned in the claims herein.

The pharmaceutical formulation and one or more other therapies can be used concurrently or sequentially. In certain embodiments, the pharmaceutical formulation and the one or more other therapies are administered in the same (“fixed”) pharmaceutical formulation. In other embodiments, the pharmaceutical formulation and the one or more other therapies are administered in different formulations. The pharmaceutical formulation of the present invention and the one or more other therapies can be administered by the same or different routes of administration to the subject considered in need thereof. Another therapy within the meaning of the present invention also comprises an administration of other oncolytical virus strains, for example recombinant NDV strains encoding and expressing IL-12 or other foreign genes than those as disclosed within the present invention or having other mutations in the NDV genome.

In still a further aspect the present invention is also concerned with a method for treating cancer, especially a cancer selected from the specific cancers as described and/or mentioned in the claims herein, in a subject considered in need thereof. The method for treating cancer utilizes a pharmaceutical formulation as described herein, especially in a therapeutically effective amount. Within the meaning of the present patent application a therapeutically effective amount also includes an effective amount for preventing a cancer, in particular a cancer selected from the specific cancers as described and/or mentioned in the claims herein.

The method of treatment comprises or consists of administering to a subject in need thereof a pharmaceutical formulation according to the present invention in a sufficient amount for infecting and destroying some or all of the cancer cells. In a specific embodiment the method for treating cancer comprises or consists of infecting a cancer cell in a subject with a pharmaceutical formulation of the present invention. In another embodiment the pharmaceutical composition of the present invention is administered to a subject in need thereof by intravenous, intraarterial, intratumoral, intramuscular, intradermal, subcutaneous, or any other medically relevant route of administration. The pharmaceutical formulation is preferably administered intratumoral or intraarterial.

Also within the scope of the present invention is a method of treating cancer, especially a cancer selected from the specific cancers as described and/or mentioned in the claims herein, which utilizes a pharmaceutical formulation according to the present invention in combination with one or more other therapies. In a preferred embodiment the pharmaceutical formulation is administered to a subject in need thereof in a therapeutically effective amount. The pharmaceutical formulation and one or more other therapies can be administered concurrently or sequentially to the subject (meaning they are jointly therapeutically active). The pharmaceutical formulation of the present invention and one or more other therapies can be administered by the same or different routes of administration to the subject considered in need thereof. Another therapy within the meaning of the present invention also comprises an administration of other oncolytical virus strains, for example recombinant NDV strains encoding and expressing IL-12 or other foreign genes than those as disclosed within the present invention or having other mutations in the NDV genome.

Another therapy may also comprise or may be radiotherapy for cancer.

In the following a method for obtaining recombinant viral genomes will be described in detail. A person skilled in the art knows how to transfer the disclosed method for obtaining mutated viral genomes not comprising a transgenic construct.

Recombinant viral genomes, which can be used to rescue virus particles, can for example be obtained by a method called ‘reverse genetics’. The infectious virus particles obtained from said full-length cDNA of the NDV strains or recombinant NDV strains have an unexpected beneficial replication capability in cell cultures, eggs or animals that are used to propagate the virus for pharmaceutical purposes, and have good or even an improved viral safety profile, giving production advantages to such improved rgNDV strains. Furthermore, the higher replication rate is also particularly useful to obtain the desired oncolytic effects in vivo in that more virus particles will be produced for subsequent rounds of infection of cancer cells that were missed in the first round of infection. What is more, if the NDV strain is recombinant, higher levels (yields) of the foreign gene or the foreign genes encoded by the transgenic construct will be achieved for providing the desired enhanced oncolytic effects in vivo. Thus, by modifying the HN protein within a transgene-expressing NDV higher yields of the transgenic construct protein can be obtained, which is very useful in anti-tumor immunotherapy. By modifying the F protein, and preferably also the L protein, as specified supra, beside the oncolytic potential also the viral safety can be improved.

The NDV strains according to the present invention can be obtained by a reverse genetic method for preparing an rgNDV, optionally encoding at least one foreign gene, preferably at least one foreign gene comprising or consisting of a nucleic acid sequence according to any of SEQ ID No. 9 to 34 of the sequence listing or parts thereof, and having improved replication in a cancer cell, and good or even improved viral safety, over a parent NDV.

The method comprises providing a nucleic acid construct comprising a HN gene with a mutation, particularly wherein the mutation in the HN gene leads to a change in the expression of the hemagglutinin-neuraminidase, wherein the amino acid, particularly phenylalanine (F), in position 277 is substituted, particularly wherein the amino acid in position 277 is substituted to an amino acid with a hydrophobic side chain, more particularly wherein the amino acid in position 277 is substituted to leucine (L) at position 277 of the HN gene product/HN protein, and comprising a F gene encoding a fusion protein/F protein with an amino acid substitution at position 117 to an amino acid with a hydroxylated side chain and/or with an amino acid substitution at position 190 to an amino acid with an aliphatic side chain and an amino acid substitution at position 289 to an amino acid with an aliphatic side chain other than leucine. Still more preferred is a substitution of the amino acid phenylalanine (F) at position 117 of the F gene product to the amino acid serine (S) and/or a substitution of the amino acid phenylalanine (F) at position 190 of the F gene product to the amino acid leucine (L) and a substitution of the amino acid leucine (L) at position 289 of the F gene product to the amino acid alanine (A).

The method further comprises a step of providing a nucleic acid encoding a rgNDV further comprising a transgenic construct encoding at least one of the foreign gene products according to any of SEQ ID No. 9 to 34 of the sequence listing or parts thereof.

In the next step of the method the nucleic acid construct with said mutations in the HN gene and the at least two mutations in the F gene at amino acid position 117 and/or position 190 and position 289 (optionally with further mutations, in particular those as specified herein) is incorporated in the nucleic acid encoding a rgNDV comprising the transgenic construct in order to obtain a full length cDNA of the NDV, which additionally comprises a transgenic construct as specified supra. This method step can result in a nucleic acid as disclosed supra.

The nucleic acid encoding a rgNDV further comprising a transgenic construct as specified supra can be obtained by generating sub-genomic cDNA fragments and assembling the full length cDNA of the recombinant NDV from these fragments.

Following these method steps the thus obtained nucleic acid encoding a mutated and optionally recombinant rgNDV according to the present invention is used to produce infectious rgNDV particles, which replication characteristics and expression rates of the encoded foreign protein(s) in cancer cells are compared with the replication characteristics and expressions rates of the parent NDV used for designing the recombinant and mutated NDV strain. Also the ICPI values of the produced infectious rgNDV particles are compared with the parent NDV.

Finally, said rgNDV can be selected for further use, when it shows an improved replication characteristic over the parent NDV and good or even an improved viral safety, as well as a sufficient expression of the gene product or gene products of the at least one foreign genes.

If it is intended to introduce further to the mutation in the HN gene and the at least two mutations in the F gene at amino acid position 117 and/or position 190 and position 289, at least one further mutation in a viral gene, preferably the M gene, the L gene and/or the P gene, the selected recombinant and mutated rgNDV comprising a transgenic construct as specified supra and a mutated HN gene is used for incorporating a nucleic acid construct encoding a mutated M gene, L gene or P gene, and the method for introducing the mutations in the HN gene and F gene is repeated respectively. Alternatively there is already provided a nucleic acid encoding a rgNDV with the transgenic construct and carrying a mutation in the M gene, F gene, L gene or P gene, particularly wherein said mutation is capable of improving oncolytic potential of said rgNDV and/or viral safety.

A NDV or rgNDV can be provided in a suitable cell or cell line, for example a HeLa cell line or in cancer cells, or an embryonated egg that is susceptible to a NDV infection. Preferably HeLa cells can be used to produce the NDV strains and Vaccinia. The reovirus is preferably produced in LLC MK2 cells.

Suitable cell culture media are known to those skilled in the art. In addition to simple basal media, complex growth media as well as serum-free media formulations are available on the market and are provided by cell culture media manufacturers. A person skilled in the art is able to select a suitable medium for each cell line. For example, he can also access information from the cell banks (e.g. ATCC, ECACC, DSMZ). For example Medium 199 can be used preferably. Medium 199 is a highly complex medium, which was first described in 1950. Medium 199 can be used with Hanks salts (350 mg/1 NaHCO₃) or with Earles salts (2.2 g/1 NaHCO₃) and a CO₂ atmosphere.

In these infected cells the virus is grown to sufficient quantities, and preferably under sufficient conditions such that the virus is free from exogenous contamination, and the progeny virus particles are collected by suitable methods well known to those skilled in the art.

A further aspect of the invention relates to a method of preparing a pharmaceutical composition according to the present invention. The pharmaceutical formulations or pharmaceutical compositions of the present invention may be manufactured in any conventional manner, which are known to those skilled in the art. For example a pharmaceutical composition according to the present invention comprising a recombinant virus particle as described within this description can be provided in a dried, preferably in a lyophilized form and can be complemented with a suitable solvent, e.g. an aqueous carrier for injection at the time when used for administration. The dried form is very stable. Thus, in one embodiment the pharmaceutical composition is present as a dried form, still more preferred in a lyophilized form, and is reconstituted before administration by adding a suitable solvent. That is the present invention also relates to the above described dried form of the pharmaceutical composition for use as a medicament, preferably for use in a method of treating cancer. The process parameters for drying, preferably lyophilisation must be chosen such that the dried/lyophilized product contains live, replication competent viral particles, which can be grown to standardized titers, when needed. A person skilled in the art knows how to set these parameters.

For administration of a dried form, the dried form is directly dissolved in a suitable solvent or pharmaceutically acceptable carrier for injection and/or stabilization of the NDV or rg NDV and/or for improving its delivery to cancer cells. The solvent or carrier is preferably an aqueous carrier, for example sterile water for injection or sterile buffered physiological saline or another, preferably isotonic buffer system having a pH in the range of 7.2 to 7.6. The reconstitution can be performed ideally at or close to the intended time of administration in order to avoid any contaminations with microbes, etc. The skilled person is familiar with the handling of pharmaceutical compositions for reconstitution and reconstituted solutions.

The pharmaceutical formulation of the present invention can be prepared by a method comprising or consisting of the steps of:
propagating an NDV, be it recombinant or not, as described above in at least one cell or embryonated egg that is susceptible to a NDV infection;
collecting the progeny virus particles, wherein the virus is grown to sufficient quantities and under sufficient conditions that the virus is free from exogenous contamination, such that the progeny virus is suitable for formulation into a pharmaceutical formulation; and
preferably manufacturing a pharmaceutical formulation comprising said progeny virus particles in the absence of or after addition of at least one pharmaceutically acceptable carrier material.

The step of collecting progeny virus particles may in one embodiment also comprise a step of processing the collected virus-containing material to enrich virus particles and/or to eliminate host cell DNA.

The pharmaceutical formulation can be provided in a vial or a pre-filled syringe, which may be provided in a blister pack. Thus, also provided by the present invention are vials or pre-filled syringes comprising the pharmaceutical formulation of the present invention, or kits comprising the vials or pre-filled syringes. In one embodiment, such a kit comprises a pre-filled syringe of the invention in a blister pack. The blister pack may itself be sterile on the inside. Such a kit may further comprise a needle for administration, instructions for use, etc.

In one embodiment, the invention provides a carton containing a vial comprising the pharmaceutical formulation according to the present invention and optionally instructions for administration. In another embodiment the invention provides a pre-filled syringe comprising the pharmaceutical formulation according to the invention contained within a blister pack, a needle and optionally instructions for administration.

In order to ensure patient safety and medicament integrity the collected viruses and the pharmaceutical formulation should be sufficiently sterile to avoid infection or other risks for patients. Sterilization can be achieved by suitable methods well known to those skilled in the art.

General Within the present invention virus quantification is done by an endpoint dilution assay according to the Reed-Muench method (Reed, L. J.; Muench, H. (1938). “A simple method of estimating fifty percent endpoints”. The American Journal of Hygiene. 27: 493-497). Determined are the 50% endpoints, that is, the concentration of a test substance that produces an effect of interest in half of the test units. The virus titer is thus indicated as the TCID₅₀ value (50% tissue culture infectious dose of a virus).

The term “comprising” as used herein means “including” unless otherwise indicated by the specific context. For example, a pharmaceutical formulation “comprising” X may include something additional, e.g. X+Y.

### FIGURES

For a better understanding of the present invention, reference is made to the following description of an exemplary embodiment thereof, and the accompanying drawing, wherein:

FIG. 1 shows a schematic presentation of the NDV reverse genetics system. The upper part shows the composition of the full-length cDNA plasmid which contains the full-length NDV cDNA (encoding the nucleocapsid protein (NP), the phosphoprotein (P), the matrix protein (M), the fusion protein (F), the hemagglutinin-neuraminidase (HN), and the RNA-dependent RNA polymerase (L)) cloned behind the bacteriophage T7 RNA Polymerase promoter (T7P; yellow triangle) and followed by a ribozyme sequence (Rz) and T7 transcription termination signal (T7T).

A suitable host cell (shaded round-cornered box) is infected with a recombinant Fowlpox virus that expresses T7 DNA-dependent RNA polymerase (Fowlpox-T7) and subsequently co-transfected with the full-length cDNA plasmid and three helper plasmids containing the genes encoding the NDV NP, P and L proteins, respectively. Transcription of the full-length cDNA results in the generation of the NDV antigenome RNA which is encapsidated by NP protein then transcribed and replicated by the RNA-leading to the generation of infectious NDV.

FIG. 2 is a schematic presentation of the genome of exemplary rgNDV-mutants which can be comprised in a pharmaceutical formulation according to the present invention. The figure shows the position of the respective foreign gene which is inserted as extra transcription unit into the rgNDV-mutant genome between the P and M genes. Instead of the shown foreign genes any one of the other foreign genes mentioned in line with the disclosure of the present invention can be used, respectively be located at the respective position.

FIG. 3 shows growth kinetics of NDV in HeLa cells. HeLa cells were infected at a multiplicity of infection (MOI) of 0.01 with the indicated viruses. At 0 h, 8 h, 24 h and 48 h after infection, the amount of infectious virus in the supernatant was determined by end-point titration on QM5 cells. MTH68: NDV strain MTH-68/H; MutHu: NDV-Mut HN(F277L)/M(G165W); rgMTH68: NDV strain MTH-68/H derived by reverse genetics from cloned full-length cDNA; rgMutHu: NDV strain NDV-Mut HN(F277L)/M(G165W) derived by reverse genetics from cloned full-length cDNA; rgMutHu(HNL277F): rgMutHu in which the amino acid mutation at position 277 in the HN gene was converted from L back to F; rgMutHu(MW165G): rgMutHu in which the amino acid mutation at position 165 in the M gene was converted from W back to G.

FIG. 4 shows the expression of CD80 in a recombinant Nothabene-2a-strain. FIG. 4a shows the recombinant strain, FIG. 4b is the negative control. For detection an anti-CD80 antibody was used.

FIG. 5 shows syncytium formation due to the F(L289A) mutation. FIG. 5a is rgNothabene-2-F2L3+F(L289A) (i.e. F3L3) according to the present invention, FIG. 5b is rgNothabene-2-F2L3.

### EXAMPLES

### Example 1. Nucleotide Sequence Analysis of Mutant NDV-Mut HN(F277L)/M(G165W)

We identified a spontaneous mutant of an oncolytic NDV strain MTH-68/H (Csatary et al., 1999, Anticancer Res. 19:635-638.; further called MTH68). The replication capacity of the mutant strain (designated NDV-Mut HN(F277L)/M(G165W) in a variety of human neoplastic cell lines, as well as autologous primary tumors, is greatly enhanced as compared to the original MTH-68/H strain (also referred to as MTH68 strain). We analyzed its nucleotide sequence and found that, compared to MTH68, NDV-HN(F277L)/M(G165W) has two nucleotide mutations, one leading to an amino acid substitution in the M protein (G165W) and the other in the HN protein (F277L).

### Example 2. A Reverse Genetics System that Allows Genetic Modification of NDV-Strains

2.1 Reverse Genetics

In order to be able to genetically modify the genome of an RNA virus such as NDV, a manipulatable genetic system must be developed that uses a copy of the full viral RNA (vRNA) genome in the form of DNA. This full-length cDNA is amenable to genetic modification by using recombinant DNA techniques. The authentic or modified cDNA can be converted back into vRNA in cells, which in the presence of the viral replication proteins results in the production of a new modified infectious virus. Such ‘reverse genetics systems’ have been developed in the last few decades for different classes of RNA viruses. This system enables the rapid and facile introduction of mutations and deletions and the insertion of a transgene transcriptional unit, thereby enabling the changing of the biological properties of the virus.

Reverse genetics systems for several NDV strains, including lentogenic as well as velogenic strains, were developed by the Central Veterinary Institute (CVI), part of Wageningen University and Research, currently Wageningen Bioveterinary Research (WBVR) under the supervision of Dr. Ben Peeters (Peeters et al., 1999, J. Virol. 73:5001-9; de Leeuw et al., 2005, J. Gen. Virol. 86:1759-69; Dortmans et al., 2009, J. Gen. Virol. 90:2746-50). In order to generate a reverse genetics system for providing the NDV nucleic acids and strains according to the present invention, a similar approach was used. Details of the procedure can be found in the above cited papers and in the paragraphs below. Briefly, the system consists of 4 components, i.e., a transcription plasmid containing the full-length (either authentic or genetically modified) cDNA of the virus, which is used to generate the vRNA, and 3 expression plasmids (‘helper plasmids’) containing the NP, P and L genes of NDV respectively, which are used to generate the vRNA-replication complex (consisting of NP, P and L proteins). Transcription of the cDNA (i.e. conversion of the cDNA into vRNA) and expression of the NP, P and L genes by the helper plasmids is driven by a T7 promoter. The corresponding T7 DNA-dependent RNA polymerase (T7-RNAPol) is provided by a helper-virus (Fowlpox-T7).

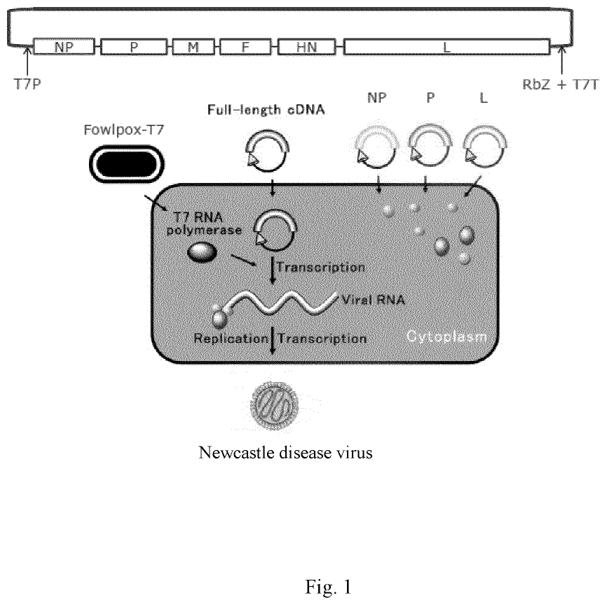

In order to rescue virus, the 4 plasmids are co-transfected into Fowlpox-T7 infected cells (FIG. 1). Three to five days after transfection, the supernatant is inoculated into specific-pathogen-free embryonated chicken eggs (ECE) and incubated for 3 days. Infectious virus that is produced by transfected cells will replicate in the ECE and progeny virus can be harvested from the allantois fluid.

In order to develop a reverse genetics system for NDV the following steps were followed:
Generation of sub-genomic NDV and foreign gene cDNA's by RT-PCR
Assembly of full-length cDNA in a transcription vector
Cloning of each of the NP, P and L genes into an expression vector
Verify nucleotide sequence of full-length cDNA and helper-plasmids
Repair nucleotide differences resulting from the cloning procedure, if necessary
Rescue of infectious virus from cDNA using co-transfection (FIG. 1)

2.2 Construction of Full-Length NDV-Mut HN(F277L)/M(G165W) cDNA, NDV-Mut HN(F277L)/M(G165W)/F(F117S)/F(F190L)/L(V757I)/L(F1551S)/L(R1700L) and helper plasmids

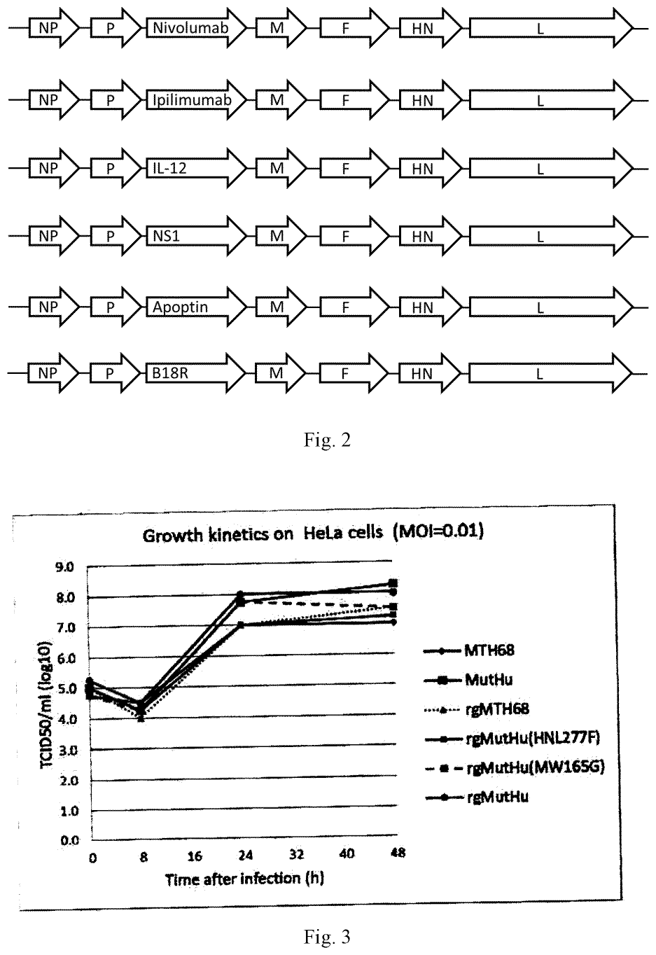

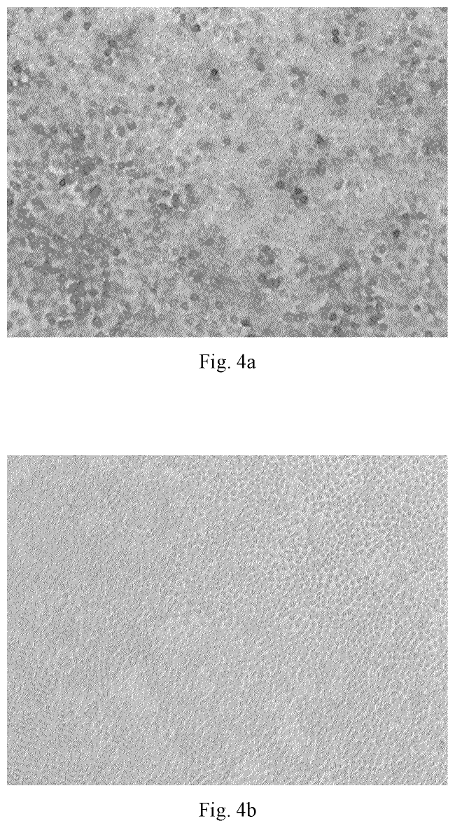

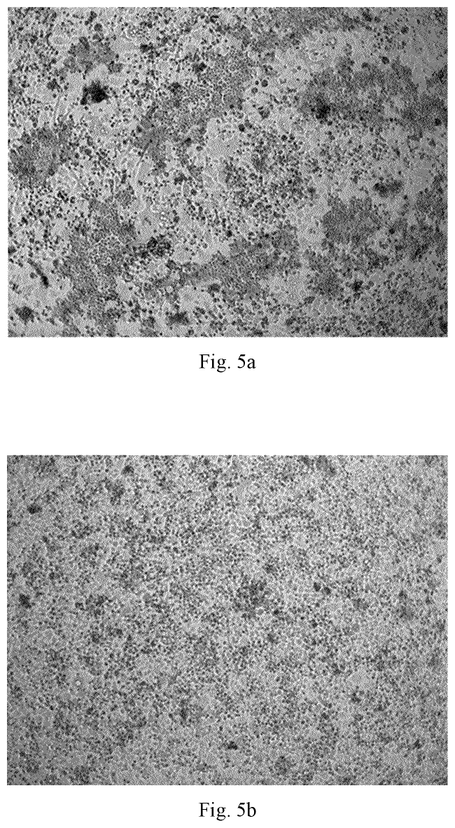

NDV-Mut HN(F277L)/M(G165W) and NDV-Mut HN(F277L)/M(G165W)/F(F117S)/F(F190L)/L(V757I)/L(F1551S)/L(R1700L) (passage 28 HeLa cells) were used for the isolation of vRNA using standard procedures. The vRNA was used to generate first-strand cDNA by means of Reverse Transcriptase followed by PCR to generate 4 sub-genomic cDNA fragments (designated C1, C2, C3 and C8). The full-length cDNA of NDV-MutHu was assembled from these fragments and cloned in the transcription vector pOLTV5 (Peeters et al., 1999, J. Virol. 73:5001-5009) by a combination of In-Fusion® cloning and classical cloning using restriction enzymes. An overview of the procedure is shown in FIG. 2, and further details can be found in Appendix 1 of this reference. The NP, P and L-genes of NDV-MutHu were obtained by RT-PCR (Appendix 1) and cloned in the expression plasmid pCVI which was derived by deletion of a ClaI restriction fragment from pCI-neo (Promega).

2.3 Nucleotide Sequence Analysis

Nucleotide sequence analysis was used to verify that the sequence of pFL-NDV Mut HN(F277L)/M(G165W) and pFL-NDV Mut HN(F277L)/M(G165W)/F(F117S)/F(F190L)/L(V757I)/L(F1551S)/L(R1700L) were correct. A few nucleotides which differed from the Reference sequence were repaired. Silent mutations (i.e., not leading to an amino acid change) may be left unchanged.

2.4 Rescue of Infectious Virus from pFL-NDV Mut HN(F277L)/M(G165W)

In order to generate infectious virus, we used the co-transfection system described above (and illustrated in FIG. 1) to transfect QM5 cells (derived from Quail) using plasmid pFL-NDV_Mut HN(F277L)/M(G165W) and the helper plasmids pCVI-NP^{MutHN(F277L)/M(G165W)}, pCVI-PMut^{HN(F277L)/M(G165W)} and pCVI-L^{MutHN(F277L)/M(G165W)} Rescue of infectious virus was successful as demonstrated by the presence of infectious virus in the allantoic fluid of ECE that were inoculated with the transfection supernatant (data not shown). The identity of the rescued virus was determined by RT-PCR followed by sequencing. The rescued virus was designated rgMut HN(F277L)/M(G165W). NDV-Mut HN(F277L)/M(G165W) and rgMut HN(F277L)/M(G165W) have similar growth kinetics in HeLa cells.

2.5 Rescue of Infectious Virus from pFL-NDV Mut HN(F277L)/M(G165W)/F(F117S)/F(F190L)/L(V757I)/L(F1551S)/L(R1700L)

In order to generate infectious virus, we used the co-transfection system described above (and illustrated in FIG. 1) to transfect QM5 cells (derived from Quail) using plasmid pFL-NDV_Mut HN(F277L)/M(G165W)/F(F117S)/F(F190L)/L(V757I)/L(F1551S)/L(R1700L) and the helper plasmids pCVI-NP^{MutHN(F277L)/M(G165W)/F(F117S)/F(F190L)/L(V757I)/L(F1551S)/L(R1700L)}, pCVI-P^{MutHN(F277L)/M(G165W)/F(F117S)/F(F190L)/L(V757I)/L(F1551S)/L(R1700L)} and pCVI-L^{Mut HN(F277L)/M(G165W)/F(F117S)/F(F190L)/L(V757I)/L(F1551S)/L(R1700L)}. Rescue of infectious virus was successful as demonstrated by the presence of infectious virus in the allantoic fluid of ECE that were inoculated with the transfection supernatant (data not shown). The identity of the rescued virus was determined by RT-PCR followed by sequencing. The rescued virus was designated rgMut HN(F277L)/M(G165W)/F(F117S)/F(F190L)/L(V757I)/L(F1551S)/L(R1700L).

### Example 3. Identify Whether One or Both of the Amino Acid Substitutions in Mut HN(F277L)/M(G165W) are Responsible for the Difference in Growth Kinetics Between Mut HN(F277L)/M(G165W) and the Parent Strain MTH68

3.1 Growth Kinetics in HeLa Cells

The rescued rg-viruses (Table 1) as well as the original Mut HN(F277L)/M(G165W) and MTH68 viruses were used to determine their growth-kinetics in HeLa cells. Briefly, 4×10⁶ HeLa cells were seeded in 25 cm² cell culture flasks and grown overnight. The cells were infected using a MOI of 0.01 (i.e., 1 infectious virus particle per 100 cells), and at 8, 24 and 48 hours after infection the virus titer in the supernatant was determined by end-point titration on QM5 cells.

The data (FIG. 3) indicate that strains Mut HN(F277L)/M(G165W) and rgMut HN(F277L)/M(G165W) yield at least 10-fold higher virus titers than MTH68. Furthermore, the data indicate that the mutation at amino acid position 277 in the HN gene is responsible for this effect. The M mutation does not seem to have an effect. This can be best seen when looking at the virus titers 24 h after infection, or even better when comparing the increase in virus titer between 8 h and 24 h (the exponential growth phase). The virus titer shows an increase of 3.5 (log 10) for Mut HN(F277L)/M(G165W), rgMut HN(F277L)/M(G165W) and rgMut HN(F277L)/M(W165G), whereas this is 2.5 for MTH68, 2.7 for rgMut HN(L277F) and 3.0 for rgMTH68 (Table 3).

rgMut HN(F277L)/M(W165G) is a strain in which the mutation in the M gene has been restored in accordance with the NDV MTH-68/H.

TABLE 1
Virus titers (log10 TCID50/ml)

Time after infection (h)

| Virus | 0 h | 8 h | 24 h | 48 h |
|---|---|---|---|---|
| MTH68 | 4.8 | 4.5 | 7.0 | 7.0 |
| Mut HN(F277L)/M(G165W) | 5.0 | 4.3 | 7.8 | 8.3 |
| rgMTH68 | 5.0 | 4.0 | 7.0 | 7.5 |
| rgMut HN(L277F) | 5.0 | 4.3 | 7.0 | 7.3 |
| rgMut M(W165G) | 4.8 | 4.3 | 7.8 | 7.5 |
| rgMutHu | 5.3 | 4.5 | 8.0 | 8.0 |

### Example 4. Generation of Recombinant NDV-Strains with Enhanced Oncolytic and Immune Stimulating Properties Due to the Expression of Different Therapeutic Proteins

The recombinant NDV-strains expressing one of the foreign genes of the present invention can be generated by means of the previously established reverse genetics system described above. The respective gene is to be inserted into the full-length cDNA of e.g. NDV-Mut HN(F277L)/M(G165W)/F(F117S)/F(F190L)/L(V757I)/L(F1551S)/L(R1700L) between the P and the M genes. To this end the open reading frames of the foreign genes may be fused via a 2A sequence. The genes were provided with the necessary NDV gene-start and gene-end sequences in order to allow transcription by the vRNA polymerase. FIG. 2 shows the final constellation of the recombinant viruses.

Infectious virus can be rescued, and virus stocks can be prepared by two passages in HeLa cells. Expression of the respective foreign gene can be determined and quantified by using a total human IgG ELISA (Invitrogen).

### Example 5. Production of Viruses and Virus Mixtures

5.1 Cell Lines and Reagents

Hela and LLC MK2 cells were grown in Medium 199 with Earle's salts and stable L-Glutamine (0.1 g/l) (Gibco) supplemented with 10% fetal bovine serum (Gibco) and Penicillin/Streptomycin (50 μg/ml) (Gibco). Cells were kept in an incubator at 37° C.

Stable glutamine is a dipeptide (Ala-Gln) that is can be used as a substitute for L-glutamine. It is much less labile in solution, is stable to heat sterilization, and is less ammoniagenic than L-glutamine.

5.2 Production of Viruses

HeLa cells were used to produce NDV mutant variants, and Vaccinia viruses. Reovirus serotype 3 was produced in LLC MK2 cells.

In order to produce viruses, cells were grown in complete medium to 90% confluence in 850 cm²-smooth roller bottles (Nunc). Cells were washed three times with PBS (Gibco) and infected with virus at multiplicity of infection (MOI) of 0.1 for 1 hour. Each roller bottle was incubated with 50 ml of minimum essential medium (MEM) (Gibco) containing indicated viruses. The inoculums were then aspirated out and cells were washed again with PBS. 80 ml of new media (minimum essential medium) supplemented with 2% human AB serum (Sigma Aldrich) were added and the infected cells were kept at 37° C. in an incubator.

The cytopathic effect (CPE) of infected cells caused by oncolytic viruses was observed every 24 hours. The infecting viruses cause the lysis of the host cells. Medium supernatants from the cultures of infected cells were collected in 50 ml Falcon tubes when all the cells died released viruses into the media. The supernatants were then kept at 4° C. for 30 minutes and stored at −80° C.

5.3 Filtration and Storage of Virus Stocks

The medium supernatants which stored at −80° C. were thawed at room temperature and centrifuged at 4000 rpm for 20 minutes at 4° C. The pellets from the centrifugation were discarded and the supernatant fractions containing viruses were filtered through 0.4 m filters (Millipore). The virus is now ready to use or can be stored at −80° C.

5.4 Production of Virus Mixtures

Each oncolytic virus was produced and filtered separately. After filtration, if desired, virus mixtures were created by the combination of different viruses with equal or different volume ratio and stored at −80° C.

### Example 6. Pharmaceutical Formulation

A New Castle Disease Virus mixture comprising
NDV-Mut HN(F277L)/M(G165W)/F(F117S)/F(F190L)/L(V757I)/L(F1551S)/L(R1700L)-Bevacizumab,
NDV-Mut HN(F277L)/M(G165W)-Ipilimumab,
NDV-Mut HN(F277L)/M(G165W)-Nivolumab,
NDV-Mut HN(F277L)/M(G165W)-NS1,
NDV-Mut HN(F277L)/M(G165W),
NDV-Mut HN(F277L)/M(G165W)/F(F117S)/F(F190L)/L(V757I)/L(F1551S)/L(R1700L), and
NDV-Mut HN(F277L)/M(G165W)/F(F117S)/F(F190L)/F(L289A)/L(V757I)/L(F1551S)/L(R1700L)

each with equal volume ratio was prepared. The viruses and the 10 ml mixture were prepared as set forth in Example 5. The virus contents before the mixture, given as TCID₅₀ per ml, for each virus were 10′. The 10 ml formulation was administered intratumoral.

### Example 7. Pharmaceutical Formulation

A virus mixture comprising
NDV-Mut HN(F277L)/M(G165W)/F(F117S)/F(F190L)/L(V757I)/L(F1551S)/L(R1700L)-Atezolizumab,
NDV-Mut HN(F277L)/M(G165W)/F(F117S)/F(F190L)/L(V757I)/L(F1551S)/L(R1700L)-Bevacizumab,
NDV-Mut HN(F277L)/M(G165W)-Ipilimumab,
NDV-Mut HN(F277L)/M(G165W)-Nivolumab,
NDV-Mut HN(F277L)/M(G165W)-NS1,
NDV-Mut HN(F277L)/M(G165W), and
Vaccina

each with equal volume ratio was prepared. The viruses and the mixture were prepared as set forth in Example 5.

In addition, a reovirus serotype 3 solution was provided in accordance with Example 5.

10 ml of the virus mixture and 10 ml of the reovirus solution were mixed. The virus contents before the mixture given as TCID₅₀ per ml were:

NDV-Mut HN(F277L)/M(G165W)/F(F117S)/F(F190L)/L(V757I)/L(F1551S)/L(R1700L)-Atezolizumab: 10⁸

NDV-Mut HN(F277L)/M(G165W)/F(F117S)/F(F190L)/L(V757I)/L(F1551S)/L(R1700L)-Bevacizumab: 10⁸

NDV-Mut HN(F277L)/M(G165W)-Ipilimumab: 10⁸

NDV-Mut HN(F277L)/M(G165W)-Nivolumab: 10⁸

NDV-Mut HN(F277L)/M(G165W)-NS1: 10⁸

NDV-Mut HN(F277L)/M(G165W): 10⁸

Vaccina: 10^{6.5}

Reovirus serotype 3: 10^{7.8}

Accordingly, in the 20 ml formulation each NDV strain was comprised in an amount of 1.4×10⁸, given as TCID₅₀, Vaccinia was comprised in an amount of 4.5×10⁶, given as TCID₅₀, and Reovirus serotype 3 was comprised in an amount of 6.3×10⁸, given as TCID₅₀.

The 20 ml formulation was administered intratumoral.

### Example 8. Pharmaceutical Formulation

A New Castle Disease Virus mixture comprising
NDV-Mut HN(F277L)/M(G165W)/F(F117S)/F(F190L)/L(V757I)/L(F1551S)/L(R1700L)-Atezolizumab,
NDV-Mut HN(F277L)/M(G165W)/F(F117S)/F(F190L)/L(V757I)/L(F1551S)/L(R1700L)-Bevacizumab,
NDV-Mut HN(F277L)/M(G165W)-Ipilimumab,
NDV-Mut HN(F277L)/M(G165W)-Nivolumab,
NDV-Mut HN(F277L)/M(G165W)-NS1,
NDV-Mut HN(F277L)/M(G165W),
NDV-Mut HN(F277L)/M(G165W)/F(F117S)/F(F190L)/L(V757I)/L(F1551S)/L(R1700L), and
NDV-Mut HN(F277L)/M(G165W)/F(F117S)/F(F190L)/F(L289A)/L(V757I)/L(F1551S)/L(R1700L)

each with equal volume ratio was prepared. The viruses and the 10 ml mixture were prepared as set forth in Example 5. The virus contents before the mixture, given as TCID₅₀ per ml, for each virus were 10′. The 10 ml formulation was administered intratumoral.

### Example 9. Pharmaceutical Formulation

A virus mixture comprising
NDV-Mut HN(F277L)/M(G165W)/F(F117S)/F(F190L)/L(V757I)/L(F1551S)/L(R1700L)-Atezolizumab,
NDV-Mut HN(F277L)/M(G165W)/F(F117S)/F(F190L)/L(V757I)/L(F1551S)/L(R1700L)-Bevacizumab,
NDV-Mut HN(F277L)/M(G165W)-Ipilimumab,
NDV-Mut HN(F277L)/M(G165W)-Nivolumab,
NDV-Mut HN(F277L)/M(G165W)-NS1,
NDV-Mut HN(F277L)/M(G165W),
NDV-Mut HN(F277L)/M(G165W)/F(F117S)/F(F190L)/L(V757I)/L(F1551S)/L(R1700L),
NDV-Mut HN(F277L)/M(G165W)/F(F117S)/F(F190L)/F(L289A)/L(V757I)/L(F1551S)/L(R1700L), and
Vaccina

each with equal volume ratio was prepared. The viruses and the mixture were prepared as set forth in Example 5.

In addition, a reovirus serotype 3 solution was provided in accordance with Example 5.

10 ml of the virus mixture and 10 ml of the reovirus solution were mixed. The virus contents before the mixture given as TCID₅₀ per ml were:

NDV-Mut HN(F277L)/M(G165W)/F(F117S)/F(F190L)/L(V757I)/L(F1551S)/L(R1700L)-Atezolizumab: 10⁸

NDV-Mut HN(F277L)/M(G165W)/F(F117S)/F(F190L)/L(V757I)/L(F1551S)/L(R1700L)-Bevacizumab: 10⁸

NDV-Mut HN(F277L)/M(G165W)-Ipilimumab: 10⁸

NDV-Mut HN(F277L)/M(G165W)-Nivolumab: 10⁸

NDV-Mut HN(F277L)/M(G165W)-NS1: 10⁸

NDV-Mut HN(F277L)/M(G165W): 10⁸

NDV-Mut HN(F277L)/M(G165W)/F(F117S)/F(F190L)/L(V757I)/L(F1551S)/L(R1700L): 10⁸

NDV-Mut HN(F277L)/M(G165W)/F(F117S)/F(F190L)/F(L289A)/L(V757I)/L(F1551S)/L(R1700L): 10⁸

Vaccina: 10^{6.5}

Reovirus serotype 3: 10^{7.8}

Accordingly, in the 20 ml formulation each NDV strain was comprised in an amount of 1.4×10⁸, Vaccinia was comprised in an amount of 4.5×10⁶ and Reovirus serotype 3 was comprised in an amount of 6.3×10⁸.

The 20 ml formulation was administered intratumoral.

### Example 10. Successful Treatment of Renal Cell Carcinoma (RCC) Stage IV

A patient was diagnosed with renal cell carcinoma (RCC) stage IV and treated with a pharmaceutical composition according to the present invention. As can be seen from table 2 below, 3 months after the treatment a decreased size, thickness and uptake, shown as the standardized uptake value (SUV) of fluorodeoxyglucose, of the tumour was detected. Thus, the patient beneficially responded to the treatment. The increase in size and uptake of the lymph nodes is believed to be the consequence of a regional hyper-immune response.

TABLE 2

| | Pre-treatment (at | Post 3-months | |
|---|---|---|---|
| Tumor load | diagnosis) | treatment | From 100% |
| Left renal mass | Size: 3908 cm³ | Size: 2165 cm³ | 55% |
| | SUV: 9.098 | SUV: 5.265 | 58% |
| Left adrenal | 0.8 cm | 0.6 cm | 75% |
| thickness | SUV: 5.457 | SUV: 3.783 | 69% |
| Left retroperitoneal | Size: 15.5 cm³ | Size: 55 cm³ | 355% |
| node | SUV: 9.969 | SUV: 15.02 | 151% |
| Right retrocrural | Size: 2.5 cm³ | Size: 11.5 cm³ | 460% |
| node | SUV: 4.915 | SUV: 6.862 | 140% |
| Paraesophageal | Size: 23 cm³ | Size: 31 cm³ | 135% |
| node | SUV: 5.625 | SUV: 7.815 | 139% |
| Left supraclavicular | Size: 3.3 cm³ | Size: 5.2 cm³ | 158% |
| node | SUV: 4.488 | SUV: 5.694 | 128% |
| Total | Size: 3.993 cm³ | Size: 2.313 cm³ | 58% |

In table 3 below the treatment scheme for renal cell carcinoma stage IV can be observed. The patient was born in 1965 and initially diagnosed with renal cell carcinoma stage IV on Feb. 13, 2020. The first treatment with oncolytic viruses started on Feb. 14, 2020.

TABLE 3
treatment scheme for renal cell carcinoma stage IV. Virus concentration in TCID50. patient born in 1965

| | | NDV | | | | | NDV- |
|---|---|---|---|---|---|---|---|
| | | mixture | REO-3 | Vaccinia | NDV-1 | NDV-2b | Nivo |
| Initial | 2020 Feb. 13 | | | | | | |
| diagnosis | | | | | | | |
| OV1 | 2020 Feb. 14 | Mixture of 7 | 6.31U + 08 | 4.52U + 06 | 1.43U + 08 | | 1.43U + 08 |
| | | with NS1 | | | | | |
| 1. PET/CT | 2020 Feb. 20 | | | | | | |
| biopsy | 2020 Mar. 2 | | | | | | |
| OV2 | 2020 Mar. 2 | Mixture of 7 | 6.31U + 08 | 4.52U + 06 | 1.43U + 08 | | 1.43U + 08 |
| | | with NS1 | | | | | |
| OV3 | 2020 Mar. 17 | Mixture of 7 | 6.31U + 08 | 4.52U + 06 | 1.43U + 08 | | 1.43U + 08 |
| | | with NS1 | | | | | |
| OV4 | 2020 Apr. 2 | Mixture of 7 | 6.31U + 08 | 4.52U + 06 | 1.43U + 08 | | 1.43U + 08 |
| | | with NS1 | | | | | |
| OV5 | 2020 Apr. 21 | Mixture of 7 | 6.31U + 08 | 4.52U + 06 | 1.43U + 08 | | 1.43U + 08 |
| | | with NS1 | | | | | |
| OV6 | 2020 May 5 | Mixture of 7 | 6.31U + 08 | 4.52U + 06 | 1.43U + 08 | | 1.43U + 08 |
| | | with NS1 | | | | | |
| 2. PET/CT | 2020 May 25 | | | | | | |
| OV7 | 2020 May 26 | Mixture of | 6.31U + 07 | 3.16U + 06 | | 1.00U + 08 | 1.00U + 08 |
| | | 101.00U + 08 | | | | | |
| OV8 | 2020 Jun. 12 | Mixture of | 6.31U + 07 | 3.16U + 06 | | 1.00U + 08 | 1.00U + 08 |
| | | 10 | | | | | |
| OV9 | 2020 Jun. 30 | Mixture of | 6.31U + 07 | 3.16U + 06 | | 1.00U + 08 | 1.00U + 08 |
| | | 10 | | | | | |
| OVIO | 2020 Jul. 24 | Mixture of | 6.31U + 07 | 3.16U + 06 | | 1.00U + 08 | 1.00U + 08 |
| | | 10 | | | | | |
| 3. PET/CT | 2020 Sep. 2 | | | | | | |
| OV11 | 2020 Oct. 8 | Mixture of | 6.31U + 07 | 3.16U + 06 | | 1.00U + 08 | 1.00U + 08 |
| | | 10 | | | | | |
| 4. PET/CT | 2020 Nov. 23 | | | | | | |

| | NDV- | NDV- | NDV- | NDV- | NDV- | NDV- |
|---|---|---|---|---|---|---|
| | Atez | Bevac | Ipi | Apoptin | B18R | IL-12 |
| Initial | | | | | | |
| diagnosis | | | | | | |
| OV1 | 1.00U + 09 | 1.00U + 09 | 1.43U + 08 | 1.43U + 08 | 1.43U + 08 | |
| 1. PET/CT | | | | | | |
| biopsy | | | | | | |
| OV2 | 1.00U + 09 | 1.00U + 09 | 1.43U + 08 | 1.43U + 08 | 1.43U + 08 | |
| OV3 | 1.00U + 09 | 1.00U + 09 | 1.43U + 08 | 1.43U + 08 | 1.43U + 08 | 1.00U + 05 |
| OV4 | 1.00U + 09 | 1.00U + 09 | 1.43U + 08 | 1.43U + 08 | 1.43U + 08 | 1.00U + 05 |
| OV5 | 1.00U + 09 | 1.00U + 09 | 1.43U + 08 | 1.43U + 08 | 1.43U + 08 | 1.00U + 05 |
| OV6 | 1.00U + 09 | 1.00U + 09 | 1.43U + 08 | 1.43U + 08 | 1.43U + 08 | 2.00U + 05 |
| 2. PET/CT | | | | | | |
| OV7 | 1.00U + 08 | 1.00U + 08 | 1.00U + 08 | 1.00U + 08 | 1.00U + 08 | 1.00U + 07 |
| OV8 | 1.00U + 08 | 1.00U + 08 | 1.00U + 08 | 1.00U + 08 | 1.00U + 08 | 1.00U + 08 |
| OV9 | 1.00U + 08 | 1.00U + 08 | 1.00U + 08 | 1.00U + 08 | 1.00U + 08 | 5.00U + 07 |
| OVIO | 1.00U + 08 | 1.00U + 08 | 1.00U + 08 | 1.00U + 08 | 1.00U + 08 | 5.00U + 07 |
| 3. PET/CT | | | | | | |
| OV11 | 1.00U + 08 | 1.00U + 08 | 1.00U + 08 | 1.00U + 08 | 1.00U + 08 | 5.00U + 07 |
| 4. PET/CT | | | | | | |

### Example 11. Successful Treatment of Metastatic Breast Cancer

A patient was diagnosed with metastatic breast cancer and treated with a pharmaceutical composition according to the present invention. As can be seen from table 3 below, in the first 4 months after treatment an initial increase in size and uptake, shown as the standardized uptake value (SUV) of fluorodeoxyglucose, can be observed, as response to an active immune response to the oncolytic viruses. But 8 months after the treatment a decreased size, thickness and uptake of the tumor was detected. These results demonstrate that the patient beneficially respond to the treatment. The CT/PET demonstrates a complete response with a disintegrating/resolving scar and no uptake in the right upper chest wall lesion, confirming the absence of cancer.

TABLE 4

| | | | Post 4 | Post 8 | Post 14 | |
|---|---|---|---|---|---|---|
| | Pre- | Post 3 | months and | months and | months and | Post 19 |
| Tumor | treatment | months | 12 days | 17 days | 21 days | months |
| load | (at diagnosis) | treatment | treatment | treatment | treatment | treatment |
| Right | Size: 2.8 cm³ | Size: 3.1 cm³ | Size: 6.1 cm³ | Size: 3.3 cm³ | Size: 2.04 cm³ | Size: 1.1cm³ |
| upper | SUV: 5.219 | SUV: 7.671 | SUV: 11.06 | SUV: 7.67 | SUV: 4.565 | SUV: 1.603 |
| chest | | | | | | |
| wall | | | | | | |
| lesion | | | | | | |
| Right | Size: 0.94 cm³ | Size: 1.4 cm³ | Size: 2.1 cm³ | Size: 1.2 cm³ | Size: 0.88 cm³ | Size: 0.63 cm³ |
| axillary | | | | | | |
| lymph | SUV: 2.423 | SUV: 4.492 | SUV: 5.908 | SUV: 2.044 | SUV: 2.349 | SUV: 1.437 |
| node | | | | | | |
| Lung | Size: 20.5 mm³ | Size: 63.2 mm³ | Size: 157 mm³ | Size: 44.5 mm³ | Size: 36.2 mm³ | Size: 24.8 mm³ |
| node 1 | | | | | | |
| Lung | Size: 37.2 mm³ | Size: 100.8 mm³ | Size: 151 mm³ | Size: 44.9 mm³ | Size: 24.8 mm³ | Size: 18.6 mm³ |
| node 2 | | | | | | |

In table 5(a+b) below the treatment scheme for breast cancer stage IV can be observed. The patient was born in 1977 and the first treatment with oncolytic viruses started on Nov. 5, 2018.

TABLE 5
a: medical examination and treatment scheme of a patient
diagnosed with metastatic breast cancer (OV = oncolytic virus)

| | | Virus mixtures in ml | | | | | |
|---|---|---|---|---|---|---|---|
| | | Mixture | Mixture | | | | |
| Medical examination | | of 7 with | of 7 with | 7 | Mixture | Mixture | Mixture |
| and treatment | | NS1 | Parvo | strains | of 10 | of 4 | of 5 |
| PET/ | 24 Oct. 2018 | | | | | | |
| CT1 | | | | | | | |
| OV1 | 5 Nov. 2018 | | | | | | |
| OV2 | 29 Nov. 2018 | | | | | | |
| OV3 | 10 Dec. 2018 | | | | | | |
| OV4 | 14 Jan. 2019 | | | | | | |
| PET/ | 28 Jan. 2019 | | | | | | |
| CT2 | | | | | | | |
| OV5 | 5 Feb. 2019 | | | | | | |
| OV6 | 25 Feb. 2019 | | | | | | |
| OV7 | 14 Mar. 2019 | | | | | | |
| OV8 | 21 Mar. 2019 | | | | | | |

| | | | |
|---|---|---|---|
| OV9 | 27 Mar. 2019 | Mixture | 20 |
| | | of 4 | |

| | |
|---|---|
| PET/ | 3 May 2019 |
| CT3 | |

| | | | | | | | | |
|---|---|---|---|---|---|---|---|---|
| OV10 | 16 May 2019 | Mixture | | | | | 40 | |
| | | of 4 | | | | | | |
| OV11 | 6 Jun. 2019 | Mixture | | | | | | 10 |
| | | of 5 | | | | | | |
| OV12 | 4 Jul. 2019 | Mixture | | 10 | | | | |
| | | of 7 + | | | | | | |
| | | Parvo | | | | | | |
| OV13 | 25 Jul. 2019 | Mixture | | 10 | | | | |
| | | of 7 + | | | | | | |
| | | Parvo | | | | | | |
| OV14 | 6 Aug. 2019 | Mixture | | 10 | | | | |
| | | of 7 + | | | | | | |
| | | Parvo | | | | | | |
| OV15 | 5 Sep. 2019 | 7 strains | | | 10 | | | |
| PET/ | 7 Oct. 2019 | | | | | | | |
| CT5 | | | | | | | | |
| OV16 | 8 Oct. 2019 | 7 strains | | | 10 | | | |
| OV17 | 7 Nov. 2019 | 7 strains | | | 10 | | | |
| OV18 | 4 Dec. 2019 | Mixture | 10 | | | | | |
| | | of 7 + | | | | | | |
| | | NS1 | | | | | | |
| OV19 | 14 Jan. 2020 | Mixture | 10 | | | | | |
| | | of 7 + | | | | | | |
| | | NS1 | | | | | | |
| OV20 | 13 Feb. 2020 | Mixture | 10 | | | | | |
| | | of 7 + | | | | | | |
| | | NS1 | | | | | | |
| OV21 | 17 Mar. 2020 | Mixture | 10 | | | | | |
| | | of 7 + | | | | | | |
| | | NS1 | | | | | | |
| OV22 | 26 May 2020 | Mixture | | | | 10 | | |
| | | of 10 | | | | | | |

b: Virus concentration in TICD50 in breast cancer treatment,
this table is to be read in combination with table 5a
Virus concentration in TICD50

| | Parotitis | | | | | NDV- | NDV- | NDV |
|---|---|---|---|---|---|---|---|---|
| VSV-2 | virus | REO-3 | Vaccinia | Parvo | NDV-1 | 2a | 2b | NS1 |
| 2.00U + 09 | | | | | | | | |
| | 3.16U + 07 | | | | | | | |
| | 6.32U + 07 | | | | | | | |
| | | 1.26U + 09 | | | | | | |
| | | | 1.58U + 07 | | | | | |
| | | | 3.16U + 07 | | | | | |
| | | | 7.32U + 06 | | 1.00U + 09 | | | |
| | | | 4.42U + 06 | 1.40U + 07 | 1.40U + 08 | | | |
| | | | 4.42U + 06 | 1.40U + 07 | 1.40U + 08 | | | |
| | | | 4.42U + 06 | 1.40U + 07 | 1.40U + 08 | | | |
| | | 6.31U + 08 | | | 1.40U + 08 | 4.42U + 09 | | 1.40U + 08 |
| | | 6.31U + 08 | | | 1.40U + 08 | 4.42U + 09 | | 1.40U + 08 |
| | | 6.31U + 08 | 3.16U + 07 | | 1.40U + 08 | 4.42U + 09 | | 1.40U + 08 |
| | | 6.31U + 08 | 4.42U + 06 | | 1.40U + 08 | | | 1.40U + 08 |
| | | 6.31U + 08 | 4.42U + 06 | | 1.40U + 08 | | | 1.40U + 08 |
| | | 6.31U + 08 | 4.42U + 06 | | 1.40U + 08 | | | 1.40U + 08 |
| | | 6.31U + 08 | 4.42U + 06 | | 1.40U + 08 | | | 1.40U + 08 |
| | | 6.31U + 07 | 3.16U + 06 | | | | 1.00U + 08 | 1.00U + 08 |

b: Virus concentration in TICD50 in breast cancer treatment,
this table is to be read in combination with table 5a
Virus concentration in TICD50

| NDV- | NDV- | | NDV- | NDV- | | NDV |
|---|---|---|---|---|---|---|
| Nivo | Atez | NDV-Beva | Ipi | Apoptin | NDV-B18R | IL-12 |
| | | | | | 1.00U+ 09 | |
| 1.00U + 09 | | | 1.00U + 09 | | | |
| 5.00U + 08 | | | 5.00U + 08 | | 5.00U + 08 | |
| 3.30U + 08 | | | 3.30U + 08 | | 3.30E | |
| 5.00U + 08 | | | 5.00U + 08 | | 5.00U + 08 | |
| 1.00U + 09 | | | 1.00U + 09 | | 1.00U + 09 | |
| 2.00U + 08 | | | 2.00U + 08 | 2.00U + 08 | 2.0U + 08 | |
| 1.40U + 08 | | | 1.40U + 08 | 1.40U + 08 | 1.40U + 08 | |
| 1.40U + 08 | | | 1.40U + 08 | 1.40U + 08 | 1.40U + 08 | |
| 1.40U + 08 | | | 1.40U + 08 | 1.40U + 08 | 1.40U + 08 | |
| 1.40U + 08 | | | 1.40U + 08 | 1.40U + 08 | 1.40U + 08 | |
| 1.40U + 08 | | | 1.40U + 08 | 1.40U + 08 | 1.40U + 08 | |
| 1.40U + 08 | | | 1.40U + 08 | 1.40U + 08 | 1.40U + 08 | |
| 1.40U + 08 | 1.00U + 09 | 1.00U + 09 | 1.40U + 08 | 1.40U + 08 | 1.40U + 08 | |
| 1.40U + 08 | 1.00U + 09 | 1.00U + 09 | 1.40U + 08 | 1.40U + 08 | 1.40U + 08 | |
| 1.40U + 08 | 1.00U + 09 | 1.00U + 09 | 1.40U + 08 | 1.40U + 08 | 1.40U + 08 | |
| 1.40U + 08 | 1.00U + 09 | 1.00U + 09 | 1.40U + 08 | 1.40U + 08 | 1.40U + 08 | 1.00U + 05 |
| 1.00U + 08 | 1.00U + 08 | 1.00U + 08 | 1.00U + 08 | 1.00U + 08 | 1.00U + 08 | 1.00U + 07 |

### REFERENCES

Blach-Olszewska et al., (1977) Why HeLa cells do not produce interferon? Arch Immunol Ther Exp (Warsz) 25:683-91.
Chng et al., (2015) Cleavage efficient 2A peptides for high level monoclonal antibody expression in CHO cells, mAbs, 7:2, 403-412; http://dx.doi.org/10.1080/19420862.2015.1008351.
Enoch et al., (1986) Activation of the Human beta-Interferon Gene Requires an Interferon-Inducible Factor, Mol. Cell. Biol. 6:801-10.
Haryadi et al., (2015) Optimization of Heavy Chain and Light Chain Signal Peptides for High Level Expression of Therapeutic Antibodies in CHO Cells. PLoS ONE 10(2): e0116878. doi:10.1371/journal.pone.0116878.
Schirrmacher, (2015) Oncolytic Newcastle disease virus as a prospective anti-cancer therapy. A biologic agent with potential to break therapy resistance. Expert Opin. Biol. Ther. 15:17 57-71
Zamarin et al., (2014) Localized oncolytic virotherapy overcomes systemic tumor resistance to immune checkpoint blockade immunotherapy. Sci. Transl. Med. 6(226).
Zamarin & Palese, (2017) Oncolytic Newcastle Disease Virus for cancer therapy: old challenges and new directions. Future Microbiol. 7: 347-67.

### APPENDIX 1: PRIMERS USED FOR THE GENERATION OF CDNA FRAGMENTS AND HELPER-PLASMIDS CDNA FRAGMENTS

| Frag- | | | |
|---|---|---|---|
| ment | Size | Primer | Sequence (5′-3′) |
| Cl | 3.6 | Noss-09 | ACGACTCACTATAGGaccaaacagagaatcc |
| | kb | (SEQ ID | gtgag |
| | | No. 40) | |
| | | Noss-121 | CCGGGAAGATCCAGGgcactcttcttgcatg |
| | | (SEQ ID | ttac |
| | | No. 41) | |
| C2 | 3.7 | Noss-122 | GGGCCTGCCTCACTAtggtggtaacatgcaa |
| | kb | (SEQ ID | gaag |
| | | No. 42) | |
| | | Noss-123 | TGCATGTTACCACCAatgtgtcattgtatcg |
| | | (SEQ ID | cttg |
| | | No. 43) | |
| C3 | 5.7 | Noss-125 | CAAGAAGGGAGATACgtaatatacaagcgat |
| | kb | (SEQ ID | acaatg |
| | | No. 44) | |
| | | Noss-126 | TCGCTTGTATATTACttgttgtagcaaagag |
| | | (SEQ ID | cacc |
| | | No. 45) | |
| C8 | 2.0 | Noss-133 | GGCCTGGATCTTCCCattatgctgtctgtat |
| | kb | (SEQ ID | acggtgc |
| | | No. 46) | |
| | | Noss-10 | ATGCCATGCCGACCCaccaaacaaagacttg |
| | | (SEQ ID | gtgaatg |
| | | No. 47) | |
| iPCR | 2.5 | Noss-17 | CCTATAGTGAGTCGTATTAATTTC |
| pOLTV5 | kb | (SEQ ID | |
| (StuI) | | No. 48) | |
| | | Noss-128 | CCTGGATCTTCCCGGGTCGG |
| | | (SEQ ID | |
| | | No. 49) | |
| iPCR | 2.5 | Noss-137 | GGGTCGGCATGGCATCTCCACC |
| pOLTV5 | kb | (SEQ ID | |
| (SmaI) | | No. 50) | |
| | | Noss-138 | GGGAAGATCCAGGCCTATAGTG |
| | | (SEQ ID | |
| | | No. 51) | |

Helper-Plasmids (Generated by In-Fusion® Cloning in pCVI)

| Gene | primer | sequence |
|---|---|---|
| NP | Noss-22 | CTCTAGAGTCGACCCttctgccaacatgtcttccg |
| | (SEQ ID | |
| | No. 52) | |
| | Noss-23 | GGGAAGCGGCCGCCCgtcggtcagtatccccagtc |
| | (SEQ ID | |
| | No. 53) | |
| P | Noss-24 | CTCTAGAGTCGACCCcagagtgaagatggccaccttc |
| | (SEQ ID | |
| | No. 54) | |
| | Noss- | GGGAAGCGGCCGCCCgtagtagtgatcagccattc |
| | 25n | |
| | (SEQ ID | |
| | No. 55) | |
| L | Noss-26 | CTCTAGAGTCGACCCgggtaggacatggcgggctc |
| | (SEQ ID | |
| | No. 56) | |
| | Noss-27 | GGGAAGCGGCCGCCCtgcctttaagagtcacagttac |
| | (SEQ ID | |
| | No. 57) | |

1. (canceled)

2. The pharmaceutical formulation according to claim 19, wherein the components are mixed in equal volume ratio.

3. The pharmaceutical formulation according to claim 19, wherein the non-recombinant NDV strain comprises a nucleic acid which is at least 70% identical to a nucleic acid sequence according to SEQ ID No. 1 to SEQ ID No. 7 of the sequence listing.

4. (canceled)

5. The pharmaceutical formulation according to claim 19, further comprising or more recombinant NDV strains comprising in their viral genome a nucleic acid sequence encoding as a foreign gene one or more selected from:
Lirilumab, an antigen-binding part of Lirilumab, a variant of Lirilumab or a variant of an antigen-binding part of Lirilumab;
Relatlimab, an antigen-binding part of Relatlimab, a variant of Relatlimab or a variant of an antigen-binding part of Relatlimab;
Monalizumab, an antigen-binding part of Monalizumab, a variant of Monalizumab or a variant of an antigen-binding part of Monalizumab;
TRX518, an antigen-binding part of TRX518, a variant of TRX518 or a variant of an antigen-binding part of TRX518; and
BMS 986178, an antigen-binding part of BMS 986178, a variant of BMS 986178 or a variant of an antigen-binding part of BMS 986178.

6. The pharmaceutical formulation according to claim 19, further comprising a recombinant NDV strains comprising in its viral genome a nucleic acid sequence encoding as a foreign gene Ramucirumab, a variant of Ramucirumab or a variant of an antigen-binding part of Ramucirumab.

7. The pharmaceutical formulation according to claim 19, wherein the formulation comprises a recombinant NDV strain comprising a nucleic acid sequence encoding interleukin-12 (IL-12), a part of interleukin-12, a variant of interleukin-12 or a variant of a part of interleukin-12.

8. The pharmaceutical formulation according to claim 19, further comprising at least one recombinant NDV strain comprising in its viral genome a nucleic acid sequence comprising at least one foreign gene, the at least one foreign gene being selected from the group consisting of:
a gene encoding the protein CD40 (cluster of differentiation 40), a part of CD40, a variant of CD40 or a variant of a part of CD40,
a gene encoding CD80, a part of CD80, a variant of CD80 or a variant of a part of CD80
a gene encoding Theralizumab, an antigen-binding part of Theralizumab, a variant of Theralizumab or a variant of an antigen-binding part of Theralizumab;
a gene encoding Gemtuzumab, an antigen-binding part of Gemtuzumab, a variant of Gemtuzumab or a variant of an antigen-binding part of Gemtuzumab;
a gene encoding an antibody, directed to CD39 or an antigen-binding part directed to CD39 (anti-CD39), having a sequence identity of at least 70% to SEQ. ID. No. 27;
a gene encoding an antibody directed to CA 15-3 or an antigen-binding part directed to CA 15-3 (anti-CA 15-3), having a sequence identity of at least 70% to SEQ. ID. No. 28;
a gene encoding an antibody directed to CA 19-9 or an antigen-binding part directed to CA 19-9 (anti-CA 19-9), having a sequence identity of at least 70% to SEQ. ID. No. 29;
a gene encoding Sofituzumab, an antigen-binding part of Sofituzumab, a variant of Sofituzumab or a variant of an antigen-binding part of Sofituzumab;
a gene encoding Cetuximab, an antigen-binding part of Cetuximab, a variant of Cetuximab or a variant of an antigen-binding part of Cetuximab,
a gene encoding a Trastuzumab, an antigen-binding part of Trastuzumab, a variant of Trastuzumab or a variant of an antigen-binding part of Trastuzumab,
a gene encoding BIL=3s, an antigen-binding part of BIL=3s, a variant of BIL=3s or a variant of an antigen-binding part of BIL=3s, P (anti a gene encoding J591, an antigen-binding part of J591, a variant of J591 or a variant of an antigen-binding part of J591;
a gene encoding a to a death TRAIL, a part of TRAIL, a variant of TRAIL or a variant of a part of TRAIL;
a gene encoding a green fluorescent protein or a part of a green fluorescent protein; and
any combination of these genes.

9. The pharmaceutical formulation according to claim 19, wherein the viral genome of at least one or each of the recombinant NDV strains further comprises a nucleic acid comprising a nucleic acid sequence encoding a matrix protein (M protein) with an amino acid substitution at position 165, where glycine (G) is substituted to tryptophane (W).

10. The pharmaceutical formulation according to claim 19 wherein the viral genome of at least one or each of the recombinant NDV strains further comprises a nucleic acid sequence encoding a fusion protein (F protein) with an amino acid substitution at position 117 where phenylalanine (F) is substituted to serine (S), and/or with an amino acid substitution at position 190, where phenylalanine (F) is substituted to leucine (L).

11. The pharmaceutical formulation according to claim 10, wherein the nucleic acid sequence encoding the fusion protein (F protein) further encodes an amino acid substitution at position 289 of the F protein, where leucine (L) is substituted to alanine (A) at position 289.

12. The pharmaceutical formulation according to claim 10, wherein the viral genome of at least one or each of the recombinant NDV strains further comprises a nucleic acid comprising a nucleic acid sequence encoding a large polymerase protein (L protein) having
an amino acid substitution at position 757, valine (V) is substituted to isoleucine (I), and/or
an amino acid substitution at position 1551 where phenylalanine (F) is substituted to serine (S), and/or
an amino acid substitution at position 1700, where arginine (R) is substituted to leucine (L).

13. The pharmaceutical formulation according to claim 12, wherein the nucleic acid sequence encoding the large polymerase protein (L protein) having further
an amino acid substitution at position 1717, where tyrosine (Y) is substituted to histidine (H), and/or
an amino acid substitution at position 1910, where glutamic acid (E) is substituted to lysine (K).

14. The pharmaceutical formulation according to claim 19, wherein the parent NDV for each of the recombinant NDV strains comprises a nucleic acid which is at least 70% identical to a nucleic acid sequence according to any one of SEQ ID No. 1 to SEQ ID No. 7 of the sequence listing.

15. The pharmaceutical formulation according to claim 19, wherein
the HN protein of at least one or each of the recombinant NDV strains comprises or consists of the amino acid sequence according to SEQ ID No. 36 of the sequence listing, and/or
wherein the F protein comprises or consists of the amino acid sequence according to SEQ ID No. 38 of the sequence listing, and/or
wherein the M protein comprises or consists of the amino acid sequence according to SEQ ID No. 37 of the sequence listing, and/or
wherein the L protein comprises or consists of the amino acid sequence according to SEQ ID No. 39 of the sequence listing.

16. The pharmaceutical formulation according to claim 19, wherein the pharmaceutical formulation comprises virus particles of each of the recombinant NDV strains an amount 10⁷ to 10⁹ of each of the recombinant NDV strains per 20 mL dose.

17. The pharmaceutical formulation according to claim 19, wherein the viral genome of the non-recombinant NDV strain:
comprises a nucleic acid sequence encoding a matrix protein (M protein) with an amino acid substitution at position 165, where glycine (G) is substituted to tryptophane (W); and/or
encodes a fusion protein (F protein) with an amino acid substitution at position 117, where phenylalanine (F) is substituted to serine (S), and/or with an amino acid substitution at position 190, where phenylalanine (F) is substituted to leucine (L), optionally further encoding
i) an amino acid substitution at position 289 of the F protein, where leucine (L) is substituted to alanine (A) and/or
ii) a large polymerase protein (L protein) having:
a) an amino acid substitution at position 757, where valine (V) is substituted to isoleucine (I), and/or
b) an amino acid substitution at position 1551, where phenylalanine (F) is substituted to serine (S), and/or
c) an amino acid substitution at position 1700, where arginine (R) is substituted to leucine (L),
the encoded large polymerase protein (L protein) optionally further having an amino acid substitution at position 1717, where tyrosine (Y) is substituted to histidine (H), and/or an amino acid substitution at position 1910, where glutamic acid (E) is substituted to lysine (K); and/or
encodes for the HN protein, which encoded HN protein comprises or consists of the amino acid sequence according to SEQ ID No. 36 of the sequence listing; and/or
encodes for the F protein, which encoded F protein comprises or consists of the amino acid sequence according to SEQ ID No. 38 of the sequence listing; and/or
encodes for the M protein, which encoded M protein comprises or consists of the amino acid sequence according to SEQ ID No. 37 of the sequence listing; and/or
encodes for the L protein, which encoded L protein comprises or consists of the amino acid sequence according to SEQ ID No. 39 of the sequence listing.

18. The pharmaceutical formulation according to claim 19, wherein the pharmaceutical formulation comprises virus particles of the reovirus type 3 in an amount of 6×10⁷ to 6×10⁹ per 20 mL dose, and/or wherein the pharmaceutical formulation comprises virus particles of the vaccinia virus in an amount of 4×10⁵ to 4×10⁷ per 20 mL dose.

19. A pharmaceutical formulation comprising:
a recombinant Newcastle Disease Virus (NDV) strain comprising a nucleic acid sequence encoding Atezolizumab, an antigen-binding part of Atezolizumab, a variant of Atezolizumab or a variant of an antigen-binding part of Atezolizumab;
a recombinant Newcastle Disease Virus (NDV) strain comprising a nucleic acid sequence encoding Ipilimumab, an antigen-binding part of Ipilimumab, a variant of Ipilimumab or a variant of an antigen-binding part of Ipilimumab;
a recombinant Newcastle Disease Virus (NDV) strain comprising a nucleic acid sequence encoding Nivolumab, an antigen-binding part of Nivolumab, a variant of Nivolumab or a variant of an antigen-binding part of Nivolumab;
a recombinant Newcastle Disease Virus (NDV) strain comprising a nucleic acid sequence encoding Bevacizumab, an antigen-binding part of Bevacizumab, a variant of Bevacizumab or a variant of an antigen-binding part of Bevacizumab;
a recombinant Newcastle Disease Virus (NDV) strain comprising a nucleic acid sequence encoding the non-structural protein NS1 of influenza A virus, a part of the non-structural protein NS1 of influenza A virus, a variant of the non-structural protein NS1 of influenza A virus or a variant of a part of the non-structural protein NS1 of influenza A virus;
a non-recombinant Newcastle Disease Virus (NDV) strain, wherein the viral genome of the non-recombinant NDV strain comprises a nucleic acid comprising a nucleic acid sequence encoding a hemagglutinin-neuramidase protein (HN protein) with an amino acid substitution at position 277, where phenylalanine (F) is substituted to an amino acid with a hydrophobic side chain;
a reovirus type 3; and
optionally a vaccinia virus.

20. A method of treating cancer in a subject of one or more indications selected from the group consisting of brain tumors, bone tumors, soft tissue tumors, gynecological tumors, gastrointestinal tumors, prostate tumors, lung tumors, ear, nose, throat tumors, tongue tumors, and skin tumors, the method comprising administering to the subject the pharmaceutical formulation according to claim 19.

21. The pharmaceutical formulation according to claim 19, wherein in the HN protein encoded by the viral genome of the non-recombinant NDV strain, phenylalanine at position 277 is substituted to leucine (L).

22. The pharmaceutical formulation according to claim 12, wherein the mutated L-gene of the at least one or each of the recombinant NDV strains encodes the L protein with the amino acid substitution at position 757, position 1551, and position 1700.
